(19) United States
(12) Patent Application Publication
(10) Pub. No.: US 2024/0174860 A1
(43) Pub. Date: May 30, 2024

(54) HIGH OLEIC OIL COMPOSITIONS AND USES THEREOF

(71) Applicant: Checkerspot, Inc., Alameda, CA (US)

(72) Inventors: Leon PARKER, San Francisco, CA (US); Kevin WARD, Emeryville, CA (US); Scott FRANKLIN, Woodside, CA (US); Mona CORREA, Hercules, CA (US); Veronica BENITES, San Francisco, CA (US)

(21) Appl. No.: 18/501,505

(22) Filed: Nov. 3, 2023

Related U.S. Application Data
(63) Continuation of application No. 18/156,929, filed on Jan. 19, 2023, now Pat. No. 11,873,405.
Application No. 18/156,929 is a continuation of application No. PCT/US22/43695, filed on Sep. 15, 2022.
(60) Provisional application No. 63/245,734, filed on Sep. 17, 2021.
(60) Provisional application No. 63/245,736, filed on Sep. 17, 2021.
(60) Provisional application No. 63/245,737, filed on Sep. 17, 2021.
(60) Provisional application No. 63/245,740, filed on Sep. 17, 2021.

(51) Int. Cl. C08L 91/00; A23D 9/02; C12P 7/6463

(52) U.S. Cl. CPC C08L 91/00; A23D 9/02; C12P 7/6463

(57) ABSTRACT

Provided herein are high oleic oil compositions. Further provided herein are methods of producing high oleic oil compositions from microorganisms and applications thereof in end products, including, for example, polyols, polyurethane products, personal care products, and food products.

### CROSS-REFERENCE

This application is a continuation of U.S. patent application Ser. No. 18/156,929 filed Jan. 19, 2023, which is a continuation of International Application No. PCT/US22/43695 filed Sep. 15, 2022, which claims the benefit of U.S. Provisional Application No. 63/245,734, U.S. Provisional Application No. 63/245,736, U.S. Provisional Application No. 63/245,737, and U.S. Provisional Application No. 63/245,740, each filed Sep. 17, 2021, each of which is incorporated herein by reference in its entirety.

### BACKGROUND

Oleaginous microorganisms have the ability to convert carbon substrates into oils, including triacylglycerides (TAGs) or lipids, and accumulate these oils intracellularly. Some microorganisms can have capacity to accumulate lipids in amounts of up to 80% dry weight. Thus, oleaginous microorganisms, including microalgae, bacteria, fungi, and yeasts, can serve as an ideal source for biobased oil production. Genetic and non-genetic modification techniques can allow for the production of non-naturally occurring oils having particular fatty acid profiles.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, the present disclosure provides a non-naturally occurring oil comprising a triacylglyceride (TAG) component and ergosterol, wherein the TAG component has a fatty acid content comprising 80% or more C18:1 fatty acids.

In some aspects, the present disclosure provides a non-naturally occurring oil, obtained through non-genetic engineering means, comprising a triacylglyceride (TAG) component and ergosterol, wherein the TAG component has a fatty acid content comprising 80% or more C18:1 fatty acids.

In some aspects, the present disclosure provides a formulation comprising a non-naturally occurring oil described herein.

In some aspects, the present disclosure provides an oleaginous, non-naturally occurring microorganism that produces a non-naturally occurring oil described herein.

In some aspects, the present disclosure provides a bioreactor comprising an oleaginous, non-naturally occurring microorganism described herein.

In some aspects, the present disclosure provides a method for producing a non-naturally occurring oil described herein, the method comprising: culturing in a bioreactor an oleaginous, non-naturally occurring microorganism described herein, thereby producing the non-naturally occurring oil.

In some aspects, the present disclosure provides an oil comprising a TAG component and at least 100 mg of ergosterol per 100 g of the oil, wherein the TAG component has a fatty acid content comprising 80% or more C18:1 fatty acids.

In some aspects, the present disclosure provides a microbial cell that produces a TAG oil comprising 80% or more C18:1 fatty acids, wherein the cell does not comprise an exogenous gene or exogenous nucleotides.

In some aspects, the present disclosure provides a method of producing an oil described herein, the method comprising: culturing a cell described herein, in a medium in a bioreactor.

In some aspects, the present disclosure provides a bioreactor comprising an oil described herein.

In some aspects, the present disclosure provides a bioreactor comprising a cell described herein.

In some aspects, the present disclosure provides a method of producing a cell, the method comprising: obtaining a microbial base strain; and subjecting the base strain to a classical strain improvement method to induce random or semi-random mutagenesis, wherein the cell produces a TAG oil comprising 80% or more C18:1 fatty acids, wherein the cell does not comprise an exogenous gene or exogenous nucleotides.

In some aspects, the present disclosure provides a cell produced by a method described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a sequence alignment of a region of the plastidic 23S rDNA between a Prototheca wickerhamii strain (UTEX 1533; SEQ ID NO:30) and a Prototheca moriformis strain (UTEX 1435; SEQ ID NO:31).

FIG. 2 shows an outline of the mutagenesis, trait selection, high-throughput, and automated screening steps of the improvement process described herein.

FIG. 3 shows a theoretical example of the enrichment strategy used to isolate cerulenin-resistant microalgae.

FIG. 4 illustrates an actual enrichment strategy used to isolate cerulenin-resistant microalgae.

FIG. 5 describes an automated glucose assay used to measure residual glucose during mutant screening.

FIG. 6 shows growth curves for selection for mutants with greater tolerance to membrane stress.

FIG. 7 shows growth curves for selection for mutants with greater tolerance to the triparanol.

FIG. 8 summarizes the classical strain improvement of Strain 2 by generation of diverse mutant libraries through exposure to different mixtures of metabolic inhibitors and herbicides. FIG. 8, Panel A shows growth results from the selection strategies described herein. FIG. 8, Panel B shows components of mixture 12 and 14 used in selection strategies described herein. FIG. 8, Panel C describes the various compounds used in selection strategies described herein.

FIG. 9 shows an overview of an example classical strain improvement strategy for generation of mutants derived from UTEX 1533.

### DETAILED DESCRIPTION

Provided herein are oil compositions having high oleic acid content, methods of making thereof, and formulations and applications thereof. Oil compositions provided herein can be produced by a microorganism that is genetically modified or non-genetically modified. Non-genetically modified microorganisms can be produced by classical strain improvement strategies such as those described herein. In turn, these non-naturally occurring microorganism can produce non-naturally occurring oils provided herein.

Genetic and non-genetic modification techniques can allow for the production of non-naturally occurring oils having particular phenotypes. While genetic engineering techniques can tend to be more targeted to phenotypes elicited in a host oleaginous microbe, classical strain improvement or other non-genetic engineering techniques can also be employed to enhance phenotypes. Enhancement of phenotypes can include elaboration of a particular fatty acid profile (e.g., high oleic acid content), yield on carbon, volumetric oil accumulation (e.g., g oil/L culture), oil productivity (e.g., g oil/L culture day), and oil as a percent dry cell weight (DCW) as a measure of strain performance.

A further benefit of classical strain improvement techniques, when used as the sole means to alter or improve strain phenotype and performance, can be realized from both a regulatory and business/marketing perspective. From a regulatory perspective, non-genetically engineered microbes may be exempt from regulatory oversight by entities such as U.S. EPA's Toxic Substances Control Act (TSCA) and the requirement to file a Microbial Commercial Activity Notice (MCAN) when the material is to be used in chemical (non-food) applications. Such dispensation extends to other geographies as well, such as Brazil, for example, where such microbes are exempt from filing a Strain Dossier with the Brazilian regulatory body (the National Technical Commission of Biosafety, Ministry of Science, Technology, Innovation, and Communications or CTNBio) that oversees industrial microbes. The avoidance of such regulatory oversight can save millions of dollars in development costs. From a marketing and consumer branding perspective, the raw materials produced by such non-genetically engineered means can meet GMO-free and organic labeling standards, as well as brands' and consumers' desires for “clean labeling”.

As used herein, the term “classical strain improvement” refers to methods of random or semi-random mutagenesis of microbes to create non-naturally occurring strains with improved properties. These methods include, but not limited to, mutagenesis of a population to create genetic variants, random selection or screening of a surviving population to identify an improved strain, and identification of improved strains by assaying fermentation broth for products. Classical strain improvement methods include exposure to UV radiation, chemical mutagens, and/or selective or enrichment agents. Classical strain improvement methods do not include recombinant genetic engineering methods targeted to one or more genomic regions, e.g., via homologous recombination.

As used herein, the term “microbial oil” refers to an oil produced or extracted from a microorganism (microbe), e.g., an oleaginous, single-celled, eukaryotic, or prokaryotic microorganism, including but not limited to, microalgae, yeast, bacteria, and fungi.

As used herein, the term “triacylglycerol”, “triglyceride”, or “TAG” refers to esters between glycerol and three saturated and/or unsaturated fatty acids. Generally, fatty acids of TAGs have chain lengths of 6 carbon atoms or more.

As used herein, the term “TAG purity”, “molecular purity”, or “oil purity” refers to the number of molecular species that make up an oil composition, on an absolute basis or present in amounts above a certain threshold. The fewer the number of TAG species in an oil, the greater the “purity” of the oil.

As used herein, the term “fatty acid profile” refers to a fatty acid composition of an oil, e.g., an oil produced by a cell provided herein or a derivative thereof. Derivatives of an oil produced by a cell provided herein include a refined, bleached, and deodorized oil. Fatty acid profiles can be determined by subjecting an oil to transesterification to generate fatty acid methyl esters (FAMEs) and subsequently quantitating fatty acid type by Gas Chromatography equipped with a Flame Ionization Detector (GC/FID).

As used herein, the term “sterol profile” refers to a sterol composition of an oil, e.g., an oil produced by a cell provided herein or a derivative thereof. Derivatives of an oil produced by a cell provided herein include a refined, bleached, and deodorized oil.

As used herein, the term “polyol” refers to triglycerols or fatty acid alcohols comprising hydroxyl functional groups. As used herein, the term “polyol derived from a TAG oil” generally refers to a polyol obtained from chemical conversion of a TAG oil, e.g., via epoxidation and ring opening, ozonolysis and reduction, or hydroformylation and reduction.

As used herein, the term “polyurethane”, “PU”, or “urethane” refers to a class of polymers comprised of carbamate (urethane) linkages formed between a polyol and an isocyanate moiety.

As used herein, the term “oleic content”, “oleic acid content”, “oleate content”, or “olein content” refers the percentage amount of oleic acid in the fatty acid profile of a substance (e.g., a TAG oil). As used herein, the term “C18:1 content” refers the percentage amount of a C18:1 fatty acid (e.g., oleic acid) in the fatty acid profile of a substance (e.g., a microbial oil).

As used herein, the term “high oleic” can refer to greater than 60% oleic acid, greater than 65% oleic acid, greater than 70% oleic acid, greater than 75% oleic acid, greater than 80% oleic acid, greater than 85% oleic acid, or greater than 90% oleic acid.

As used herein, “sequence identity” refers to a percentage of identical amino acid residues between two sequences being compared after an optimal alignment of sequences. An optimal alignment of sequences may be produced manually or by means of computer programs that use a sequence alignment algorithm (e.g., ClustalW, T-coffee, COBALT, BestFit, FASTA, BLASTP, BLASTN, and TFastA). Sequence identity can be calculated by determining the number of identical positions between the two sequences being compared, dividing this number by the number of positions compared, and multiplying the result obtained by 100 to obtain the sequence identity between the two sequences.

As used herein, the term “about” refers to ±10% from the value provided.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

### Microbial Cells and Oils Produced Therefrom.

An oil provided herein is obtained from a non-genetically modified microorganism (microbe), for example, oleaginous microalgae, yeast, or bacteria. In some embodiments, the non-genetically modified microorganism is a microalgal cell. In some embodiments, the microalgal cell is a non-genetically modified Prototheca sp. strain. The non-genetically modified Prototheca sp. strain can be produced by one or more classical strain improvement strategies described herein.

In some embodiments, a cell provided herein does not comprise an exogenous gene or exogenous nucleotides that encodes for an exogenous protein or gene. For example, a cell provided herein does not comprise an exogenous gene in a lipid biosynthetic pathway. In some embodiments, a cell provided herein does not comprise a genetic disruption of one or more alleles of an endogenous gene in a lipid biosynthetic pathway. In some embodiments, a cell provided herein does not comprise a heterologous insertion within a genomic region that encodes for an endogenous gene in a lipid biosynthetic pathway. Non-limiting examples of genes involved in lipid biosynthesis include acyl-ACP thioesterase (FAT), delta-12 fatty acid desaturase (FAD), ketoacyl-ACP synthase (KAS), stearoyl-ACP desaturase (SAD), lysophosphatidic acid acyltransferase (LPAAT), ketoacyl-CoA reductase (KCR), hydroxyacyl-CoA dehydratase (HACD), and enoyl-CoA reductase (ECR).

In some embodiments, a cell provided herein does not comprise an exogenous acyl-ACP thioesterase gene. In some embodiments, a cell provided herein does not comprise a genetic disruption of one or more alleles of an endogenous acyl-ACP thioesterase gene. In some embodiments, a cell provided herein does not comprise a heterologous insertion within a genomic region that encodes for an endogenous acyl-ACP thioesterase gene. In some embodiments, the endogenous acyl-ACP thioesterase gene is FATA.

In some embodiments, a cell provided herein does not comprise an exogenous fatty acid desaturase gene. In some embodiments, a cell provided herein does not comprise a genetic disruption of one or more alleles of an endogenous fatty acid desaturase gene. In some embodiments, a cell provided herein does not comprise a heterologous insertion within a genomic region that encodes for an endogenous fatty acid desaturase gene. In some embodiments, the endogenous fatty acid desaturase gene is a delta-12 fatty acid desaturase. In some embodiments, the endogenous fatty acid desaturase gene is FAD2.

In some embodiments, a cell provided herein does not comprise an exogenous ketoacyl-ACP synthase gene. In some embodiments, a cell provided herein does not comprise a genetic disruption of one or more alleles of an endogenous ketoacyl-ACP synthase gene. In some embodiments, a cell provided herein does not comprise a heterologous insertion within a genomic region that encodes for an endogenous ketoacyl-ACP synthase gene. In some embodiments, the endogenous ketoacyl-ACP synthase gene is KASI, KASII, or KASIII.

In some embodiments, a cell provided herein does not comprise an exogenous stearoyl-ACP desaturase gene. In some embodiments, a cell provided herein does not comprise a genetic disruption of one or more alleles of an endogenous stearoyl-ACP desaturase gene. In some embodiments, a cell provided herein does not comprise a heterologous insertion within a genomic region that encodes for an endogenous stearoyl-ACP desaturase gene. In some embodiments, the endogenous stearoyl-ACP desaturase gene is SAD2.

In some embodiments, a cell provided herein does not comprise an exogenous lysophosphatidic acid acyltransferase gene. In some embodiments, a cell provided herein does not comprise a genetic disruption of one or more alleles of an endogenous lysophosphatidic acid acyltransferase gene. In some embodiments, a cell provided herein does not comprise a heterologous insertion within a genomic region that encodes for an endogenous lysophosphatidic acid acyltransferase gene.

In some embodiments, a cell provided herein does not comprise a genetic disruption of one or more alleles within 1.5 kb of an endogenous V-type proton ATPase catalytic subunit A isoform 1 gene or 6S genomic region. In some embodiments, a cell provided herein does not comprise a heterologous insertion within 1.5 kb of an endogenous V-type proton ATPase catalytic subunit A isoform 1 gene or 6S genomic region.

In some embodiments, a cell provided herein does not comprise a genetic disruption of one or more alleles within 1.5 kb of an endogenous DAO1B gene. In some embodiments, a cell provided herein does not comprise a heterologous insertion within 1.5 kb of an endogenous DAO1B gene.

In some embodiments, a cell provided herein does not comprise a genetic disruption of one or more alleles within 1.5 kb of an endogenous Thi4 gene. In some embodiments, a cell provided herein does not comprise a heterologous insertion within 1.5 kb of an endogenous Thi4 gene.

Accordingly, a cell provided herein comprises uninterrupted sequences of endogenous genes or genomic regions, including FAD2, FATA1, KASII, SAD2, V-type proton ATPase catalytic subunit A isoform 1 (6S), DAO1B, and Thi4 described herein.

In some embodiments, a cell provided herein comprises at least 80%, at least 85%, at least 90%, at least 95%, or at least 99% sequence identity to any one of SEQ ID NO:1-26. In some embodiments, a cell provided herein comprises any one of SEQ ID NO:1-26 (genomic regions of CHK22 and CHK80). In some embodiments, a cell provided herein comprises SEQ ID NO: 1-26 (genomic regions of CHK22 and CHK80).

In some embodiments, a cell provided herein comprises at least 80%, at least 85%, at least 90%, at least 95%, or at least 99% sequence identity to any one of SEQ ID NO:1, 3, 5, 7, 9, 11, 13, 15, 18, 19, 21, 23, and 25 (genomic regions of CHK80). In some embodiments, a cell provided herein comprises any one of SEQ ID NO:1, 3, 5, 7, 9, 11, 13, 15, 18, 19, 21, 23, and 25 (genomic regions of CHK80). In some embodiments, a cell provided herein comprises SEQ ID NO:1, 3, 5, 7, 9, 11, 13, 15, 18, 19, 21, 23, and 25 (genomic regions of CHK80).

In some embodiments, an oil provided herein is produced by microalgae. In some embodiments, the microalgae is a species of a genus selected from the group consisting of: Chlorella sp., Pseudochlorella sp., Heterochlorella sp., Prototheca sp., Arthrospira sp., Euglena sp., Nannochloropsis sp., Phaeodactylum sp., Chlamydomonas sp., Scenedesmus sp., Ostreococcus sp., Selenastrum sp., Haematococcus sp., Nitzschia, Dunaliella, Navicula sp., Trebouxia sp., Pseudotrebouxia sp., Vavicula sp., Bracteococcus sp., Gomphonema sp., Watanabea, sp., Botryococcus sp., Tetraselmis sp., and Isochrysis sp. In some embodiments, the microalgae is Prototheca sp. In some embodiments, the microalgae is P. moriformis. In some embodiments, the microalgae is P. wickerhamii. In some embodiments, a cell provided herein is derived from a UTEX 1435 base strain. In some embodiments, a cell provided herein is derived from a UTEX 1533 base strain. In some embodiments, a cell provided herein is derived from a base strain having a 23S ribosomal DNA sequence that is at least 80%, at least 85%, at least 90%, at least 95%, or 100% identity to SEQ ID NO:30 or SEQ ID NO:31. In some embodiments, a cell provided herein has a 23S ribosomal DNA sequence that is at least 80%, at least 85%, at least 90%, at least 95%, or 100% identity to SEQ ID NO:30 or SEQ ID NO:31.

In some embodiments, an oil provided herein is produced by oleaginous yeast. In some embodiments, the oleaginous yeast is a species of a genus selected from the group consisting of: Candida sp., Cryptococcus sp., Debaromyces sp., Endomycopsis sp., Geotrichum sp., Hyphopichia sp., Lipomyces sp., Pichia, sp., Rodosporidium sp., Rhodotorula sp., Sporobolomyces sp., Starmerella sp., Torulaspora sp., Trichosporon sp., Wickerhamomyces sp., Yarrowia sp., and Zygoascus sp.

In some embodiments, an oil provided herein is obtained from or produced by oleaginous bacteria. In some embodiments, the oleaginous bacteria is a species selected from the group consisting of: Flavimonas oryzihabitans, Pseudomonas aeruginosa, Morococcus sp., Rhodobacter sphaeroides, Rhodococcus opacus, Rhodococcus erythropolis, Streptomyces jeddahensis, Ochrobactrum sp., Arthrobacter sp., Nocardia sp., Mycobacteria sp., Gordonia sp., Catenisphaera sp., and Dietzia sp.

Further provided herein are bioreactors comprising a non-naturally occurring microorganism provided herein. For example, these bioreactors comprise an oleaginous, non-naturally occurring microorganism and an oil produced by the microorganism.

While in many embodiments, an oil provided herein is obtained from a non-genetically modified microorganism or a classically-improved microorganism, in other embodiments, an oil provided herein is obtained from a genetically modified microorganism, for example, oleaginous microalgae, yeast, or bacteria. In some embodiments, an oil provided herein is obtained from a classically-improved microorganism that is then genetically modified to produce a genetically modified microorganism. In some embodiments, the genetically modified microorganism is a genetically modified Prototheca sp. strain.

### Classical Strain Improvement.

Classical strain improvement strategies can be used to select for organisms having desired phenotypes, e.g., high oleic oil production. Classical strain improvement (also called “mutation breeding”) involves exposing organisms to chemicals or radiation to generate mutants with desirable traits. These classical strain improvement methods introduce random or semi-random mutations, which can thereby allow selection of strains exhibiting desirable traits as a result of random mutagenesis. Several iterations of mutagenesis and selection can be performed with one or more mutagens to arrive to a strain having desirable phenotypes. Ultraviolet (UV) light can be used to introduce random mutations within a microorganism's nuclear genome. Chemical mutagens include compounds which inhibit or disrupt biosynthetic processes of a microorganism, e.g., antibiotics, antifungals, or carcinogens. Non-limiting examples of chemical mutagens include ICR-191, ethyl methanesulfonate (EMS), and 4-nitroquinoline-1-oxide (4-NQO). Non-limiting examples of chemical mutagens also include acridine mutagens, amino acid analogs, fatty acid biosynthesis inhibitors, cholesterol biosynthetic inhibitors, mTOR inhibitors, and membrane solubilizing agents. Combinations of chemical mutagens can also be used simultaneously to induce mutagenesis. Following mutagenesis, selective or enrichment agents can be used to select or enrich for strains of interest. Non-limiting examples of enrichment agents include L-canavanine, cerulenin, triparanol, clomiphene, clomiphene citrate, clotrimazole, terfenadine, fluphenazine, AZD8055, BASF 13-338, cafenstrole, clomiphene, PF-042110, and phenethyl alcohol.

Methods provided herein include classical strain improvement methods to improve strain productivity, carbon yield, and oleic acid content. Glucose consumption rate can be highly predictive indicator of lipid titer. As such, glucose consumption rate can be used as an enrichment tool in the mutant selection process. The methods provided herein can further include one or more of determining a total lipid titer of the cell, determining a fatty acid profile of the oil, or determining a C18:1 (e.g., oleic acid) content of the oil. Further, the methods provided herein can include assaying cell media to determine glucose consumption rate, total lipid titer, a fatty acid profile, and/or a C18:1 (e.g., oleic acid) content.

### High Oleic Oils of the Disclosure.

The complexity and physical properties of an oil can be evaluated by the fatty acid profile and the TAG profile of the TAG component of an oil. The fatty acid profile is a measure of fatty acid composition, and can be determined by subjecting an oil to transesterification to generate fatty acid methyl esters (FAMEs) and subsequently quantitating fatty acid type by Gas Chromatography equipped with a Flame Ionization Detector (GC/FID). Accordingly, fatty acid content can be determined by GC/FID. Since TAGs comprise of three fatty acids arrayed along the glycerol backbone in the triglyceride molecule, the number of possible distinct regioisomers of TAGs can be defined by the number of fatty acid species in the oil raised to the third power. The TAG profile provides relative amounts of various TAG species in an oil, which can be determined by subjecting the oil to TAG fractionation using Liquid Chromatography/Time of Flight-Mass Spectrometry (LC/TOF-MS) equipped with an Atmospheric Pressure Chemical Ionization (APCI) source.

In some embodiments, an oil provided herein has a high oleic acid content and low saturated fatty acid content. For example, the fatty acid content of the TAG component of an oil provided herein can be high in oleic acid and low in saturated fatty acids.

Non-limiting examples of monounsaturated fatty acids include C10:1, C12:1, C14:1, C16:1, C17:1, C18:1, C18:1-OH, C20:1, C22:1, and C24:1. In some embodiments, an oil provided herein comprises one or more of C10:1, C12:1, C14:1, C16:1, C17:1, C18:1, C18:1-OH, C20:1, C22:1, or C24:1 fatty acids. In some embodiments, an oil provided herein has a monounsaturated fatty acid content of at least 60%, at least 70%, at least 80%, or at least 90%. For example, an oil provided herein has a C18:1 content of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

In some embodiments, an oil provided herein has a fatty acid content comprising 60% or more, 70% or more, 80% or more, or 90% or more of C18:1 fatty acids. In some embodiments, the C18:1 fatty acids comprise oleic acid. In some embodiments, the C18:1 fatty acids comprise at least 90% oleic acid. In some embodiments, the C18:1 fatty acids comprise at least 95% oleic acid. In some embodiments, the C18:1 fatty acids comprise at least 99% oleic acid.

An oil provided herein has a C18:1 content of from 60-100%, 60-70%, 70-80%, 80-90%, 85-90%, or 80-100%. In some embodiments, an oil provided herein has a C18:1 content of at least 60%, at least 70%, at least 80%, or at least 90%. For example, an oil provided herein has a C18:1 content of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

An oil provided herein has an oleic content of from 60-100%, 60-70%, 70-80%, 80-90%, 85-90%, or 80-100%. In some embodiments, an oil provided herein has an oleic content of at least 60%, at least 70%, at least 80%, or at least 90%. For example, an oil provided herein has an oleic content of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

An oil provided herein has a triolein (OOO) content of from 50-100%, 60-100%, 60-90%, 60-80%, 60-70%, 50-70%, or 60-65%. In some embodiments, an oil provided herein has a triolein content of at least 30%, at least 40%, at least 50%, at least 60%, at least 70%, at least 80%, or at least 90%. For example, an oil provided herein has a triolein content of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, or more.

An oil provided herein has a C16:1 content of from 0.1-5%, 0.1-2%, 0.1-1%, 0.1-0.5%, or 0.1-0.2%. In some embodiments, an oil provided herein has a C16:1 content of greater than 0.1%, greater than 0.2%, greater than 0.3%, greater than 0.4%, or greater than 0.5%. In some embodiments, an oil provided herein has a C16:1 content of less than 0.5%, less than 0.4%, less than 0.3%, less than 0.2%, or less than 0.1%. In some embodiments, this oil has a C16:1 content of greater than 0%. In some embodiments, an oil provided herein has a C16:1 content of about 0.1% or about 0.2%.

An oil provided herein has a C20:1 content of from 0.1-5%, 0.1-2%, 0.1-1%, 0.5-2%, 0.5-1%, or 1-2%. In some embodiments, an oil provided herein has a C20:1 content of greater than 0.1%, greater than 0.2%, greater than 0.3%, greater than 0.4%, greater than 0.5%, greater than 0.6%, greater than 0.7%, greater than 0.8%, greater than 0.9%, or greater than 1%. In some embodiments, an oil provided herein has a C20:1 content of less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1%. In some embodiments, this oil has a C20:1 content of greater than 0%. In some embodiments, an oil provided herein has a C20:1 content of about 1% or about 1.5%.

Non-limiting examples of saturated fatty acids include C10:0, C12:0, C14:0, C16:0, C17:0, C18:0, C20:0, C22:0, and C24:0. In some embodiments, an oil provided herein comprises one or more of C10:0, C12:0, C14:0, C16:0, C17:0, C18:0, C20:0, C22:0, or C24:0 fatty acids. In some embodiments, an oil provided herein has a saturated fatty acid content of less than 30%, less than 20%, less than 10%, less than 9%, less than 8%, less than 7%, less than 6%, less than 5%, less than 4%, less than 3%, less than 2%, or less than 1%. For example, an oil provided herein has a saturated fatty acid content of less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, or less. In some embodiments, this oil has a saturated fatty acid content of greater than 0%. In some embodiments, an oil provided herein has a saturated fatty acid content of about 0%.

An oil provided herein has a C14:0 content of from 0.1-1%, 0.1-0.5%, 0.1-0.2%, or 0.1-0.3%. In some embodiments, an oil provided herein has a C14:0 content of less than 1%, less than 0.9%, less than 0.8%, less than 0.7%, less than 0.6%, less than 0.5%, less than 0.4%, less than 0.3%, less than 0.2%, or less than 0.1%. In some embodiments, this oil has a C14:0 content of greater than 0%. In some embodiments, an oil provided herein has a C14:0 content of about 0%.

An oil provided herein has a C16:0 content of from 1-10%, 1-5%, 3-5%, or 34%. In some embodiments, an oil provided herein has a C16:0 content of less than 10%, less than 9%, less than 8%, less than 7%, less than 6%, less than 5%, less than 4%, less than 3%, less than 2%, or less than 1%. In some embodiments, this oil has a C16:0 content of greater than 0%. In some embodiments, an oil provided herein has a C16:0 content of greater than 3%. In some embodiments, an oil provided herein has a C16:0 content of about 0%.

An oil provided herein has a C18:0 content of from 1-10%, 1-5%, 2-5%, or 2-3%. In some embodiments, an oil provided herein has a C18:0 content of less than 5%, less than 4%, less than 3%, less than 2%, or less than 1%. In some embodiments, this oil has a C18:0 content of greater than 0%. In some embodiments, an oil provided herein has a C18:0 content of about 0%.

An oil provided herein has a C20:0 content of from 1-10%, 1-5%, 1-3%, 1-2%, or 2-3%. In some embodiments, an oil provided herein has a C20:0 content of less than 5%, less than 4%, less than 3%, less than 2%, or less than 1%. In some embodiments, this oil has a C20:0 content of greater than 0%. In some embodiments, an oil provided herein has a C20:0 content of about 0%.

Non-limiting examples of polyunsaturated fatty acids include C18:2, C18:3, C18:3 alpha, C18:3 gamma, and C22:2. In some embodiments, an oil provided herein comprises one or more of C18:2, C18:3, C18:3 alpha, C18:3 gamma, or C22:2 fatty acids. In some embodiments, an oil provided herein has a polyunsaturated fatty acid content of less than 30%, less than 20%, less than 10%, less than 9%, less than 8%, less than 7%, less than 6%, less than 5%, less than 4%, less than 3%, less than 2%, or less than 1%. For example, an oil provided herein has a polyunsaturated fatty acid content of less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, or less. In some embodiments, this oil has a polyunsaturated fatty acid content of greater than 0%. In some embodiments, an oil provided herein has a polyunsaturated fatty acid content of about 0%.

An oil provided herein has a C18:2 content of from 1-10% or 5-10%. In some embodiments, an oil provided herein has a C18:2 content of less than 10%, less than 9%, less than 8%, less than 7%, less than 6%, less than 5%, less than 4%, less than 3%, less than 2%, or less than 1%. In some embodiments, this oil has a C18:2 content of greater than 0%. In some embodiments, an oil provided herein has a C18:2 content of about 0%.

In some embodiments, the fatty acid content of a TAG component comprises one or more of C14:0, C16:0, C16:1, C18:0, C18:1, C18:2, C18:3 alpha, C20:0, or C20:1 fatty acids. In some embodiments, the fatty acid content of the TAG component comprises C16:0, C18:0, C18:1, and C18:2 fatty acids.

The fatty acid content of the TAG component can comprise 1-10%, 1-5%, or 3-5% of C16:0 fatty acids. In some embodiments, the fatty acid content of a TAG component comprises 3% or more of C16:0 fatty acids. In some embodiments, the fatty acid content of a TAG component comprises more than 3% of C16:0 fatty acids.

The fatty acid content of the TAG component can comprise 1-5%, 2-5%, or 2-3% of C18:0 fatty acids. In some embodiments, the fatty acid content of the TAG component comprises 2% or more of C18:0 fatty acids. In some embodiments, the fatty acid content of the TAG component comprises more than 2% of C18:0 fatty acids. In some embodiments, the fatty acid content of the TAG component comprises less than 3% of C18:0 fatty acids.

The fatty acid content of the TAG component can comprise 1-10%, 1-5%, or 5-10% of C18:2 fatty acids. In some embodiments, the fatty acid content of the TAG component comprises 5% or more of C18:2 fatty acids. In some embodiments, the fatty acid content of the TAG component comprises more than 5% of C18:2 fatty acids.

In addition to the fatty acid profile, an oil can be further evaluated by the sterol profile or composition. Sterol composition can be determined by mass spectrometry, for example, gas chromatography-mass spectrometry (GC-MS); liquid chromatography-mass spectrometry (LC-MS); tandem mass spectrometry (MS/MS), and coupled liquid and gas chromatography with subsequent flame ionization detection (LC-GC-FID). Concentration of the different sterols present in an oil can be expressed as mg sterol/100 g of oil. Non-limiting examples of sterols include brassicasterol; campesterol; stigmasterol; beta-sitosterol (0-sitosterol); ergosterol; ergosta-5,7,9(11),22-tetraen-3-ol,(3β,22E), ergosta-7,22-dien-3-ol, (3β,22E); ergost-8(14)-en-3-ol, (3β); ergosta-5,8-dien-3-ol, (3β); 5.xi.-ergost-7-en-3β-ol; 9,19-cyclolanost-24-en-3-ol, (3β); and 9,19-cyclolanostan-3-ol, 24-methylene-, (3β).

In some embodiments, an oil provided herein comprises one or more of ergosterol; ergosta-5,8-dien-3-ol, (3β); 5.xi.-ergost-7-en-3β-ol; 9,19-cyclolanost-24-en-3-ol, (3β); and 9,19-cyclolanostan-3-ol, 24-methylene-, (3β).

An oil provided herein comprises 50-200 mg, 100-200 mg, 100-150 mg, 100-130 mg, 150-200 mg, 160-180 mg, or 160-170 mg of ergosterol per 100 g of the oil. In some embodiments, an oil provided herein comprises more than 50 mg, more than 100 mg, more than 150 mg, or more than 160 mg of ergosterol per 100 g of the oil. In some embodiments, an oil provided herein comprises no more than 170 mg of ergosterol per 100 g of the oil. In some embodiments, the sterol content (as a percentage of total sterols) of an oil provided herein comprises 40-80%, 40-70%, 50-70%, 50-60%, 40-50%, at least 40%, at least 50%, or at least 60% ergosterol on a weight-by-weight basis.

An oil provided herein comprises 0-10 mg, 0-5 mg, 0-2 mg, or 0-1 mg of campesterol per 100 g of the oil. In some embodiments, an oil provided herein comprises no more than 10 mg, no more than 9 mg, no more than 8 mg, no more than 7 mg, no more than 6 mg, no more than 5 mg, no more than 4 mg, no more than 3 mg, no more than 2 mg, or no more than 1 mg of campesterol per 100 g of the oil. In some embodiments, an oil provided herein does not comprise campesterol. In some embodiments, an oil provided herein does not contain a detectable level of campesterol. In some embodiments, the sterol content (as a percentage of total sterols) of an oil provided herein comprises about 0% campesterol on a weight-by-weight basis.

An oil provided herein can comprise 0-10 mg, 0-5 mg, 0-2 mg, or 0-1 mg of brassicasterol per 100 g of the oil. In some embodiments, an oil provided herein does not contain brassicasterol. In some embodiments, an oil provided herein does not contain a detectable level of brassicasterol. In some embodiments, the sterol content (as a percentage of total sterols) of an oil provided herein comprises about 0% brassicasterol on a weight-by-weight basis.

An oil provided herein comprises 0-10 mg, 0-5 mg, 0-2 mg, or 0-1 mg of stigmasterol per 100 g of the oil. In some embodiments, an oil provided herein comprises no more than 10 mg, no more than 9 mg, no more than 8 mg, no more than 7 mg, no more than 6 mg, no more than 5 mg, no more than 4 mg, no more than 3 mg, no more than 2 mg, or no more than 1 mg of stigmasterol per 100 g of the oil. In some embodiments, an oil provided herein does not contain stigmasterol. In some embodiments, an oil provided herein does not contain a detectable level of stigmasterol. In some embodiments, the sterol content (as a percentage of total sterols) of an oil provided herein comprises about 0% stigmasterol on a weight-by-weight basis.

An oil provided herein can comprise 0-10 mg, 0-5 mg, 0-2 mg, or 0-1 mg of 3-sitosterol per 100 g of the oil. In some embodiments, an oil provided herein comprises no more than 20 mg, no more than 15 mg, no more than 10 mg, no more than 9 mg, no more than 8 mg, no more than 7 mg, no more than 6 mg, no more than 5 mg, no more than 4 mg, no more than 3 mg, no more than 2 mg, or no more than 1 mg of β-sitosterol per 100 g of the oil. In some embodiments, an oil provided herein does not contain β-sitosterol. In some embodiments, an oil provided herein does not contain a detectable level of β-sitosterol. In some embodiments, the sterol content (as a percentage of total sterols) of an oil provided herein comprises about 0% β-sitosterol on a weight-by-weight basis.

An oil provided herein can comprise 0.1-100 mg, 0.1-50 mg, 1-20 mg, 1-50 mg, 10-50 mg, 10-30 mg, or 10-20 mg of ergosta-5,8-dien-3-ol, (3β)-per 100 g of the oil. In some embodiments, an oil provided herein comprises more than 1 mg, more than 5 mg, more than 10 mg, more than 15 mg, more than 20 mg, more than 25 mg, more than 30 mg, more than 35 mg, more than 40 mg, more than 45 mg, or more than 50 mg of ergosta-5,8-dien-3-ol, (3β)-per 100 g of the oil. In some embodiments, the sterol content (as a percentage of total sterols) of an oil provided herein comprises 1-10%, 5-10%, at least 1%, at least 2%, at least 3%, at least 4%, at least 5%, at least 6%, or at least 7% ergosta-5,8-dien-3-ol, (3β)-on a weight-by-weight basis.

An oil provided herein can comprise 0.1-100 mg, 0.1-50 mg, 1-50 mg, 10-50 mg, 20-50 mg, 30-50 mg, or 30-40 mg of 5.xi.-ergost-7-en-3β-ol, (3β) per 100 g of the oil. In some embodiments, an oil provided herein comprises more than 1 mg, more than 5 mg, more than 10 mg, more than 15 mg, more than 20 mg, more than 25 mg, or more than 30 mg of 5.xi.-ergost-7-en-3β-ol, (3β) per 100 g of the oil. In some embodiments, the sterol content (as a percentage of total sterols) of an oil provided herein comprises 1-20%, 1-10%, 10-20%, at least 1%, at least 2%, at least 3%, at least 4%, at least 5%, at least 6%, at least 7%, at least 8%, at least 9%, at least 10%, at least 11%, or at least 12% 5.xi.-ergost-7-en-3β-ol, (3β) on a weight-by-weight basis.

An oil provided herein can comprise 0.1-100 mg, 0.1-50 mg, 1-100 mg, 1-50 mg, 50-100 mg, 50-70 mg, 60-70 mg, 30-50 mg, or 30-40 mg of 9,19-cyclolanost-24-en-3-ol, (3β) per 100 g of the oil. In some embodiments, an oil provided herein comprises more than 20 mg, more than 30 mg, more than 40 mg, more than 50 mg, or more than 60 mg of 9,19-cyclolanost-24-en-3-ol, (3β) per 100 g of the oil. In some embodiments, the sterol content (as a percentage of total sterols) of an oil provided herein comprises 1-50%, 10-40%, 10-30%, 10-20%, 20-30%, at least 5%, at least 10%, at least 11%, at least 12%, at least 13%, at least 14%, at least 15%, at least 16%, at least 17%, at least 18%, at least 19%, or at least 20% 9,19-cyclolanost-24-en-3-ol, (3β) on a weight-by-weight basis.

An oil provided herein can comprise 0.1-100 mg, 0.1-50 mg, 1-100 mg, 1-50 mg, 1-20 mg, or 10-20 mg of 9,19-cyclolanostan-3-ol, 24-methylene-, (3β) per 100 g of the oil. In some embodiments, an oil provided herein comprises more than 1 mg, more than 2 mg, more than 3 mg, more than 4 mg, more than 5 mg, more than 6 mg more than 7 mg, more than 8 mg, more than 9 mg, more than 10 mg, or more than 11 mg of 9,19-cyclolanostan-3-ol, 24-methylene-, (3β) per 100 g of the oil. In some embodiments, the sterol content (as a percentage of total sterols) of an oil provided herein comprises 1-10%, 1-5%, 5-10%, at least 1%, at least 2%, at least 3%, at least 4%, or at least 5% 9,19-cyclolanostan-3-ol, 24-methylene-, (3β) on a weight-by-weight basis.

### Polyol Applications and End Products.

The oils described herein can be used or formulated with one or more excipients for a variety of applications, including but not limited to, process oils (e.g., for tires), waxes, lubricants, polyols, macrodiols, polyesterdiols, and polyurethane products, e.g., hard foams, soft foams, cast polyurethanes, thermoplastic polyurethanes (TPUs), elastomers, adhesives, coatings, laminates, films, and dispersions. Polyurethane products can be used to construct aerospace, automotive, medical, electronic, building and construction goods; sporting goods or recreational equipment, e.g., skis, snowboards, sidewalls, boating equipment, kayaks; and other consumer goods, e.g., industrial containers, coolers, mattresses, leather goods, apparel, footwear, mannequins, and phone cases. These polyurethane applications can serve as sustainable alternatives to petroleum-based, non-renewable materials, such as acrylonitrile butadiene styrene (ABS), ultra-high molecular weight polyethylene (UHMWPE), or high density polyethylene (HDPE).

Oil provided herein can have improved production efficiency and a TAG composition that is enhanced for improved control of hydroformylation chemistry for generating polyols. These characteristics of microbial oil result in a greater degree of hydroxyl group (—OH) functionality relative to oils with greater TAG heterogeneity (hence, lower purity) and/or diversity (e.g., oilseed or plant derived oils). Thus, polyols produced from hydroformylation of high oleic oils can be preferable in generating polymers, including in instances where physical properties of a polymer can be compromised by molecular impurities, such as non-hydroxylated fatty acids that may be present in oils having a more diverse or heterogeneous TAG profile.

Polyols derived from hydroformylation of a high oleic oil can be particularly useful for producing polyurethane materials. For example, oils provided herein can have relatively low TAG diversity, low fatty acid diversity, and the majority of fatty acids present in the oils may be unsaturated fatty acids. A higher ratio of unsaturated fatty acid to saturated fatty acid can allow for increased chemical reactivity at the double bonds. Oils having low TAG diversity and a high proportion of unsaturated fatty acids can be especially desirable in production of polyurethanes because hydroformylation of such a mixture yields a greater percentage of fatty acids that can participate in crosslinking reactions with isocyanates. Unlike unsaturated fatty acids, saturated fatty acids do not contain carbon-carbon double bounds and cannot participate in crosslinking reactions with isocyanates. Thus, polyols generated from hydroformylation of unsaturated fatty acids from high oleic oil can yield polyurethane materials having superior properties.

The hydroxyl functionality can be introduced into oils provided herein via a chemical conversion of the TAG component. This conversion requires the presence of a double bond on the acyl moiety of the fatty acid, e.g., an olefinic group, which can be accomplished using several different chemistries including, for example:
i) Epoxidation in the presence of hydrogen peroxide and acid catalyst, followed by ring opening with reagents, such as water, hydrogen, methanol, ethanol, or other polyols. These chemistries result in secondary hydroxyl moieties, and are therefore less reactive, for example, with isocyanate or methyl esters.
ii) Ozonolysis by molecular oxygen results in the formation of ozonides, which upon further oxidation results in scission at the double bond and formation of di-acids, carboxylic acids, and, upon reduction with hydrogen, aldehydes. Ozonolysis and reduction of oleic acid, for example, produces azaleic acid, pelargonic acid, and pelargonaldehyde, respectively.
iii) Hydroformylation with synthesis gas (syngas), using rhodium or cobalt catalysts to form the aldehyde at the olefinic group, followed by reduction of the aldehyde to alcohol in the presence of hydrogen.

While typically carried out in organic solvent, processes that utilize aqueous systems have been developed to improve sustainability of these chemistries. Of the chemistries described above, only hydroformylation results in the preservation of fatty acid length and formation of primary hydroxyl moieties. Primary hydroxyl functionalities are highly desirable due to increased reactivity compared to secondary hydroxyl moieties. Furthermore, only olefinic fatty acids with a double bond that is converted into a site possessing hydroxyl functionality, through epoxidation and ring opening, ozonolysis, or hydroformylation/reduction, can participate in subsequent downstream chemistries, i.e., reaction with an isocyanate moiety to form a urethane linkage or reaction with methyl esters to form polyesters. All other fatty acids, namely, fully saturated fatty acids that do not contain carbon-carbon double bonds, cannot participate in crosslinking reactions with isocyanates. Hence, saturated fatty acids can compromise the structural integrity and degrade performance of the polymer produced therefrom.

### Personal Care Product Applications.

The oils described herein can be used or formulated with one or more excipients for a variety of personal care product applications, including but not limited to, cosmetics, creams, face creams, hand creams, balms, lip balms, serums, face serums, body oils, hair oils, soaps, shampoos, conditioners.

### Food Applications.

The oils described herein can be used or formulated with one or more excipients for a variety of food applications, including but not limited to, food products, e.g., food-grade oils, cooking oil, frying oils, coatings, salad dressings, spreads, frozen desserts, pharmaceuticals, nutritional supplements, nutraceuticals, meal replacements, infant formulas, beverages, flavoring agents, and food additives.

### EXAMPLES

### Example 1. Generation of a Classically Improved Microalgal Strain Capable of Producing a Triglyceride Oil that is Highly Enriched in Oleic Acid

### Strain Acquisition, Isolation, and Characterization.

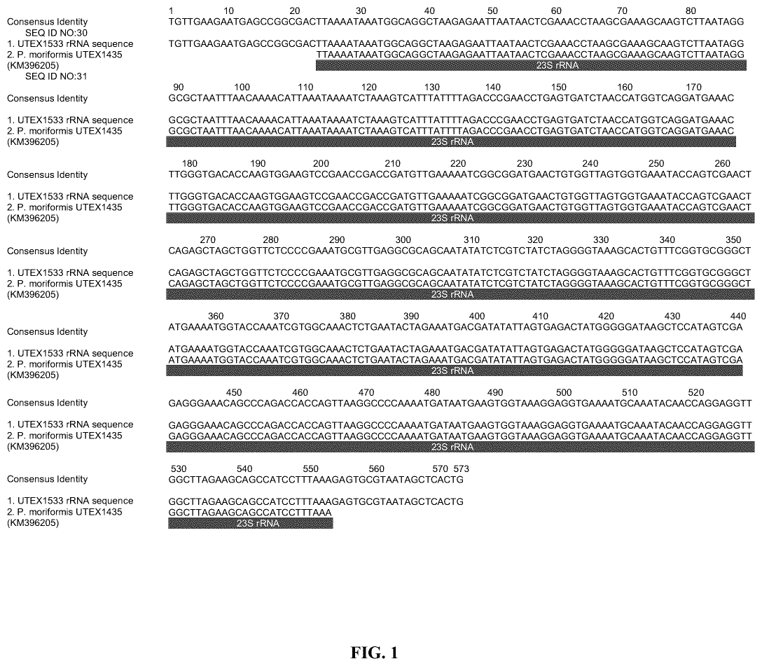

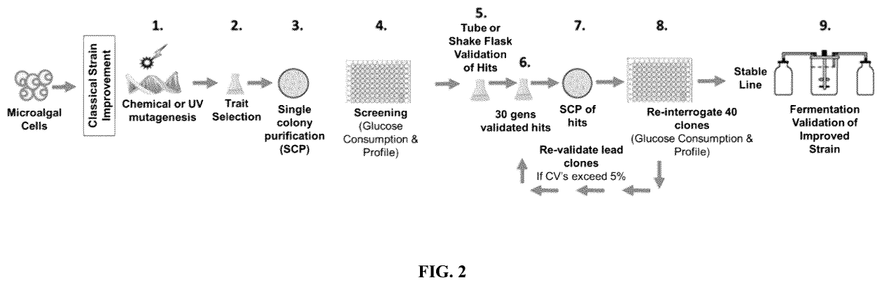

A Prototheca wickerhamii strain (UTEX 1533) was obtained from the University of Texas at Austin Culture Collection of Algae (UTEX). Analysis of the 23S ribosomal DNA (rDNA) sequence of UTEX 1533 (SEQ ID NO:30) suggests that UTEX 1533 is very closely related to a Prototheca moriformis strain, UTEX 1435 (SEQ ID NO:31). The 23S rDNA sequence of UTEX 1533 has 100% sequence identity with the published 23S rDNA sequence from UTEX 1435 (FIG. 1). The UTEX 1533 strain acquired from UTEX was verified as axenic through repeated passages on solid media containing various antibiotic and antifungal agents, followed by passage on antibiotic-free solid media to confirm a single cell type and a homogeneous morphology. This parent strain was given the designation “Strain 0” and also known as CHK22.

### First Round of Mutagenesis.

Classical strain improvement of Strain 0 was undertaken in an effort to improve strain productivity, carbon yield, and oleic acid content. First, Strain 0 was exposed to UV light to introduce a diverse collection of random mutations within the nuclear genome. To accomplish this, Strain 0 cells were grown to mid-log phase in 50 mL of vegetative growth media (M21 seed media) in a 250-mL baffle flask at 28° C./200 rpm. The cells were then washed in seed media and 3×10⁷ cells were spread on a 150-mm solid tryptone soy agar (TSA)+5 g/L glucose plate+1.5% agar and allowed to dry. The cells on the plate were then exposed to 25,000 μJoules of UV radiation (approximately 8.02 sec) using a Stratalinker® 1800 UV Crosslinker system. Following UV exposure, the cells were scraped off of the solid media surface with a sterile cell scraper and into M21 seed media lacking both sugar and nitrogen. These cells were then washed extensively in the same nitrogen-free and sugar-free media, and then collected in a sterile 0.2-micron filter sterilization unit by vacuum filtration. Light exposure was kept to a minimum during this process to reduce the frequency of light-dependent repair of UV damage. The cells were washed off of the sterile filter and re-suspended in M20+5 g/L glucose pre-seed media formulated with 10 mM L-serine as a nitrogen source, in lieu of the typical ammonium sulfate. Half of the mutagenized cell population was exposed to the acridine mutagen ICR-191 in M20+5 g/L glucose pre-seed media for 24 h at 28° C. with 200 rpm agitation. ICR-191 can introduce both double strand breaks and frame-shift mutations when used as a single agent, and significant chromosomal rearrangements when used in conjunction with other mutagens, including UV radiation.

The culture of Strain 0 in M22 pre-seed media formulated with 10 mM L-serine as a nitrogen source was previously shown to have little impact or no impact growth. Nonetheless, amino acid uptake studies in related microalgae have suggested that culture under such conditions stimulates the uptake of L-arginine. After 16-24 h of incubation at 28° C./200 rpm agitation, a portion of the mutagenized and mock-mutagenized cultures were independently plated to M20+5 g/L glucose+10 mM L-serine+100 μg/mL L-canavanine+0.8% agarose solid media. L-canavanine is a toxic analog of L-arginine that disrupts protein synthesis and leads to growth arrest. It is possible, albeit exceedingly rare, (i.e., approximately 1 in 10⁸ to 1 in 109) to spontaneously obtain L-canavanine-resistant (L-can^{R}) colonies when plating otherwise wild-type populations of cells onto L-canavanine-containing media in the presence of L-arginine. Based on this possibility, the frequency of L-can^{R} individuals observed in UV-mutagenized versus mock-mutagenized cell populations can serve as a rough measure of the efficiency of a given mutagenesis at increasing the rate of mutation over background. In the case of this UV-mutagenesis campaign, L-can^{R} colonies were obtained at a rate >10,000 times more frequently in the UV-mutagenized population than in the mock-mutagenized population, indicating a significant mutagenic load.

The mutagenesis, trait selection, high-throughput, and automated screening steps of the improvement process are outlined in FIG. 2. Briefly, algal cells in log phase of growth were subjected to mutagenesis by means of chemicals or UV light (1). Cells were then sub-cultured into lipid production medium where the cells were subjected to selection/enrichment strategies (2). A non-mutagenized strain was run in parallel with the mutagenized strain until the growth in lipid production medium (as determined by an increase in A750) of the mutant strain exceeded that of the non-mutagenized control, as determined by a statistical analysis. Strains were then plated to solid medium to obtain clonal isolates (3), followed by the interrogation of these isolates in lipid production medium in a 96-well plate format (4). Using glucose consumption as a surrogate for oil production, high glucose consuming strains were validated in lipid production medium in a tube or shake flask format (5). Isolates that were successfully validated were sub-cultured for multiple generations to stabilize mutations (6), followed by purification of clonal isolates (7) and subsequent re-interrogation in lipid production medium (8). Clones deemed to be phenotypically stable were then ready for validation in fermentation (9), while clones that still show variability in (8) are passaged once more (6) to generate stable lines.

### Enrichment for Changes in Fatty Acid Profiles Using Cerulenin, an Inhibitor of KASI/KASII.

Cerulenin is an antifungal antibiotic reported to inhibit fatty acid and sterol biosynthesis. Data from a number of studies suggest that cerulenin can specifically inhibit both the 0-ketoacyl-ACP synthase I (KASI) and the 0-ketoacyl-ACP synthase II (KASII) enzymes in plants, bacteria, and fungi. KASI catalyzes the two carbon at a time condensation reaction that generates 6-16 carbon long fatty acids. KASII functions in the condensation of palmitoyl-ACP with malonyl-ACP to yield stearoyl-ACP, which is rapidly desaturated to oleate. UV-induced mutants of Strain 0 that show resistance to cerulenin could achieve this resistance through mutations that increase KASI/KASII activity, a phenotype that could be highly beneficial for oleate production. In order to isolate cerulenin-resistant Strain 0 mutants, the approach illustrated in FIG. 2 was utilized. Briefly, the UV-mutagenized and mock mutagenized populations described previously were allowed to incubate in 5 mL M20 pre-seed media at 28° C./200 rpm for an additional 48 hours after sampling for the L-canavanine resistance test (72 h total). An 8% (v/v) inoculum from each culture was used to initiate a 10 mL seed culture in standard M21 seed media in a 50-mL bioreactor tube. These seed cultures were incubated for 24-26 h hour to mid-log phase, after which the cultures were divided and used to perform a 0.8% inoculation of different wells of a 96-well block containing 0.5 mL of M22 lipid production media and varying amounts of cerulenin or DMSO as a vehicle control. These lipid cultures were incubated for approximately 72 hours. Growth was then assessed by measuring optical density at 750 nm wavelength (OD750) in a Molecular Devices Spectramax M5 plate reader.

While growth of the vast majority of the cells can be negatively impacted by cerulenin, a very small subset of cells with beneficial mutations within the mutant population can be resistant to the antibiotic. To increase the likelihood of isolating these resistant cells, wells in which inhibition of growth was >50% of the growth observed in DMSO-treated controls were collected onto sterile filters, extensively washed in M20 pre-seed media, and then used to initiate another round of M20 pre-seed culture. These cells were then subjected to another round of seed and then lipid culture in the presence of differing amounts of cerulenin or DMSO, as was performed in the first round of enrichment for cerulenin resistance. This cycle was repeated until a “differentiation event” was observed in which the MIC50 of the mutant population is higher than that of the mock mutagenized population. Wells in which statistically significant differences were observed were pooled, collected on sterile filters, washed extensively, and then diluted and plated for single colonies on 10-cm M20+5 g/L glucose+1.5% agar plates. Clonally isolated mutants from the cerulenin-resistant population constituted a cerulenin-resistant “mutant library”.

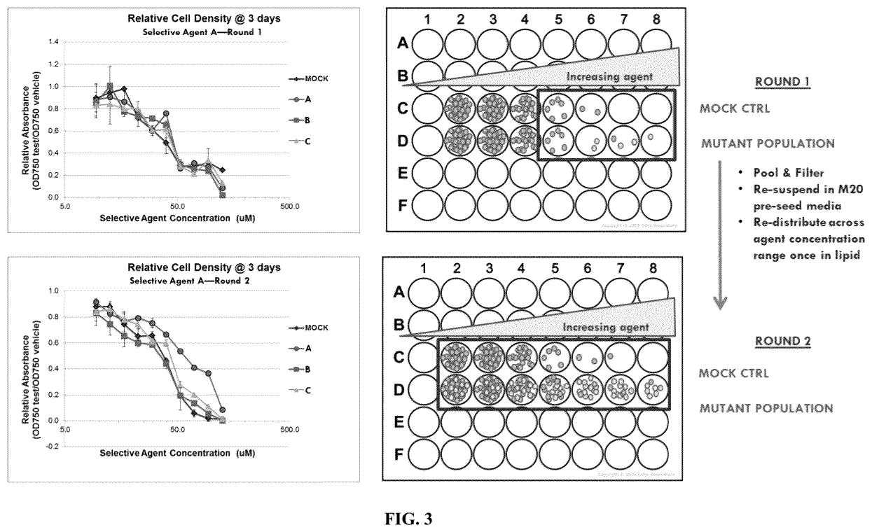

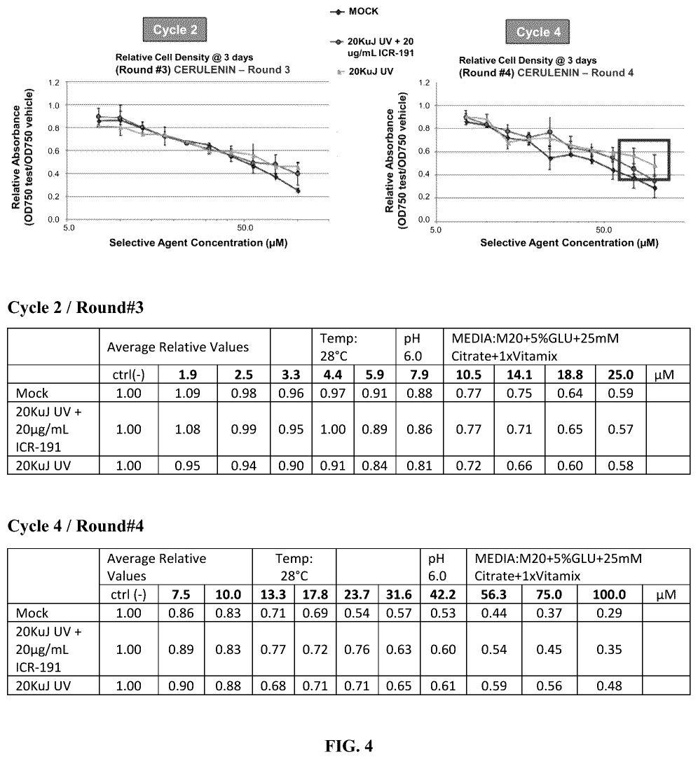

Using the UV-mutagenized and UV+ICR-191-mutagenized library of Strain 0, four cycles of enrichment were conducted as described at which time a very small but statistically significant increase in the MIC50 was observed only in the UV-mutagenized mutant population when compared to the mock-mutagenized controls (FIG. 3). Cells in these wells were pooled, collected with 0.2-μm filter sterilization units, washed with media, and then re-suspended in media and counted. These cells were then spread onto M20+glucose+1.5% agar solid media to clonally isolate individual mutants.

FIG. 3 shows a prophetic example of the enrichment strategy used to isolate cerulenin-resistant microalgae. As illustrated in FIG. 3, a portion of a 96-deep well block shows wells containing microalgae, with grey circles representing wild type, cerulenin-sensitive a portion of a 96-deep well blocks with wells containing microalgae, with grey circles representing wild type, cerulenin-sensitive cells, and yellow circles representing cerulenin-resistant cells. When resistant cells are collected in the first round of enrichment and then redistributed across the cerulenin concentration gradient in a second round of enrichment, cells with a high tolerance for the cerulenin are present in concentrations that easily out-compete the sensitive cells in the population. These wells should thus have higher levels of growth than the mock-mutagenized populations of cells that have undergone a similar treatment. This “differentiation event” indicates the outgrowth of cerulenin resistance.

FIG. 4 illustrates an actual enrichment strategy used to isolate cerulenin-resistant microalgae. After the first round of exposure to cerulenin, very little difference is observed between the UV-mutagenized and UV+ICR-191-mutagenized populations and the mock-mutagenized controls (left panel). After two additional round of drug exposure, the UV-mutagenized population shows slightly higher growth even at the highest cerulenin concentration then the mock mutagenized controls (right panel; red box). Wells containing these more cerulenin resistant cells were pooled, collected, and washed on sterile filters, and diluted and spread on solid media to isolate single colonies.

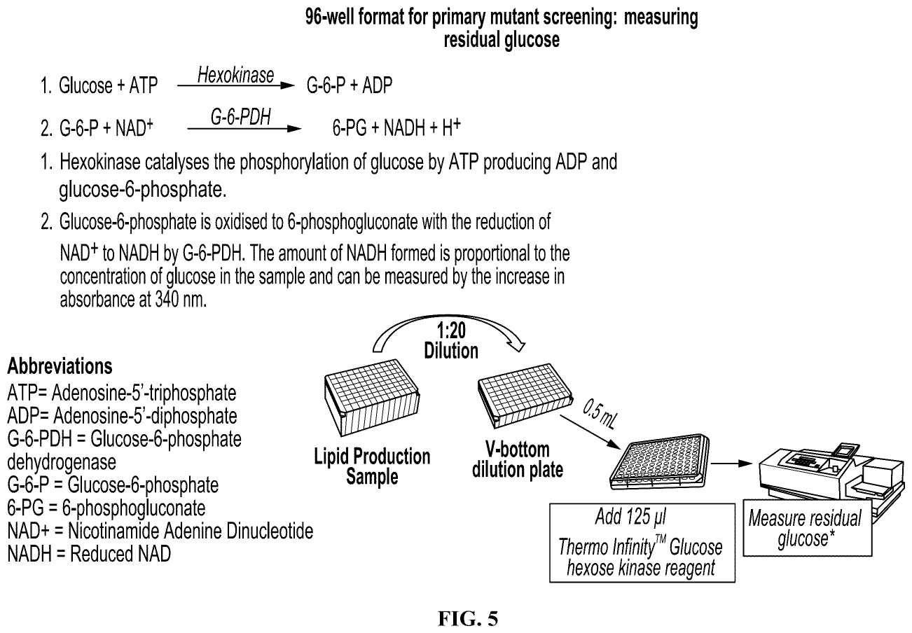

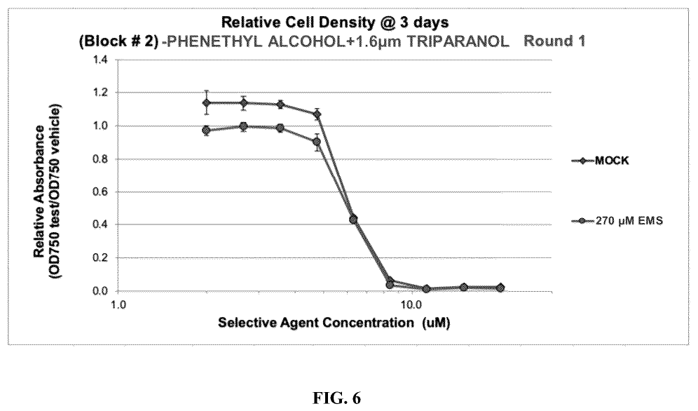

Colonies arising from individual mutant clones were picked from the solid media plates for screening using a Picolo™ automated colony picking inoculation tool integrated into a TECAN Freedom EVO® 200 automated liquid handling system. Each selected colony was used to inoculate both a single well of an archival 96-well solid media M20+5 g/L glucose+0.8% agarose storage plate, as well as the corresponding well of a 96-well deep well block containing 0.5 mL of M20 pre-seed media. Pre-seed cultures were incubated at 28° C./900 rpm/80% humidity for 72 h. An 8% (v/v) inoculum from each culture was then used to initiate a seed culture in 0.5 mL of standard M21 seed media in corresponding wells of 96-deep well block. These seed cultures were incubated at 28° C./200 rpm/80% humidity for 24-26 h to mid-log phase, and then the cultures were used to perform a 0.8% inoculation of corresponding wells of a 96-deep well block containing 0.5 mL of M22 lipid production media. Lipid cultures were incubated for 72 h and then samples from each culture were assessed for rate of glucose consumption using an automated glucose assay described in FIG. 5 to measure residual glucose in each well. Individual clones with glucose consumption rates greater than or equal to the average consumption rate of the Strain 0 controls were selected for further analysis. Approximately 125 μL of broth from each of the selected lipid cultures were frozen at −80° C. for approximately 1 hour and lyophilized overnight and then submitted for quantitative FAME analysis of fatty acid composition by GC-FID. A number of clones were identified with significant increases in oleate levels and glucose consumption rates equal to or greater than Strain 0 (TABLE 1). These primary hits were re-screened in a tube assay to assess oleate production and lipid titer following 5-days in lipid culture (TABLE 2). Three mutants, Strain 0_G07, Strain 0_E05, and Strain 0_E06, had lipid titers that were comparable to the Strain 0 parental strain while manifesting 9-10% more oleate content.

TABLE 1

| Strain | % Oleate in Block |
|---|---|
| Strain 0_H07 | 60.14 |
| Strain 0_G07 | 69.38 |
| Strain 0_E05 | 68.96 |
| Strain 0_H05 | 71.96 |
| Strain 0_E06 | 69.92 |
| Strain 0_G08 | 71.84 |
| Strain 0 | 59.34 |

TABLE 1 summarizes oleate content of select mutant strains exhibiting higher glucose consumption in a 96-well block lipid assay. Mutant strains (approximately 2,200 isolates) were grown in lipid production medium (0.5 mL) with shaking (900 rpm) for 72 hours at 28° C. at which point glucose consumption was measured. Isolates with high glucose consumption levels were further interrogated for fatty acid composition with lead strains shown here.

TABLE 2

| | Avg | Avg | Oil | Avg | Avg | Avg |
|---|---|---|---|---|---|---|
| Strain | DCW | Oil | % CV | NLB | PCP | % Oleate |
| Strain 0_H07 | 15.5 | 10.8 | 4.5 | 4.7 | 2.3 | 58.82 |
| Strain 0_G07 | 15.1 | 10.1 | 2.6 | 5.0 | 2.0 | 67.60 |
| Strain 0_E05 | 15.2 | 10.3 | 1.1 | 4.9 | 2.1 | 67.87 |
| Strain 0_H05 | 8.2 | 5.1 | 10.7 | 3.0 | 1.7 | 69.16 |
| Strain 0_E06 | 15.1 | 10.2 | 2.3 | 4.8 | 2.1 | 69.24 |
| Strain 0_G08 | 9.1 | 5.6 | 4.2 | 3.5 | 1.6 | 68.89 |
| Strain 0 | 15.0 | 10.3 | 2.6 | 4.7 | 2.2 | 58.45 |

TABLE 2 summarizes the performance of select mutant strains in a tube-based assay based on average dry cell weight (Avg DCW), average oil titer (g/L) (Avg Oil), oil titer g/L, % coefficient of variance (Oil % CV), average non lipid biomass (Avg NLB), average per cell production (oil titer/NLB-Avg PCP), and average % oleate content (Avg % Oleate). Mutants described in TABLE 1 were re-interrogated in a larger tube-based format in which isolates were grown in duplicate in 10 mL of lipid production medium in a 50-mL bioreactor with shaking (200 rpm) for 120 hours at 28° C. at which point 1 mL of biomass was removed, applied to a polycarbonate filter, washed with an equal volume of Milli Q water, and placed in tared glass vial at −80° C. for 30 minutes. Vials containing filters and frozen biomass were lyophilized to dryness overnight, weights were recorded, and filters with dried biomass were subjected to direct transesterification followed by GC/FID to quantitate FAMEs. Mutants highlighted in bold showed both high productivity and high oleate levels.

The phenotypic stability of each of the most promising mutants (highlighted in bold in TABLE 2) was evaluated by repeated sub-culturing in vegetative growth media (M21+40 g/L glucose seed media) until 30 or more population doublings had occurred. Cultures were then plated to M20+5 g/L glucose+0.8% agarose solid media. Six colonies were picked to a solid media archival storage plate and were subsequently analyzed in duplicate 10-mL lipid production assays conducted in 50-mL bioreactor tubes at 28° C./200 rpm. One of the strains showed a coefficient of variance (% CV) of less than 3% for oil titer and less than 1% in oleate levels across all six sub-clones. This strain was deemed phenotypically stable and cryo-preserved as strain “Strain 1”. The other mutant clones were not phenotypically stable and were reserved for additional rounds of sub-culturing and phenotypic evaluation or subject to additional round of classical strain improvement.

TABLE 3 summarizes the results of tube-based assays assessing stability of classically improved high oleic strains. Tube-based assays in which six isolates from each strain were grown in duplicate in 10 mL of lipid production medium. Cultures were grown with shaking (200 rpm) for 120 hours at 28° C. at which point 1 mL of biomass was removed, applied to a polycarbonate filter, washed with an equal volume of Milli Q water, and placed in tared glass vial at −80° C. for 30 minutes. Vials containing filters and frozen biomass were lyophilized to dryness overnight, their weights recorded and filters with dried biomass were subjected to direct transesterification followed by GC/FID to quantitate FAMEs. Strain 0_G07 was determined to be stable and was cryo-preserved as “Strain 1”.

TABLE 3

| Strains | | | | | | | | | |
|---|---|---|---|---|---|---|---|---|---|
| in | | Avg | Avg | Avg | Avg | Avg | Avg | Avg | |
| Duplicate | Strain | DCW | Oil | PCP | NLB | C18:1 | C16:1 | C18:2 | |
| 6 | Strain 0_G07_A1-5, B1 | 14.3 | 9.8 | 2.2 | 4.5 | 68.3 | 18.5 | 7.6 | Stable; |
| | STDEV | 0.2 | 0.2 | 0.2 | 0.1 | 0.1 | 0.1 | 0.1 | banked |
| | % cv | 1.4 | 1.8 | 3 | 2.2 | 0.2 | 0.7 | 1.1 | as “Strain |
| | | | | | | | | | 1” |
| 6 | Strain 0_E05_A7-11, B8 | 14.5 | 9.9 | 2.2 | 4.6 | 68.6 | 18.2 | 7.5 | Unstable |
| | STDEV | 0.5 | 0.4 | 0.2 | 0.3 | 0.2 | 0.2 | 0.1 | |
| | % cv | 3.7 | 3.5 | 6.9 | 7.0 | 0.3 | 0.9 | 1.0 | |
| 6 | Strain 0_E06_A1-5, B1 | 14.5 | 10.0 | 2.2 | 4.8 | 70.2 | 16.4 | 7.6 | Unstable |
| | STDEV | 0.9 | 0.6 | 0.1 | 4.5 | 0.2 | 0.1 | 0.1 | |
| | % cv | 6.2 | 5.8 | 3.8 | 0.4 | 0.2 | 0.4 | 0.8 | |
| 1 | Strain 0 | 14.8 | 10.3 | 2.3 | 4.4 | 59.0 | 27.1 | 7.0 | |
| | STDEV | 0.21 | 0.07 | 0.07 | 0.15 | 0.36 | 0.32 | 0.01 | |
| | % cv | 1.4 | 0.6 | 2.7 | 3.3 | 0.6 | 1.2 | 0.2 | |

Following the cryo-preservation of Strain 1, a series of fermentation development runs were conducted to ascertain which process conditions would yield the highest oleic acid content compared to the parental base strain, Strain 0, which elaborates ca. 582 oleic acid in low cell density fermentations, and ca. 6900 oleic acid under an optimized high cell density, high oleic process in a 0.5-L bioreactor. As illustrated in TABLE 4, feeding a macronutrient bolus (Mg⁺⁺ and PO₄⁻) or administering a complete media replacement bolus, had little impact on oleic accumulation. In contrast, increasing the dissolved oxygen (DO) from 30 to 50%, further elevated the oleic acid content by roughly 3%. Additional attempts at stressing Strain 1 by reducing the run temperature after the base transition from 28° C. to 25° C. showed no additional impact on oleic acid levels.

TABLE 4

| | | | DO | | | | | | | |
|---|---|---|---|---|---|---|---|---|---|---|
| | | | set | | | | | Peak oil | Final oil | Final |
| | Run | Nitrogen | point | | | DCW | Oil | production | production | yield |
| Strain | No. | (mM) | (%) | Bolus | C18:1 | (g/L) | (g/L) | (g/Ld) | (g/Ld) | (g/g) |
| Strain 0 | 19003 | 220 | 30 | Mg⁺⁺, PO₄⁻⁻⁻ | 69.54 | 142.9 | 83.5 | 17.97 | 14.52 | .209 |
| Strain 0 | 19004 | 220 | 30 | Mg⁺⁺, PO₄⁻⁻⁻ | 68.86 | 142.8 | 84.1 | 17.15 | 14.62 | .219 |
| Strain 1 | 19009 | 220 | 30 | Mg⁺⁺, PO₄⁻⁻⁻ | 73.41 | 140.8 | 81.8 | 15.10 | 13.77 | .218 |
| Strain 1 | 19010 | 220 | 30 | Mg⁺⁺, PO₄⁻⁻⁻ | 73.68 | 149.6 | 85.9 | 16.29 | 14.47 | .223 |
| Strain 1 | 19013 | 170 | 30 | Complete | 73.16 | 170.2 | 103.1 | 21.9 | 17.86 | .222 |
| | | | | media | | | | | | |
| | | | | replacement | | | | | | |
| Strain 1 | 19014 | 170 | 30 | Complete | 73.25 | 167.8 | 103.8 | 21.4 | 18.1 | .232 |
| | | | | media | | | | | | |
| | | | | replacement | | | | | | |
| Strain 1 | 19019 | 220 | 50 | Mg⁺⁺, PO₄⁻⁻⁻ | 75.31 | 132.1 | 68.7 | 12.77 | 12 | .186 |
| | | | | 25° C. | | | | | | |
| Strain 1 | 19020 | 220 | 50 | Mg⁺⁺, PO₄⁻⁻⁻ | 75.38 | 125.5 | 61.9 | 12.89 | 10.87 | .182 |
| | | | | 25° C. | | | | | | |

TABLE 4 shows fermentation development runs for Strain 1 in comparison with the highest oleic content observed in runs of Strain 0 (all at 0.5-L scale). All runs were carried out at 0.5-L scale. Nitrogen refers to the total nitrogen used in the process (batched+fed ammonia). DO refers to dissolved oxygen. Mg⁺⁺ and PO₄⁻ bolus refers to a bolus (equivalent to the amount of each macronutrient that is batched in the initial vessel seed medium) that is administered just after base transition. Complete media replacement refers to administering a bolus (just after base transition) containing all batched media components with the exception of nitrogen. All broth samples for analysis were applied to a polycarbonate filter, washed with an equal volume of Milli Q water, and placed in a tared glass vial at −80° C. for 30 minutes. Vials containing filters and frozen biomass were lyophilized to dryness overnight, weights were recorded, and filters with dried biomass were subjected to direct transesterification followed by GC/FID to quantitate FAMEs.

### Example 2. Generation of a Classically Improved Microalgal Strain Capable of Producing a Triglyceride Oil that is Highly Enriched in Oleic Acid

Challenging Membrane Integrity to Enrich for Mutants with Increased Oleate Levels.

The mutant strain, Strain 0_E06 (Strain E06), while not phenotypically stable, consistently achieved higher oleate levels than the mutant lineage that gave rise to Strain 1 (TABLE 3). As such, this mutant lineage was selected for further rounds of classical strain improvement, and designated as “Strain K”. Following overnight growth in M21+40 g/L glucose seed media to mid-log phase, the E06 mutant was exposed to 270 mM ethyl methanesulfonate (EMS) in 0.1 M potassium phosphate buffer (pH 7.0) for 45 min at room temperature in dark and with periodic gentle agitation. Mutagenesis was terminated with the addition of a sodium thiosulfate solution to final concentration of 3% (w/v). In parallel, a mock-mutagenized control cell population was taken through all the same manipulations, except for exposure to EMS. Cells were collected using 0.2-micron filter sterilization units, washed extensively with water, and then washed with and into 5 mL M20+5 g/L glucose pre-seed media. Pre-seed cultures were incubated at 28° C./900 rpm/80% humidity for 72 h. An 8% (v/v) inoculum from each culture was then used to initiate a seed culture in 10 mL of standard M21+5 g/L glucose seed media in 50-mL bioreactor tubes. These seed cultures were incubated at 28° C./900 rpm/80% humidity for 24-26 h to mid-log phase, and then the cultures were used to perform a 0.8% inoculation of replicate wells in a 96-deep well block containing 0.5 mL of M22 lipid production media formulated with 1.6 μM triparanol and varying concentrations of the membrane fluidizing chemical, phenethyl alcohol, in a range from 2-20 mM. Triparanol is a 24-dehydroreductase inhibitor that disrupts production of cholesterol in mammalian cells. Triparanol can inhibit phytosterol biosynthesis in some microalgae. These lipid cultures were incubated for approximately 72 hours at 28° C./900 rpm/80% humidity. Growth was then assessed by measuring optical density at 750 nm wavelength (OD750) in a Molecular Devices Spectramax M5 plate reader. Cells from lipid cultures with 50% or greater inhibition of growth relative to vehicle (DMSO)-treated controls as measured by optical density (FIG. 6) were pooled in sterile 0.2-micron filter sterilization units, washed extensively with fresh M20+5 g/L glucose pre-seed media, and used to initiate a 10 mL M20+5 g/L glucose pre-seed cultures in 50-mL bioreactor tubes. The surviving population was then subjected to a second round of exposure to a combination of 5 μM triparanol and a range of phenethyl alcohol concentrations, ranging from 4-8 mM, again under lipid production culture conditions. Surviving cells were collected, washed, and recovered into M21+5 g/L glucose seed media, allowed to grow overnight to mid-log phase and then cryopreserved with the addition of dimethyl sulfoxide to 5% (v/v) and flash freezing. This expanded population of rare, surviving mutants was subsequently interrogated for the ability to survive under lipid production conditions in the presence of increasing levels of triparanol.

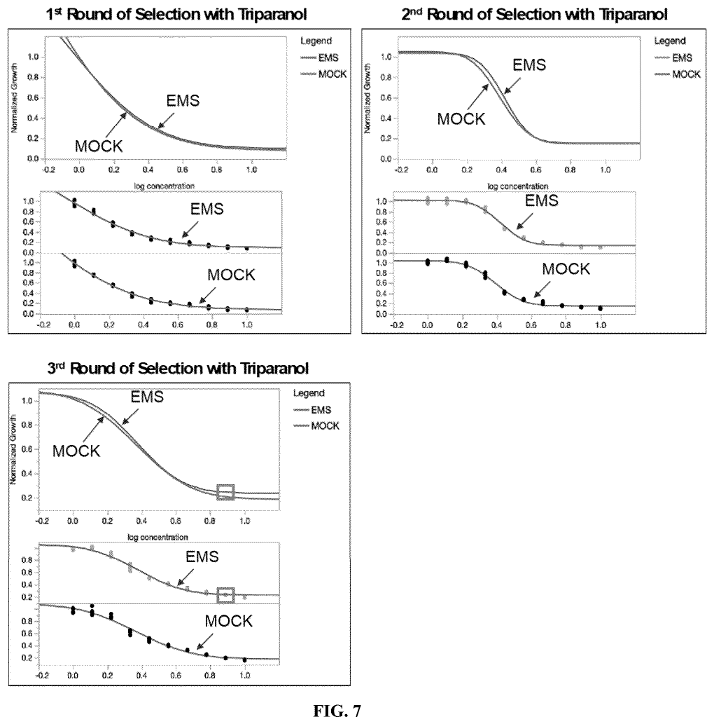

The cryo-vials were thawed and used to initiate a 10 mL pre-seed culture in bioreactor tube, which was incubated for 72 h at 28° C./200 rpm. Two rounds of selection and enrichment with increasing amounts of triparanol were conducted following the strategy outlined in FIG. 3. Following the third cycle of selection and enrichment, the mutant population began to significantly differentiate from the mock-mutagenized population when growth was measured during lipid production (FIG. 7). This population was collected on sterile filters, diluted, and spread on solid media. The resulting clonal isolates were screened in our automated 96-well block-based lipid production and glucose consumption assay. A number of strains with increased oleate levels were identified among the mutant clones (TABLE 5). Strains validated in repeat assays were then assessed for phenotypic stability. Stable strains were cryo-preserved.

TABLE 5

| Strain | % Oleate in Block |
|---|---|
| Strain K_C08 | 72.8 |
| Strain K _B01 | 72.2 |
| Strain K _C01 | 72.4 |
| Strain K _D03 | 72.3 |
| Strain K _D09 | 74.5 |
| Strain K _D11 | 72.6 |
| Strain 0 (average) | 58.2 |

TABLE 5 summarizes the oleate content of select mutant strains exhibiting higher glucose consumption in a 96-well block lipid assay. Mutant strains (approximately 2,200 isolates) were grown in lipid production medium (0.5 mL) with shaking (900 rpm) for 72 hours at 28° C. at which point glucose consumption was measured. Isolates with high glucose consumption levels were further interrogated for fatty acid composition with lead strains shown here.

FIG. 6 shows growth curves for selection for mutants with greater tolerance to membrane stress. Cells were exposed to a fixed concentration (1.6 μM) of triparanol and increasing concentrations of the membrane fluidizer phenethyl alcohol during a 3-day lipid production culture in a 96-deep well block. Significant inhibition of growth occurred at phenethyl alcohol concentrations >3.6 mM. Rare surviving cells were recovered by sterile filtration from wells treated with >6.3 mM phenethyl alcohol, washed with and collected from the filter in M20+glucose pre-seed media and the cultures used to initiate another round of selection for mutants with greater tolerance of membrane stress.

FIG. 7 shows growth curves for selection for mutants with greater tolerance to the triparanol. Cells were exposed to increasing amounts of triparanol during a 3-day lipid production culture in a 96-deep well block. Significant inhibition of growth occurred at phenethyl alcohol concentrations >2 μM. Rare surviving cells presumed to be in wells where 50% inhibition was observed were recovered by sterile filtration, washed with and collected from the filter in M20+glucose pre-seed media. The cultures were then used to initiate another round of selection. By the third round of selection (right panels), a subtle difference in the growth of the mutant population compared to the mock-mutagenized controls was observed. Cells were collected from the replicated wells treated with 7.7 μM triparanol (green boxes) and were plated to non-selective media to generate a mutant library for screening.

Lead strains where subsequently evaluated side-by-side in 10 mL lipid production cultures experiment conducted in 50-mL bioreactor tubes. As shown in TABLE 6, the best performing strain, designated as “Strain 2”, manifested >26% more oleate than Strain 1. Strain 2 also achieved a 15% higher lipid titer than Strain 1. Based on its performance in low cell density lipid assays in tubes, the performance of Strain 2 was then evaluated under high cell density fermentation conditions.

TABLE 6

| | Avg | Avg | Avg | | |
|---|---|---|---|---|---|
| | DCW | Oil Titer | NLB | Avg | % |
| Strain | (g/L) | (g/L) | (g/L) | PCP | C18:1 |
| Strain 0 | 15.0 | 10.4 | 4.6 | 2.2 | 58.98 |
| Strain 1 | 14.8 | 10.3 | 4.5 | 2.3 | 67.67 |
| Strain K_stbE06 | 16.60 | 12.0 | 4.6 | 2.6 | 72.16 |
| Strain K_stbE07 | 16.63 | 11.9 | 4.7 | 2.5 | 72.65 |
| Strain 2 | 17.3 | 11.7 | 5.6 | 2.1 | 74.59 |
| (Strain K_ stbA02) | | | | | |
| Strain K_stbC04 | 16.1 | 11.4 | 4.7 | 2.4 | 74.59 |

TABLE 6 summarizes results of tube based assays on classically improved derivatives of Strain 1 showing increased C18:1 content. All strains were run under standard lipid production conditions in which strains were grown in duplicate in 10 mL of lipid production medium. Cultures were grown with shaking (200 rpm) for 120 hours at 28° C. at which point 1 mL of biomass was removed, applied to a polycarbonate filter, washed with an equal volume of Milli Q water, and placed in a tared glass vial at −80° C. for 30 minutes. Vials containing filters and frozen biomass were lyophilized to dryness overnight, weights were recorded, and filters with dried biomass were subjected to direct transesterification followed by GC/FID to quantitate FAMEs.

A series of development runs utilizing Strain 2 were conducted in an effort to explore whether process conditions could further elevate C18:1 levels relative to the ca. 73-74% oleic obtained in tube and block based assays as described above. The results of these efforts are illustrated in TABLE 7 below. Reference runs showing Strain 0 and Strain 1 are also displayed, illustrating the best high oleic processes for these two strains as well. As shown in the data, the 220 mM nitrogen process that incorporated a Mg⁺⁺ and PO₄⁻ bolus post base transition, delivered the highest oleic content. At approximately 81% oleic content, a 5% increase in oleate over the previous high oleic strain, Strain 1, was achieved.

TABLE 7

| | | | DO set | | | | | Peak oil | Final oil | Final |
|---|---|---|---|---|---|---|---|---|---|---|
| | | Nitrogen | point | | | DCW | Oil | production | production | yield |
| Strain | Run No. | (mM) | (%) | Bolus | C18:1 | (g/L) | (g/L) | (g/Ld) | (g/Ld) | (g/g) |
| Strain 0 | 19003 | 220 | 30 | Mg⁺⁺, PO4⁻⁻⁻ | 69.54 | 142.9 | 83.5 | 17.97 | 14.52 | .209 |
| Strain 0 | 19004 | 220 | 30 | Mg⁺⁺, PO4⁻⁻⁻ | 68.86 | 142.8 | 84.1 | 17.15 | 14.62 | .219 |
| Strain 2 | 19021 | 220 | 50 | Mg⁺⁺, PO4⁻⁻⁻ | 80.56 | 135.2 | 71.3 | 15.88 | 12.39 | .191 |
| | | | | 2 min. starve | | | | | | |
| Strain 2 | 19022 | 220 | 50 | Mg⁺⁺, PO4⁻⁻⁻ | 80.80 | 144.9 | 76.1 | 17.52 | 13.23 | .201 |
| | | | | 2 min. starve | | | | | | |
| Strain 2 | 19023 | 220 | 50 | Mg⁺⁺, PO4⁻⁻⁻ | 72.15 | 61.4 | 20.5 | 5.2 | 4.50 | .100 |
| | | | | 4 min. starve | | | | | | |
| Strain 2 | 19024 | 220 | 50 | Mg⁺⁺, PO4⁻⁻⁻ | 65.2 | 49.4 | 13.9 | 3.0 | 3.10 | .080 |
| | | | | 4 min. starve | | | | | | |
| Strain 2 | 19025 | 220 | 50 | Mg⁺⁺, PO4⁻⁻⁻ | 80.63 | 138.3 | 80.7 | 17.57 | 14.00 | .200 |
| Strain 2 | 19026 | 220 | 50 | Mg⁺⁺, PO4⁻⁻⁻ | 80.85 | 136.1 | 78.1 | 17.58 | 13.53 | .205 |
| Strain 2 | 19027 | 220 | 50 | Mg⁺⁺, PO4⁻⁻⁻ | 80.86 | 141.2 | 80.2 | 16.25 | 13.75 | .192 |
| | | | | 2^{nd} Mg⁺⁺ | | | | | | |
| Strain 2 | 19028 | 220 | 50 | Mg⁺⁺, PO4⁻⁻⁻ | 81.29 | 137.8 | 78.8 | 16.68 | 13.51 | .207 |
| | | | | 2^{nd} Mg⁺⁺ | | | | | | |
| Strain 2 | 19029 | 170 | 50 | Complete | 78.30 | 169.0 | 109.0 | 26.58 | 19.74 | .234 |
| | | | | media | | | | | | |
| | | | | replacement | | | | | | |
| Strain 2 | 19030 | 170 | 50 | Complete | 78.25 | 168.0 | 112.2 | 25.58 | 17.18 | .245 |
| | | | | media | | | | | | |
| | | | | replacement | | | | | | |

TABLE 7 summarizes results of fermentation development runs for Strain 2 in comparison with the highest oleic content observed for Strain 0 and Strain 1 (all at 0.5-L scale). All runs were carried out at 0.5-L scale. Nitrogen refers to the total nitrogen used in the process (batched+fed ammonia). DO refers to dissolved oxygen. Mg⁺⁺ and PO₄⁻ bolus refers to a bolus (equivalent to the amount of each macronutrient that is batched in the initial vessel seed medium) that is administered just after base transition. Complete media replacement refers to administering a bolus (just after base transition) containing all batched media components with the exception of nitrogen. All broth samples for analysis were applied to a polycarbonate filter, washed with an equal volume of Milli Q water, and placed in a pre-weighed glass vial at −80° C. for 30 minutes. Vials containing filters and frozen biomass were lyophilized to dryness overnight, weights were recorded, and filters with dried biomass were subjected to direct transesterification followed by GC/FID to quantitate FAMEs.

### Example 3. Generation of a Classically Improved Microalgal Strain Capable of Producing a Triglyceride Oil that is Highly Enriched in Oleic Acid

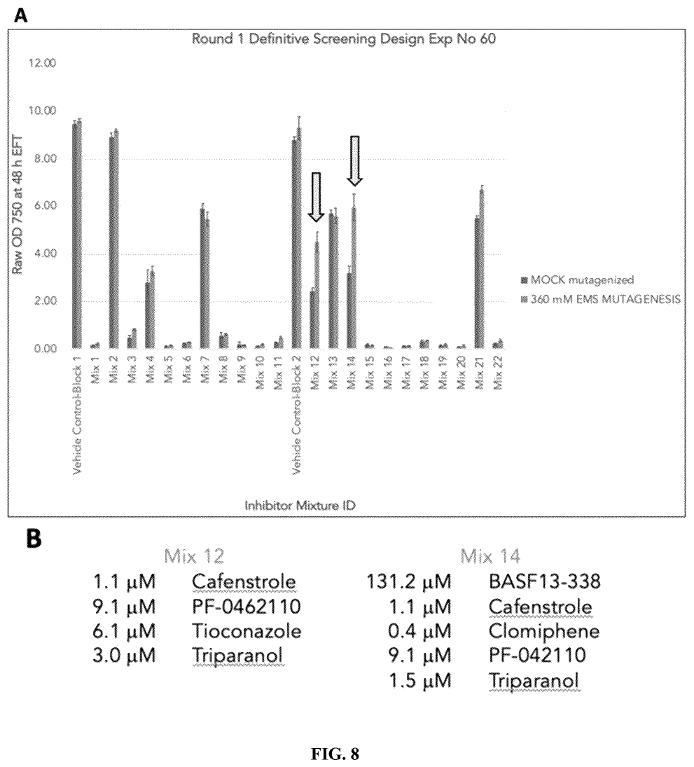

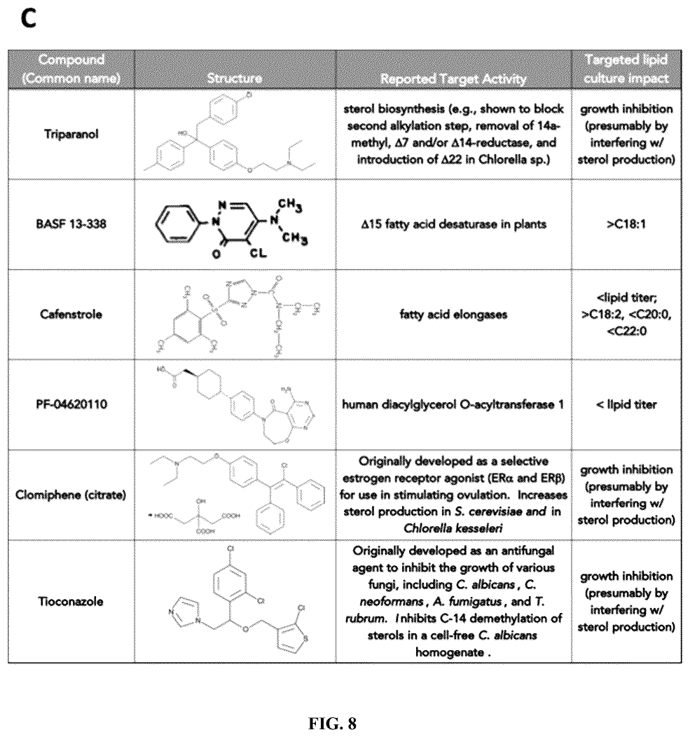

To further increase the oleate levels achieved in Strain 2 by classical strain improvement methods, a number of different cocktails of metabolic inhibitors and herbicides were tested with the goal of increasing genetic diversity in mutant libraries and biasing mutant populations toward higher oleate and/or increased lipid production. To generate a diverse library of mutants, Strain 2 was exposed to 360 mM ethyl methane sulfonate for 1 hour at room temperature, followed by inactivation of the mutagen and recovery of the cells after culture for 24 h. The resulting mutant population was subjected to a single round of enrichment with different cocktails of selective agents (FIG. 8, Panel A) that impact disparate aspects of fatty acid, triacylglycerol, or sterol production. Two selective agent cocktail-treated mutant cultures exposed to inhibitor mixtures 12 and 14 (FIG. 8, Panels A and B) grew >1.5-fold more than their paired, mock-treated control populations and were thus considered to have differentiated phenotypically. The mutant libraries derived from these cultures were plated on solid media to isolate individual mutant clones. These clones were initially screened for glucose consumption rates and those showing a statistically significant increase in glucose consumption compared to controls were analyzed for fatty acid profile. One isolate, M05_H03, showed a significant increase in oleate compared to the Strain 2 parental strain and was evaluated for phenotypic stability (TABLE 8). In initial stability evaluations, some instability in both profile and glucose consumption was observed. As such, the sub-clone manifesting the highest oleate level, M05_H03.D02, was selected for additional multi-generational sub-culture, and subsequently a re-evaluation of phenotypic stability. However, the sub-clone was shown to be unstable and was designated as “Strain 3”.

TABLE 8

| | | | | | | | Glucose |
|---|---|---|---|---|---|---|---|
| | | | | | | | consumption |
| Strain | C14:0 | C16:0 | C18:0 | C18:1 | C18:2 | C18:3 | (g/L * D) |
| Strain 0* | 1.43 ± 0.02 | 27.56 ± 0.09 | 3.48 ± 0.05 | 58.29 ± 0.21 | 7.32 ± 0.07 | 0.52 ± 0.01 | 6.4 ± 0.2 |
| Strain 2 ** | 1.01 ± 0.02 | 13.91 ± 0.22 | 3.13 ± 0.06 | 72.84 ± 0.31 | 7.53 ± 0.12 | 0.39 ± 0.01 | 7.4 ± 0.2 |
| Strain 3 | 0.64 | 8.22 | 2.79 | 78.89 | 8.21 | 0.31 | 6.0 |
| (M05_H03.D02) | | | | | | | |
| M05_H03.D03 | 0.65 | 8.22 | 3.01 | 78.62 | 8.32 | 0.40 | 6.8 |
| M05_H03.H04 | 0.68 | 8.33 | 2.84 | 78.56 | 8.19 | 0.37 | 6.0 |
| M05_H03.C05 | 0.86 | 10.99 | 3.41 | 75.48 | 7.88 | .40 | 6.0 |
| M05_H03.C04 | 0.66 | 8.13 | 2.97 | 78.45 | 8.30 | 0.42 | 7.2 |

*N = 8; α = 0.05
**N = 40; α = 0.05

TABLE 8 shows representative fatty acid profiles for sub-clones of a promising Strain 2 mutant generated through an additional round of classical strain improvement. All strains were run under standard lipid production conditions in a 96-well block format in which cells are grown in 0.5 mL of lipid production media. Cultures were grown with shaking (900 rpm) for 72 hours at 28° C., at which point 125 μL of biomass was removed, transferred to glass micro-vials, and frozen −80° C. for 30-60 minutes. The micro-vials containing frozen biomass were lyophilized to dryness overnight and then subjected to direct transesterification followed by GC/FID to quantitate FAMEs. The sub-clone, M05_H03.C05 (highlighted in bold italics), showed a significant deviation in fatty acid profile from the other sub-clones of the original mutant that was identified. Note that the glucose consumption rate in grams/liter*day (g/L*D) correlated linearly with lipid titer in this assay and also showed significant variability in this mutant clone. Given this phenotypic instability, one of the sub-clones that produced the highest level of oleate, M05_H03.D02 (also highlighted in bold), was selected for additional sub-culture stability re-evaluation.

FIG. 8 summarizes the classical strain improvement of Strain 2 by generation of diverse mutant libraries through exposure to different mixtures of metabolic inhibitors and herbicides. Following mutagenesis with ethyl methane sulfonate (EMS) and recovery, the resulting mutated cell populations were cultured under standard 96-well block lipid production conditions in 0.5 mL of lipid production media containing different combinations of inhibitors and/or herbicides. Mock-mutagenized cultures taken through all the same manipulations except for exposure to EMS were exposed to the same mixtures of inhibitors and herbicides in parallel. Cultures were grown with shaking (900 rpm) for 72 hours at 28° C. after which samples were removed from each well and diluted. Growth was assessed through optical density measurement at 750 nm in a 96-well plate reader (FIG. 8, Panel A). Error bars in the bar plot for each inhibitor mixture show the standard error derived from 4 replicate wells subjected to the same treatment. Cultures treated with inhibitor mixtures that showed a >1.5-fold higher level of growth compared to the mock-mutagenized control (yellow arrows; student t-test assuming unequal variances, α=0.05) were plated to solid media to generate clonal mutant libraries and screened. Mixtures 12 and 14 contained different combinations and concentrations of metabolic inhibitors (FIG. 8, Panel B) whose reported target activity and hypothesized mechanism of action in the lipid assay are described in the table in FIG. 8, Panel C.

### Example 4. Generation of a Classically Improved Microalgal Strain Capable of Producing a Triglyceride Oil that is Highly Enriched in Oleic Acid

As an alternative to stabilizing Strain 3, a new round of mutagenesis was pursued for Strain 2 utilizing treatment of cells with the mutagen 4-nitroquinoline-1-oxide (4-NQO) for 5 minutes at 28° C. Mutagenized cells were enriched by growing under conditions of limited glucose (14 g/L) for three days, then the cells were subjected to fraction over a 60% Percoll/0.15 M NaCl density gradient. Cells recovered from a density zone of 1.06 g/mL were plated and assessed for glucose consumption and fatty acid profile. One of these clones was subsequently stabilized and given the strain designation “Strain 4”.

### Example 5. Generation of a Classically Improved Microalgal Strain Capable of Producing a Triglyceride Oil that is Highly Enriched in Oleic Acid

A subpopulation of the Strain 4 lineage was concurrently subjected to an enrichment strategy employing one round of enrichment in an inhibitor cocktail (Inhibitor Cocktail 2 in FIG. 8, Panel B) comprised of 0.115 μM clomiphene citrate, 12.35 μM terfenadine, 18.582 μM fluphenazine, and 1.654 μM triparanol. Cells recovered from the enrichment were plated and subsequently assessed for glucose consumption and fatty acid profile in a 72-hour, 96-well block based format. One of these clones was subsequently stabilized and given the strain designation “Strain 5”.

### Example 6. Generation of a Classically Improved Microalgal Strain Capable of Producing a Triglyceride Oil that is Highly Enriched in Oleic Acid

Strain 5 was subjected to another round of mutagenesis with increasing concentrations and exposure time to 4-NQO (37 μM for 30 minutes at 28° C.). This population of cells was subsequently subdivided and grown in standard lipid production medium supplemented with a range of cerulenin concentrations (7-50 μM). Cells from all concentrations were pooled and fractionated over a 60% Percoll/0.15 M NaCl density gradient. Oil laden cells recovered from a density zone of 1.02 g/mL were plated and assessed for glucose consumption and fatty acid profile. One of these clones was subsequently stabilized and given the strain designation “Strain 6”.

### Example 7. Generation of a Classically Improved Microalgal Strain Capable of Producing a Triglyceride Oil that is Highly Enriched in Oleic Acid

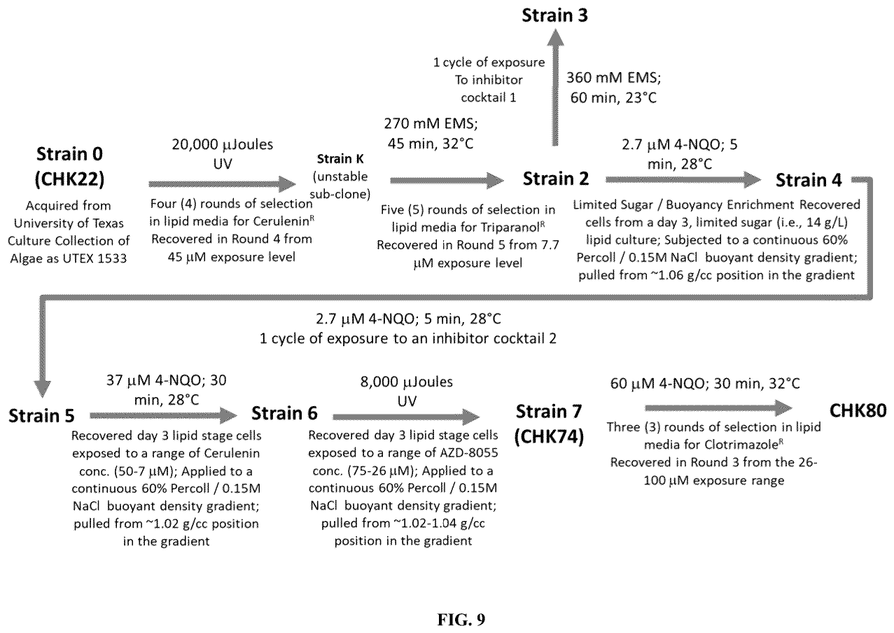

Strain 6 was subsequently mutagenized with 8,000 μJoules of UV radiation. The resulting population of cells were subdivided and grown in standard lipid production medium supplemented with a range of concentrations (26-75 μM) of the mTOR (mammalian target of rapamycin) ATP-competitive inhibitor, AZD8055. Cells from all concentrations were pooled and fractionated over a 60% Percoll/0.15 M NaCl density gradient. Oil laden cells recovered from a density zone of 1.02-1.04 g/mL were plated and assessed for glucose consumption and fatty acid profile. One of these clones was subsequently stabilized and given the strain designation “Strain 7”. TABLE 9 shows the fatty acid profiles of strains depicted in FIG. 9 from a 3-day, 96-well block lipid assay.

TABLE 9

| Sample | | | | | | | C18:3 | | |
|---|---|---|---|---|---|---|---|---|---|
| Name | C14:0 | C16:0 | C16:1 | C18:0 | C18:1 | C18:2 | alpha | C20:0 | C20:1 |
| Strain 0 | 1.38 | 27.40 | 0.85 | 3.33 | 58.32 | 6.70 | 0.51 | 0.40 | 0.20 |
| Strain K | 0.76 | 15.21 | 0.61 | 3.53 | 70.62 | 7.23 | 0.51 | 0.42 | 0.16 |
| Strain 2 | 1.00 | 13.84 | 0.59 | 2.89 | 73.21 | 7.48 | 0.36 | 0.15 | 0.12 |
| Strain 3 | 0.87 | 9.76 | 0.51 | 2.43 | 74.83 | 10.30 | 0.48 | 0.21 | 0.33 |
| Strain 4 | 0.52 | 11.09 | 0.58 | 2.38 | 77.53 | 6.98 | 0.29 | 0.14 | 0.29 |
| Strain 5 | 0.51 | 8.45 | 0.42 | 2.77 | 78.78 | 7.90 | 0.38 | 0.12 | 0.37 |
| Strain 6 | 0.31 | 5.75 | 0.30 | 2.67 | 81.45 | 7.83 | 0.34 | 0.13 | 0.66 |
| Strain 7 | 0.28 | 5.29 | 0.27 | 2.45 | 82.58 | 7.78 | 0.34 | 0.11 | 0.63 |

FIG. 9 shows an overview of classical strain improvement and provenance of Strain 7 by generation of mutants derived from UTEX 1533 using mutagens and a series of selective agents and enrichment strategies. Inhibitor cocktail 1 (Strain 2 to Strain 3) comprised of 131.2 μM BASF 13-338, 1.1 μM cafenstrole, 0.4 μM clomiphene, 9.1 μM PF-042110, and 1.5 μM triparanol. Inhibitor cocktail 2 (Strain 4 to Strain 5) comprised of 0.115 μM clomiphene citrate, 12.35 μM terfenadine, 18.582 μM fluphenazine, and 1.654 μM triparanol.

TABLE 10 shows the fatty acid profiles of various strains described herein.

TABLE 10

| | | | | | | Titer |
|---|---|---|---|---|---|---|
| Strain | Mutagen | Enrichment | Scale | Process | % Oleic | (g/L) |
| Strain 0 | NA | NA | 10 mL | Low Cell | 59.61 | 9.2 |
| | | | | Density | (±0.30) | (±0.3) |
| Strain 0 | | | 1 L | High | 62.08 | 105.4 |
| | | | | Productivity | | |
| Strain 0 | | | 1 L | High Oleic | 69.07 | 86.1 |
| Strain 2 | EMS | Triparanol | 10 mL | Low Cell | 74.22 | 10.1 |
| | | | | Density | (±0.23) | (±0.4) |
| Strain 2 | | | 1 L | High | 74.21 | 123.4 |
| | | | | Productivity | | |
| Strain 2 | | | 1 L | High Oleic | 80.22 | 98.4 |
| Strain 2 | | | 300 L | High Oleic | 83.75 | 77.5 |
| Strain 4 | Strain 2 treated | LSBE (Limited | 10 mL | Low Cell | 77.75 | 10.1 |
| | with 4-NQO | Sugar Buoyancy | | Density | (±0.14) | (±0.5) |
| Strain 4 | (Nitroquinoline | Enrichment) | 1 L | High | 76.32 | 118.1 |
| | 4-oxide) | | | Productivity | | |
| Strain 4 | | | 1 L | High Oleic | 79.89 | 79.7 |
| Strain 5 | Strain 4 treated | DOE mix (1.654 | 10 mL | Low Cell | 81.87 | 10.3 |
| | with 2.7 NQO | μM Triparanol | | Density | (±0.11) | (±0.4) |
| | | 0.115 μM | | | | |
| Strain 5 | | Clomiphene | 1 L | High | 80.24 | 135.2 |
| | | 12.35 μM | | Productivity | | |
| | | Terfenadine | | | | |
| Strain 5 | | 18.582 μM | 1 L | High Oleic | 82.86 | 85.4 |
| | | Fluphenazine) | | | | |

### Example 8. Generation of a Classically Improved Microalgal Strain (CHK80) Capable of Producing a Triglyceride Oil that is Highly Enriched in Oleic Acid

Strain 7 (CHK74) described in Example 7 was further subjected to classical strain improvement to produce the strain “CHK80”. CHK74 was mutagenized for 30 min at 32° C. with 40 μM or 60 μM 4-NQO or subjected to sham mutagenesis with the addition of only the mutagen solvent, 0.06% DMSO. The two mutagenized and the mock-mutagenized populations were each independently cultured in lipid production media supplemented with different concentrations of the inhibitor, clomiphene, along with 2.44 g/L Tween-40, 2.49 g/L Tween-60, & 0.2 g/L linoleic acid. The Tween-emulsified linoleic acid was added to enrich for mutants that inherently produce lower levels of C18:2. After the first cycle of exposure, mutant cells exposed to concentrations of clomiphene where growth was reduced >50% based on OD750 measurements were collected, pooled, washed free of the inhibitor, and subjected to another round of clomiphene exposure for 72 h in lipid media with linoleic supplementation, each time increasing the range of clomiphene exposures. By the third cycle of exposure, a resistant population arose from the 60 μM 4-NQO mutant library compared to both the 40 μM 4-NQO mutant population or the mock-mutagenized controls (FIG. 9). This population was collected, washed free of inhibitor, expanded in pre-seed media for 3 days and plated for single colonies. Mutant clones were then picked and assessed for glucose consumption and fatty acid profile in a 72 h, 96-well block-based lipid production assay. A mutant identified in the screen was subsequently stabilized and given the strain designation “CHK80”.

TABLE 11 shows sequence alignments of genes involved in fatty acid biosynthesis in CHK80 and CHK22 (Strain 0; original progenitor strain): FAD2-1, FAD2-2, FATA1-1, FATA1-2, KASII-1, KASII-2, SAD2-1, and SAD2-2.

TABLE 11
FAD2-1_CHK80 cggcgcgctgcttcgcgtgccgggtgcagcaatcagatccaagtctgacgacttgcgcgc
(SEQ ID NO: 1)
FAD2-1_CHK22 cggcgcgctgcttcgcgtgccgggtgcagcaatcagatccaagtctgacgacttgcgcgc
(SEQ ID NO: 2)
FAD2-1_CHK80 acgcgccggatccttcaattccaaagtgtcgtccgcgtgcgcttcttcgccttcgtcctc
FAD2-1_CHK22 acgcgccggatccttcaattccaaagtgtcgtccgcgtgcgcttcttcgccttcgtcctc
FAD2-1_CHK80 ttgaacatccagcgacgcaagcgcagggcgctgggcggctggcgtcccgaaccggcctcg
FAD2-1_CHK22 ttgaacatccagcgacgcaagcgcagggcgctgggcggctggcgtcccgaaccggcctcg
FAD2-1_CHK80 gcgcacgcggctgaaattgccgatgtcggcaatgtagtgccgctccgcccacctctcaat
FAD2-1_CHK22 gcgcacgcggctgaaattgccgatgtcggcaatgtagtgccgctccgcccacctctcaat
FAD2-1_CHK80 taagtttttcagcgcgtggttgggaatgatctgcgctcatggggcgaaagaaggggttca
FAD2-1_CHK22 taagtttttcagcgcgtggttgggaatgatctgcgctcatggggcgaaagaaggggttca
FAD2-1_CHK80 gaggtgctttattgttactcgactgggcgtaccagcattcgtgcatgactgattatacat
FAD2-1_CHK22 gaggtgctttattgttactcgactgggcgtaccagcattcgtgcatgactgattatacat
FAD2-1_CHK80 acaaaagtacagctcgcttcaatgccctgcgattcctactcccgagcgagcactcctctc
FAD2-1_CHK22 acaaaagtacagctcgcttcaatgccctgcgattcctactcccgagcgagcactcctctc
FAD2-1_CHK80 accgtcgggttgcttcccacgaccacgccggtaagagggtctgtggcctcgcgcccctcg
FAD2-1_CHK22 accgtcgggttgcttcccacgaccacgccggtaagagggtctgtggcctcgcgcccctcg
FAD2-1_CHK80 cgagcgcatctttccagccacgtctgtatgattttgcgctcatacgtctggcccgtcgac
FAD2-1_CHK22 cgagcgcatctttccagccacgtctgtatgattttgcgctcatacgtctggcccgtcgac
FAD2-1_CHK80 cccaaaatgacgggatcctgcataatatcgcccgaaatgggatccaggcattcgtcagga
FAD2-1_CHK22 cccaaaatgacgggatcctgcataatatcgcccgaaatgggatccaggcattcgtcagga
FAD2-1_CHK80 ggcgtcagccccgcgggagatgccggtcccgccgcattggaaaggtgtagagggggtgaa
FAD2-1_CHK22 ggcgtcagccccgcgggagatgccggtcccgccgcattggaaaggtgtagagggggtgaa
FAD2-1_CHK80 tcccccatttcatgaaatgcttggtcaacgatggtgcgcattcgtgcaaagtgaatatgg
FAD2-1_CHK22 tcccccatttcatgaaatgcttggtcaacgatggtgcgcattcgtgcaaagtgaatatgg
FAD2-1_CHK80 ggtcacgcggtggacgaacgcggagggggcatgaccgaatctaggctcgcattcctcaga
FAD2-1_CHK22 ggtcacgcggtggacgaacgcggagggggcatgaccgaatctaggctcgcattcctcaga
FAD2-1_CHK80 tcacttcatgccggcggtccggggtttgcgcgtcgcgcaaggctacgtctccctagccgc
FAD2-1_CHK22 tcacttcatgccggcggtccggggtttgcgcgtcgcgcaaggctacgtctccctagccgc
FAD2-1_CHK80 tgcgcaccacgcgtgcgacgcggaggccatcttccggagcaacgaccatggattgtctta
FAD2-1_CHK22 tgcgcaccacgcgtgcgacgcggaggccatcttccggagcaacgaccatggattgtctta
FAD2-1_CHK80 gcgatcgcacgaatgagtgctagtgagtcgtacgctcgacccagtcgctcgcaggagaag
FAD2-1_CHK22 gcgatcgcacgaatgagtgctagtgagtcgtacgctcgacccagtcgctcgcaggagaag
FAD2-1_CHK80 gcggcagctgccgagcttcggcttaccagtcgtgactcgtatgtgatcaggaatcattgg
FAD2-1_CHK22 gcggcagctgccgagcttcggcttaccagtcgtgactcgtatgtgatcaggaatcattgg
FAD2-1_CHK80 cattggtagcattataattcggcttccgcgctgcgtatgggcatggcaatgtctcatgca
FAD2-1_CHK22 cattggtagcattataattcggcttccgcgctgcgtatgggcatggcaatgtctcatgca
FAD2-1_CHK80 gtcgatcttagtcaaccaattttgggtggccaggtccgggcgaccgggctccgtgtcgcc
FAD2-1_CHK22 gtcgatcttagtcaaccaattttgggtggccaggtccgggcgaccgggctccgtgtcgcc
FAD2-1_CHK80 gggcaccacctcctgccaggagtagcagggccgccctctcgtcccgacgttggcccactg
FAD2-1_CHK22 gggcaccacctcctgccaggagtagcagggccgccctctcgtcccgacgttggcccactg
FAD2-1_CHK80 aataccgtggcttcgagccctacatgatgggctgcctagtcgggcgggacgcgcaactgc
FAD2-1_CHK22 aataccgtggcttcgagccctacatgatgggctgcctagtcgggcgggacgcgcaactgc
FAD2-1_CHK80 ccgcgcgatctgggggctggtctgaatccttcaggcgggtgttacccgagaaagaaaggg
FAD2-1_CHK22 ccgcgcgatctgggggctggtctgaatccttcaggcgggtgttacccgagaaagaaaggg
FAD2-1_CHK80 tgccgatttcaaagcagacccatgtgccgggccctgtggcctgtgttggcgcctatgtag
FAD2-1_CHK22 tgccgatttcaaagcagacccatgtgccgggccctgtggcctgtgttggcgcctatgtag
FAD2-1_CHK80 tcaccccccctcacccaattgtcgccagtttgcgcactccataaactcaaaacagcagct
FAD2-1_CHK22 tcaccccccctcacccaattgtcgccagtttgcgcactccataaactcaaaacagcagct
FAD2-1_CHK80 tctgagctgcgctgttcaagaacacctctggggtttgctcacccgcgaggtcgacgccca
FAD2-1_CHK22 tctgagctgcgctgttcaagaacacctctggggtttgctcacccgcgaggtcgacgccca
FAD2-1_CHK80 gcATGGCTATCAAGACGAACAGGCAGCCTGTGGAGAAGCCTCCGTTCACGATCGGGACGC
FAD2-1_CHK22 gcATGGCTATCAAGACGAACAGGCAGCCTGTGGAGAAGCCTCCGTTCACGATCGGGACGC
FAD2-1_CHK80 TGCGCAAGGCCATCCCCGCGCACTGTTTCGAGCGCTCGGCGCTTCGTAGCAGCATGTACC
FAD2-1_CHK22 TGCGCAAGGCCATCCCCGCGCACTGTTTCGAGCGCTCGGCGCTTCGTAGCAGCATGTACC
FAD2-1_CHK80 TGGCCTTTGACATCGCGGTCATGTCCCTGCTCTACGTCGCGTCGACGTACATCGACCCTG
FAD2-1_CHK22 TGGCCTTTGACATCGCGGTCATGTCCCTGCTCTACGTCGCGTCGACGTACATCGACCCTG
FAD2-1_CHK80 CACCGGTGCCTACGTGGGTCAAGTACGGCATCATGTGGCCGCTCTACTGGTTCTTCCAGG
FAD2-1_CHK22 CACCGGTGCCTACGTGGGTCAAGTACGGCATCATGTGGCCGCTCTACTGGTTCTTCCAGG
FAD2-1_CHK80 TGTGTTTGAGGGTTTTGGTTGCCCGTATTGAGGTCCTGGTGGCGCGCATGGAGGAGAAGG
FAD2-1_CHK22 TGTGTTTGAGGGTTTTGGTTGCCCGTATTGAGGTCCTGGTGGCGCGCATGGAGGAGAAGG
FAD2-1_CHK80 CGCCTGTCCCGCTGACCCCCCCGCCGACCCTCCCCCCACCTTCCAGGGCGCCTTCGGCAC
FAD2-1_CHK22 CGCCTGTCCCGCTGACCCCCCCGCCGACCCTCCCCC-ACCTTCCAGGGCGCCTTCGGCAC
FAD2-1_CHK80 GGGTGTCTGGGTGTGCGCGCACGAGTGCGGTCACCAGGCCTTTTCCTCCAGCCAGGCCAT
FAD2-1_CHK22 GGGTGTCTGGGTGTGCGCGCACGAGTGCGGTCACCAGGCCTTTTCCTCCAGCCAGGCCAT
FAD2-1_CHK80 CAACGACGGCGTGGGCCTGGTGTTCCACAGCCTGCTGCTGGTGCCCTACTACTCCTGGAA
FAD2-1_CHK22 CAACGACGGCGTGGGCCTGGTGTTCCACAGCCTGCTGCTGGTGCCCTACTACTCCTGGAA
FAD2-1_CHK80 GCACTCGCACCGCCGCCACCACTCCAACACGGGGTGCCTGGACAAGGACGAGGTGTTTGT
FAD2-1_CHK22 GCACTCGCACCGCCGCCACCACTCCAACACGGGGTGCCTGGACAAGGACGAGGTGTTTGT
FAD2-1_CHK80 GCCGCCGCACCGTGCGGTGGCGCACGAGGGCCTGGAGTGGGAGGAGTGGCTGCCCATCCG
FAD2-1_CHK22 GCCGCCGCACCGTGCGGTGGCGCACGAGGGCCTGGAGTGGGAGGAGTGGCTGCCCATCCG
FAD2-1_CHK80 CATGGGCAAGGTGCTGGTCACCTTGACCCTGGGCTGGCCGCTGTACCTCATGTTCAACGT
FAD2-1_CHK22 CATGGGCAAGGTGCTGGTCACCTTGACCCTGGGCTGGCCGCTGTACCTCATGTTCAACGT
FAD2-1_CHK80 CGCCTCCCGCCCTTACCCGCGCTTCGCCAACCACTTTGACCCGTGGTCGCCCATCTTCAG
FAD2-1_CHK22 CGCCTCCCGCCCTTACCCGCGCTTCGCCAACCACTTTGACCCGTGGTCGCCCATCTTCAG
FAD2-1_CHK80 CAAGCGCGAGCGCATCGAGGTGGTCATCTCCGACCTCGCGTTGGTGGCGGTGCTCAGCGG
FAD2-1_CHK22 CAAGCGCGAGCGCATCGAGGTGGTCATCTCCGACCTCGCGTTGGTGGCGGTGCTCAGCGG
FAD2-1_CHK80 GCTCAGCGTGCTGGGCCGCACCATGGGCTGGGCCTGGCTGGTCAAGACCTACGTGGTGCC
FAD2-1_CHK22 GCTCAGCGTGCTGGGCCGCACCATGGGCTGGGCCTGGCTGGTCAAGACCTACGTGGTGCC
FAD2-1_CHK80 CTACATGATCGTGAACATGTGGCTGGTGCTCATCACGCTGCTCCAGCACACGCACCCGGC
FAD2-1_CHK22 CTACATGATCGTGAACATGTGGCTGGTGCTCATCACGCTGCTCCAGCACACGCACCCGGC
FAD2-1_CHK80 CCTGCCGCACTACTTCGAGAAGGACTGGGACTGGCTACGCGGCGCCATGGCCACCGTCGA
FAD2-1_CHK80 CCTGCCGCACTACTTCGAGAAGGACTGGGACTGGCTACGCGGCGCCATGGCCACCGTCGA
FAD2-1_CHK80 CCGCTCCATGGGCCCGCCCTTCATGGACAGCATCCTGCACCACATCTCCGACACCCACGT
FAD2-1_CHK22 CCGCTCCATGGGCCCGCCCTTCATGGACAGCATCCTGCACCACATCTCCGACACCCACGT
FAD2-1_CHK80 GCTGCACCACCTCTTCAGCACCATCCCGCACTACCACGCCGAGGAGGCCTCCGCCGCCAT
FAD2-1_CHK22 GCTGCACCACCTCTTCAGCACCATCCCGCACTACCACGCCGAGGAGGCCTCCGCCGCCAT
FAD2-1_CHK80 CCGGCCCATCCTGGGCAAGTACTACCAATCCGACAGCCGCTGGGTCGGCCGCGCCCTGTG
FAD2-1_CHK22 CCGGCCCATCCTGGGCAAGTACTACCAATCCGACAGCCGCTGGGTCGGCCGCGCCCTGTG
FAD2-1_CHK80 GGAGGACTGGCGCGACTGCCGCTACGTCGTCCCCGACGCGCCCGAGGACGACTCCGCGCT
FAD2-1_CHK22 GGAGGACTGGCGCGACTGCCGCTACGTCGTCCCCGACGCGCCCGAGGACGACTCCGCGCT
FAD2-1_CHK80 CTGGTTCCACAAGTGAgcgcgcctgcgcgaggacgcagaacaacgctgccgccgtgtctt
FAD2-1_CHK22 CTGGTTCCACAAGTGAgcgcgcctgcgcgaggacgcagaacaacgctgccgccgtgtctt
FAD2-1_CHK80 ttgcacgcgcgactccggcgcttcgctggtggcacccccataaagaaaccctcaattctg
FAD2-1_CHK22 ttgcacgcgcgactccggcgcttcgctggtggcacccccataaagaaaccctcaattctg
FAD2-1_CHK80 tttgtggaagacacggtgtacccccacccacccacctgcacctctattattggtattatt
FAD2-1_CHK22 tttgtggaagacacggtgtacccccacccacccacctgcacctctattattggtattatt
FAD2-1_CHK80 gacgcgggagtgggcgttgtaccctacaacgtagcttctctagttttcagctggctccca
FAD2-1_CHK22 gacgcgggagtgggcgttgtaccctacaacgtagcttctctagttttcagctggctccca
FAD2-1_CHK80 ccattgtaaattcatgctagaatagtgcgtggttatgtgagaggtatagtgtgtctgagc
FAD2-1_CHK22 ccattgtaaattcatgctagaatagtgcgtggttatgtgagaggtatagtgtgtctgagc
FAD2-1_CHK80 agacggggcgggatgcatgtcgtggtggtgatctttggctcaaggcgtcgtcgacgtgac
FAD2-1_CHK22 agacggggcgggatgcatgtcgtggtggtgatctttggctcaaggcgtcgtcgacgtgac
FAD2-1_CHK80 gtgcccgatcatgagagcaataccgcgctcaaagccgacgcatagcctttactccgcaat
FAD2-1_CHK22 gtgcccgatcatgagagcaataccgcgctcaaagccgacgcatagcctttactccgcaat
FAD2-1_CHK80 ccaaacgactgtcgctcgtattttttggatatctattttaaagagcgagcacagcgccgg
FAD2-1_CHK22 ccaaacgactgtcgctcgtattttttggatatctattttaaagagcgagcacagcgccgg
FAD2-1_CHK80 gcatgggcctgaaaggcctcgcggccgtgctcgtggtgggggccgcgagcgcgtggggca
FAD2-1_CHK22 gcatgggcctgaaaggcctcgcggccgtgctcgtggtgggggccgcgagcgcgtggggca
FAD2-1_CHK80 tcgcggcagtgcaccaggcgcagacggaggaacgcatggtgcgtgcgcaatataagatac
FAD2-1_CHK22 tcgcggcagtgcaccaggcgcagacggaggaacgcatggtgcgtgcgcaatataagatac
FAD2-1_CHK80 atgtattgttgtctgtactataatgctagagcatcaccaggggcttagtcatcgcacctg
FAD2-1_CHK22 atgtattgttgtctgtactataatgctagagcatcaccaggggcttagtcatcgcacctg
FAD2-1_CHK80 ctttggtcattacagaaattgcacaagggcgtcctccgggatgaggagatgtaccagctc
FAD2-1_CHK22 ctttggtcattacagaaattgcacaagggcgtcctccgggatgaggagatgtaccagctc
FAD2-1_CHK80 aagctggagcggcttcgagccaagcaggagcgcggcgcatgacgacctatccacatgtga
FAD2-1_CHK22 aagctggagcggcttcgagccaagcaggagcgcggcgcatgacgacctatccacatgtga
FAD2-1_CHK80 caaaggggtctgggtcgtacgaatcgacaaaccagtcaggagacagcggggtccatgagt
FAD2-1_CHK22 caaaggggtctgggtcgtacgaatcgacaaaccagtcaggagacagcggggtccatgagt
FAD2-1_CHK80 tggcccgctcgcagcttcagcgctttgagcatcgcggcattgtccacgatccaaggctcc
FAD2-1_CHK22 tggcccgctcgcagcttcagcgctttgagcatcgcggcattgtccacgatccaaggctcc
FAD2-1_CHK80 cgcagcgcctccttgggacccctgtgcggagaagcgtgtgccgagagggctgtgagggcg
FAD2-1_CHK22 cgcagcgcctccttgggacccctgtgcggagaagcgtgtgccgagagggctgtgagggcg
FAD2-1_CHK80 gaggctggaggagaggctgcgaatcattgcgctgcctaggcgtctgccgagtagtagcac
FAD2-1_CHK22 gaggctggaggagaggctgcgaatcattgcgctgcctaggcgtctgccgagtagtagcac
FAD2-1_CHK80 accacgcgccggtgctttcatttcattgtgtccgttgctgtaatgcatgcgagcaatcct
FAD2-1_CHK22 accacgcgccggtgctttcatttcattgtgtccgttgctgtaatgcatgcgagcaatcct
FAD2-1_CHK80 gcactaaaagctgcgatcaccacctcggcgttgttggtctctagtaccgctcacctgacc
FAD2-1_CHK22 gcactaaaagctgcgatcaccacctcggcgttgttggtctctagtaccgctcacctgacc
FAD2-1_CHK80 acgcgcacggaagtatggcggtcggcgacggcctgctgcacgtggcagtgcagatccacg
FAD2-1_CHK22 acgcgcacggaagtatggcggtcggcgacggcctgctgcacgtggcagtgcagatccacg
FAD2-1_CHK80 tggcccaggagcaggtctcgcagctgcaacaccgcgccctcgtgctcgccggggtccatc
FAD2-1_CHK22 tggcccaggagcaggtctcgcagctgcaacaccgcgccctcgtgctcgccggggtccatc
FAD2-1_CHK80 cagtccataccccgcgccgggccctcgagccggacggccagcagccagggccgggggtcc
FAD2-1_CHK22 cagtccataccccgcgccgggccctcgagccggacggccagcagccagggccgggggtcc
FAD2-1_CHK80 gcgccgacgcccggcggccccgccgcgcagctggggtcggccaggaagagccactggacg
FAD2-1_CHK22 gcgccgacgcccggcggccccgccgcgcagctggggtcggccaggaagagccactggacg
FAD2-1_CHK80 gccggcccgggcgagccccgcgccggcgtgggacccacccgcagcaccaccgcctgctcc
FAD2-1_CHK22 gccggcccgggcgagccccgcgccggcgtgggacccacccgcagcaccaccgcctgctcc
FAD2-1_CHK80 aggtccacgtcgctcggcggcaggcccgcgtgcggcgcctgcgccagcgtccagggcagg
FAD2-1_CHK22 aggtccacgtcgctcggcggcaggcccgcgtgcggcgcctgcgccagcgtccagggcagg
FAD2-1_CHK80 cggggccgggcgtgcatg
FAD2-1_CHK22 cggggccgggcgtgcatg
FAD2-2_CHK80 cggctcgctgctttgcgtgccgggtgcagcgatcagatccaagtctgacgacttgtgctg
(SEQ ID NO: 3)
FAD2-2_CHK22 cggctcgctgctttgcgtgccgggtgcagcgatcagatccaagtctgacgacttgtgctg
(SEQ ID NO: 4)
FAD2-2_CHK80 atgtactgtgtcctttgagtccagagcgccggccgcacgcgcttcttccccttcttcttc
FAD2-2_CHK22 atgtactgtgtcctttgagtccagagcgccggccgcacgcgcttcttccccttcttcttc
FAD2-2_CHK80 ctctcgaacatccagcgatgcaagtgcagggcgctgggcggctggcgtcccgaaccggcc
FAD2-2_CHK22 ctctcgaacatccagcgatgcaagtgcagggcgctgggcggctggcgtcccgaaccggcc
FAD2-2_CHK80 tcggcgcacgcggctgaaattgccaatgtcggcaatgtagtgccgctcggcccatccctc
FAD2-2_CHK22 tcggcgcacgcggctgaaattgccaatgtcggcaatgtagtgccgctcggcccatccctc
FAD2-2_CHK80 gatcaagtttttcagcgcgtggttggggatgatctgcgctcatgggaagataaaaggggt
FAD2-2_CHK22 gatcaagtttttcagcgcgtggttggggatgatctgcgctcatgggaagataaaaggggt
FAD2-2_CHK80 tctgaggtggttgattggtactttaccggaagtactcatattcatacatgactgatccca
FAD2-2_CHK22 tctgaggtggttgattggtactttaccggaagtactcatattcatacatgactgatccca
FAD2-2_CHK80 cacaaaaacaaagctcacttcaaagaaccgcgcatgtctactccccagcaatcacttcgc
FAD2-2_CHK22 cacaaaaacaaagctcacttcaaagaaccgcgcatgtctactccccagcaatcacttcgc
FAD2-2_CHK80 tcaccgtcgggttgcttcccacgacaacgccggtgagagggtcggtggcctcgcgacctt
FAD2-2_CHK22 tcaccgtcgggttgcttcccacgacaacgccggtgagagggtcggtggcctcgcgacctt
FAD2-2_CHK80 cgcgggcacatctttccagccatgtctgtataatctcacgctcatacgtctggcccgtcg
FAD2-2_CHK22 cgcgggcacatctttccagccatgtctgtataatctcacgctcatacgtctggcccgtcg
FAD2-2_CHK80 accccaaaatgacgggatcctgcatgatatcgcccgagatggggtccaggcattcctctg
FAD2-2_CHK22 accccaaaatgacgggatcctgcatgatatcgcccgagatggggtccaggcattcctctg
FAD2-2_CHK80 gaggcgtcagccctgcgggagatgccggtcccaccgcattggaaaggcacaaagggggtg
FAD2-2_CHK22 gaggcgtcagccctgcgggagatgccggtcccaccgcattggaaaggcacaaagggggtg
FAD2-2_CHK80 aatcccccatttcatgaaattgttggtcagcgatggtgcgcactcgtgcgcaatgaatat
FAD2-2_CHK22 aatcccccatttcatgaaattgttggtcagcgatggtgcgcactcgtgcgcaatgaatat
FAD2-2_CHK80 ggggtcacgcggtggacgaacgcggagggggcctggccgaatctaggcttgcattcctca
FAD2-2_CHK22 ggggtcacgcggtggacgaacgcggagggggcctggccgaatctaggcttgcattcctca
FAD2-2_CHK80 gatcactttctgccggcggtccggggtttgcgcgtcgcgcaacgctccgtctccctagcc
FAD2-2_CHK22 gatcactttctgccggcggtccggggtttgcgcgtcgcgcaacgctccgtctccctagcc
FAD2-2_CHK80 gctgcgcaccgcgcgtgcgacgcgaaggtcattttccagaacaacgaccatggcttgtct
FAD2-2_CHK22 gctgcgcaccgcgcgtgcgacgcgaaggtcattttccagaacaacgaccatggcttgtct
FAD2-2_CHK80 tagcgatcgctcgaatgactgctagtgagtcgtacgctcgacccagtcgctcgcaggaga
FAD2-2_CHK22 tagcgatcgctcgaatgactgctagtgagtcgtacgctcgacccagtcgctcgcaggaga
FAD2-2_CHK80 acgcggcaactgccgagcttcggcttgccagtcgtgactcgtatgtgatcaggaatcatt
FAD2-2_CHK22 acgcggcaactgccgagcttcggcttgccagtcgtgactcgtatgtgatcaggaatcatt
FAD2-2_CHK80 ggcattggtagcattataattcggcttccgcgctgtttatgggcatggcaatgtctcatg
FAD2-2_CHK22 ggcattggtagcattataattcggcttccgcgctgtttatgggcatggcaatgtctcatg
FAD2-2_CHK80 cagtcgaccttagtcaaccaattctgggtggccagctccgggcgaccgggctccgtgtcg
FAD2-2_CHK22 cagtcgaccttagtcaaccaattctgggtggccagctccgggcgaccgggctccgtgtcg
FAD2-2_CHK80 ccgggcaccacctcctgccatgagtaacagggccgccctctcctcccgacgttggcccac
FAD2-2_CHK22 ccgggcaccacctcctgccatgagtaacagggccgccctctcctcccgacgttggcccac
FAD2-2_CHK80 tgaataccgtgtcttggggccctacatgatgggctgcctagtcggcggggacgcgcaact
FAD2-2_CHK22 tgaataccgtgtcttggggccctacatgatgggctgcctagtcggcggggacgcgcaact
FAD2-2_CHK80 gcccgcgcaatctgggacgtggtctgaatcctccaggcgggtttccccgagaaagaaagg
FAD2-2_CHK22 gcccgcgcaatctgggacgtggtctgaatcctccaggcgggtttccccgagaaagaaagg
FAD2-2_CHK80 gtgccgatttcaaagcagagccatgtgccgggccctgtggcctgtgttggcgcctatgta
FAD2-2_CHK22 gtgccgatttcaaagcagagccatgtgccgggccctgtggcctgtgttggcgcctatgta
FAD2-2_CHK80 gtcaccccccctcacccaattgtcgccagtttgcgcaatccataaactcaaaactgcagc
FAD2-2_CHK22 gtcaccccccctcacccaattgtcgccagtttgcgcaatccataaactcaaaactgcagc
FAD2-2_CHK80 ttctgagctgcgctgttcaagaacacctctggggtttgctcacccgcgaggtcgacgccc
FAD2-2_CHK22 ttctgagctgcgctgttcaagaacacctctggggtttgctcacccgcgaggtcgacgccc
FAD2-2_CHK80 agcATGGCTATCAAGACGAACAGGCAGCCTGTGGAGAAGCCTCCGTTCACGATCGGGACG
FAD2-2_CHK22 agcATGGCTATCAAGACGAACAGGCAGCCTGTGGAGAAGCCTCCGTTCACGATCGGGACG
FAD2-2_CHK80 CTGCGCAAGGCCATCCCCGCGCACTGTTTCGAGCGCTCGGCGCTTCGTAGCAGCATGTAC
FAD2-2_CHK22 CTGCGCAAGGCCATCCCCGCGCACTGTTTCGAGCGCTCGGCGCTTCGTAGCAGCATGTAC
FAD2-2_CHK80 CTGGCCTTTGACATCGCGGTCATGTCCCTGCTCTACGTCGCGTCGACGTACATCGACCCT
FAD2-2_CHK22 CTGGCCTTTGACATCGCGGTCATGTCCCTGCTCTACGTCGCGTCGACGTACATCGACCCT
FAD2-2_CHK80 GCGCCGGTGCCTACGTGGGTCAAGTATGGCGTCATGTGGCCGCTCTACTGGTTCTTCCAG
FAD2-2_CHK22 GCGCCGGTGCCTACGTGGGTCAAGTATGGCGTCATGTGGCCGCTCTACTGGTTCTTCCAG
FAD2-2_CHK80 GTGTGTGTGAGGGTTGTGGTTGCCCGTATCGAGGTCCTGGTGGCGCGCATGGGGGAGAAG
FAD2-2_CHK22 GTGTGTGTGAGGGTTGTGGTTGCCCGTATCGAGGTCCTGGTGGCGCGCATGGGGGAGAAG
FAD2-2_CHK80 GCGCCTGTCCCGCTGACCCCCCCGGCTACCCTCCCGGCACCTTCCAGGGCGCCTTCGGCA
FAD2-2_CHK22 GCGCCTGTCCCGCTGACCCCCCCGGCTACCCTCCCGGCACCTTCCAGGGCGCCTTCGGCA
FAD2-2_CHK80 CGGGTGTCTGGGTGTGCGCGCACGAGTGCGGCCACCAGGCCTTTTCCTCCAGCCAGGCCA
FAD2-2_CHK22 CGGGTGTCTGGGTGTGCGCGCACGAGTGCGGCCACCAGGCCTTTTCCTCCAGCCAGGCCA
FAD2-2_CHK80 TCAACGACGGCGTGGGCCTGGTGTTCCACAGCCTGCTGCTGGTGCCCTACTACTCCTGGA
FAD2-2_CHK22 TCAACGACGGCGTGGGCCTGGTGTTCCACAGCCTGCTGCTGGTGCCCTACTACTCCTGGA
FAD2-2_CHK80 AGCACTCGCACCGCCGCCACCACTCCAACACGGGGTGCCTGGACAAGGACGAGGTGTTTG
FAD2-2_CHK22 AGCACTCGCACCGCCGCCACCACTCCAACACGGGGTGCCTGGACAAGGACGAGGTGTTTG
FAD2-2_CHK80 TGCCGCCGCACCGCGCAGTGGCGCACGAGGGCCTGGAGTGGGAGGAGTGGCTGCCCATCC
FAD2-2_CHK22 TGCCGCCGCACCGCGCAGTGGCGCACGAGGGCCTGGAGTGGGAGGAGTGGCTGCCCATCC
FAD2-2_CHK80 GCATGGGCAAGGTGCTGGTCACCCTGACCCTGGGCTGGCCGCTGTACCTCATGTTCAACG
FAD2-2_CHK22 GCATGGGCAAGGTGCTGGTCACCCTGACCCTGGGCTGGCCGCTGTACCTCATGTTCAACG
FAD2-2_CHK80 TCGCCTCGCGGCCGTACCCGCGCTTCGCCAACCACTTTGACCCGTGGTCGCCCATCTTCA
FAD2-2_CHK22 TCGCCTCGCGGCCGTACCCGCGCTTCGCCAACCACTTTGACCCGTGGTCGCCCATCTTCA
FAD2-2_CHK80 GCAAGCGCGAGCGCATCGAGGTGGTCATCTCCGACCTGGCGCTGGTGGCGGTGCTCAGCG
FAD2-2_CHK22 GCAAGCGCGAGCGCATCGAGGTGGTCATCTCCGACCTGGCGCTGGTGGCGGTGCTCAGCG
FAD2-2_CHK80 GGCTCAGCGTGCTGGGCCGCACCATGGGCTGGGCCTGGCTGGTCAAGACCTACGTGGTGC
FAD2-2_CHK22 GGCTCAGCGTGCTGGGCCGCACCATGGGCTGGGCCTGGCTGGTCAAGACCTACGTGGTGC
FAD2-2_CHK80 CCTACCTGATCGTGAACATGTGGCTCGTGCTCATCACGCTGCTCCAGCACACGCACCCGG
FAD2-2_CHK22 CCTACCTGATCGTGAACATGTGGCTCGTGCTCATCACGCTGCTCCAGCACACGCACCCGG
FAD2-2_CHK80 CGCTGCCGCACTACTTCGAGAAGGACTGGGACTGGCTGCGCGGCGCCATGGCCACCGTGG
FAD2-2_CHK22 CGCTGCCGCACTACTTCGAGAAGGACTGGGACTGGCTGCGCGGCGCCATGGCCACCGTGG
FAD2-2_CHK80 ACCGCTCCATGGGCCCGCCCTTCATGGACAACATCCTGCACCACATCTCCGACACCCACG
FAD2-2_CHK22 ACCGCTCCATGGGCCCGCCCTTCATGGACAACATCCTGCACCACATCTCCGACACCCACG
FAD2-2_CHK80 TGCTGCACCACCTCTTCAGCACCATCCCGCACTACCACGCCGAGGAGGCCTCCGCCGCCA
FAD2-2_CHK22 TGCTGCACCACCTCTTCAGCACCATCCCGCACTACCACGCCGAGGAGGCCTCCGCCGCCA
FAD2-2_CHK80 TCAGGCCCATCCTGGGCAAGTACTACCAGTCCGACAGCCGCTGGGTCGGCCGCGCCCTGT
FAD2-2_CHK22 TCAGGCCCATCCTGGGCAAGTACTACCAGTCCGACAGCCGCTGGGTCGGCCGCGCCCTGT
FAD2-2_CHK80 GGGAGGACTGGCGCGACTGCCGCTACGTCGTCCCGGACGCGCCCGAGGACGACTCCGCGC
FAD2-2_CHK22 GGGAGGACTGGCGCGACTGCCGCTACGTCGTCCCGGACGCGCCCGAGGACGACTCCGCGC
FAD2-2_CHK80 TCTGGTTCCACAAGTGAgtgagtgagtcgctcactcagcgcgcctgcgcggggatgcgga
FAD2-2_CHK22 TCTGGTTCCACAAGTGAgtgagtgagtcgctcactcagcgcgcctgcgcggggatgcgga
FAD2-2_CHK80 acgccgccgccgccttgtcttttgcacgcgcgactccgtcgcttcgcgggtggcaccccc
FAD2-2_CHK22 acgccgccgccgccttgtcttttgcacgcgcgactccgtcgcttcgcgggtggcaccccc
FAD2-2_CHK80 attgaaaaaaacctcaattctgtttgtggaagacacggtgtacccccaaccacccacctg
FAD2-2_CHK22 attgaaaaaaacctcaattctgtttgtggaagacacggtgtacccccaaccacccacctg
FAD2-2_CHK80 cacctctattattggtattattgacgcgggagcgggcgttgtactctacaacgtagcgtc
FAD2-2_CHK22 cacctctattattggtattattgacgcgggagcgggcgttgtactctacaacgtagcgtc
FAD2-2_CHK80 tctggttttcagctggctcccaccattgtaaattcttgctaaaatagtgcgtggttatgt
FAD2-2_CHK22 tctggttttcagctggctcccaccattgtaaattcttgctaaaatagtgcgtggttatgt
FAD2-2_CHK80 gagaggtatggtgtaacagggcgtcagtcatgttggttttcgtgctgatctcgggcacaa
FAD2-2_CHK22 gagaggtatggtgtaacagggcgtcagtcatgttggttttcgtgctgatctcgggcacaa
FAD2-2_CHK80 ggcgtcgtcgacgtgacgtgcccgtgatgagagcaataccgcgctcaaagccgacgcatg
FAD2-2_CHK22 ggcgtcgtcgacgtgacgtgcccgtgatgagagcaataccgcgctcaaagccgacgcatg
FAD2-2_CHK80 gcctttactccgcactccaaacgactgtcgctcgtatttttcggatatctattttttaag
FAD2-2_CHK22 gcctttactccgcactccaaacgactgtcgctcgtatttttcggatatctattttttaag
FAD2-2_CHK80 agcgagcacagcgccgggcatgggcctgaaaggcctcgcggccgtgctcgtggtgggggc
FAD2-2_CHK22 agcgagcacagcgccgggcatgggcctgaaaggcctcgcggccgtgctcgtggtgggggc
FAD2-2_CHK80 cgcgagcgcgtggggcatcgcggcagtgcaccaggcgcagacggaggaacgcatggtgag
FAD2-2_CHK22 cgcgagcgcgtggggcatcgcggcagtgcaccaggcgcagacggaggaacgcatggtgag
FAD2-2_CHK80 tgcgcatcacaagatgcatgtcttgttgtctgtactataatgctagagcatcaccagggg
FAD2-2_CHK22 tgcgcatcacaagatgcatgtcttgttgtctgtactataatgctagagcatcaccagggg
FAD2-2_CHK80 cttagtcatcgcacctgctttggtcattacagaaattgcacaagggcgtcctccgggatg
FAD2-2_CHK22 cttagtcatcgcacctgctttggtcattacagaaattgcacaagggcgtcctccgggatg
FAD2-2_CHK80 aggagatgtaccagctcaagctggagcggcttcgagccaagcaggagcgcggcgcatgac
FAD2-2_CHK22 aggagatgtaccagctcaagctggagcggcttcgagccaagcaggagcgcggcgcatgac
FAD2-2_CHK80 gacctacccacatgcgacaaaggggtctgggtcgtacgacaaaccagtcaggaggcggcg
FAD2-2_CHK22 gacctacccacatgcgacaaaggggtctgggtcgtacgacaaaccagtcaggaggcggcg
FAD2-2_CHK80 gggtccatgagctggcccgctcgcagcttcagcgcttcgagcatcgcggcgttgtccgcg
FAD2-2_CHK22 gggtccatgagctggcccgctcgcagcttcagcgcttcgagcatcgcggcgttgtccgcg
FAD2-2_CHK80 atccaagcctcccgcagcgcctccttggggcccctgtgcggagaggcgggtgccgagagg
FAD2-2_CHK22 atccaagcctcccgcagcgcctccttggggcccctgtgcggagaggcgggtgccgagagg
FAD2-2_CHK80 gctgtgagggcggaggctggaggacaggctgcgaatcgtcgcgctgcctaggcgtctgcc
FAD2-2_CHK22 gctgtgagggcggaggctggaggacaggctgcgaatcgtcgcgctgcctaggcgtctgcc
FAD2-2_CHK80 gaggagaagcacaccacgcgccgccgctttcatttcattgtgcccgttgctgtaatgcat
FAD2-2_CHK22 gaggagaagcacaccacgcgccgccgctttcatttcattgtgcccgttgctgtaatgcat
FAD2-2_CHK80 gcgagcgatcctgcactaaagctgcgatcgccacctcagcgttgttcgtctctagcaccg
FAD2-2_CHK22 gcgagcgatcctgcactaaagctgcgatcgccacctcagcgttgttcgtctctagcaccg
FAD2-2_CHK80 ctcacctgaccacgcgcacggaagagtggcggtcggcgacggcctgctgcacgtggcagt
FAD2-2_CHK22 ctcacctgaccacgcgcacggaagagtggcggtcggcgacggcctgctgcacgtggcagt
FAD2-2_CHK80 gcaggtccacgtggccgaggagcaggtctcgcagctgcagcaccgcgccctcgtgctcgc
FAD2-2_CHK22 gcaggtccacgtggccgaggagcaggtctcgcagctgcagcaccgcgccctcgtgctcgc
FAD2-2_CHK80 cggggtccatccagtccatgccccgcgccgggccctcgagccggacggccagcagccagg
FAD2-2_CHK22 cggggtccatccagtccatgccccgcgccgggccctcgagccggacggccagcagccagg
FAD2-2_CHK80 gccgggggtccgcgccgacgcccggcggccccgccgcgcagctggggtcggccaggaaga
FAD2-2_CHK22 gccgggggtccgcgccgacgcccggcggccccgccgcgcagctggggtcggccaggaaga
FAD2-2_CHK80 gccactgggcggccggcccgggcgagccccgcgccggcgcgggacccacccgcagcacca
FAD2-2_CHK22 gccactgggcggccggcccgggcgagccccgcgccggcgcgggacccacccgcagcacca
FAD2-2_CHK80 ccgcctggtccaggtcgacgtcgctcggcggcaggcccgcgtgcggcgcctgcgccagcg
FAD2-2_CHK22 ccgcctggtccaggtcgacgtcgctcggcggcaggcccgcgtgcggcgcctgcgccagcg
FAD2-2_CHK80 tccagggcaggcgggggcgcgcgtgca
FAD2-2_CHK22 tccagggcaggcgggggcgcgcgtgca
FATA1-1_CHK80 gcaatggcgctcggtacagggtctgcgtccgtgctgggctccctctcctacgatgcacaa
(SEQ ID NO: 5)
FATA1-1_CHK22 gcaatggcgctcggtacagggtctgcgtccgtgctgggctccctctcctacgatgcacaa
(SEQ ID NO: 6)
FATA1-1_CHK80 gggagcgccccggccagctcagcgcgtccacaacctcccctcgtcacacacacacctgcg
FATA1-1_CHK22 gggagcgccccggccagctcagcgcgtccacaacctcccctcgtcacacacacacctgcg
FATA1-1_CHK80 gaaccaggccgcccatttgctgcttgagcatgccttgcatcatgtccgggtttcccatca
FATA1-1_CHK22 gaaccaggccgcccatttgctgcttgagcatgccttgcatcatgtccgggtttcccatca
FATA1-1_CHK80 tatcgttgaggttcttgggctccagcttctgctccagcacaccatcctgtcgatcgaaga
FATA1-1_CHK22 tatcgttgaggttcttgggctccagcttctgctccagcacaccatcctgtcgatcgaaga
FATA1-1_CHK80 gaaggagacatgtgtacattattggtgtgagggcgctgaatcggccatttttta-aatga
FATA1-1_CHK22 gaaggagacatgtgtacattattggtgtgagggcgctgaatcggccattttttaaaatga
FATA1-1_CHK80 tcacgctcatgccaatagacgcggcacataacgacgttcaaacccccgccaaagccgcgg
FATA1-1_CHK22 tcacgctcatgccaatagacgcggcacataacgacgttcaaacccccgccaaagccgcgg
FATA1-1_CHK80 acaaccccatccctccacaccccccacacaaagaacccgccaccgcttaccttgcccacg
FATA1-1_CHK22 acaaccccatccctccacaccccccacacaaagaacccgccaccgcttaccttgcccacg
FATA1-1_CHK80 aggtaggcctttcgttgcgcaaaaccggcctcggtgatgaatgcatgcccgttcctgacg
FATA1-1_CHK22 aggtaggcctttcgttgcgcaaaaccggcctcggtgatgaatgcatgcccgttcctgacg
FATA1-1_CHK80 agcgctgcccgggccaacacgctcttttgctgcgtctcctcaggcttgggggcctccttg
FATA1-1_CHK22 agcgctgcccgggccaacacgctcttttgctgcgtctcctcaggcttgggggcctccttg
FATA1-1_CHK80 ggcttgggtgccgccatgatctgcgcgcatcagagaaacgttgctggt-aaaaggagcgc
FATA1-1_CHK22 ggcttgggtgccgccatgatctgcgcgcatcagagaaacgttgctggtaaaaaggagcgc
FATA1-1_CHK80 ccggctgcgcaatatatatataggcatgccaacacagcccaacctcactcgggagcccgt
FATA1-1_CHK22 ccggctgcgcaatatatatataggcatgccaacacagcccaacctcactcgggagcccgt
FATA1-1_CHK80 cccaccacccccaagtcgcgtgccttgacggcatactgctgcagaagcttcatgagaatg
FATA1-1_CHK22 cccaccacccccaagtcgcgtgccttgacggcatactgctgcagaagcttcatgagaatg
FATA1-1_CHK80 atgccgaacaagaggggcacgaggacccaatcccggacatccttgtcgataatgatctcg
FATA1-1_CHK22 atgccgaacaagaggggcacgaggacccaatcccggacatccttgtcgataatgatctcg
FATA1-1_CHK80 tgagtccccatcgtccgcccgacgctccggggagcccgccgatgctcaagacgagagggc
FATA1-1_CHK22 tgagtccccatcgtccgcccgacgctccggggagcccgccgatgctcaagacgagagggc
FATA1-1_CHK80 cctcgaccaggaggggctggcccgggcgggcactggcgtcgaaggtgcgcccgtcgttcg
FATA1-1_CHK22 cctcgaccaggaggggctggcccggggcggcactggcgtcgaaggtgcgcccgtcgttcg
FATA1-1_CHK80 cctgcagtcctatgccacaaaacaagtcttctgacggggtgcgtttgctcccgtgcgggc
FATA1-1_CHK22 cctgcagtcctatgccacaaaacaagtcttctgacggggtgcgtttgctcccgtgcgggc
FATA1-1_CHK80 aggcaacagaggtattcaccctggtcatggggagatcggcgatcgagctgggataagaga
FATA1-1_CHK22 aggcaacagaggtattcaccctggtcatggggagatcggcgatcgagctgggataagaga
FATA1-1_CHK80 tacggtcccgcgcaaggatcgctcatcctggtctgagccggacagtcattctggcaagca
FATA1-1_CHK22 tacggtcccgcgcaaggatcgctcatcctggtctgagccggacagtcattctggcaagca
FATA1-1_CHK80 atgacaacttgtcaggaccggaccgtgccatatatttctcacctagcgccgcaaaaccta
FATA1-1_CHK22 atgacaacttgtcaggaccggaccgtgccatatatttctcacctagcgccgcaaaaccta
FATA1-1_CHK80 acaatttgggagtcactgtgccactgagttcgactggtagctgaatggagtcgctgctcc
FATA1-1_CHK22 acaatttgggagtcactgtgccactgagttcgactggtagctgaatggagtcgctgctcc
FATA1-1_CHK80 actaaacgaattgtcagcaccgccagccggccgaggacccgagtcatagcgagggtagta
FATA1-1_CHK22 actaaacgaattgtcagcaccgccagccggccgaggacccgagtcatagcgagggtagta
FATA1-1_CHK80 gcgcgccatggcaccgaccagcctgcttgccagtactggcgtctcttccgcttctctgtg
FATA1-1_CHK22 gcgcgccatggcaccgaccagcctgcttgccagtactggcgtctcttccgcttctctgtg
FATA1-1_CHK80 gtcctctgcgcgctccagcgcgtgcgcttttccggtggatcatgcggtccgtggcgcacc
FATA1-1_CHK22 gtcctctgcgcgctccagcgcgtgcgcttttccggtggatcatgcggtccgtggcgcacc
FATA1-1_CHK80 gcagcggccgctgcccatgcagcgccgctgcttccgaacagtggcggtcagggccgcacc
FATA1-1_CHK22 gcagcggccgctgcccatgcagcgccgctgcttccgaacagtggcggtcagggccgcacc
FATA1-1_CHK80 cgcggtagccgtccgtccggaacccgcccaagagttttgggagcagcttgagctctgcaa
FATA1-1_CHK22 cgcggtagccgtccgtccggaacccgcccaagagttttgggagcagcttgagccctgcaa
FATA1-1_CHK80 gATGGCGGAGGACAAGCGCATCTTCCTGGAGGAGCACCGGTGCGTGGAGGTCCGGGGCTG
FATA1-1_CHK22 gATGGCGGAGGACAAGCGCATCTTCCTGGAGGAGCACCGGTGCGTGGAGGTCCGGGGCTG
FATA1-1_CHK80 ACCGGCCGTCGCATTCAACGTAATCAATCGCATGATGATCAGAGGACACGAAGTCTTGGT
FATA1-1_CHK22 ACCGGCCGTCGCATTCAACGTAATCAATCGCATGATGATCAGAGGACACGAAGTCTTGGT
FATA1-1_CHK80 GGCGGTGGCCAGAAACACTGTCCATTGCAAGGGCATAGGGATGCGTTCCTTCACCTCTCA
FATA1-1_CHK22 GGCGGTGGCCAGAAACACTGTCCATTGCAAGGGCATAGGGATGCGTTCCTTCACCTCTCA
FATA1-1_CHK80 TTTCTCATTTCTGAATCCCTCCCTGCTCACTCTTTCTCCTCCTCCTTCCCGTTCACGCAG
FATA1-1_CHK22 TTTCTCATTTCTGAATCCCTCCCTGCTCACTCTTTCTCCTCCTCCTTCCCGTTCACGCAG
FATA1-1_CHK80 CATTCGGGGCAACGAGGTGGGCCCCTCGCAGCGGCTGACGATCACGGCGGTGGCCAACAT
FATA1-1_CHK22 CATTCGGGGCAACGAGGTGGGCCCCTCGCAGCGGCTGACGATCACGGCGGTGGCCAACAT
FATA1-1_CHK80 CCTGCAGGAGGCGGCGGGCAACCACGCGGTGGCCATGTGGGGCCGGAGCTCGGAGGGTTT
FATA1-1_CHK22 CCTGCAGGAGGCGGCGGGCAACCACGCGGTGGCCATGTGGGGCCGGAGCTCGGAGGGTTT
FATA1-1_CHK80 CGCGACGGACCCGGAGCTGCAGGAGGCGGGTCTCATCTTTGTGATGACGCGCATGCAGAT
FATA1-1_CHK22 CGCGACGGACCCGGAGCTGCAGGAGGCGGGTCTCATCTTTGTGATGACGCGCATGCAGAT
FATA1-1_CHK80 CCAGATGTACCGCTACCCGCGCTGGGGCGACCTGATGCAGGTGGAGACCTGGTTCCAGAC
FATA1-1_CHK22 CCAGATGTACCGCTACCCGCGCTGGGGCGACCTGATGCAGGTGGAGACCTGGTTCCAGAC
FATA1-1_CHK80 GGCGGGCAAGCTGGGCGCGCAGCGCGAGTGGGTGCTGCGCGACAAGCTGACCGGCGAGGC
FATA1-1_CHK22 GGCGGGCAAGCTGGGCGCGCAGCGCGAGTGGGTGCTGCGCGACAAGCTGACCGGCGAGGC
FATA1-1_CHK80 GCTGGGCGCGGCCACCTCGAGCTGGGTCATGATCAACATCCGCACGCGCCGGCCGTGCCG
FATA1-1_CHK22 GCTGGGCGCGGCCACCTCGAGCTGGGTCATGATCAACATCCGCACGCGCCGGCCGTGCCG
FATA1-1_CHK80 CATGCCGGAGCTCGTCCGCGTCAAGTCGGCCTTCTTCGCGCGCGAGCCGCCGCGCCTGGC
FATA1-1_CHK22 CATGCCGGAGCTCGTCCGCGTCAAGTCGGCCTTCTTCGCGCGCGAGCCGCCGCGCCTGGC
FATA1-1_CHK80 GCTGCCGCCCGCGGTCACGCGTGCCAAGCTGCCCAACATCGCGACGCCGGCGCCGCTGCG
FATA1-1_CHK22 GCTGCCGCCCGCGGTCACGCGTGCCAAGCTGCCCAACATCGCGACGCCGGCGCCGCTGCG
FATA1-1_CHK80 CGGGCACCGCCAGGTCGCGCGCCGCACCGACATGGACATGAACGGGCACGTGAACAACGT
FATA1-1_CHK22 CGGGCACCGCCAGGTCGCGCGCCGCACCGACATGGACATGAACGGGCACGTGAACAACGT
FATA1-1_CHK80 GGCCTACCTGGCCTGGTGCCTGGAGGCCGTGCCCGAGCACGTCTTCAGCGACTACCACCT
FATA1-1_CHK22 GGCCTACCTGGCCTGGTGCCTGGAGGCCGTGCCCGAGCACGTCTTCAGCGACTACCACCT
FATA1-1_CHK80 CTACCAGATGGAGATCGACTTCAAGGCCGAGTGCCACGCGGGCGACGTCATCTCCTCCCA
FATA1-1_CHK22 CTACCAGATGGAGATCGACTTCAAGGCCGAGTGCCACGCGGGCGACGTCATCTCCTCCCA
FATA1-1_CHK80 GGCCGAGCAGATCCCGCCCCAGGAGGCGCTCACGCACAACGGCGCCGGCCGCAACCCCTC
FATA1-1_CHK22 GGCCGAGCAGATCCCGCCCCAGGAGGCGCTCACGCACAACGGCGCCGGCCGCAACCCCTC
FATA1-1_CHK80 CTGCTTCGTCCATAGCATTCTGCGCGCCGAGACCGAGCTCGTCCGCGCGCGAACCACATG
FATA1-1_CHK22 CTGCTTCGTCCATAGCATTCTGCGCGCCGAGACCGAGCTCGTCCGCGCGCGAACCACATG
FATA1-1_CHK80 GTCGGCCCCCATCGACGCGCCCGCCGCCAAGCCGCCCAAGGCGAGCCACTGAggacaggg
FATA1-1_CHK22 GTCGGCCCCCATCGACGCGCCCGCCGCCAAGCCGCCCAAGGCGAGCCACTGAggacaggg
FATA1-1_CHK80 tggttggctggatggggaaacgctggtcgcgggattcgatcctgctgcttatatcctccc
FATA1-1_CHK22 tggttggctggatggggaaacgctggtcgcgggattcgatcctgctgcttatatcctccc
FATA1-1_CHK80 tggaagcacacccacgactctgaagaagaaaacgtgcacacacacaacccaaccggccga
FATA1-1_CHK22 tggaagcacacccacgactctgaagaagaaaacgtgcacacacacaacccaaccggccga
FATA1-1_CHK80 atatttgcttccttatcccgggtccaagagagactgcgatgcccccctcaatcagcatcc
FATA1-1_CHK22 atatttgcttccttatcccgggtccaagagagactgcgatgcccccctcaatcagcatcc
FATA1-1_CHK80 tcctccctgccgcttcaatcttccctgcttgcctgcgcccgcggtgcgccgtctgcccgc
FATA1-1_CHK22 tcctccctgccgcttcaatcttccctgcttgcctgcgcccgcggtgcgccgtctgcccgc
FATA1-1_CHK80 ccagtcagtcactcctgcacaggccccttgtgcgcagtgctcctgtaccctttaccgctc
FATA1-1_CHK22 ccagtcagtcactcctgcacaggccccttgtgcgcagtgctcctgtaccctttaccgctc
FATA1-1_CHK80 cttccattctgcgaggccccctattgaatgtattcgttgcctgtgtggccaagcgggctg
FATA1-1_CHK22 cttccattctgcgaggccccctattgaatgtattcgttgcctgtgtggccaagcgggctg
FATA1-1_CHK80 ctgggcgcgccgccgtcgggcagtgctcggcgactttggcggaagccgattgttcttctg
FATA1-1_CHK22 ctgggcgcgccgccgtcgggcagtgctcggcgactttggcggaagccgattgttcttctg
FATA1-1_CHK80 taagccacgcgcttgctgctttgggaagagaaggggggggtactgaatggatgaggagga
FATA1-1_CHK22 taagccacgcgcttgctgctttgggaagagaaggggggggtactgaatggatgaggagga
FATA1-1_CHK80 gaaggaggggtattggtattatctgagttggggaggcagggagagttggaaaatgtaagt
FATA1-1_CHK22 gaaggaggggtattggtattatctgagttggggaggcagggagagttggaaaatgtaagt
FATA1-1_CHK80 ggcacgacgggcaaggagaatggtgagcatgtgcatggtgatgtcgttggtcgaggacga
FATA1-1_CHK22 ggcacgacgggcaaggagaatggtgagcatgtgcatggtgatgtcgttggtcgaggacga
FATA1-1_CHK80 tcctgcacgcgtgtatctgatgtagaatacggcaatcaccctagtctacatctatacctt
FATA1-1_CHK22 tcctgcacgcgtgtatctgatgtagaatacggcaatcaccctagtctacatctatacctt
FATA1-1_CHK80 ctccgtataacgccctttccaaatgccctcccgtttctctcctattcttgatccacatga
FATA1-1_CHK22 ctccgtataacgccctttccaaatgccctcccgtttctctcctattcttgatccacatga
FATA1-1_CHK80 tgaccctggcactatttcaagggctggacatttcaagaaggtttgcgtatctgaagaagg
FATA1-1_CHK22 tgaccctggcactatttcaagggctggacatttcaagaaggtttgcgtatctgaagaagg
FATA1-1_CHK80 attggtttggagaggtggccgatgaaagtggggtcaagctgcgtggagccctctgcacgg
FATA1-1_CHK22 attggtttggagaggtggccgatgaaagtggggtcaagctgcgtggagccctctgcacgg
FATA1-1_CHK80 atttctatggtaatctgcgtccacgtcatcagtagccgtacgcctctgcggcgtcgccgt
FATA1-1_CHK22 atttctatggtaatctgcgtccacgtcatcagtagccgtacgcctctgcggcgtcgccgt
FATA1-1_CHK80 gctcctcagccgccgcttcaccaccagccaggagccccatacgcacgcgctcccagacgg
FATA1-1_CHK22 gctcctcagccgccgcttcaccaccagccaggagccccatacgcacgcgctcccagacgg
FATA1-1_CHK80 cgcgcgcgacggggacgatcatgagcagcgcgatctggggcgtgtcgtgcgagctgagcc
FATA1-1_CHK22 cgcgcgcgacggggacgatcatgagcagcgcgatctggggcgtgtcgtgcgagctgagcc
FATA1-1_CHK80 gcacaaccacgttgccctggccgcgcgtggtgtactggcagttggtcgagaccatggtgt
FATA1-1_CHK22 gcacaaccacgttgccctggccgcgcgtggtgtactggcagttggtcgagaccatggtgt
FATA1-1_CHK80 cggacttgaagtaggagccgtcaaagcgcgggaggcgcgtctcggccgagacgttgccgc
FATA1-1_CHK22 cggacttgaagtaggagccgtcaaagcgcgggaggcgcgtctcggccgagacgttgccgc
FATA1-1_CHK80 cgatgaccaggctctggcgcttctggcgcgggtcgcggccctgccactgcgcggagccgc
FATA1-1_CHK22 cgatgaccaggctctggcgcttctggcgcgggtcgcggccctgccactgcgcggagccgc
FATA1-1_CHK80 ccaggagcaggcgcgactcgtcacggccgcgcagcttgacgtcggccgccagcgccgtgc
FATA1-1_CHK22 ccaggagcaggcgcgactcgtcacggccgcgcagcttgacgtcggccgccagcgccgtgc
FATA1-1_CHK80 ccggctcgtaggccgaccccatcttgacgaagaggcggccgatcgctgcgcggaccttga
FATA1-1_CHK22 ccggctcgtaggccgaccccatcttgacgaagaggcggccgatcgctgcgcggaccttga
FATA1-1_CHK80 ccgcgtcggacaggcgcaggcgctcgtccagcttgaccccgagcgccgcgttgccgggct
FATA1-1_CHK22 ccgcgtcggacaggcgcaggcgctcgtccagcttgaccccgagcgccgcgttgccgggct
FATA1-1_CHK80 tgaacggcagcgagaactcctcgcccagcttggagccgagcaggcccagcgccagcttct
FATA1-1_CHK22 tgaacggcagcgagaactcctcgcccagcttggagccgagcaggcccagcgccagcttct
FATA1-1_CHK80 gccgcttgccgcgcgcgcgcaggcgcgagtcgacgcgcagcgtgtagag
FATA1-1_CHK22 gccgcttgccgcgcgcgcgcaggcgcgagtcgacgcgcagcgtgtagag
FATA1-2_CHK80 gcaatggcgctctgtgcagggtctgtgtccggtctccctctcctacgatgcacaaggcaa
(SEQ ID NO: 7)
FATA1-2_CHK22 gcaatggcgctctgtgcagggtctgtgtccggtctccctctcctacgatgcacaaggcaa
(SEQ ID NO: 8)
FATA1-2_CHK80 cgcctttgctgactcagcgcgtccacaacctcccctcgtcacacacacctgcggaaccag
FATA1-2_CHK22 cgcctttgctgactcagcgcgtccacaacctcccctcgtcacacacacctgcggaaccag
FATA1-2_CHK80 gccgcccatttgctgcttgagcatgccttgcatcatgtccgggttgcccatcatatcgtt
FATA1-2_CHK22 gccgcccatttgctgcttgagcatgccttgcatcatgtccgggttgcccatcatatcgtt
FATA1-2_CHK80 gaggttcttgggctccagtttctgctccagcacgccatcctgtcggtcgaagaggaggag
FATA1-2_CHK22 gaggttcttgggctccagtttctgctccagcacgccatcctgtcggtcgaagaggaggag
FATA1-2_CHK80 acatgtaagcgttgttgggatgagggttgctaaattggccattatttttaatgatcacgc
FATA1-2_CHK22 acatgtaagcgttgttgggatgagggttgctaaattggccattatttttaatgatcacgc
FATA1-2_CHK80 tcgtgccaatagacgcggcacattgcgatgttcaaactcccgtcaaagccgcggacaacc
FATA1-2_CHK22 tcgtgccaatagacgcggcacattgcgatgttcaaactcccgtcaaagccgcggacaacc
FATA1-2_CHK80 acatccctccacaccccacacacaaagaacccgccaccgctcaccttgcccacgaggtag
FATA1-2_CHK22 acatccctccacaccccacacacaaagaacccgccaccgctcaccttgcccacgaggtag
FATA1-2_CHK80 gcctttcgttgcgcaaacccggcctcggtgatgaatgcgtgcccgttcctgacgagcgct
FATA1-2_CHK22 gcctttcgttgcgcaaacccggcctcggtgatgaatgcgtgcccgttcctgacgagcgct
FATA1-2_CHK80 gcccgggccaacacgctcttttgctgcgtctcctcaggcttgggggcctccttgggcttg
FATA1-2_CHK22 gcccgggccaacacgctcttttgctgcgtctcctcaggcttgggggcctccttgggcttg
FATA1-2_CHK80 ggggccgccatgatctgcgcgcaccagagagaagtcattggtgaaaaggagcgcccggct
FATA1-2_CHK22 ggggccgccatgatctgcgcgcaccagagagaagtcattggtgaaaaggagcgcccggct
FATA1-2_CHK80 gcgctatatatatatatatatgctcaggccaataaagtccaacctcactggggagccccg
FATA1-2_CHK22 gcgctatatatatatatatatgctcaggccaataaagtccaacctcactggggagccccg
FATA1-2_CHK80 tcccaccacccccaagtcgcgtaccttgacggcatactgctgcagaagcttcatgaggat
FATA1-2_CHK22 tcccaccacccccaagtcgcgtaccttgacggcatactgctgcagaagcttcatgaggat
FATA1-2_CHK80 gatgccgaacaagaggggcacaaggacccaatcccggacatccttgtcgataatgatctc
FATA1-2_CHK22 gatgccgaacaagaggggcacaaggacccaatcccggacatccttgtcgataatgatctc
FATA1-2_CHK80 gtgagtccccatcgtccgcccgacgctgcggggagcccgccgatgctcaagacgagaggg
FATA1-2_CHK22 gtgagtccccatcgtccgcccgacgctgcggggagcccgccgatgctcaagacgagaggg
FATA1-2_CHK80 ccctcgaccaggaggggctggcccgggcgggcactggcgtcgaaggtgcgcccgtcgttc
FATA1-2_CHK22 ccctcgaccaggaggggctggcccgggcgggcactggcgtcgaaggtgcgcccgtcgttc
FATA1-2_CHK80 gcctgcagtccgatgacacaaaacaagtcttctgacggggtgcgtttgctcccgtgcggg
FATA1-2_CHK22 gcctgcagtccgatgacacaaaacaagtcttctgacggggtgcgtttgctcccgtgcggg
FATA1-2_CHK80 caggcaacagaggtattcaacctggtcatggcgagatcggcgatcgagctgggataagag
FATA1-2_CHK22 caggcaacagaggtattcaacctggtcatggcgagatcggcgatcgagctgggataagag
FATA1-2_CHK80 atacggtcccgcgcgaggatcgctcatcctggtctgagccggacagtcattctggcaagc
FATA1-2_CHK22 atacggtcccgcgcgaggatcgctcatcctggtctgagccggacagtcattctggcaagc
FATA1-2_CHK80 aatgacaacttgccaggaccggaccgtgccatatatttctcacctagcgcggcaaaacct
FATA1-2_CHK22 aatgacaacttgccaggaccggaccgtgccatatatttctcacctagcgcggcaaaacct
FATA1-2_CHK80 aacaatttggaagtcactgtgccactgagctcgactggtagctgaatggagtcgctgctc
FATA1-2_CHK22 aacaatttggaagtcactgtgccactgagctcgactggtagctgaatggagtcgctgctc
FATA1-2_CHK80 cactaatcgaattgtcagcaccgccagccggccgaggacccgagtcatagcgagggtagt
FATA1-2_CHK22 cactaatcgaattgtcagcaccgccagccggccgaggacccgagtcatagcgagggtagt
FATA1-2_CHK80 agcgcgccatggcaccgaccagcctgcttgcccgtactggcgtctcttccgcttctctgt
FATA1-2_CHK22 agcgcgccatggcaccgaccagcctgcttgcccgtactggcgtctcttccgcttctctgt
FATA1-2_CHK80 gctcctctacgcgctccggcgcgtgcgcttttccggtggatcatgcggtccgtggcgcac
FATA1-2_CHK22 gctcctctacgcgctccggcgcgtgcgcttttccggtggatcatgcggtccgtggcgcac
FATA1-2_CHK80 cgcagcggccgctgcccatgcagcgccgctgcttccgaacagtggctgtcagggccgcac
FATA1-2_CHK22 cgcagcggccgctgcccatgcagcgccgctgcttccgaacagtggctgtcagggccgcac
FATA1-2_CHK80 ccgcagtagccgtccgtccggaacccgcccaagagttttgggagcagcttgagccctgca
FATA1-2_CHK22 ccgcagtagccgtccgtccggaacccgcccaagagttttgggagcagcttgagccctgca
FATA1-2_CHK80 agATGGCGGAGGACAAGCGCATCTTCCTGTAGGAGCACCGGTGCGCGGAGGTCCGGGGCT
FATA1-2_CHK22 agATGGCGGAGGACAAGCGCATCTTCCTGGAGGAGCACCGGTGCGCGGAGGTCCGGGGCT
FATA1-2_CHK80 GACCGGCCGTCGCATTCAACGTAATCAATCGCATGATGATCACAGGACGCGACGTCTTGG
FATA1-2_CHK22 GACCGGCCGTCGCATTCAACGTAATCAATCGCATGATGATCACAGGACGCGACGTCTTGG
FATA1-2_CHK80 TGGCGGTGGCCAGGGACACTGCCCATTGCACAGGCATAGGAATGCGTTCCTTCTCATTTC
FATA1-2_CHK22 TGGCGGTGGCCAGGGACACTGCCCATTGCACAGGCATAGGAATGCGTTCCTTCTCATTTC
FATA1-2_CHK80 TCAGTTTTCTGAGCCCCTCCCTCTTCACTCTTTCTCCTCCTCCTCCCCTCTCACGCAGCA
FATA1-2_CHK22 TCAGTTTTCTGAGCCCCTCCCTCTTCACTCTTTCTCCTCCTCCTCCCCTCTCACGCAGCA
FATA1-2_CHK80 TTCGTGGCAACGAGGTGGGCCCCTCGCAGCGGCTGACGATCACGGCGGTGGCCAACATCC
FATA1-2_CHK22 TTCGTGGCAACGAGGTGGGCCCCTCGCAGCGGCTGACGATCACGGCGGTGGCCAACATCC
FATA1-2_CHK80 TGCAGGAGGCGGCGGGCAACCACGCGGTGGCCATGTGGGGTCGGAGCTCGGAGGGTTTCG
FATA1-2_CHK22 TGCAGGAGGCGGCGGGCAACCACGCGGTGGCCATGTGGGGTCGGAGCTCGGAGGGTTTCG
FATA1-2_CHK80 CGACGGACCCGGAGCTGCAGGAGGCGGGCCTCATCTTTGTGATGACGCGCATGCAGATCC
FATA1-2_CHK22 CGACGGACCCGGAGCTGCAGGAGGCGGGCCTCATCTTTGTGATGACGCGCATGCAGATCC
FATA1-2_CHK80 AAATGTACCGCTACCCGCGCTGGGGCGACCTGATGCAGGTGGAGACCTGGTTCCAGACGG
FATA1-2_CHK22 AAATGTACCGCTACCCGCGCTGGGGCGACCTGATGCAGGTGGAGACCTGGTTCCAGACGG
FATA1-2_CHK80 CGGGCAAGCTAGGCGCGCAGCGCGAGTGGGTGCTGCGCGACAAGCTGACCGGCGAGGCGC
FATA1-2_CHK22 CGGGCAAGCTAGGCGCGCAGCGCGAGTGGGTGCTGCGCGACAAGCTGACCGGCGAGGCGC
FATA1-2_CHK80 TGGGCGCGGCCACCTCCAGCTGGGTCATGATCAACATCCGCACGCGCCGGCCGTGCCGCA
FATA1-2_CHK22 TGGGCGCGGCCACCTCCAGCTGGGTCATGATCAACATCCGCACGCGCCGGCCGTGCCGCA
FATA1-2_CHK80 TGCCCGAGCTCGTCCGCGTCAAGTCGGCCTTCTTCGCGCGCGAGCCGCCGCGCCTGGCGC
FATA1-2_CHK22 TGCCCGAGCTCGTCCGCGTCAAGTCGGCCTTCTTCGCGCGCGAGCCGCCGCGCCTGGCGC
FATA1-2_CHK80 TGCCGCCCACGGTCACGCGCGCCAAGCTGCCCAACATCGCGACGCCGGCGCCGCTGCGCG
FATA1-2_CHK22 TGCCGCCCACGGTCACGCGCGCCAAGCTGCCCAACATCGCGACGCCGGCGCCGCTGCGCG
FATA1-2_CHK80 GGCACCGCCAGGTCGCGCGCCGCACCGACATGGACATGAACGGCCACGTGAACAACGTTG
FATA1-2_CHK22 GGCACCGCCAGGTCGCGCGCCGCACCGACATGGACATGAACGGCCACGTGAACAACGTTG
FATA1-2_CHK80 CCTACCTGGCCTGGTGCCTGGAGGCCGTGCCCGAGCACGTCTTCAGCGACTACCACCTCT
FATA1-2_CHK22 CCTACCTGGCCTGGTGCCTGGAGGCCGTGCCCGAGCACGTCTTCAGCGACTACCACCTCT
FATA1-2_CHK80 ACCAGATGGAGATCGACTTCAAGGCCGAGTGCCACGCGGGCGACGTCATCTCCTCCCAGG
FATA1-2_CHK22 ACCAGATGGAGATCGACTTCAAGGCCGAGTGCCACGCGGGCGACGTCATCTCCTCCCAGG
FATA1-2_CHK80 CCGAGCAGATCCCGCCCCAGGAGGCGCTCACGCACAACGGCGCCGGCCGCAACCCCTCCT
FATA1-2_CHK22 CCGAGCAGATCCCGCCCCAGGAGGCGCTCACGCACAACGGCGCCGGCCGCAACCCCTCCT
FATA1-2_CHK80 GCTTCGTCCACAGCATTCTGCGCGCCGAGACCGAGCTCGTCCGCGCGCGCACCACCTGGT
FATA1-2_CHK22 GCTTCGTCCACAGCATTCTGCGCGCCGAGACCGAGCTCGTCCGCGCGCGCACCACCTGGT
FATA1-2_CHK80 CGGCCCCCGTCGACGCGCCCGCCGCCAAGCCGCCCAAGGCGAGCCACTGAgcagaggaga
FATA1-2_CHK22 CGGCCCCCGTCGACGCGCCCGCCGCCAAGCCGCCCAAGGCGAGCCACTGAgcagaggaga
FATA1-2_CHK80 gggtggctggtagttgcgggatggctggtcgcccgtcgatcctgctgctgctattgtctc
FATA1-2_CHK22 gggtggctggtagttgcgggatggctggtcgcccgtcgatcctgctgctgctattgtctc
FATA1-2_CHK80 ctcctgcacaagcccacccacgactccgaagaagaagaagaaaacgcgcacacacacaac
FATA1-2_CHK22 ctcctgcacaagcccacccacgactccgaagaagaagaagaaaacgcgcacacacacaac
FATA1-2_CHK80 ccaaccggccgaatatttgcttccttatcccgggtccaagagagacggcgatgcccccct
FATA1-2_CHK22 ccaaccggccgaatatttgcttccttatcccgggtccaagagagacggcgatgcccccct
FATA1-2_CHK80 caatcagcctcctcctccctgccgctccaatcttccctgcttgcatgcgcccgcgagagg
FATA1-2_CHK22 caatcagcctcctcctccctgccgctccaatcttccctgcttgcatgcgcccgcgagagg
FATA1-2_CHK80 ctgtctgcgcgccccgtcagtcactccccgtgcagacgcctcgtgctcggtgctcctgta
FATA1-2_CHK22 ctgtctgcgcgccccgtcagtcactccccgtgcagacgcctcgtgctcggtgctcctgta
FATA1-2_CHK80 tcctttaccgctcctttcattctgcgaggccccctgttgaatgtattcgttgcctgtgtg
FATA1-2_CHK22 tcctttaccgctcctttcattctgcgaggccccctgttgaatgtattcgttgcctgtgtg
FATA1-2_CHK80 gccaagcgcgctgctgggcgcgccgccgtcgggcggtgctcggcgactctggcggaagcc
FATA1-2_CHK22 gccaagcgcgctgctgggcgcgccgccgtcgggcggtgctcggcgactctggcggaagcc
FATA1-2_CHK80 ggttgttcttctgtaagccacgcgcttgctgcttttggaaaagaggggggtttactgaat
FATA1-2_CHK22 ggttgttcttctgtaagccacgcgcttgctgcttttggaaaagaggggggtttactgaat
FATA1-2_CHK80 ggaggaggagcaggataattggtagtatctgagttgttggggaggcaggaagagttggga
FATA1-2_CHK22 ggaggaggagcaggataattggtagtatctgagttgttggggaggcaggaagagttggga
FATA1-2_CHK80 aatggaagtggcacgatgggcaaggagaatggtgagcatgcgcagggtgatgtcgttggc
FATA1-2_CHK22 aatggaagtggcacgatgggcaaggagaatggtgagcatgcgcagggtgatgtcgttggc
FATA1-2_CHK80 cgagaacgatcctgcactcccgtaagtagaatacggcaatcacgttgatctacatctcta
FATA1-2_CHK22 cgagaacgatcctgcactcccgtaagtagaatacggcaatcacgttgatctacatctcta
FATA1-2_CHK80 acttctccatttaaagccctttccacatgccctcgcgtttgcatcctacaccctttgatc
FATA1-2_CHK22 acttctccatttaaagccctttccacatgccctcgcgtttgcatcctacaccctttgatc
FATA1-2_CHK80 cacatgattgatgaccctggcactatcttaagggctggacatttcaagaaggtttgcgta
FATA1-2_CHK22 cacatgattgatgaccctggcactatcttaagggctggacatttcaagaaggtttgcgta
FATA1-2_CHK80 tctgaagaagggctggtttgaaggggtcgccgatgagagtgggaacaagctgcgaggatg
FATA1-2_CHK22 tctgaagaagggctggtttgaaggggtcgccgatgagagtgggaacaagctgcgaggatg
FATA1-2_CHK80 cctctgcgcgaattcctaaaggagtaatctgcgtccacgtcatcagtagccgtaggcctc
FATA1-2_CHK22 cctctgcgcgaattcctaaaggagtaatctgcgtccacgtcatcagtagccgtaggcctc
FATA1-2_CHK80 tgcgacgtcgccgtgctcctcggccgccgcttcgccgccgcccagcagccccagacgcac
FATA1-2_CHK22 tgcgacgtcgccgtgctcctcggccgccgcttcgccgccgcccagcagccccagacgcac
FATA1-2_CHK80 gcgctcccagacggcgcgcgcgacggggacgatcatgagcagcgcgatctggggcgtgtc
FATA1-2_CHK22 gcgctcccagacggcgcgcgcgacggggacgatcatgagcagcgcgatctggggcgtgtc
FATA1-2_CHK80 gtgcgagctgagccggacgaccacgttgccctggccgcgcgtggtgtactggcagtttgc
FATA1-2_CHK22 gtgcgagctgagccggacgaccacgttgccctggccgcgcgtggtgtactggcagtttgc
FATA1-2_CHK80 cgagaccatggtgtctgacttgaagtaggagccgtcaaagcgcgggaggcgcgtctcggc
FATA1-2_CHK22 cgagaccatggtgtctgacttgaagtaggagccgtcaaagcgcgggaggcgcgtctcggc
FATA1-2_CHK80 cgagacgttgccgccgacgaccaggctctggcgcttctgacgcgggtcgcggccctgcca
FATA1-2_CHK22 cgagacgttgccgccgacgaccaggctctggcgcttctgacgcgggtcgcggccctgcca
FATA1-2_CHK80 ctgcgcggagcctcccaggagcaggcgcgcctcgtcgcggctgcgcagcttgaggtcggc
FATA1-2_CHK22 ctgcgcggagcctcccaggagcaggcgcgcctcgtcgcggctgcgcagcttgaggtcggc
FATA1-2_CHK80 cgccagcgccgtgcccggctcgtaggccgaccccatcttgacgaagaggcggccgaccgc
FATA1-2_CHK22 cgccagcgccgtgcccggctcgtaggccgaccccatcttgacgaagaggcggccgaccgc
FATA1-2_CHK80 cgcgcggagcttgaccgcgtcggacaggcgcaggcgctcgtccagcttgaccccgagcgc
FATA1-2_CHK22 cgcgcggagcttgaccgcgtcggacaggcgcaggcgctcgtccagcttgaccccgagcgc
FATA1-2_CHK80 cgcgttgccgggcttgaacggcagcgagaactcctcgcccagcttggagccgagcaggcc
FATA1-2_CHK22 cgcgttgccgggcttgaacggcagcgagaactcctcgcccagcttggagccgagcaggcc
FATA1-2_CHK80 cagcgccagcttctgccgcttgccgcgcgcgcgcaggcgcgagtcgacgcgcagcgtgta
FATA1-2_CHK22 cagcgccagcttctgccgcttgccgcgcgcgcgcaggcgcgagtcgacgcgcagcgtgta
FATA1-2_CHK80 gag
FATA1-2_CHK22 gag
KASII-1_CHK80 gcctgcctcgccaactgcgagttctgcgcgcgcggcgggcgcgagggccgcgacggcgcg
(SEQ ID NO: 9)
KASII-1_CHK22 gcctgcctcgccaactgcgagttctgcgcgcgcggcgggcgcgagggccgcgacggcgcg
(SEQ ID NO: 10)
KASII-1_CHK80 ccggggttcaccgcctcggtcgaggagctctgggagcggcggcgaacagagtttgtgcac
KASII-1_CHK22 ccggggttcaccgcctcggtcgaggagctctgggagcggcggcgaacagagtttgtgcac
KASII-1_CHK80 ctcccggagcaggagtttgccggcatcatgctcgtcatcatgccctgccgctctgacaag
KASII-1_CHK22 ctcccggagcaggagtttgccggcatcatgctcgtcatcatgccctgccgctctgacaag
KASII-1_CHK80 tcgaaaaaaggcgtcgccaagtacgcaagcgggggtggggtgggttgggggtggtggcga
KASII-1_CHK22 tcgaaaaaaggcgtcgccaagtacgcaagcgggggtggggtgggttgggggtggtggcga
KASII-1_CHK80 tgcgatgaaaatggtggtgttggtggttccgattggcgtgtcggaattttgtgaactgag
KASII-1_CHK22 tgcgatgaaaatggtggtgttggtggttccgattggcgtgtcggaattttgtgaactgag
KASII-1_CHK80 cggcactctgcggctctgccggcgcatgcctagtccagttttcgatgtgcgctatcatgt
KASII-1_CHK22 cggcactctgcggctctgccggcgcatgcctagtccagttttcgatgtgcgctatcatgt
KASII-1_CHK80 accacgccgcatgcgaatctcaccctttcttctccgccccaggtatggtcacctgagcga
KASII-1_CHK22 accacgccgcatgcgaatctcaccctttcttctccgccccaggtatggtcacctgagcga
KASII-1_CHK80 cggcctgttgcacctggtgctcatcaggcggtgcagccgcctccagtacctcaagtttct
KASII-1_CHK22 cggcctgttgcacctggtgctcatcaggcggtgcagccgcctccagtacctcaagtttct
KASII-1_CHK80 gctcaggatgtcgcacatcggccttgaggcgggcgggcagcacgggtcctacatccaggt
KASII-1_CHK22 gctcaggatgtcgcacatcggccttgaggcgggcgggcagcacgggtcctacatccaggt
KASII-1_CHK80 cttgcctgcccacgcggtgcacatcgaagcggtacgagcgtggatcgggaggcacttgac
KASII-1_CHK22 cttgcctgcccacgcggtgcacatcgaagcggtacgagcgtggatcgggaggcacttgac
KASII-1_CHK80 agtcgcgtcgccgaacaagggacgggtaccctgaggagcttgttggaccgctgattcact
KASII-1_CHK22 agtcgcgtcgccgaacaagggacgggtaccctgaggagcttgttggaccgctgattcact
KASII-1_CHK80 ccctgacctcccttttctctttccaaaacaggttggccaggagagccactggaacgtcga
KASII-1_CHK22 ccctgacctcccttttctctttccaaaacaggttggccaggagagccactggaacgtcga
KASII-1_CHK80 tggggagctcattcaaagccgcacgataaacgcgcagctccaccgcggagtgatcgatgt
KASII-1_CHK22 tggggagctcattcaaagccgcacgataaacgcgcagctccaccgcggagtgatcgatgt
KASII-1_CHK80 gtttgcccgaggcgtggagggctgacgtgcgcgaaactagctgggggcccccattcccgc
KASII-1_CHK22 gtttgcccgaggcgtggagggctgacgtgcgcgaaactagctgggggcccccattcccgc
KASII-1_CHK80 cctttaaacgcctgcctcctggtccccgaccggtgctggcggccctgatcaattcgtcca
KASII-1_CHK22 cctttaaacgcctgcctcctggtccccgaccggtgctggcggccctgatcaattcgtcca
KASII-1_CHK80 ttccgttttattctttgacaatgagcgcctcatcccagtgccaccgcccatccccaaatt
KASII-1_CHK22 ttccgttttattctttgacaatgagcgcctcatcccagtgccaccgcccatccccaaatt
KASII-1_CHK80 gttcctctcaaacctctcagatacctcccttcaaactgctcccaagagtgcacgagtact
KASII-1_CHK22 gttcctctcaaacctctcagatacctcccttcaaactgctcccaagagtgcacgagtact
KASII-1_CHK80 tgtaatgttatgcgaccgctgttcacaatgtagtcggcatgcttgtgtgagcgcgttcga
KASII-1_CHK22 tgtaatgttatgcgaccgctgttcacaatgtagtcggcatgcttgtgtgagcgcgttcga
KASII-1_CHK80 aactcgctcagcccgtcggcctgcccatggacgtcccctgatcgctttatgcccaccgca
KASII-1_CHK22 aactcgctcagcccgtcggcctgcccatggacgtcccctgatcgctttatgcccaccgca
KASII-1_CHK80 tggaccgcgacacgccagttttcggattcaatagcaacgaatacgcatcgataattctgt
KASII-1_CHK22 tggaccgcgacacgccagttttcggattcaatagcaacgaatacgcatcgataattctgt
KASII-1_CHK80 gacattgcatgcctcaccgcgtgaattgctgtcccaaacgtaagcattatcatggctcgg
KASII-1_CHK22 gacattgcatgcctcaccgcgtgaattgctgtcccaaacgtaagcattatcatggctcgg
KASII-1_CHK80 tcacgcgatcctggatccggggatcctggaccgctggtggagagcgctgccgtcggattg
KASII-1_CHK22 tcacgcgatcctggatccggggatcctggaccgctggtggagagcgctgccgtcggattg
KASII-1_CHK80 gtggcaagcaagattgcgcaggttggcgaagggagagaccaaaaccggaggctggaagcg
KASII-1_CHK22 gtggcaagcaagattgcgcaggttggcgaagggagagaccaaaaccggaggctggaagcg
KASII-1_CHK80 ggcacaacatcgtattattgcgtatagtagagcagtggcagtcgcatttcgaggtccgca
KASII-1_CHK22 ggcacaacatcgtattattgcgtatagtagagcagtggcagtcgcatttcgaggtccgca
KASII-1_CHK80 acggatctcgcaagctcgctacgctcacagtaggagataggggaccactgcccctgccag
KASII-1_CHK22 acggatctcgcaagctcgctacgctcacagtaggagataggggaccactgcccctgccag
KASII-1_CHK80 aATGGTCGCGACCCTGTCCCTCGCCGGCCCCGCCTGCAACACGCAGTGCGTATCCAGCAA
KASII-1_CHK22 aATGGTCGCGACCCTGTCCCTCGCCGGCCCCGCCTGCAACACGCAGTGCGTATCCAGCAA
KASII-1_CHK80 GCGGGTTGTCGCCTTCAACCGCCCCCATGTTGGCGTCCGGGCTCGATCAGGTGCGCTGAG
KASII-1_CHK22 GCGGGTTGTCGCCTTCAACCGCCCCCATGTTGGCGTCCGGGCTCGATCAGGTGCGCTGAG
KASII-1_CHK80 GGGGGTTTGGTGGGCCCGCGCCTCTGGGCCCGTGTCGGCCGTGCGGACGTGGGGCCCGGG
KASII-1_CHK22 GGGGGTTTGGTGGGCCCGCGCCTCTGGGCCCGTGTCGGCCGTGCGGACGTGGGGCCCGGG
KASII-1_CHK80 GTAGTGGATCAGCAGGGGTTGCATGCAAATGCCTATACCGGCGATTGAATAGCGATGAAC
KASII-1_CHK22 GTAGTGGATCAGCAGGGGTTGCATGCAAATGCCTATACCGGCGATTGAATAGCGATGAAC
KASII-1_CHK80 GGGATACGGTTGCGCTCACTCCATGCCCATGCGACCCCGTTTCTGTCCGCCAGCCGTGGT
KASII-1_CHK22 GGGATACGGTTGCGCTCACTCCATGCCCATGCGACCCCGTTTCTGTCCGCCAGCCGTGGT
KASII-1_CHK80 CGCCCGAGCTGCGAAGCGGGACCCCACCCAGCGCATTGTGATCACCGGAATGGGCGTGGC
KASII-1_CHK22 CGCCCGAGCTGCGAAGCGGGACCCCACCCAGCGCATTGTGATCACCGGAATGGGCGTGGC
KASII-1_CHK80 CTCCGTGTTTGGCAACGATGTCGAGACCTTTTACGACAAGCTTCTGGAAGGAACGAGCGG
KASII-1_CHK22 CTCCGTGTTTGGCAACGATGTCGAGACCTTTTACGACAAGCTTCTGGAAGGAACGAGCGG
KASII-1_CHK80 CGTGGACCTGATTTCCAGGTGCGTAGGTCCTTGGATGAATGCGTCTAGGTTGCGAGGTGA
KASII-1_CHK22 CGTGGACCTGATTTCCAGGTGCGTAGGTCCTTGGATGAATGCGTCTAGGTTGCGAGGTGA
KASII-1_CHK80 CTGGCCAGGAAGCAGCAGGCTTGGGGTTTGGTGTTCTGATTTCTGGTAATTTGAGGTTTC
KASII-1_CHK22 CTGGCCAGGAAGCAGCAGGCTTGGGGTTTGGTGTTCTGATTTCTGGTAATTTGAGGTTTC
KASII-1_CHK80 ATTATAAGATTCTGTACGGTCTTGTTTCGAAAACATGCAACAACTCCACACACACACACT
KASII-1_CHK22 ATTATAAGATTCTGTACGGTCTTGTTTCGAAAACATGCAACAACTCCACACACACACACT
KASII-1_CHK80 CCTCTCAACTGAGTCTGCAGGTTTGACATCTCCGAGTTCCCGACCAAGTTTGCGGCGCAG
KASII-1_CHK22 CCTCTCAACTGAGTCTGCAGGTTTGACATCTCCGAGTTCCCGACCAAGTTTGCGGCGCAG
KASII-1_CHK80 ATCACCGGCTTCTCCGTGGAGGACTGCGTGGACAAGAAGAACGCGCGGCGGTACGACGAC
KASII-1_CHK22 ATCACCGGCTTCTCCGTGGAGGACTGCGTGGACAAGAAGAACGCGCGGCGGTACGACGAC
KASII-1_CHK80 GCGCTGTCGTACGCGATGGTGGCCTCCAAGAAGGCCCTGCGCCAGGCAGGCCTGGAGAAG
KASII-1_CHK22 GCGCTGTCGTACGCGATGGTGGCCTCCAAGAAGGCCCTGCGCCAGGCAGGCCTGGAGAAG
KASII-1_CHK80 GACAAGTGCCCCGAGGGCTACGGGGCGCTGGACAAGACGCGCACGGGCGTGCTGGTCGGC
KASII-1_CHK22 GACAAGTGCCCCGAGGGCTACGGGGCGCTGGACAAGACGCGCACGGGCGTGCTGGTCGGC
KASII-1_CHK80 TCGGGCATGGGCGGGCTGACGGTCTTCCAGGACGGCGTCAAGGCGCTGGTGGAGAAGGGC
KASII-1_CHK22 TCGGGCATGGGCGGGCTGACGGTCTTCCAGGACGGCGTCAAGGCGCTGGTGGAGAAGGGC
KASII-1_CHK80 TACAAGAAGATGAGCCCCTTCTTCATCCCCTACGCCATCACCAACATGGGCTCCGCGCTG
KASII-1_CHK22 TACAAGAAGATGAGCCCCTTCTTCATCCCCTACGCCATCACCAACATGGGCTCCGCGCTG
KASII-1_CHK80 GTGGGCATCGACCAGGGCTTCATGGGCCCCAACTACTCCGTCTCCACAGCCTGCGCGACG
KASII-1_CHK22 GTGGGCATCGACCAGGGCTTCATGGGCCCCAACTACTCCGTCTCCACAGCCTGCGCGACG
KASII-1_CHK80 TCCAACTACGCATTTGTGAACGCGGCCAACCACATCCGCAAGGGCGACGCGGACGTCATG
KASII-1_CHK22 TCCAACTACGCATTTGTGAACGCGGCCAACCACATCCGCAAGGGCGACGCGGACGTCATG
KASII-1_CHK80 GTCGTCGGCGGCACCGAGGCCTCCATCGTGCCCGTGGGCCTGGGCGGCTTTGTGGCCTGC
KASII-1_CHK22 GTCGTCGGCGGCACCGAGGCCTCCATCGTGCCCGTGGGCCTGGGCGGCTTTGTGGCCTGC
KASII-1_CHK80 CGCGCGCTGTCCACGCGCAACGACGAGCCCAAGCGCGCGAGCCGGCCGTGGGACGAGGGC
KASII-1_CHK22 CGCGCGCTGTCCACGCGCAACGACGAGCCCAAGCGCGCGAGCCGGCCGTGGGACGAGGGC
KASII-1_CHK80 CGCGACGGCTTTGTGATGGGCGAGGGCGCGGCCGTGCTGGTCATGGAGTCGCTGGAGCAC
KASII-1_CHK22 CGCGACGGCTTTGTGATGGGCGAGGGCGCGGCCGTGCTGGTCATGGAGTCGCTGGAGCAC
KASII-1_CHK80 GCGCAGAAGCGTGGCGCGACCATCCTGGGCGAGTACCTGGGCGGCGCCATGACCTGCGAC
KASII-1_CHK22 GCGCAGAAGCGTGGCGCGACCATCCTGGGCGAGTACCTGGGCGGCGCCATGACCTGCGAC
KASII-1_CHK80 GCGCACCACATGACGGACCCGCACCCCGAGGGCCTGGGCGTGAGCACCTGCATCCGCCTG
KASII-1_CHK22 GCGCACCACATGACGGACCCGCACCCCGAGGGCCTGGGCGTGAGCACCTGCATCCGCCTG
KASII-1_CHK80 GCGCTCGAGGACGCCGGCGTCTCGCCCGACGAGGTCAACTACGTCAACGCGCACGCCACC
KASII-1_CHK22 GCGCTCGAGGACGCCGGCGTCTCGCCCGACGAGGTCAACTACGTCAACGCGCACGCCACC
KASII-1_CHK80 TCCACCCTGGTGGGCGACAAGGCCGAGGTGCGCGCGGTCAAGTCGGTCTTTGGCGACATG
KASII-1_CHK22 TCCACCCTGGTGGGCGACAAGGCCGAGGTGCGCGCGGTCAAGTCGGTCTTTGGCGACATG
KASII-1_CHK80 AAGGGTATCAAGATGAACGCCACCAAGAGTATGATCGGGCACTGCCTGGGCGCCGCCGGC
KASII-1_CHK22 AAGGGTATCAAGATGAACGCCACCAAGAGTATGATCGGGCACTGCCTGGGCGCCGCCGGC
KASII-1_CHK80 GGCATGGAGGCCGTCGCGACGCTCATGGCCATCCGCACCGGCTGGGTGCACCCCACCATC
KASII-1_CHK22 GGCATGGAGGCCGTCGCGACGCTCATGGCCATCCGCACCGGCTGGGTGCACCCCACCATC
KASII-1_CHK80 AACCACGACAACCCCATCGCCGAGGTCGATGGCCTGGACGTCGTCGCCAACGCCAAGGCC
KASII-1_CHK22 AACCACGACAACCCCATCGCCGAGGTCGATGGCCTGGACGTCGTCGCCAACGCCAAGGCC
KASII-1_CHK80 CAGCACGACATCAACGTCGCCATCTCCAACTCCTTCGGCTTTGGCGGGCACAACTCCGTC
KASII-1_CHK22 CAGCACGACATCAACGTCGCCATCTCCAACTCCTTCGGCTTTGGCGGGCACAACTCCGTC
KASII-1_CHK80 GTCGCCTTTGCGCCCTTCCGCGAGTAGgtgaagcgagcgtgctttgctgaggagggaggc
KASII-1_CHK22 GTCGCCTTTGCGCCCTTCCGCGAGTAGgtgaagcgagcgtgctttgctgaggagggaggc
KASII-1_CHK80 ggggtgcgagcgctctggccgtgcgcgcgatactctccccgcatgagcagactcctcgtg
KASII-1_CHK22 ggggtgcgagcgctctggccgtgcgcgcgatactctccccgcatgagcagactcctcgtg
KASII-1_CHK80 ccacgcccgaatctacttgtcaacgagcaactgtgtgttttgtccgtggccaatcttatt
KASII-1_CHK22 ccacgcccgaatctacttgtcaacgagcaactgtgtgttttgtccgtggccaatcttatt
KASII-1_CHK80 atttctccgactgtggccgtactctgtttggctgtgcaagcaccccaggaactcttttgt
KASII-1_CHK22 atttctccgactgtggccgtactctgtttggctgtgcaagcaccccaggaactcttttgt
KASII-1_CHK80 tgagcggggggttatcgtagagagggtttgcgaaatgggcaccgatcggaaggccttgca
KASII-1_CHK22 tgagcggggggttatcgtagagagggtttgcgaaatgggcaccgatcggaaggccttgca
KASII-1_CHK80 aacgtggcggtcattggccgacatgatttccgtggattcactgatgcggcattgcttacc
KASII-1_CHK22 aacgtggcggtcattggccgacatgatttccgtggattcactgatgcggcattgcttacc
KASII-1_CHK80 attcatattgaaaagacagccttgcaacctacaatttgatggagcaaatacatatatata
KASII-1_CHK22 attcatattgaaaagacagccttgcaacctacaatttgatggagcaaatacatatatata
KASII-1_CHK80 cggggccgacgtgtcagatggccgttgcgctcttgagctccacggcccgacacagctgct
KASII-1_CHK22 cggggccgacgtgtcagatggccgttgcgctcttgagctccacggcccgacacagctgct
KASII-1_CHK80 cgatagcttcctccagcggcaggcagccgcgctggccgtcagcgtacgtgcgcaccgcca
KASII-1_CHK22 cgatagcttcctccagcggcaggcagccgcgctggccgtcagcgtacgtgcgcaccgcca
KASII-1_CHK80 gggtcgagctcccggcctcttccgggccgatgaccgccatgacggggactttggccctct
KASII-1_CHK22 gggtcgagctcccggcctcttccgggccgatgaccgccatgacggggactttggccctct
KASII-1_CHK80 cggcgttgcgaacgagcttgcccatggaggcgccgccgggcgcgagctccacgcgcaggc
KASII-1_CHK22 cggcgttgcgaacgagcttgcccatggaggcgccgccgggcgcgagctccacgcgcaggc
KASII-1_CHK80 cgcgcgcgcgcatggcggctgccaccgtctccatgtacggccgcaccgcgtccgtcgtgg
KASII-1_CHK22 cgcgcgcgcgcatggcggctgccaccgtctccatgtacggccgcaccgcgtccgtcgtgg
KASII-1_CHK80 gcagcagtcgcacctgctccggggccagccagagcgggaaggcgcccgcgtagtgctcga
KASII-1_CHK22 gcagcagtcgcacctgctccggggccagccagagcgggaaggcgcccgcgtagtgctcga
KASII-1_CHK80 tcaggatgccgaagaagcgctccagcgagccgagcagggcgcggtgcaccatgatgggcc
KASII-1_CHK22 tcaggatgccgaagaagcgctccagcgagccgagcagggcgcggtgcaccatgatgggcc
KASII-1_CHK80 gctggcggctgccgtcggccgccgcgtactcgagcccgaaccgctcgggcaggttgaagt
KASII-1_CHK22 gctggcggctgccgtcggccgccgcgtactcgagcccgaaccgctcgggcaggttgaagt
KASII-1_CHK80 ccagctgcaccgtggagcactgccacttgcgcccgagcgcgtcctggatcttgacgtcga
KASII-1_CHK22 ccagctgcaccgtggagcactgccacttgcgcccgagcgcgtcctggatcttgacgtcga
KASII-1_CHK80 tcttcgggccgtaaaaggcgccgccgccgacgtccaccgtgaaggcccagccctgtcgat
KASII-1_CHK22 tcttcgggccgtaaaaggcgccgccgccgacgtccaccgtgaaggcccagccctgtcgat
KASII-1_CHK80 gaatgattgttgggagggggggtttcgaggttggatgacaccacaataatatagtcagcg
KASII-1_CHK22 gaatgattgttgggagggggggtttcgaggttggatgacaccacaataatatagtcagcg
KASII-1_CHK80 acacgcgtgctgcacgataattaccagtacagactcggcgatgataacaccacccttttc
KASII-1_CHK22 acacgcgtgctgcacgataattaccagtacagactcggcgatgataacaccacccttttc
KASII-1_CHK80 atcaaccgaaatgagccgttccctgttgacaaacacacacgcacacacaccttgtcgttg
KASII-1_CHK22 atcaaccgaaatgagccgttccctgttgacaaacacacacgcacacacaccttgtcgttg
KASII-1_CHK80 agcgcttcagcgagcgccgcctcggccctggcccagtcctcgtcggacccgaccgactcg
KASII-1_CHK22 agcgcttcagcgagcgccgcctcggccctggcccagtcctcgtcggacccgaccgactcg
KASII-1_CHK80 tccggccgcgtggagaggttgacctcgaaccggcacgcgtcgaagccaaaggcgcggaag
KASII-1_CHK22 tccggccgcgtggagaggttgacctcgaaccggcacgcgtcgaagccaaaggcgcggaag
KASII-1_CHK80 acgcgctccgtgaggtccagcacggcccggatctcggccgcgatctgctccggcaggcag
KASII-1_CHK22 acgcgctccgtgaggtccagcacggcccggatctcggccgcgatctgctccggcaggcag
KASII-1_CHK80 aagatgtgggcgtcgtcctgcgtgaagccgcgcacgcggaagaggccgtgcatggtgccg
KASII-1_CHK22 aagatgtgggcgtcgtcctgcgtgaagccgcgcacgcggaagaggccgtgcatggtgccg
KASII-1_CHK80 gagcgctcgtagcggtagacagtgcccagctccgcccagcgcagcggcaggtcgcggtag
KASII-1_CHK22 gagcgctcgtagcggtagacagtgcccagctccgcccagcgcagcggcaggtcgcggtag
KASII-1_CHK80 ctgacgggcctggccgcgtagacgctga
KASII-1_CHK22 ctgacgggcctggccgcgtagacgctga
KASII-2_CHK80 gcctgcctcgccaactgcgagttctgcgcgcgcggcgggcgcgagggccgcgacggcgcg
(SEQ ID NO: 11)
KASII-2_CHK22 gcctgcctcgccaactgcgagttctgcgcgcgcggcgggcgcgagggccgcgacggcgcg
(SEQ ID NO: 12)
KASII-2_CHK80 gcggggttcaccgcctcggccgaggagctctgggagcggcggcgagcagagtttgtgcac
KASII-2_CHK22 gcggggttcaccgcctcggccgaggagctctgggagcggcggcgagcagagtttgtgcac
KASII-2_CHK80 ctcccggagcaggagtttgcaggcatcatgctcgtcatcatgccctgccgctctgacaag
KASII-2_CHK22 ctcccggagcaggagtttgcaggcatcatgctcgtcatcatgccctgccgctctgacaag
KASII-2_CHK80 tcgaaaaagggcgtcgccaagtacgcaagcacagggggtggggtggggtgggggtggtgg
KASII-2_CHK22 tcgaaaaagggcgtcgccaagtacgcaagcacagggggtggggtggggtgggggtggtgg
KASII-2_CHK80 cggtgcgatgaaaatggtggtgtttggtggttcacattggcgtgtcggagttttgtgaag
KASII-2_CHK22 cggtgcgatgaaaatggtggtgtttggtggttcacattggcgtgtcggagttttgtgaag
KASII-2_CHK80 tgagcttcactctgcggctctcccggcgcatgcctagtccagttttcgatgtgcgctatc
KASII-2_CHK22 tgagcttcactctgcggctctcccggcgcatgcctagtccagttttcgatgtgcgctatc
KASII-2_CHK80 gtgtaccacgccgcgtgcgaatcccaccctttcttctccgccccaggtatggtcacctga
KASII-2_CHK22 gtgtaccacgccgcgtgcgaatcccaccctttcttctccgccccaggtatggtcacctga
KASII-2_CHK80 gcgacggcctgctgcacctggtgctcatcaggcggtgcagccgcctccagtacctcaagt
KASII-2_CHK22 gcgacggcctgctgcacctggtgctcatcaggcggtgcagccgcctccagtacctcaagt
KASII-2_CHK80 ttctgctcaggatgtcgcacatcggcctcgaggcgggcgggcagcacgggtcctacatcc
KASII-2_CHK22 ttctgctcaggatgtcgcacatcggcctcgaggcgggcgggcagcacgggtcctacatcc
KASII-2_CHK80 aggtcctgcccgcccacgcggtgcacatcgaagcggtacgagcgtggatcgggaggcact
KASII-2_CHK22 aggtcctgcccgcccacgcggtgcacatcgaagcggtacgagcgtggatcgggaggcact
KASII-2_CHK80 tgacagtcgcgtcgcccgacaaggcacgggcgccctgaggagcttgttgggccgctgatt
KASII-2_CHK22 tgacagtcgcgtcgcccgacaaggcacgggcgccctgaggagcttgttgggccgctgatt
KASII-2_CHK80 cacttccctggcctcccctttctctttccaaaccaggttggccaggagagccactggaac
KASII-2_CHK22 cacttccctggcctcccctttctctttccaaaccaggttggccaggagagccactggaac
KASII-2_CHK80 gtcgatggggagctcattcaaagccgcacgatcaacgcgcagctccaccgcggagtgatc
KASII-2_CHK22 gtcgatggggagctcattcaaagccgcacgatcaacgcgcagctccaccgcggagtgatc
KASII-2_CHK80 gacgtgtttgccagaggcgtggagggctgacgagcgcgaaactagctgggggccccattc
KASII-2_CHK22 gacgtgtttgccagaggcgtggagggctgacgagcgcgaaactagctgggggccccattc
KASII-2_CHK80 ccgcccttgaaacgcctgcctcctgggccctggccggtgctggcggccctgaccgattcg
KASII-2_CHK22 ccgcccttgaaacgcctgcctcctgggccctggccggtgctggcggccctgaccgattcg
KASII-2_CHK80 tccattccgttttattctttgacaatgagcgcctcatcccagtgcctcatcccagtgcca
KASII-2_CHK22 tccattccgttttattctttgacaatgagcgcctcatcccagtgcctcatcccagtgcca
KASII-2_CHK80 ccgcccatccccaaattgttcctctcaaacctctcagata-cccccttcaaactgctccc
KASII-2_CHK22 ccgcccatccccaaattgttcctctcaaacctctcagataccccccttcaaactgctccc
KASII-2_CHK80 aagagtgcacgagtatttgtaatgttatacgaccgctgttcacaatgtagtcggcatgct
KASII-2_CHK22 aagagtgcacgagtatttgtaatgttatacgaccgctgttcacaatgtagtcggcatgct
KASII-2_CHK80 tgtatgagcctgttcgaaactcgctcagcccgtcggcctgcccatggacctcccctgatc
KASII-2_CHK22 tgtatgagcctgttcgaaactcgctcagcccgtcggcctgcccatggacctcccctgatc
KASII-2_CHK80 gcttcacgcccaccgcatggactgcgacacgccagttttgggattcaatatcaaaaaata
KASII-2_CHK22 gcttcacgcccaccgcatggactgcgacacgccagttttgggattcaatatcaaaaaata
KASII-2_CHK80 cgcttcggtaattctgtaacgttgcatgcctcaccgcgtgaattgctgtcccaaacgtaa
KASII-2_CHK22 cgcttcggtaattctgtaacgttgcatgcctcaccgcgtgaattgctgtcccaaacgtaa
KASII-2_CHK80 gcatcatcgtggctcggtcacgcgatcctggatccggggatcctagaccgctggtggaga
KASII-2_CHK22 gcatcatcgtggctcggtcacgcgatcctggatccggggatcctagaccgctggtggaga
KASII-2_CHK80 gcgctgccgtcggattggtggcaagtaagattgcgcaggttggcgaagggagagaccaaa
KASII-2_CHK22 gcgctgccgtcggattggtggcaagtaagattgcgcaggttggcgaagggagagaccaaa
KASII-2_CHK80 accggaggctggaagcgggcacaacatcgtattattgcgtatagtagagcagtggcagtc
KASII-2_CHK22 accggaggctggaagcgggcacaacatcgtattattgcgtatagtagagcagtggcagtc
KASII-2_CHK80 gcatttcgaggtccgcaacggatctcgcaagctcgctacgctcacagtaggagaaagggg
KASII-2_CHK22 gcatttcgaggtccgcaacggatctcgcaagctcgctacgctcacagtaggagaaagggg
KASII-2_CHK80 accactgcccctgccagaATGGTCGCGACCCTCTCCCTCGCCGGCCCCGCCTGCAACACG
KASII-2_CHK22 accactgcccctgccagaATGGTCGCGACCCTCTCCCTCGCCGGCCCCGCCTGCAACACG
KASII-2_CHK80 CAGTGCGTATCCGGCAAGCGGGCTGTCGCCTTCAACCGCCCCCATGTTGGCGTCCGGGCT
KASII-2_CHK22 CAGTGCGTATCCGGCAAGCGGGCTGTCGCCTTCAACCGCCCCCATGTTGGCGTCCGGGCT
KASII-2_CHK80 CGATCAGGTGCGCTGAGGGGGGTTTGGTGTGCCCGCGCCTCTGGGCCCGTGTCGGCCGTG
KASII-2_CHK22 CGATCAGGTGCGCTGAGGGGGGTTTGGTGTGCCCGCGCCTCTGGGCCCGTGTCGGCCGTG
KASII-2_CHK80 CGGACGTGGGGCCCTGGGCAGTGGATCAGCAGGGTTTGCGTGCAAATGCCTATACCGGCG
KASII-2_CHK22 CGGACGTGGGGCCCTGGGCAGTGGATCAGCAGGGTTTGCGTGCAAATGCCTATACCGGCG
KASII-2_CHK80 ATTGAATAGCGATGAACGGGATACGGTTGCGCTCACTCCATGCCCATGCGACCCCGTTTC
KASII-2_CHK22 ATTGAATAGCGATGAACGGGATACGGTTGCGCTCACTCCATGCCCATGCGACCCCGTTTC
KASII-2_CHK80 TGTCCGCCAGCCGTGGTCGCCCGGGCTGCGAAGCGGGACCCCACCCAGCGCATTGTGATC
KASII-2_CHK22 TGTCCGCCAGCCGTGGTCGCCCGGGCTGCGAAGCGGGACCCCACCCAGCGCATTGTGATC
KASII-2_CHK80 ACCGGAATGGGCGTGGCCTCCGTGTTTGGCAACGATGTCGAGACCTTTTACAACAAGCTT
KASII-2_CHK22 ACCGGAATGGGCGTGGCCTCCGTGTTTGGCAACGATGTCGAGACCTTTTACAACAAGCTT
KASII-2_CHK80 CTGGAAGGAACGAGCGGCGTGGACCTGATTTCCAGGTGCGTAGGTCCTTGGATGCATGCG
KASII-2_CHK22 CTGGAAGGAACGAGCGGCGTGGACCTGATTTCCAGGTGCGTAGGTCCTTGGATGCATGCG
KASII-2_CHK80 TCTAGGTTGGGAGGCGGCTGGCGAGGAAGCAGCAGGCTTGGGGTTTGGTGTTCCGATTTC
KASII-2_CHK22 TCTAGGTTGGGAGGCGGCTGGCGAGGAAGCAGCAGGCTTGGGGTTTGGTGTTCCGATTTC
KASII-2_CHK80 TGGCAATTTGAGGTTTCATTGTGAGATTCTATGCGGTCTTGTTTCGAAAACATGCAACAA
KASII-2_CHK22 TGGCAATTTGAGGTTTCATTGTGAGATTCTATGCGGTCTTGTTTCGAAAACATGCAACAA
KASII-2_CHK80 CTCCACACACACACACTCCTCTCCACCAACTCTGCAGGTTTGACATCTCCGAGTTCCCGA
KASII-2_CHK22 CTCCACACACACACACTCCTCTCCACCAACTCTGCAGGTTTGACATCTCCGAGTTCCCGA
KASII-2_CHK80 CCAAGTTTGCGGCGCAGATCACCGGCTTCTCCGTGGAGGACTGCGTGGACAAGAAGAACG
KASII-2_CHK22 CCAAGTTTGCGGCGCAGATCACCGGCTTCTCCGTGGAGGACTGCGTGGACAAGAAGAACG
KASII-2_CHK80 CGCGGCGGTACGACGACGCGCTGTCGTACGCGATGGTGGCCTCCAAGAAGGCCCTGCGCC
KASII-2_CHK22 CGCGGCGGTACGACGACGCGCTGTCGTACGCGATGGTGGCCTCCAAGAAGGCCCTGCGCC
KASII-2_CHK80 AGGCGGGACTGGAGAAGGACAAGTGCCCCGAGGGCTACGGAGCGCTGGATAAGACGCGCG
KASII-2_CHK22 AGGCGGGACTGGAGAAGGACAAGTGCCCCGAGGGCTACGGAGCGCTGGATAAGACGCGCG
KASII-2_CHK80 CGGGCGTGCTGGTCGGCTCGGGCATGGGCGGGCTGACGGTCTTCCAGGACGGCGTCAAGG
KASII-2_CHK22 CGGGCGTGCTGGTCGGCTCGGGCATGGGCGGGCTGACGGTCTTCCAGGACGGCGTCAAGG
KASII-2_CHK80 CGCTGGTGGAGAAGGGCTACAAGAAGATGAGCCCCTTCTTCATCCCCTACGCCATCACCA
KASII-2_CHK22 CGCTGGTGGAGAAGGGCTACAAGAAGATGAGCCCCTTCTTCATCCCCTACGCCATCACCA
KASII-2_CHK80 ACATGGGCTCCGCGCTGGTGGGCATCGACCAGGGCTTCATGGGGCCCAACTACTCCGTCT
KASII-2_CHK22 ACATGGGCTCCGCGCTGGTGGGCATCGACCAGGGCTTCATGGGGCCCAACTACTCCGTCT
KASII-2_CHK80 CCACGGCCTGCGCGACCTCCAACTACGCCTTTGTGAACGCGGCCAACCACATCCGCAAGG
KASII-2_CHK22 CCACGGCCTGCGCGACCTCCAACTACGCCTTTGTGAACGCGGCCAACCACATCCGCAAGG
KASII-2_CHK80 GCGACGCGGACGTCATGGTCGTGGGCGGCACCGAGGCCTCCATCGTGCCCGTGGGCCTGG
KASII-2_CHK22 GCGACGCGGACGTCATGGTCGTGGGCGGCACCGAGGCCTCCATCGTGCCCGTGGGCCTGG
KASII-2_CHK80 GCGGCTTTGTGGCCTGCCGCGCGCTGTCCACGCGCAACGACGAGCCCAAGCGCGCGAGCC
KASII-2_CHK22 GCGGCTTTGTGGCCTGCCGCGCGCTGTCCACGCGCAACGACGAGCCCAAGCGCGCGAGCC
KASII-2_CHK80 GGCCGTGGGACGAGGGCCGCGACGGCTTCGTGATGGGCGAGGGCGCGGCCGTGCTGGTCA
KASII-2_CHK22 GGCCGTGGGACGAGGGCCGCGACGGCTTCGTGATGGGCGAGGGCGCGGCCGTGCTGGTCA
KASII-2_CHK80 TGGAGTCGCTGGAGCACGCGCAGAAGCGCGGCGCGACCATCCTGGGCGAGTACCTGGGGG
KASII-2_CHK22 TGGAGTCGCTGGAGCACGCGCAGAAGCGCGGCGCGACCATCCTGGGCGAGTACCTGGGGG
KASII-2_CHK80 GCGCCATGACCTGCGACGCGCACCACATGACGGACCCGCACCCCGAGGGCCTGGGCGTGA
KASII-2_CHK22 GCGCCATGACCTGCGACGCGCACCACATGACGGACCCGCACCCCGAGGGCCTGGGCGTGA
KASII-2_CHK80 GCACCTGCATCCGCCTGGCGCTCGAGGACGCCGGCGTCTCGCCCGACGAGGTCAACTACG
KASII-2_CHK22 GCACCTGCATCCGCCTGGCGCTCGAGGACGCCGGCGTCTCGCCCGACGAGGTCAACTACG
KASII-2_CHK80 TCAACGCGCACGCCACCTCCACCCTGGTGGGCGACAAGGCCGAGGTGCGCGCGGTCAAGT
KASII-2_CHK22 TCAACGCGCACGCCACCTCCACCCTGGTGGGCGACAAGGCCGAGGTGCGCGCGGTCAAGT
KASII-2_CHK80 CGGTCTTTGGCGACATGAAGGGCATCAAGATGAACGCCACCAAGTCCATGATCGGGCACT
KASII-2_CHK22 CGGTCTTTGGCGACATGAAGGGCATCAAGATGAACGCCACCAAGTCCATGATCGGGCACT
KASII-2_CHK80 GCCTGGGCGCCGCCGGCGGCATGGAGGCCGTCGCCACGCTCATGGCCATCCGCACCGGCT
KASII-2_CHK22 GCCTGGGCGCCGCCGGCGGCATGGAGGCCGTCGCCACGCTCATGGCCATCCGCACCGGCT
KASII-2_CHK80 GGGTGCACCCCACCATCAACCACGACAACCCCATCGCCGAGGTCGACGGCCTGGACGTCG
KASII-2_CHK22 GGGTGCACCCCACCATCAACCACGACAACCCCATCGCCGAGGTCGACGGCCTGGACGTCG
KASII-2_CHK80 TCGCCAACGCCAAGGCCCAGCACAAAATCAACGTCGCCATCTCCAACTCCTTCGGCTTCG
KASII-2_CHK22 TCGCCAACGCCAAGGCCCAGCACAAAATCAACGTCGCCATCTCCAACTCCTTCGGCTTCG
KASII-2_CHK80 GCGGGCACAACTCCGTCGTCGCCTTTGCGCCCTTCCGCGAGTAGgcggagcgagcgcgct
KASII-2_CHK22 GCGGGCACAACTCCGTCGTCGCCTTTGCGCCCTTCCGCGAGTAGgcggagcgagcgcgct
KASII-2_CHK80 tggctgaggagggaggcggggtgcgagccctttggctgcgcgcgatactctccccgcacg
KASII-2_CHK22 tggctgaggagggaggcggggtgcgagccctttggctgcgcgcgatactctccccgcacg
KASII-2_CHK80 agcagactccacgcgcctgaatctacttgtcaacgagcaaccgtgtgttttgtccgtggc
KASII-2_CHK22 agcagactccacgcgcctgaatctacttgtcaacgagcaaccgtgtgttttgtccgtggc
KASII-2_CHK80 cattcttattatttctccgactgtggccgtactctgtttggctgtgcaagcaccccagga
KASII-2_CHK22 cattcttattatttctccgactgtggccgtactctgtttggctgtgcaagcaccccagga
KASII-2_CHK80 actcttttgtcgagcggggggtgtcgtagagagggtccgcgaaacaggcaccgatcgcga
KASII-2_CHK22 actcttttgtcgagcggggggtgtcgtagagagggtccgcgaaacaggcaccgatcgcga
KASII-2_CHK80 gcctcgtggtggtcattgttcgacatgattcccggagattcactgatgcggcattgctta
KASII-2_CHK22 gcctcgtggtggtcattgttcgacatgattcccggagattcactgatgcggcattgctta
KASII-2_CHK80 ccattcatttgaaaagacagcttgcaacctacaattcgaaggagcgaatacatatatata
KASII-2_CHK22 ccattcatttgaaaagacagcttgcaacctacaattcgaaggagcgaatacatatatata
KASII-2_CHK80 cggggccggcgtgtcagatcgccgtttcgctcttgagctccacggcccgacacagctgct
KASII-2_CHK22 cggggccggcgtgtcagatcgccgtttcgctcttgagctccacggcccgacacagctgct
KASII-2_CHK80 cgatgacttcctccagcggcaggcagccgcgctggccgtcggcgtacgtgcgcaccgcca
KASII-2_CHK22 cgatgacttcctccagcggcaggcagccgcgctggccgtcggcgtacgtgcgcaccgcca
KASII-2_CHK80 gggtcgagctcccggcctcttccgggccgatgaccgccatgacggggaccttggccctct
KASII-2_CHK22 gggtcgagctcccggcctcttccgggccgatgaccgccatgacggggaccttggccctct
KASII-2_CHK80 cggcgttgcggatgagcttgcccatggaggccccgccgggcgcgagctccacgcgcaggc
KASII-2_CHK22 cggcgttgcggatgagcttgcccatggaggccccgccgggcgcgagctccacgcgcaggc
KASII-2_CHK80 cgcgcgcgcgcatggcgtctgccaccgtctccatgtagggccgcaccgcgtccgtcgtgg
KASII-2_CHK22 cgcgcgcgcgcatggcgtctgccaccgtctccatgtagggccgcaccgcgtccgtcgtgg
KASII-2_CHK80 gcagcaggcgcacctgctccggggccagccagagcgggaaggcgcccgcgtagtgctcga
KASII-2_CHK22 gcagcaggcgcacctgctccggggccagccagagcgggaaggcgcccgcgtagtgctcga
KASII-2_CHK80 tcaggatgccgaagaagcgctccagcgagccgagcagggcgcggtgaatcatgatgggcc
KASII-2_CHK22 tcaggatgccgaagaagcgctccagcgagccgagcagggcgcggtgaatcatgatgggcc
KASII-2_CHK80 gctggcggctgccgtcggcggccgcgtactcgagcccaaaccgctcgggcaaattgaagt
KASII-2_CHK22 gctggcggctgccgtcggcggccgcgtactcgagcccaaaccgctcgggcaaattgaagt
KASII-2_CHK80 ccagctgcaccgtggagcactgccacttgcgcccgagcgcgtcctggatcttgacgtcga
KASII-2_CHK22 ccagctgcaccgtggagcactgccacttgcgcccgagcgcgtcctggatcttgacgtcga
KASII-2_CHK80 tcttcgggccgtaaaaggcgccgccgccggcgtccaccgtgaaggcccagccctgtcgat
KASII-2_CHK22 tcttcgggccgtaaaaggcgccgccgccggcgtccaccgtgaaggcccagccctgtcgat
KASII-2_CHK80 gaatgattcgtggttgagagggcggatttttcgaggtctggatgatgatacggtatgcgc
KASII-2_CHK22 gaatgattcgtggttgagagggcggatttttcgaggtctggatgatgatacggtatgcgc
KASII-2_CHK80 cacaataatactacactcgcgccagtcagcgacacgcgtgctgcacaatgcctactacta
KASII-2_CHK22 cacaataatactacactcgcgccagtcagcgacacgcgtgctgcacaatgcctactacta
KASII-2_CHK80 tagactcggtgatgatgataccacccttttcatcgaacgagatgggccattccctactga
KASII-2_CHK22 tagactcggtgatgatgataccacccttttcatcgaacgagatgggccattccctactga
KASII-2_CHK80 caaacacacacgcaccttgtcgttcagcgcttcggcgagcgccgcctcggccctggccca
KASII-2_CHK22 caaacacacacgcaccttgtcgttcagcgcttcggcgagcgccgcctcggccctggccca
KASII-2_CHK80 gtcctcgtcggacccgaccgactcgtccggccgcgtggagaggttgacctcgaaccggcg
KASII-2_CHK22 gtcctcgtcggacccgaccgactcgtccggccgcgtggagaggttgacctcgaaccggcg
KASII-2_CHK80 cgcgtcgaagccaaaggcgcggaagacgcgctccgtgaggtccagcacggcccggatctc
KASII-2_CHK22 cgcgtcgaagccaaaggcgcggaagacgcgctccgtgaggtccagcacggcccggatctc
KASII-2_CHK80 ggccgcgatctgctccggcaggcagaagacgtgggcgtcgtcctgcgtgaagccgcgcac
KASII-2_CHK22 ggccgcgatctgctccggcaggcagaagacgtgggcgtcgtcctgcgtgaagccgcgcac
KASII-2_CHK80 gcggaagaggccgtgcatggtgccggagcgctcgtagcggtagaccgtgcccagctccgc
KASII-2_CHK22 gcggaagaggccgtgcatggtgccggagcgctcgtagcggtagaccgtgcccagctccgc
KASII-2_CHK80 ccagcgcagcggcaggtcgcggtagctgacgggccgggccgcgtagacgctga
KASII-2_CHK22 ccagcgcagcggcaggtcgcggtagctgacgggccgggccgcgtagacgctga
SAD2-1_CHK80 aaacccgatgatgcccagcgcggagctcagcatgtcgccgagcatcgctggataggacga
(SEQ ID NO: 13)
SAD2-1_CHK22 aaacccgatgatgcccagcgcggagctcagcatgtcgccgagcatcgctggataggacga
(SEQ ID NO: 14)
SAD2-1_CHK80 attggcagggtaccacctgaagaatgggaggcaggtgctgctgattacgagtgtggaaaa
SAD2-1_CHK22 attggcagggtaccacctgaagaatgggaggcaggtgctgctgattacgagtgtggaaaa
SAD2-1_CHK80 gaaagggggcagagagccgtcctcagatccgaccactatgcaggtagccgctcgcccgtg
SAD2-1_CHK22 gaaagggggcagagagccgtcctcagatccgaccactatgcaggtagccgctcgcccgtg
SAD2-1_CHK80 cccgcctggctgaatattgacacatgcccatcaaggcaggcaggcatttctgtgcatgca
SAD2-1_CHK22 cccgcctggctgaatattgacacatgcccatcaaggcaggcaggcatttctgtgcatgca
SAD2-1_CHK80 ccaaacccataatcttcgaaaacacacagcatgtaccaacgcacgcgtaaaagttggggt
SAD2-1_CHK22 ccaaacccataatcttcgaaaacacacagcatgtaccaacgcacgcgtaaaagttggggt
SAD2-1_CHK80 gctgccagtgcgtcatgccaggcatgatgtgctcctgcacatcggccatgatctcctcca
SAD2-1_CHK22 gctgccagtgcgtcatgccaggcatgatgtgctcctgcacatcggccatgatctcctcca
SAD2-1_CHK80 tcgtctcgggcgtctccggcgcctggtccgggagccgttccgccagatacccagacgcca
SAD2-1_CHK22 tcgtctcgggcgtctccggcgcctggtccgggagccgttccgccagatacccagacgcca
SAD2-1_CHK80 cctccgacctcacggggtacttttcgagcgactgtcggtagtcgacgatcgcgtccacca
SAD2-1_CHK22 cctccgacctcacggggtacttttcgagcgactgtcggtagtcgacgatcgcgtccacca
SAD2-1_CHK80 tggagtagccgaggcgccggaactggcgtgacggagggagaacagggggggttgatgatc
SAD2-1_CHK22 tggagtagccgaggcgccggaactggcgtgacggagggagaacagggggggttgatgatc
SAD2-1_CHK80 acacgccagtctcacaacgcatgtaagacccgttggattatgagtataatcatgcattac
SAD2-1_CHK22 acacgccagtctcacaacgcatgtaagacccgttggattatgagtataatcatgcattac
SAD2-1_CHK80 tagttgggtgagcgccaggcataaggcacaccgacgtcgatggcacgagcaactcccgca
SAD2-1_CHK22 tagttgggtgagcgccaggcataaggcacaccgacgtcgatggcacgagcaactcccgca
SAD2-1_CHK80 tcatatttcctattgtcctaacgccaagccggtcaccacccgcatgctcgtactacagcg
SAD2-1_CHK22 tcatatttcctattgtcctaacgccaagccggtcaccacccgcatgctcgtactacagcg
SAD2-1_CHK80 cacgcaccgcttcgtgatccaccgggtgaacgtagtcctcgacggaaacatctggttcgg
SAD2-1_CHK22 cacgcaccgcttcgtgatccaccgggtgaacgtagtcctcgacggaaacatctggttcgg
SAD2-1_CHK80 gcctcctgcttgcactcccgcccatgccgacaacctttctgctgttaccacgacccacaa
SAD2-1_CHK22 gcctcctgcttgcactcccgcccatgccgacaacctttctgctgttaccacgacccacaa
SAD2-1_CHK80 tgcaacgcgacacgaccgtgtgggactgatcggttcactgcacctgcatgcaattgtcac
SAD2-1_CHK22 tgcaacgcgacacgaccgtgtgggactgatcggttcactgcacctgcatgcaattgtcac
SAD2-1_CHK80 aagcgcttactccaattgtattcgtttgttttctgggagcagttgctcgaccgcccgcgt
SAD2-1_CHK22 aagcgcttactccaattgtattcgtttgttttctgggagcagttgctcgaccgcccgcgt
SAD2-1_CHK80 cccgcaggcagcgatgacgtgtgcgtggcctgggtgtttcgtcgaaaggccagcaaccct
SAD2-1_CHK22 cccgcaggcagcgatgacgtgtgcgtggcctgggtgtttcgtcgaaaggccagcaaccct
SAD2-1_CHK80 aaatcgcaggcgatccggagattgggatctgatccgagtttggaccagatccgccccgat
SAD2-1_CHK22 aaatcgcaggcgatccggagattgggatctgatccgagtttggaccagatccgccccgat
SAD2-1_CHK80 gcggcacgggaactgcatcgactcggcgcggaacccagctttcgtaaatgccagattggt
SAD2-1_CHK22 gcggcacgggaactgcatcgactcggcgcggaacccagctttcgtaaatgccagattggt
SAD2-1_CHK80 gtccgatacctggatttgccatcagcgaaacaagacttcagcagcgagcgtatttggcgg
SAD2-1_CHK22 gtccgatacctggatttgccatcagcgaaacaagacttcagcagcgagcgtatttggcgg
SAD2-1_CHK80 gcgtgctaccagggttgcatacattgcccatttctgtctggaccgctttactggcgcaga
SAD2-1_CHK22 gcgtgctaccagggttgcatacattgcccatttctgtctggaccgctttactggcgcaga
SAD2-1_CHK80 gggtgagttgatggggttggcaggcatcgaaacgcgcgtgcatggtgtgcgtgtctgttt
SAD2-1_CHK22 gggtgagttgatggggttggcaggcatcgaaacgcgcgtgcatggtgtgcgtgtctgttt
SAD2-1_CHK80 tcggctgcacgaattcaatagtcggatgggcgacggtagaattgggtgtggcgctcgcgt
SAD2-1_CHK22 tcggctgcacgaattcaatagtcggatgggcgacggtagaattgggtgtggcgctcgcgt
SAD2-1_CHK80 gcatgcctcgccccgtcgggtgtcatgaccgggactggaatcccccctcgcgaccatctt
SAD2-1_CHK22 gcatgcctcgccccgtcgggtgtcatgaccgggactggaatcccccctcgcgaccatctt
SAD2-1_CHK80 gctaacgctcccgactctcccgaccgcgcgcaggatagactcttgttcaaccaatcgaca
SAD2-1_CHK22 gctaacgctcccgactctcccgaccgcgcgcaggatagactcttgttcaaccaatcgaca
SAD2-1_CHK80 ATGGCGTCTGCCGTCACCTTTGCGTGCGCCCCTCCCCGCGGCGCGGTCGCCGCGCCGGGT
SAD2-1_CHK22 ATGGCGTCTGCCGTCACCTTTGCGTGCGCCCCTCCCCGCGGCGCGGTCGCCGCGCCGGGT
SAD2-1_CHK80 CGCCGCGCTGCCTCGCGTCCCCTGGTGGTGCGCGCGGTCGCCAGCGAGGCCCCGCTGGGC
SAD2-1_CHK22 CGCCGCGCTGCCTCGCGTCCCCTGGTGGTGCGCGCGGTCGCCAGCGAGGCCCCGCTGGGC
SAD2-1_CHK80 GTTCCGCCCTCGGTGCAGCGCCCCTCCCCCGTGGTCTACTCCAAGCTGGACAAGCAGCAC
SAD2-1_CHK22 GTTCCGCCCTCGGTGCAGCGCCCCTCCCCCGTGGTCTACTCCAAGCTGGACAAGCAGCAC
SAD2-1_CHK80 CGCCTGACGCCCGAGCGCCTGGAGCTGGTGCAGAGCATGGGGCAGTTTGCGGAGGAGAGG
SAD2-1_CHK22 CGCCTGACGCCCGAGCGCCTGGAGCTGGTGCAGAGCATGGGGCAGTTTGCGGAGGAGAGG
SAD2-1_CHK80 GTGCTGCCCGTGCTGCACCCCGTGGACAAGCTGTGGCAGCCGCAGGACTTTTTGCCCGAC
SAD2-1_CHK22 GTGCTGCCCGTGCTGCACCCCGTGGACAAGCTGTGGCAGCCGCAGGACTTTTTGCCCGAC
SAD2-1_CHK80 CCCGAGTCGCCCGACTTCGAGGATCAGGTGGCGGAGCTGCGCGCGCGCGCCAAGGACCTG
SAD2-1_CHK22 CCCGAGTCGCCCGACTTCGAGGATCAGGTGGCGGAGCTGCGCGCGCGCGCCAAGGACCTG
SAD2-1_CHK80 CCCGACGAGTACTTTGTGGTGCTGGTGGGGGACATGATCACGGAGGAGGCGCTGCCGACC
SAD2-1_CHK22 CCCGACGAGTACTTTGTGGTGCTGGTGGGGGACATGATCACGGAGGAGGCGCTGCCGACC
SAD2-1_CHK80 TACATGGCCATGCTCAACACGCTGGACGGCGTGCGCGACGACACGGGCGCGGCCGACCAC
SAD2-1_CHK22 TACATGGCCATGCTCAACACGCTGGACGGCGTGCGCGACGACACGGGCGCGGCCGACCAC
SAD2-1_CHK80 CCGTGGGCGCGCTGGACGCGGCAGTGGGTGGCCGAGGAGAACCGGCACGGCGACCTGCTG
SAD2-1_CHK22 CCGTGGGCGCGCTGGACGCGGCAGTGGGTGGCCGAGGAGAACCGGCACGGCGACCTGCTG
SAD2-1_CHK80 AACAAGTACTGCTGGCTGACGGGGCGCGTCAACATGCGGGCCGTGGAGGTGACCATCAAC
SAD2-1_CHK22 AACAAGTACTGCTGGCTGACGGGGCGCGTCAACATGCGGGCCGTGGAGGTGACCATCAAC
SAD2-1_CHK80 AACCTGATCAAGAGCGGCATGAACCCGCAGACGGACAACAACCCTTATTTGGGGTTCGTC
SAD2-1_CHK22 AACCTGATCAAGAGCGGCATGAACCCGCAGACGGACAACAACCCTTATTTGGGGTTCGTC
SAD2-1_CHK80 TACACCTCCTTCCAGGAGCGCGCCACCAAGTACAGCCACGGCAACACCGCGCGCCTTGCG
SAD2-1_CHK22 TACACCTCCTTCCAGGAGCGCGCCACCAAGTACAGCCACGGCAACACCGCGCGCCTTGCG
SAD2-1_CHK80 GCCGAGCACGGCGACAAGAACCTGAGCAAGATCTGCGGGCTGATCGCCAGCGACGAGGGC
SAD2-1_CHK22 GCCGAGCACGGCGACAAGAACCTGAGCAAGATCTGCGGGCTGATCGCCAGCGACGAGGGC
SAD2-1_CHK80 CGGCACGAGATCGCCTACACGCGCATCGTGGACGAGTTCTTCCGCCTCGACCCCGAGGGC
SAD2-1_CHK22 CGGCACGAGATCGCCTACACGCGCATCGTGGACGAGTTCTTCCGCCTCGACCCCGAGGGC
SAD2-1_CHK80 GCCGTCGCCGCCTACGCCAACATGATGCGCAAGCAGATCACCATGCCCGCGCACCTCATG
SAD2-1_CHK22 GCCGTCGCCGCCTACGCCAACATGATGCGCAAGCAGATCACCATGCCCGCGCACCTCATG
SAD2-1_CHK80 GACGACATGGGCCACGGCGAGGCCAACCCGGGCCGCAACCTCTTCGCCGACTTCTCCGCG
SAD2-1_CHK22 GACGACATGGGCCACGGCGAGGCCAACCCGGGCCGCAACCTCTTCGCCGACTTCTCCGCG
SAD2-1_CHK80 GTCGCCGAGAAGATCGACGTCTACGACGCCGAGGACTACTGCCGCATCCTGGAGCACCTC
SAD2-1_CHK22 GTCGCCGAGAAGATCGACGTCTACGACGCCGAGGACTACTGCCGCATCCTGGAGCACCTC
SAD2-1_CHK80 AACGCGCGCTGGAAGGTGGACGAGCGCCAGGTCAGCGGCCAGGCCGCCGCGGACCAGGAG
SAD2-1_CHK22 AACGCGCGCTGGAAGGTGGACGAGCGCCAGGTCAGCGGCCAGGCCGCCGCGGACCAGGAG
SAD2-1_CHK80 TACGTCCTGGGCCTGCCCCAGCGCTTCCGGAAACTCGCCGAGAAGACCGCCGCCAAGCGC
SAD2-1_CHK22 TACGTCCTGGGCCTGCCCCAGCGCTTCCGGAAACTCGCCGAGAAGACCGCCGCCAAGCGC
SAD2-1_CHK80 AAGCGCGTCGCGCGCAGGCCCGTCGCCTTCTCCTGGATCTCCGGGCGCGAGATCATGGTC
SAD2-1_CHK22 AAGCGCGTCGCGCGCAGGCCCGTCGCCTTCTCCTGGATCTCCGGGCGCGAGATCATGGTC
SAD2-1_CHK80 TAGggagcgacgagtgtgcgtgcggggctggcgggagtgggacgccctcctcgctcctct
SAD2-1_CHK22 TAGggagcgacgagtgtgcgtgcggggctggcgggagtgggacgccctcctcgctcctct
SAD2-1_CHK80 ctgttctgaacggaacaatcggccaccccgcgctacgcgccacgcatcgagcaacgaaga
SAD2-1_CHK22 ctgttctgaacggaacaatcggccaccccgcgctacgcgccacgcatcgagcaacgaaga
SAD2-1_CHK80 aaaccccccgatgataggttgcggtggctgccgggatatagatccggccgcacatcaaag
SAD2-1_CHK22 aaaccccccgatgataggttgcggtggctgccgggatatagatccggccgcacatcaaag
SAD2-1_CHK80 ggcccctccgccagagaagaagctcctttcccagcagactccttctgctgccaaaacact
SAD2-1_CHK22 ggcccctccgccagagaagaagctcctttcccagcagactccttctgctgccaaaacact
SAD2-1_CHK80 tctctgtccacagcaacaccaaaggatgaacagatcaacttgcgtctccgcgtagcttcc
SAD2-1_CHK22 tctctgtccacagcaacaccaaaggatgaacagatcaacttgcgtctccgcgtagcttcc
SAD2-1_CHK80 tcggctagcgtgcttgcaacaggtccctgcactattatcttcctgctttcctctgaatta
SAD2-1_CHK22 tcggctagcgtgcttgcaacaggtccctgcactattatcttcctgctttcctctgaatta
SAD2-1_CHK80 tgcggcaggcgagcgctcgctctggcgagcgctccttcgcgccgccctcgctgatcgagt
SAD2-1_CHK22 tgcggcaggcgagcgctcgctctggcgagcgctccttcgcgccgccctcgctgatcgagt
SAD2-1_CHK80 gtacagtcaatgaatggtcctgggcgaagaacgagggaatttgtgggtaaaacaagcatc
SAD2-1_CHK22 gtacagtcaatgaatggtcctgggcgaagaacgagggaatttgtgggtaaaacaagcatc
SAD2-1_CHK80 gtctctcaggccccggcgcagtggccgttaaagtccaagaccgtgaccaggcagcgcagc
SAD2-1_CHK22 gtctctcaggccccggcgcagtggccgttaaagtccaagaccgtgaccaggcagcgcagc
SAD2-1_CHK80 gcgtccgtgtgcgggccctgcctggcggctcggcgtgccaggctcgagagcagctccctc
SAD2-1_CHK22 gcgtccgtgtgcgggccctgcctggcggctcggcgtgccaggctcgagagcagctccctc
SAD2-1_CHK80 aggtcgccttggacggcctctgcgaggccggtgagggcctgcaggagcgcctcgagcgtg
SAD2-1_CHK22 aggtcgccttggacggcctctgcgaggccggtgagggcctgcaggagcgcctcgagcgtg
SAD2-1_CHK80 gcagtggcggtcgtatccgggtcgccggtcaccgcctgcgactcgccatccgaagagcca
SAD2-1_CHK22 gcagtggcggtcgtatccgggtcgccggtcaccgcctgcgactcgccatccgaagagcca
SAD2-1_CHK80 tcgtcgtcgtctcccagactggacgcgctcgccatcggccccgccgatgcagaacacgcc
SAD2-1_CHK22 tcgtcgtcgtctcccagactggacgcgctcgccatcggccccgccgatgcagaacacgcc
SAD2-1_CHK80 tctgcgctcgccgtgcgcagcgcggcgcgcagcgctgcgcgcgccagggccgcgtccagc
SAD2-1_CHK22 tctgcgctcgccgtgcgcagcgcggcgcgcagcgctgcgcgcgccagggccgcgtccagc
SAD2-1_CHK80 aggcccatcgccagctcccggccgcgagggccggccagcgcctcaaagccgccggcgcgg
SAD2-1_CHK22 aggcccatcgccagctcccggccgcgagggccggccagcgcctcaaagccgccggcgcgg
SAD2-1_CHK80 gcggccgccaccaccgaggccagcgcggcgcgcgcgccctccagggtcggcgcccgggtc
SAD2-1_CHK22 gcggccgccaccaccgaggccagcgcggcgcgcgcgccctccagggtcggcgcccgggtc
SAD2-1_CHK80 acggcgtcgcgcgcccaggcggccagcgccgccccccgtagaacgcagtgcgcctccatc
SAD2-1_CHK22 acggcgtcgcgcgcccaggcggccagcgccgccccccgtagaacgcagtgcgcctccatc
SAD2-1_CHK80 gaccgcacaaagtgggagacccggtggctcgtggggtcgtagggccgcacgcggcggcgc
SAD2-1_CHK22 gaccgcacaaagtgggagacccggtggctcgtggggtcgtagggccgcacgcggcggcgc
SAD2-1_CHK80 gcgggcctcgtggttgcgcgcgtgctgctccgcagccccgcaatcgtcacgctcccttgc
SAD2-1_CHK22 gcgggcctcgtggttgcgcgcgtgctgctccgcagccccgcaatcgtcacgctcccttgc
SAD2-1_CHK80 cgatccacgctcgcggcggccagcctggcccaggccagctggaggcccaggcgcatgctg
SAD2-1_CHK22 cgatccacgctcgcggcggccagcctggcccaggccagctggaggcccaggcgcatgctg
SAD2-1_CHK80 gcgcggacctgctccagaaaggggccgtcgcccagcatgcacagcggccaggggagcgtc
SAD2-1_CHK22 gcgcggacctgctccagaaaggggccgtcgcccagcatgcacagcggccaggggagcgtc
SAD2-1_CHK80 aggtcgaaccgcaggcgctccagcgtggcgacgcgcggaggctcgcccgggagcggccag
SAD2-1_CHK22 aggtcgaaccgcaggcgctccagcgtggcgacgcgcggaggctcgcccgggagcggccag
SAD2-1_CHK80 ggacgagcgggcgattgggtggccggcggcgaataaccagggtgcactccgtcagcagcc
SAD2-1_CHK22 ggacgagcgggcgattgggtggccggcggcgaataaccagggtgcactccgtcagcagcc
SAD2-1_CHK80 tcctccgacctcacacccccctgcctcttgagctggcggcaccgggccgcgatggccgca
SAD2-1_CHK22 tcctccgacctcacacccccctgcctcttgagctggcggcaccgggccgcgatggccgca
SAD2-1_CHK80 ccggcgctgtccagcacgcccgcttccacagccacgtgcagctcgatgccggcgacgtcc
SAD2-1_CHK22 ccggcgctgtccagcacgcccgcttccacagccacgtgcagctcgatgccggcgacgtcc
SAD2-1_CHK80 gtctc
SAD2-1_CHK22 gtctc
SAD2-2_CHK80 ggagctcagcatgtcg-ccagcatcgctggataagacgaattggcagggtaccacctgaa
(SEQ ID NO: 15)
SAD2-2_CHK22 ggagctcagcatgtcgcccagcatcgccggataagacgaattggcagggtaccacctgaa
(SEQ ID NO: 16)
SAD2-2_CHK80 gaatgggaggcaggtgttgctgattacgagtgtgtaaaagaaaggggtagagagccgtcc
SAD2-2_CHK22 gaatgggaggcaggtgttgttgattatgagtgtgtaaaagaaaggggtagagagccgtcc
SAD2-2_CHK80 tcagatccgaccactatgcaggtagccgctcgcccatgcccgcctggctgaatattgaca
SAD2-2_CHK22 tcagatccgactactatgcaggtagccgctcgcccatgcccgcctggctgaatattgatg
SAD2-2_CHK80 catgcccatcaaggcaggcaggcatttctgtgcatgcaccaagcccacaatcttccacaa
SAD2-2_CHK22 catgcccatcaaggcaggcaggcatttctgtgcacgcaccaagcccacaatcttccacaa
SAD2-2_CHK80 cacacagcatgtaccaacgcacgcgtaaaagttggggtgctgccagtgcgtcatgccagg
SAD2-2_CHK22 cacacagcatgtaccaacgcacgcgtaaaagttggggtgctgccagtgcgtcatgccagg
SAD2-2_CHK80 catgatgtgctcctgcacatcggccatgatctcctccatcgtctcgggtgtctccggcgc
SAD2-2_CHK22 catgatgtgctcctgcacatccgccatgatctcctccatcgtctcgggtgtttccggcgc
SAD2-2_CHK80 ctggtccgggagccgttccgccagatacccagacgccacctccgacctcacggggtactt
SAD2-2_CHK22 ctggtccgggagccgttccgccagatacccagacgccacctccgacctcacggggtactt
SAD2-2_CHK80 ttcgagcgactgtcggtagtcgacgatcgcgtccaccatggagtagccgaggcgccggaa
SAD2-2_CHK22 ttcgagcgtctgccggtagtcgacgatcgcgtccaccatggagtagccgaggcgccggaa
SAD2-2_CHK80 ctggcgtgacggagggaggagagggaggagagag-----------gggggggggatgatc
SAD2-2_CHK22 ctggcgtgacggagggaggagagggaggagagagagggggggggggggggggggatgatt
SAD2-2_CHK80 acacgccagtctcacaacgcatgtaagacccgtttgattatgagtacaatcatgcactac
SAD2-2_CHK22 acacgccagtctcacaacgcatgcaagacccgtttgattatgagtacaatcatgcactac
SAD2-2_CHK80 tagttggatgagcgccaggcataaggcacaccgacgtcgatggcacgagcaactcccgca
SAD2-2_CHK22 tagatggatgagcgccaggcataaggcacaccgacgttgatggcatgagcaactcccgca
SAD2-2_CHK80 tcatatttcctattgtcctcacgccaagccggtcaccatccgcatgctcgtattacagcg
SAD2-2_CHK22 tcatatttcctattgtcctcacgccaagccggtcaccatccgcatgctcatattacagcg
SAD2-2_CHK80 cacgcaccgcttcgtgatccaccgggtgaacgtagtcctcgacggaaacatctggttcgg
SAD2-2_CHK22 cacgcaccgcttcgtgatccaccgggtgaacgtagtcctcgacggaaacatctggctcgg
SAD2-2_CHK80 gcctcgtgctggcactccctcccatgccgacaacctttctgctgtcaccacgacccacga
SAD2-2_CHK22 gcctcgtgctggcactccctcccatgccgacaacctttctgctgtcaccacgacccacga
SAD2-2_CHK80 tgcaacgcgacacga-ccggtgggactgatcggttcactgcacctgcatgcaattgtcac
SAD2-2_CHK22 tgcaacgcgacacgacccggtgggactgatcggttcactgcacctgcatgcaattgtcac
SAD2-2_CHK80 aagcgcatactccaatcgtatccgtttgatttctgtgaaaactcgctcgaccgcccgcgt
SAD2-2_CHK22 aagcgcatactccaatcgtatccgtttgatttctgtgaaaactcgctcgaccgcccgcgt
SAD2-2_CHK80 cccgcaggcagcgatgacgtgtgcgtggcctgggtgtttcgtcgaaaggccagcaacccc
SAD2-2_CHK22 cccgcaggcagcgatgacgtgtgcgtgacctgggtgtttcgtcgaaaggccagcaacccc
SAD2-2_CHK80 aaatcgcaggcgatccggagattgggatctgatccgag-ttggaccagatccccc-cgat
SAD2-2_CHK22 aaatcgcaggcgatccggagattgggatctgatccgagcttggaccagatcccccacgat
SAD2-2_CHK80 gcggcacgggaactgcatcgactcggcgcggaacccagctttcgtaaatgccagattggt
SAD2-2_CHK22 gcggcacgggaactgcatcgactcggcgcggaacccagctttcgtaaatgccagattggt
SAD2-2_CHK80 gtccgataccttgatttgccatcagcgaaacaagacttcagcagcgagcgtatttggcgg
SAD2-2_CHK22 gtccgataccttgatttgccatcagcgaaacaagacttcagcagcgagcgtatttggcgg
SAD2-2_CHK80 gcgtgctaccagggttgcatacattgcccatttctgtctggaccgctttactggcgcaga
SAD2-2_CHK22 gcgtgctaccagggttgcatacattgcccatttctgtctggaccgctttaccggcgcaga
SAD2-2_CHK80 gggtgagttgatggggttggcaggcatcgaaacgcgcgtgcatggtgtgtgtgtctgttt
SAD2-2_CHK22 gggtgagttgatggggttggcaggcatcgaaacgcgcgtgcatggtgtgtgtgtctgttt
SAD2-2_CHK80 tcggctgcacaat-tcaatagtcggatgggcgacggtagaattgggtgttgcgctcgcgt
SAD2-2_CHK22 tcggctgcacaatttcaatagtcggatgggcgacggtagaattgggtgttgcgctcgcgt
SAD2-2_CHK80 gcatgcctcgccccgtcgggtgtcatgaccgggactggaatcccccctcgcgaccctctt
SAD2-2_CHK22 gcatgcctcgccccgtcgggtgtcatgaccgggactggaatcccccctcgcgaccctcct
SAD2-2_CHK80 gctaacgctcccgactctcccgcccgcgcgcaggatagactctagttcaaccaatcgaca
SAD2-2_CHK22 gctaacgctcccgactctcccgcccgcgcgcaggatagactctagttcaaccaatcgaca
SAD2-2_CHK80 ATGGCGTCTGCCGTCACCTTTGCGTGCGCCCCTCCCCGCGGCGCGGTCGCCGCGCCGGGT
SAD2-2_CHK22 ATGGCGTCTGCCGTCACCTTTGCGTGCGCCCCTCCCCGCGGCGCGGTCGCCGCGCCGGGT
SAD2-2_CHK80 CGCCGCGCTGCCTCGCGTCCCCTGGTGGTGCGCGCCGTCGCCAGCGAGGCCCCGCTGGGC
SAD2-2_CHK22 CGCCGCGCTGCCTCGCGTCCCCTGGTGGTGCACGCCGTCGCCAGCGAGGCCCCGCTGGGC
SAD2-2_CHK80 GTGCCGCCCTCGGTGCAGCGCCCCTCCCCCGTGGTCTACTCCAAGCTGGACAAGCAACAC
SAD2-2_CHK22 GTGCCGCCCTCGGTGCAGCGCCCCTCCCCCGTGGTCTACTCCAAGCTGGACAAGCAACAC
SAD2-2_CHK80 CGCCTGACGCCCGAGCGCCTGGAGCTGGTGCAGAGCATGGGTCAGTTTGCGGAGGAGAGG
SAD2-2_CHK22 CGCCTGACGCCCGAGCGCCTGGAGCTGGTGCAGAGCATGGGTCAGTTTGCGGAGGAGAGG
SAD2-2_CHK80 GTGCTCCCCGTGCTGCACCCCGTGGACAAGCTGTGGCAGCCGCAGGACTTTTTGCCCGAC
SAD2-2_CHK22 GTGCTCCCCGTGCTGCACCCCGTGGACAAGCTGTGGCAGCCGCAGGACTTCCTGCCCGAC
SAD2-2_CHK80 CCCGAGTCGCCCGACTTCGAGGACCAGGTGGCGGAGCTGCGCGCGCGCGCCAAGGACCTG
SAD2-2_CHK22 CCCGAGTCGCCCGACTTCGAGGACCAGGTGGCGGAGCTGCGCGCGCGCGCCAAGGACCTG
SAD2-2_CHK80 CCCGACGAGTACTTTGTGGTGCTGGTGGGCGACATGATCACGGAGGAGGCGCTGCCGACC
SAD2-2_CHK22 CCCGACGAGTACTTTGTGGTGCTGGTGGGCGACATGATCACGGAGGAGGCGCTGCCGACC
SAD2-2_CHK80 TACATGGCCATGCTCAACACCTTGGACGGTGTGCGCGACGACACGGGCGCGGCTGACCAC
SAD2-2_CHK22 TACATGGCCATGCTCAACACCTTGGACGGTGTGCGCGACGACACGGGCGCGGCTGACCAC
SAD2-2_CHK80 CCGTGGGCGCGCTGGACGCGGCAGTGGGTGGCCGAGGAGAACCGGCACGGCGACCTGCTG
SAD2-2_CHK22 CCGTGGGCGCGCTGGACGCGGCAGTGGGTGGCCGAGGAGAACCGGCACGGCGACCTGCTG
SAD2-2_CHK80 AACAAGTACTGTTGGCTGACGGGGCGCGTCAACATGCGGGCCGTGGAGGTGACCATCAAC
SAD2-2_CHK22 AACAAGTACTGTTGGCTGACGGGGCGCGTCAACATGCGGGCCGTGGAGGTGACCATCAAC
SAD2-2_CHK80 AACCTGATCAAGAGCGGCATGAACCCGCAGACGGACAACAACCCTTACTTGGGCTTCGTC
SAD2-2_CHK22 AACCTGATCAAGAGCGGCATGAACCCGCAGACGGACAACAACCCTTACTTGGGCTTCGTC
SAD2-2_CHK80 TACACCTCCTTCCAGGAGCGCGCGACCAAGTACAGCCACGGCAACACCGCGCGCCTGGCG
SAD2-2_CHK22 TACACCTCCTTCCAGGAGCGCGCGACCAAGTACAGCCACGGCAACACCGCGCGCCTGGCG
SAD2-2_CHK80 GCCGAGCACGGCGACAAGGGCCTGAGCAAGATCTGCGGGCTGATCGCCAGCGACGAGGGC
SAD2-2_CHK22 GCCGAGCACGGCGACAAGGGCCTGAGCAAGATCTGCGGGCTGATCGCCAGCGACGAGGGC
SAD2-2_CHK80 CGGCACGAGATCGCCTACACGCGCATCGTGGACGAGTTCTTCCGCCTCGACCCCGAGGGC
SAD2-2_CHK22 CGGCACGAGATCGCCTACACGCGCATCGTGGACGAGTTCTTCCGCCTCGACCCCGAGGGC
SAD2-2_CHK80 GCCGTCGCCGCCTACGCCAACATGATGCGCAAGCAGATCACCATGCCCGCGCACCTCATG
SAD2-2_CHK22 GCCGTCGCCGCCTACGCCAACATGATGCGCAAGCAGATCACCATGCCCGCGCACCTCATG
SAD2-2_CHK80 GACGACATGGGCCACGGCGAGGCCAACCCGGGCCGCAACCTCTTCGCCGACTTCTCCGCC
SAD2-2_CHK22 GACGACATGGGCCACGGCGAGGCCAACCCGGGCCGCAACCTCTTCGCCGACTTCTCCGCC
SAD2-2_CHK80 GTCGCCGAGAAGATCGACGTCTACGACGCCGAGGACTACTGCCGCATCCTGGAGCACCTC
SAD2-2_CHK22 GTCGCCGAGAAGATCGACGTCTACGACGCCGAGGACTACTGCCGCATCCTGGAGCACCTC
SAD2-2_CHK80 AACGCGCGCTGGAAGGTGGACGAGCGCCAGGTCAGCGGCCAGGCCGCCGCGGACCAGGAG
SAD2-2_CHK22 AACGCGCGCTGGAAGGTGGACGAGCGCCAGGTCAGCGGCCAGGCCGCCGCGGACCAGGAG
SAD2-2_CHK80 TACGTTCTGGGCCTGCCCCAGCGCTTCCGGAAACTCGCCGAGAAGACCGCCGCCAAGCGC
SAD2-2_CHK22 TACGTTCTGGGCCTGCCCCAGCGCTTCCGGAAACTCGCCGAGAAGACCGCCGCCAAGCGC
SAD2-2_CHK80 AAGCGCGTCGCGCGCAGGCCCGTCGCCTTCTCCTGGATCTCCGGACGCGAGATTATGGTC
SAD2-2_CHK22 AAGCGCGTCGCGCGCAGGCCCGTCGCCTTCTCCTGGATCTCCGGACGCGAGATTATGGTC
SAD2-2_CHK80 TAGggaggtacgagcgcgcgcgagggattggtgggagtgggacgcgctcgtcgctccttt
SAD2-2_CHK22 TAGggaggtacgagcgcgcgcgagggattggtgggagtgggacgcgctcgtcgctccttt
SAD2-2_CHK80 ctattctgaagggaagattggccaccccgctccacgcgccacgcatcgagcaacgaagaa
SAD2-2_CHK22 ctattctgaagggaagattggccaccccgctccacgcgccacgcatcgagcaacgaagaa
SAD2-2_CHK80 aaccccccgatgataggttgcagtggctgccgagatatagatccggctgcacgtcaaagg
SAD2-2_CHK22 aaccccccgatgataggttgcagtggctgccgagatatagatccggctgcacgtcaaagg
SAD2-2_CHK80 gcccctcggccagagaagaagctctttt-ccagcgaccgcagactccttctgccaaaaac
SAD2-2_CHK22 gcccctcggccagagaagaagctcttttcccagcgaccgcagactccttctgccaaaaac
SAD2-2_CHK80 actcttctctgtccacagcaacaccaatggatggacagatcaacttgtgtcttcgcgtag
SAD2-2_CHK22 actcttctctgtccacagcaacaccaatggatggacagatcaacatgtgtcttcgcgtag
SAD2-2_CHK80 cttcctcggctagcgtgcttgcaacaggtccctgcactattatcctcctgctttcctctg
SAD2-2_CHK22 cttcctcggctagcgtgcttgcaacaggtccctgcactattatcctcctgctttcctctg
SAD2-2_CHK80 aattatgcggcaggcgagcgctcgctcttgcgagcgctccttcgcgccgccctcgctgat
SAD2-2_CHK22 aattatgcggcaggcgagcgctcgctcttgcgagcgctccttcgcgccgccctcgctgat
SAD2-2_CHK80 cgagtgtacagtcaatgaatggtcctgggcgaagaacgagggaatttgtgggcgagagag
SAD2-2_CHK22 cgagtgtacagtcaatgaatggtcctgggcgaagaacgagggaatttgtgggcgagagag
SAD2-2_CHK80 catcgtctctcaggccccagcgcagtggccgttaaagtccaagaccgtgaccaggcagcg
SAD2-2_CHK22 catcgtctctcaggccccagcgcagtggccgttaaagtccaagaccgtgaccaggcagcg
SAD2-2_CHK80 catcgcgtccgtgtgcgggccctgcctggcggctcggcgcgccaggctcgagagcagctc
SAD2-2_CHK22 catcgcgtccgtgtgcgggccctgcctggcggctcggcgcgccaggctcgagagcagctc
SAD2-2_CHK80 cctcaggtcgccttggacagcctctgcgaggccggtgagggtctgcaggagcgcctcgag
SAD2-2_CHK22 cctcaggtcgccttggacagcctctgcgaggccggtgagggtctgcaggagcgcctcgag
SAD2-2_CHK80 cgtggcagtggcggttgtatccgggtcgccggtcatcgactgcgactcgccatccgaaga
SAD2-2_CHK22 cgtggcagtggcggttgtatccgggtcgccggtcatcgactgcgactcgccatccgaaga
SAD2-2_CHK80 gccatcgtcgtcgtctcccagactggaagcgctcgccatcggccccgccgatacagaaaa
SAD2-2_CHK22 gccatcgtcgtcgtctcccagactggaagcgctcgccatcggccccgccgatacagaaaa
SAD2-2_CHK80 cgcctctgcgctcgccgtgcgcagcgcggcgcgcaccagggccgcgtccagcagacccat
SAD2-2_CHK22 cgcctctgcgctcgccgtgcgcagcgcggcgcgcaccagggccgcgtccagcagacccat
SAD2-2_CHK80 cgccagctcccggccgcgagggccggccagcgcctcaaagccgccggcgcgggcggccgc
SAD2-2_CHK22 cgccagctcccggccgcgagggccggccagcgcctcaaagccgccggcgcgggcggccgc
SAD2-2_CHK80 cgccgccgaggccagcgcggcgcgcgcgccctccagggtcggcgcccgggtcacggcgtc
SAD2-2_CHK22 cgccgccgaggccagcgcggcgcgcgcgccctccagggtcggcgcccgggtcacggcgtc
SAD2-2_CHK80 gcgcgcccaggcggccagcgccgccccccgcaggacgcagtgcgcctccatcgaccgcac
SAD2-2_CHK22 gcgcgcccaggcggccagcgccgccccccgcaggacgcagtgcgcctccatcgaccgcac
SAD2-2_CHK80 aaagtgggagacccggtggctcgtggggtcgtagggccgcacgcggcggcgcgcgggcct
SAD2-2_CHK22 aaagtgggagacccggtggctcgtggggtcgtagggccgcacgcggcggcgcgcgggcct
SAD2-2_CHK80 cgtgaacgtgcgtttgccattccgcagccctgcggtcgtcacactcccttgccgatccac
SAD2-2_CHK22 cgtgaacgtgcgtttgccattccgcagccctgcggtcgtcacactcccttgccgatccac
SAD2-2_CHK80 gctcgcggcggccagcctggcccaggccagctggaggcccaggagcatgccggcgcggac
SAD2-2_CHK22 gctcgcggcggccagcctggcccaggccagctggaggcccaggagcatgccggcgcggac
SAD2-2_CHK80 ctgctccagaaaggggccgttgcccagcatgcacagtggccaggggagcgtcaggtggaa
SAD2-2_CHK22 ctgctccagaaaggggccgttgcccagcatgcacagtggccaggggagcgtcaggtggaa
SAD2-2_CHK80 ccgcaggcgctccagcgtggcgacgcgcggaggctcgcccgggagcggccagggacgcgt
SAD2-2_CHK22 ccgcaggcgctccagcgtggcgacgcgcggaggctcgcccgggagcggccagggacgcgt
SAD2-2_CHK80 ggacgattgggtggcg-gcatcgaagtgctggagcgcactccgtcagcggcctccgccga
SAD2-2_CHK22 ggacgattgggtggcgggcatcgaagtgctggagcgcactccgtcagcggcctccgccga
SAD2-2_CHK80 cgccgcacccccctgcctcttgagctggcggcaccgggccgcgatggccgcaccggcgct
SAD2-2_CHK22 cgccgcacccccctgcctcttgagctggcggcaccgggccgcgatggccgcaccggcgct
SAD2-2_CHK80 gtccagcacgcccgcttccacagccacgtgcagctcgatgccggcgacgtctgtctccat
SAD2-2_CHK22 gtccagcacgcccgcttccacagccacgtgcagctcgatgccggcgacgtctgtctccat
SAD2-2_CHK80 ca
SAD2-2_CHK22 ca
Key:
Non-coding: lowercase, non-italicized, non-bold
Promoter: italicized, non-bold
Coding: CAPITAL, BOLD, NON-ITALICIZED
Intron: CAPITAL, NON-BOLD, NON-ITALICIZED
SNP difference: underlined a c g t

TABLE 12 shows sequence alignments of genomic regions of CHK80 and CHK22: 6S, DAO1B, FAD2, FATA1, and Thi4.

TABLE 12

| | |
|---|---|
| 6S_CHK22 | acgaattgttgctttgaagggtgctgaatggcaccagatactgggtcggtggcatttgct |
| | (SEQ ID NO: 17) |
| 6S_CHK80 | acgaattgttgctttgaagggtgctgaatggcaccagatactgggtcggtggcatttgct |
| | (SEQ ID NO: 18) |
| 6S_CHK22 | caggcgtcatttgtaggttctgccaataataattccatgaggacagcaagccattgacaa |
| 6S_CHK80 | caggcgtcatttgtaggttctgccaataataattccatgaggacagcaagccattgacaa |
| 6S_CHK22 | gcccactgactcgaattttagagcaacctacgagaccacaccatccgtgcacgcacctcg |
| 6S_CHK80 | gcccactgactcgaattttagagcaacctacgagaccacaccatccgtgcacgcacctcg |
| 6S_CHK22 | aggctgtttttggatacgtgcagccgagcgcatggcccgtcgccgtcctcggtagaggga |
| 6S_CHK80 | aggctgtttttggatacgtgcagccgagcgcatggcccgtcgccgtcctcggtagaggga |
| 6S_CHK22 | gggcagcacagggccctctctttctttttgactggccaagctggcacgctttcggctgcc |
| 6S_CHK80 | gggcagcacagggccctctctttctttttgactggccaagctggcacgctttcggctgcc |
| 6S_CHK22 | gggatcaggtgagctttcgtcggcgggggcgagcagggccaccaggtcgaggacctccct |
| 6S_CHK80 | gggatcaggtgagctttcgtcggcgggggcgagcagggccaccaggtcgaggacctccct |
| 6S_CHK22 | ctccaggcagccaatggtgtggagcaggcctctatgctggtgatgagccgcttgctgctc |
| 6S_CHK80 | ctccaggcagccaatggtgtggagcaggcctctatgctggtgatgagccgcttgctgctc |
| 6S_CHK22 | ctggctgtcggcaccggtcctcctggatggcggaggtggcgcaagaacacccgtgtgtcg |
| 6S_CHK80 | ctggctgtcggcaccggtcctcctggatggcggaggtggcgcaagaacacccgtgtgtcg |
| 6S_CHK22 | cacgttcatcaacacgtcttgcgcagtgtcgcacaccgcaaggacgcaggtactttaaaa |
| 6S_CHK80 | cacgttcatcaacacgtcttgcgcagtgtcgcacaccgcaaggacgcaggtactttaaaa |
| 6S_CHK22 | agacacaagcgtgacatgtgcgtgatcattgcaacggctgccgcagaggacttaccccag |
| 6S_CHK80 | agacacaagcgtgacatgtgcgtgatcattgcaacggctgccgcagaggacttaccccag |
| 6S_CHK22 | ctcagcaagcagtgttgagacggccttggccaccccatcgagctcagcgagcagcttttc |
| 6S_CHK80 | ctcagcaagcagtgttgagacggccttggccaccccatcgagctcagcgagcagcttttc |
| 6S_CHK22 | gtgctggaaggccaggacctcgttttcttgctgggtctccacggctcgccgtcgggattc |
| 6S_CHK80 | gtgctggaaggccaggacctcgttttcttgctgggtctccacggctcgccgtcgggattc |
| 6S_CHK22 | ctggagggaggagggagcccaggggagagtggtgttgtgatgacgacgcgtgtgcgcaga |
| 6S_CHK80 | ctggagggaggagggagcccaggggagagtggtgttgtgatgacgacgcgtgtgcgcaga |
| 6S_CHK22 | ggcagtaggatgctcatgagtgcagcctctttgcagccaggtttgcctgtcgcacagact |
| 6S_CHK80 | ggcagtaggatgctcatgagtgcagcctctttgcagccaggtttgcctgtcgcacagact |
| 6S_CHK22 | tgagaggcaccacccaaagtctactatccaaataatgctccagagcatcagcaatgttgg |
| 6S_CHK80 | tgagaggcaccacccaaagtctactatccaaataatgctccagagcatcagcaatgttgg |
| 6S_CHK22 | ccaccttcagtaccaacgctcccaccctcgtctgcgcacctgcgctgcccgcccccgcgc |
| 6S_CHK80 | ccaccttcagtaccaacgctcccaccctcgtctgcgcacctgcgctgcccgcccccgcgc |
| 6S_CHK22 | ggcctcggcagcgacggacgcgcccgccgcgccctcggcggcctccgcctgcgcctcgag |
| 6S_CHK80 | ggcctcggcagcgacggacgcgcccgccgcgccctcggcggcctccgcctgcgcctcgag |
| 6S_CHK22 | cgcctcacgttccctttccagcgccgccagcgaggcctggacgtccaccaggtgcttgaa |
| 6S_CHK80 | cgcctcacgttccctttccagcgccgccagcgaggcctggacgtccaccaggtgcttgaa |
| 6S_CHK22 | ccgagcaaagttgcgctcctgcagcccggccaggttggccaccagctcctccatgctgga |
| 6S_CHK80 | ccgagcaaagttgcgctcctgcagcccggccaggttggccaccagctcctccatgctgga |
| 6S_CHK22 | gtagcctgtggccgtggagatcttctggaaagcgtcggtgatggcctgctcgcgctgcgc |
| 6S_CHK80 | gtagcctgtggccgtggagatcttctggaaagcgtcggtgatggcctgctcgcgctgcgc |
| 6S_CHK22 | gtgcgacgcaaagtggacgcctggtgaggccgcgccctggccgtcgagggaggacccccc |
| 6S_CHK80 | gtgcgacgcaaagtggacgcctggtgaggccgcgccctggccgtcgagggaggacccccc |
| 6S_CHK22 | gcccggcccggcctcggcggcgcccgccgcgccgcccagcgccctcgcccccgccggcgc |
| 6S_CHK80 | gcccggcccggcctcggcggcgcccgccgcgccgcccagcgccctcgcccccgccggcgc |
| 6S_CHK22 | gccgccgtgcgcgtccagccgcacgggcgctgggtagtcctgcgagaagagctgggccat |
| 6S_CHK80 | gccgccgtgcgcgtccagccgcacgggcgctgggtagtcctgcgagaagagctgggccat |
| 6S_CHK22 | ctcctgctcgcgcgcggcacgctccgcgcggcgatcggcgtcccgggtcgcgcgctcctg |
| 6S_CHK80 | ctcctgctcgcgcgcggcacgctccgcgcggcgatcggcgtcccgggtcgcgcgctcctg |
| 6S_CHK22 | ctggatgagcgccgccagccgccgccactcctgctcgagcgcgcccgactcgcgctccgc |
| 6S_CHK80 | ctggatgagcgccgccagccgccgccactcctgctcgagcgcgcccgactcgcgctccgc |
| 6S_CHK22 | ctGCGCCCGCGCGTGCGCCGCCAGCGCCTTGGCCTTTTCGCCGCGCTCGTGCGCGTCGCT |
| 6S_CHK80 | ctGCGCCCGCGCGTGCGCCGCCAGCGCCTTGGCCTTTTCGCCGCGCTCGTGCGCGTCGCT |
| 6S_CHK22 | GATGTCCATCACCAGGTCCATGAGGTCTGCCTTGCGCCGGCTGAGCCACTGCTTCGTCCG |
| 6S_CHK80 | GATGTCCATCACCAGGTCCATGAGGTCTGCCTTGCGCCGGCTGAGCCACTGCTTCGTCCG |
| 6S_CHK22 | GGCGGCCAAGAGGAGCATGAGGGAGGACTCCTGGTCCAGGGTCCTGACGTGGTCGCGGCT |
| 6S_CHK80 | GGCGGCCAAGAGGAGCATGAGGGAGGACTCCTGGTCCAGGGTCCTGACGTGGTCGCGGCT |
| 6S_CHK22 | CTGGGAGCGGGCCAGCATCATCTGGCTCTGCCGCACCGAGGCCGCCTCCAACTGGTCCTC |
| 6S_CHK80 | CTGGGAGCGGGCCAGCATCATCTGGCTCTGCCGCACCGAGGCCGCCTCCAACTGGTCCTC |
| 6S_CHK22 | CAGCAGCCGCAGTCGCCGCCGACCCTGGCAGAGGAAGACAGGTGAGGGGGGTATGAATTG |
| 6S_CHK80 | CAGCAGCCGCAGTCGCCGCCGACCCTGGCAGAGGAAGACAGGTGAGGGGGGTATGAATTG |
| 6S_CHK22 | TACAGAACAACCACGAGCCTTGTCTAGGCAGAATCCCTACCAGTCATGGCTTTACCTGGA |
| 6S_CHK80 | TACAGAACAACCACGAGCCTTGTCTAGGCAGAATCCCTACCAGTCATGGCTTTACCTGGA |
| 6S_CHK22 | TGACGGCCTGCGAACAGCTGTCCAGCGACCCTCGCTGCCGCCGCTTCTCCCGCACGCTTC |
| 6S_CHK80 | TGACGGCCTGCGAACAGCTGTCCAGCGACCCTCGCTGCCGCCGCTTCTCCCGCACGCTTC |
| 6S_CHK22 | TTTCCAGCACCGTGATGGCGCGAGCCAGCGCCGCACGCTGGCGCTGCGCTTCGCCGATCT |
| 6S_CHK80 | TTTCCAGCACCGTGATGGCGCGAGCCAGCGCCGCACGCTGGCGCTGCGCTTCGCCGATCT |
| 6S_CHK22 | GAGGACAGTCGGGGAACTCTGATCAGTCTAAACCCCCTTGCGCGTTAGTGTTGCCATCCT |
| 6S_CHK80 | GAGGACAGTCGGGGAACTCTGATCAGTCTAAACCCCCTTGCGCGTTAGTGTTGCCATCCT |
| 6S_CHK22 | TTGCAGACCGGTGAGAGCCGACTTGTTGTGCGCCACCCCCCACACCACCTCCTCCCAGAC |
| 6S_CHK80 | TTGCAGACCGGTGAGAGCCGACTTGTTGTGCGCCACCCCCCACACCACCTCCTCCCAGAC |
| 6S_CHK22 | CAATTCTGTCACCTTTTTGGCGAAGGCATCGGCCTCGGCCTGCAGAGAGGACAGCAGTGC |
| 6S_CHK80 | CAATTCTGTCACCTTTTTGGCGAAGGCATCGGCCTCGGCCTGCAGAGAGGACAGCAGTGC |
| 6S_CHK22 | CCAGCCGCTGGGGGTTGGCGGATGCACGCTCAGCTTGTTTTCCAGAAGGAGTTGCTCCTT |
| 6S_CHK80 | CCAGCCGCTGGGGGTTGGCGGATGCACGCTCAGCTTGTTTTCCAGAAGGAGTTGCTCCTT |
| 6S_CHK22 | GAGCCTTTCATTCTCAGCCTCGATAACCTCCAAAGCCGCTCTAATTGTGGAGGGGGTTCG |
| 6S_CHK80 | GAGCCTTTCATTCTCAGCCTCGATAACCTCCAAAGCCGCTCTAATTGTGGAGGGGGTTCG |
| 6S_CHK22 | AATTTAAAAGCTTGGAATGTTGGTTCGTGCGTCTGGAACAAGCCCAGACTTGTTGCTCAC |
| 6S_CHK80 | AATTTAAAAGCTTGGAATGTTGGTTCGTGCGTCTGGAACAAGCCCAGACTTGTTGCTCAC |
| 6S_CHK22 | TGGGAAAAGGACCATCAGCTCCAAAAAACTTGCCGCTCAAACCGCGTACCTCTGCTTTCG |
| 6S_CHK80 | TGGGAAAAGGACCATCAGCTCCAAAAAACTTGCCGCTCAAACCGCGTACCTCTGCTTTCG |
| 6S_CHK22 | CGCAATCTGCCCTGTTGAAATCGCCACCACATTCATATTGTGACGCTTGAGCAGTCTGTA |
| 6S_CHK80 | CGCAATCTGCCCTGTTGAAATCGCCACCACATTCATATTGTGACGCTTGAGCAGTCTGTA |
| 6S_CHK22 | ATTGCCTCAGAATGTGGAATCATCTGCCCCCTGTGCGAGCCCATGCCAGGCATGTCGCGG |
| 6S_CHK80 | ATTGCCTCAGAATGTGGAATCATCTGCCCCCTGTGCGAGCCCATGCCAGGCATGTCGCGG |
| 6S_CHK22 | GCGAGGACACCCGCCACTCGTACAGCAGACCATTATGCTACCTCACAATAGTTCATAACA |
| 6S_CHK80 | GCGAGGACACCCGCCACTCGTACAGCAGACCATTATGCTACCTCACAATAGTTCATAACA |
| 6S_CHK22 | GTGACCATATTTCTCGAAGCTCCCCAACGAGCACCTCCATGCTCTGAGTGGCCACCCCCC |
| 6S_CHK80 | GTGACCATATTTCTCGAAGCTCCCCAACGAGCACCTCCATGCTCTGAGTGGCCACCCCCC |
| 6S_CHK22 | GGCCCTGGTGCTTGCGGAGGGCAGGTCAACCGGCATGGGGCTACCGAAATCCCCGACCGG |
| 6S_CHK80 | GGCCCTGGTGCTTGCGGAGGGCAGGTCAACCGGCATGGGGCTACCGAAATCCCCGACCGG |
| 6S_CHK22 | ATCCCACCACCCCCGCGATGGGAAGAATCTCTCCCCGGGATGTGGGCCCACCACCAGCAC |
| 6S_CHK80 | ATCCCACCACCCCCGCGATGGGAAGAATCTCTCCCCGGGATGTGGGCCCACCACCAGCAC |
| 6S_CHK22 | AACCTGCTGGCCCAGGCGAGCGTCAAACCATACCACACAAATATCCTTGGCATCGGCCCT |
| 6S_CHK80 | AACCTGCTGGCCCAGGCGAGCGTCAAACCATACCACACAAATATCCTTGGCATCGGCCCT |
| 6S_CHK22 | GAATTCCTTCTGCCGCTCTGCTACCCGGTGCTTCTGTCCGAAGCAGGGGTTGCTAGGGAT |
| 6S_CHK80 | GAATTCCTTCTGCCGCTCTGCTACCCGGTGCTTCTGTCCGAAGCAGGGGTTGCTAGGGAT |
| 6S_CHK22 | CGCTCCGAGTCCGCAAACCCTTGTCGCGTGGCGGGGCTTGTTCGAGCTcgcagcatcgct |
| 6S_CHK80 | CGCTCCGAGTCCGCAAACCCTTGTCGCGTGGCGGGGCTTGTTCGAGCTcgcagcatcgct |
| 6S_CHK22 | tgcacgggggctatcttcaagtagtcgggaagcatgggtcgcgacgtcgagacgggcgag |
| 6S_CHK80 | tgcacgggggctatcttcaagtagtcgggaagcatgggtcgcgacgtcgagacgggcgag |
| 6S_CHK22 | cgggcctatgggtgagtcggccgtgcatggggagggctagcgccagggctcgggtcgccc |
| 6S_CHK80 | cgggcctatgggtgagtcggccgtgcatggggagggctagcgccagggctcgggtcgccc |
| 6S_CHK22 | cgatggccggggtctttgctcggggtgcacgcggccgtgggtgcgagacaggttgcgtga |
| 6S_CHK80 | cgatggccggggtctttgctcggggtgcacgcggccgtgggtgcgagacaggttgcgtga |
| 6S_CHK22 | cgaatttcttggcacccccccacggatgcctcacgcgggcgcccccctccctcgcaggta |
| 6S_CHK80 | cgaatttcttggcacccccccacggatgcctcacgcgggcgcccccctccctcgcaggta |
| 6S_CHK22 | cgtccgcaaggtgtccggcccggtggtggtggccagcagcatgagcgggtctgccatgta |
| 6S_CHK80 | cgtccgcaaggtgtccggcccggtggtggtggccagcagcatgagcgggtctgccatgta |
| 6S_CHK22 | cgagctggtccgcgtgggcgcggacaagctgattggcgagatcattcgcctcgagggaga |
| 6S_CHK80 | cgagctggtccgcgtgggcgcggacaagctgattggcgagatcattcgcctcgagggaga |
| 6S_CHK22 | cacggcgaccatccaggtgaggtggcgggggaggctgcttgagtggaagtggggcgcaca |
| 6S_CHK80 | cacggcgaccatccaggtgaggtggcgggggaggctgcttgagtggaagtggggcgcaca |
| 6S_CHK22 | accctccattggttggcggccaaggatgcagggaagcgaatgtcgagcattgcgcgcttt |
| 6S_CHK80 | accctccattggttggcggccaaggatgcagggaagcgaatgtcgagcattgcgcgcttt |
| 6S_CHK22 | tgcttgctgacatatcgttgcttgcaccccatgtattctccaggtgtacgaggatacctc |
| 6S_CHK80 | tgcttgctgacatatcgttgcttgcaccccatgtattctccaggtgtacgaggatacctc |
| 6S_CHK22 | gggcctgacggtgggcgacacggtcgtccgctccggcaaggtgtggagggtgtgagagtt |
| 6S_CHK80 | gggcctgacggtgggcgacacggtcgtccgctccggcaaggtgtggagggtgtgagagtt |
| 6S_CHK22 | gatgtatatgctttggtcgcccccttgctctggcacgtcagaggcgcaccttcagtgctc |
| 6S_CHK80 | gatgtatatgctttggtcgcccccttgctctggcacgtcagaggcgcaccttcagtgctc |
| 6S_CHK22 | acaagtggtgccgttagtgaaactgaacaagtgcttgactgtgtaggatttccgctcgcc |
| 6S_CHK80 | acaagtggtgccgttagtgaaactgaacaagtgcttgactgtgtaggatttccgctcgcc |
| 6S_CHK22 | cctttcttaccctctcccctttctctctttctccacccctcaaaatctccagcccctgtc |
| 6S_CHK80 | cctttcttaccctctcccctttctctctttctccacccctcaaaatctccagcccctgtc |
| 6S_CHK22 | tgtggagctgggccccggcatcatgggcaccatctttgacggcatccagcgcccgctcaa |
| 6S_CHK80 | tgtggagctgggccccggcatcatgggcaccatctttgacggcatccagcgcccgctcaa |
| 6S_CHK22 | gtcgatcgcggtggacagcgattcgtgcttcatcccgcgcggcgtggacgtgccggccct |
| 6S_CHK80 | gtcgatcgcggtggacagcgattcgtgcttcatcccgcgcggcgtggacgtgccggccct |
| 6S_CHK22 | ggaccgcaaggcctcgtgggagtttgacccggtgtcaagcttcaaggtgggcgaccgcat |
| 6S_CHK80 | ggaccgcaaggcctcgtgggagtttgacccggtgtcaagcttcaaggtgggcgaccgcat |
| 6S_CHK22 | cacgggcggcgacatctacggcgtggtgcacgagaacacgctgatggagcacaaggtgct |
| 6S_CHK80 | cacgggcggcgacatctacggcgtggtgcacgagaacacgctgatggagcacaaggtgct |
| 6S_CHK22 | gcttccgccgggcgcgcgcggcaccatctcctacatcgcgccggcggggagctacagcat |
| 6S_CHK80 | gcttccgccgggcgcgcgcggcaccatctcctacatcgcgccggcggggagctacagcat |
| 6S_CHK22 | cacggacaagatcatcgaggtcgagtttggcggcgcgcgcaaggagtactccatgctgca |
| 6S_CHK80 | cacggacaagatcatcgaggtcgagtttggcggcgcgcgcaaggagtactccatgctgca |
| 6S_CHK22 | gctctggcccgtgcgcgcgccgcgccccgtggcgcagaagctgctggccaacacgccgct |
| 6S_CHK80 | gctctggcccgtgcgcgcgccgcgccccgtggcgcagaagctgctggccaacacgccgct |
| 6S_CHK22 | gctgacggggcagcgcgtgctggacgcgctcttccccggcgtgctgggcgggacgtgcgc |
| 6S_CHK80 | gctgacggggcagcgcgtgctggacgcgctcttccccggcgtgctgggcgggacgtgcgc |
| 6S_CHK22 | catcccgggcgcgtttggctgcggcaagacggtcatctcgcaggcgctgtcaaagtacag |
| 6S_CHK80 | catcccgggcgcgtttggctgcggcaagacggtcatctcgcaggcgctgtcaaagtacag |
| 6S_CHK22 | taactcggagggcatcatctacgtcggctgcggcgagcgcggcaacgagatggccgaggt |
| 6S_CHK80 | taactcggagggcatcatctacgtcggctgcggcgagcgcggcaacgagatggccgaggt |
| 6S_CHK22 | gctcatggacttccccgcgctgaccatgaccatgccggacgggcgcgaggagagcatcat |
| 6S_CHK80 | gctcatggacttccccgcgctgaccatgaccatgccggacgggcgcgaggagagcatcat |
| 6S_CHK22 | gcagcgcaccacgctcgtggccaacacctccaacatgcccgtcgccgcgcgcgaggccag |
| 6S_CHK80 | gcagcgcaccacgctcgtggccaacacctccaacatgcccgtcgccgcgcgcgaggccag |
| 6S_CHK22 | catctaca |
| 6S_CHK80 | catctaca |
| DAO1B_CHK80 | gcaatctcccgcccaacctccaccagccggcgcaggacctggagcgcgaaagaggatttc |
| | (SEQ ID NO: 19) |
| DAO1B_CHK22 | gcaatctcccgcccaacctccaccagccggcgcaggacctggagcgcgaaagaggatttc |
| | (SEQ ID NO: 20) |
| DAO1B_CHK80 | gggggtgtgggggagagtgagcgcgatggaagacactgatttgcataaacaacctttggc |
| DAO1B_CHK22 | ggg-gtgtgggggagagtgagcgcgatggaagacactgatttgcataaacaacctttggc |
| DAO1B_CHK80 | cgctttcaaagcaccacctcaccaccataaaaccctccctcccatgcataccttgatgcc |
| DAO1B_CHK22 | cgctttcaaagcaccacctcaccaccataaaaccctccctcccatgcataccttgatgcc |
| DAO1B_CHK80 | tgccgagatggcctgggcttggtggaccatggccatgtagtccggcgtggtcgcgatgga |
| DAO1B_CHK22 | tgccgagatggcctgggcttggtggaccatggccatgtagtccggcgtggtcgcgatgga |
| DAO1B_CHK80 | cgccgcacgttgtgcgatgagcgccttgcccccgtcctcgtgcccgagcccgcgtccggg |
| DAO1B_CHK22 | cgccgcacgttgtgcgatgagcgccttgcccccgtcctcgtgcccgagcccgcgtccggg |
| DAO1B_CHK80 | cggctcggccagccccgccagcagccccgggtccagctcggcgttgagggagcgctttgc |
| DAO1B_CHK22 | cggctcggccagccccgccagcagccccgggtccagctcggcgttgagggagcgctttgc |
| DAO1B_CHK80 | cagcgcacggatggactcgtacggcagcgtgatgaggtggagcaggctgtcgagcaggcc |
| DAO1B_CHK22 | cagcgcacggatggactcgtacggcagcgtgatgaggtggagcaggctgtcgagcaggcc |
| DAO1B_CHK80 | cggctgcgaccagagcatggtgaggatgcgcgccgccgcgggcgtgccgggctgcaccgc |
| DAO1B_CHK22 | cggctgcgaccagagcatggtgaggatgcgcgccgccgcgggcgtgccgggctgcaccgc |
| DAO1B_CHK80 | cagcagcaacgcggcgaccgtctcggccgcaaacaccgccaggaagcagccctccgaggt |
| DAO1B_CHK22 | cagcagcaacgcggcgaccgtctcggccgcaaacaccgccaggaagcagccctccgaggt |
| DAO1B_CHK80 | cgggccgggctccagcaggggaccgcaggcgtgcactatgtccaggcgctccgtgatgat |
| DAO1B_CHK22 | cgggccgggctccagcaggggaccgcaggcgtgcactatgtccaggcgctccgtgatgat |
| DAO1B_CHK80 | gtgcatggccagcggcagcagcgagcgcagcacgctgctggccaggtgcgtgagcgtgct |
| DAO1B_CHK22 | gtgcatggccagcggcagcagcgagcgcagcacgctgctggccaggtgcgtgagcgtgct |
| DAO1B_CHK80 | cgtgccggccgagtccgaggtctcgtccgtggccaggtccatgggcggcagcgtgcgctc |
| DAO1B_CHK22 | cgtgccggccgagtccgaggtctcgtccgtggccaggtccatgggcggcagcgtgcgctc |
| DAO1B_CHK80 | gctcgcctggtgcgccgtccagctgcgccggtggcacagcagccgcagcaccgcgtggaa |
| DAO1B_CHK22 | gctcgcctggtgcgccgtccagctgcgccggtggcacagcagccgcagcaccgcgtggaa |
| DAO1B_CHK80 | gagcggcaggcagcccgcctccaccgccgcggcggcggccggggcgccgccctcctgcag |
| DAO1B_CHK22 | gagcggcaggcagcccgcctccaccgccgcggcggcggccggggcgccgccctcctgcag |
| DAO1B_CHK80 | gctggcgcgcaggatggccgccgactgccgcgccgccgacagctgctgcctgtgtctcaa |
| DAO1B_CHK22 | gctggcgcgcaggatggccgccgactgccgcgccgccgacagctgctgcctgtgtctcaa |
| DAO1B_CHK80 | aagctgcgacagcgtcggagagatgtcctgcggctccggctccgggatgtgctggatgcc |
| DAO1B_CHK22 | aagctgcgacagcgtcggagagatgtcctgcggctccggctccgggatgtgctggatgcc |
| DAO1B_CHK80 | acgcacaccggtcgcaatcacctgttggataatgggtggcaggatggttggttaggttct |
| DAO1B_CHK22 | acgcacaccggtcgcaatcacctgttggataatgggtggcaggatggttggttaggttct |
| DAO1B_CHK80 | gatgcggtcggggaggcggataaggtcacagccgcgtgatcttgctgaccaaacactgct |
| DAO1B_CHK22 | gatgcggtcggggaggcggataaggtcacagccgcgtgatcttgctgaccaaacactgct |
| DAO1B_CHK80 | ggccacccaacaaaagtcgattgccaccgatgcacacggcatggcgcccagacagaagct |
| DAO1B_CHK22 | ggccacccaacaaaagtcgattgccaccgatgcacacggcatggcgcccagacagaagct |
| DAO1B_CHK80 | cagcataccctcaggagggccagggccaggccaggccacatgtgcagctggaagccgggc |
| DAO1B_CHK22 | cagcataccctcaggagggccagggccaggccaggccacatgtgcagctggaagccgggc |
| DAO1B_CHK80 | tgggctgaagcatgggccagcacgctcagggcatggttttgaaaggcaggaacgtggctt |
| DAO1B_CHK22 | tgggctgaagcatgggccagcacgctcagggcatggttttgaaaggcaggaacgtggctt |
| DAO1B_CHK80 | gtcacgtcaacaaccagggaagaggcctgcgacatcaaggggcaaagttggaatgttcag |
| DAO1B_CHK22 | gtcacgtcaacaaccagggaagaggcctgcgacatcaaggggcaaagttggaatgttcag |
| DAO1B_CHK80 | catcatcagcctgggaccaatactctgctcacggttcagggcagaaaggtggcatagtca |
| DAO1B_CHK22 | catcatcagcctgggaccaatactctgctcacggttcagggcagaaaggtggcatagtca |
| DAO1B_CHK80 | gggcctcgttcagaatttcaagtcccagtttaaacttcgtacctccgcgaggctgatccc |
| DAO1B_CHK22 | gggcctcgttcagaatttcaagtcccagtttaaacttcgtacctccgcgaggctgatccc |
| DAO1B_CHK80 | ctcgccctgagcggactggggccggggagccggggtgggaccggtggtcacctgcgccat |
| DAO1B_CHK22 | ctcgccctgagcggactggggccggggagccggggtgggaccggtggtcacctgcgccat |
| DAO1B_CHK80 | gGCTAGCCCGCACCCTCGTTGATCTGGGAGCCCTGCGCAGCCCCTTAAATCATCTCAGTC |
| DAO1B_CHK22 | gGCTAGCCCGCACCCTCGTTGATCTGGGAGCCCTGCGCAGCCCCTTAAATCATCTCAGTC |
| DAO1B_CHK80 | AGGTTTCTGTGTTCAACTGAGCCTAAAGGGCTTTCGTCATGCGCACGAGCACACGTATAT |
| DAO1B_CHK22 | AGGTTTCTGTGTTCAACTGAGCCTAAAGGGCTTTCGTCATGCGCACGAGCACACGTATAT |
| DAO1B_CHK80 | CGGCCACGCAGTTTCTCAAAAGCGGTAGAACAGTTCGCGAGCCCTCGTAGGTCGAAAACT |
| DAO1B_CHK22 | CGGCCACGCAGTTTCTCAAAAGCGGTAGAACAGTTCGCGAGCCCTCGTAGGTCGAAAACT |
| DAO1B_CHK80 | TGCGCCAGTACTATTAAATTAAATTAATTGATCGAACGAGACGCGAAACTTTTGCAGAAT |
| DAO1B_CHK22 | TGCGCCAGTACTATTAAATTAAATTAATTGATCGAACGAGACGCGAAACTTTTGCAGAAT |
| DAO1B_CHK80 | GCCACCGAGTTTGCCCAGAGAATGGGAGTGGCGCCATTCACCATCCGCCTGTGCCCGGCT |
| DAO1B_CHK22 | GCCACCGAGTTTGCCCAGAGAATGGGAGTGGCGCCATTCACCATCCGCCTGTGCCCGGCT |
| DAO1B_CHK80 | TGATTCGCCGAGACGATGGACGGCGAGACCAGGGAGCGGCTTGCGAGCCCCGAGCCGGTA |
| DAO1B_CHK22 | TGATTCGCCGAGACGATGGACGGCGAGACCAGGGAGCGGCTTGCGAGCCCCGAGCCGGTA |
| DAO1B_CHK80 | GCAGGAACAATGATCGACAATCTTCCTGTCCAATTACTGGCAACCATTAGAAAGAGCCGG |
| DAO1B_CHK22 | GCAGGAACAATGATCGACAATCTTCCTGTCCAATTACTGGCAACCATTAGAAAGAGCCGG |
| DAO1B_CHK80 | AGCGCGTTGAAAGTCTGCAATCGAGTAATTTTTCGATACGTCGGGCCTGCTGAACCCTAA |
| DAO1B_CHK22 | AGCGCGTTGAAAGTCTGCAATCGAGTAATTTTTCGATACGTCGGGCCTGCTGAACCCTAA |
| DAO1B_CHK80 | GGCTCCGGACTTTGTTTAAGGCGATCCAAGATGCACGCGGCCCCAGGCACGTATCTCAAG |
| DAO1B_CHK22 | GGCTCCGGACTTTGTTTAAGGCGATCCAAGATGCACGCGGCCCCAGGCACGTATCTCAAG |
| DAO1B_CHK80 | CACAAACCCCAGCCTTAGTTTCGAGACTTTGGGAGATAGCGACCGATATCTAGTTTGGCA |
| DAO1B_CHK22 | CACAAACCCCAGCCTTAGTTTCGAGACTTTGGGAGATAGCGACCGATATCTAGTTTGGCA |
| DAO1B_CHK80 | TTTTGTATATTAATTACCTCAAGCAATGGAGCGCTCTGATGCGGTGCAGCGTCGGCTGCA |
| DAO1B_CHK22 | TTTTGTATATTAATTACCTCAAGCAATGGAGCGCTCTGATGCGGTGCAGCGTCGGCTGCA |
| DAO1B_CHK80 | GCACCTGGCAGTGGCGCTAGGGTCGCCCTATCGCTCGGAACCTGGTCAGCTGGCTCCCGC |
| DAO1B_CHK22 | GCACCTGGCAGTGGCGCTAGGGTCGCCCTATCGCTCGGAACCTGGTCAGCTGGCTCCCGC |
| DAO1B_CHK80 | CTCCTGCTCAGCCTCTTCCAGCCGTAGCGTCTGCGTGTTGGGAGCTGGAGTCGTGGGCTT |
| DAO1B_CHK22 | CTCCTGCTCAGCCTCTTCCAGCCGTAGCGTCTGCGTGTTGGGAGCTGGAGTCGTGGGCTT |
| DAO1B_CHK80 | GACGACGGCGCTGCAGCTGTTGCAGGATGTGCCTGGCGTGCGCGTTCACGTCGTGGCTGA |
| DAO1B_CHK22 | GACGACGGCGCTGCAGCTGTTGCAGGATGTGCCTGGCGTGCGCGTTCACGTCGTGGCTGA |
| DAO1B_CHK80 | GAAATATGGCGACGAAACGTTGACGGCTGGGGCCGGCGGGCTGTGGATGCCATACGCATT |
| DAO1B_CHK22 | GAAATATGGCGACGAAACGTTGACGGCTGGGGCCGGCGGGCTGTGGATGCCATACGCATT |
| DAO1B_CHK80 | GGGTACGCGGCCATTGGATGGGATTGATAGGCTTATGGAGGGATAATAGAGTTTTTGCCG |
| DAO1B_CHK22 | GGGTACGCGGCCATTGGATGGGATTGATAGGCTTATGGAGGGATAATAGAGTTTTTGCCG |
| DAO1B_CHK80 | GATCCAACGCATGTGGATGCGGTATCCCGGTGGGCTGAAAGTGTGGAAGGATAGTGCATT |
| DAO1B_CHK22 | GATCCAACGCATGTGGATGCGGTATCCCGGTGGGCTGAAAGTGTGGAAGGATAGTGCATT |
| DAO1B_CHK80 | GGCTATTCACATGCACTGCCCACCCCTTTTGGCAGGAAATGTGCCGGCATCGTTGGTGCA |
| DAO1B_CHK22 | GGCTATTCACATGCACTGCCCACCCCTTTTGGCAGGAAATGTGCCGGCATCGTTGGTGCA |
| DAO1B_CHK80 | CCGATGGGGAAAATCGACGTTCGACCACTACATGAAGATTTATACGTCTGAAGATGCAGC |
| DAO1B_CHK22 | CCGATGGGGAAAATCGACGTTCGACCACTACATGAAGATTTATACGTCTGAAGATGCAGC |
| DAO1B_CHK80 | GACTGCGGGTGCGAAACGGATGACGGTTTGGTCGTGTATGTCACAGCATGTGCTGGATCT |
| DAO1B_CHK22 | GACTGCGGGTGCGAAACGGATGACGGTTTGGTCGTGTATGTCACAGCATGTGCTGGATCT |
| DAO1B_CHK80 | TGCGGGCTAACTCCCCCTGCCACGGCCCATTGCAGGTGTCATGTTGACTGGAGGGTACGA |
| DAO1B_CHK22 | TGCGGGCTAACTCCCCCTGCCACGGCCCATTGCAGGTGTCATGTTGACTGGAGGGTACGA |
| DAO1B_CHK80 | CCTTTCGTCCGTCAAATTCCCAGAGGAGGACCCGCTCTGGGCCGACATTGTGCCCACTtt |
| DAO1B_CHK22 | CCTTTCGTCCGTCAAATTCCCAGAGGAGGACCCGCTCTGGGCCGACATTGTGCCCACTtt |
| DAO1B_CHK80 | tcgccgcctgggcaaggcagagctcctggcctacgaccccagcggcaagtcgatcgacgg |
| DAO1B_CHK22 | tcgccgcctgggcaaggcagagctcctggcctacgaccccagcggcaagtcgatcgacgg |
| DAO1B_CHK80 | ctacggcttcaccaccatcatcacgtacgatgctcaatgaaaggggagttcattgccttc |
| DAO1B_CHK22 | ctacggcttcaccaccatcatcacgtacgatgctcaatgaaaggggagttcattgccttc |
| DAO1B_CHK80 | actagagaaacatacatgggtctatgtaaacttgatcgatctccgaatttcctcttgttg |
| DAO1B_CHK22 | actagagaaacatacatgggtctatgtaaacttgatcgatctccgaatttcctcttgttg |
| DAO1B_CHK80 | tgtctctggttgtgcagggaaggtcgcctctacctgccctggctgatgcagcagattcaa |
| DAO1B_CHK22 | tgtctctggttgtgcagggaaggtcgcctctacctgccctggctgatgcagcagattcaa |
| DAO1B_CHK80 | ggcttgggggggacctttgaacggcgccgcatctccagcctgtcggagctgaaggagtat |
| DAO1B_CHK22 | ggcttgggggggacctttgaacggcgccgcatctccagcctgtcggagctgaaggagtat |
| DAO1B_CHK80 | gatgccatcgtcaattgcacaggtgggttggagttagtccaggagatgtcgatagaccaa |
| DAO1B_CHK22 | gatgccatcgtcaattgcacaggtgggttggagttagtccaggagatgtcgatagaccaa |
| DAO1B_CHK80 | tgcaaatcgtgtgcaacgcattagacccaatcactgcctccctcgccgctctcaggcctg |
| DAO1B_CHK22 | tgcaaatcgtgtgcaacgcattagacccaatcactgcctccctcgccgctctcaggcctg |
| DAO1B_CHK80 | gaggcgccaaagctggtgcaggacgagtccatgtacccggtgcgcgggcacgttctgcgc |
| DAO1B_CHK22 | gaggcgccaaagctggtgcaggacgagtccatgtacccggtgcgcgggcacgttctgcgc |
| DAO1B_CHK80 | gtacgggcgccctgggtccgccactacatcaaccgcgacgggggcacctacatcatcccc |
| DAO1B_CHK22 | gtacgggcgccctgggtccgccactacatcaaccgcgacgggggcacctacatcatcccc |
| DAO1B_CHK80 | aacacggacacggtggtgctgggcggcatcacgcaaaagggcaactggtccctcgagccg |
| DAO1B_CHK22 | aacacggacacggtggtgctgggcggcatcacgcaaaagggcaactggtccctcgagccg |
| DAO1B_CHK80 | accgaggaggatcggcgcgggatcctggagcgctgctacgagatcctgcccagcctgcgc |
| DAO1B_CHK22 | accgaggaggatcggcgcgggatcctggagcgctgctacgagatcctgcccagcctgcgc |
| DAO1B_CHK80 | aaggcgccgatcctgcgcgagtgggtcgggctccggcccggccgcccagacatccgcctg |
| DAO1B_CHK22 | aaggcgccgatcctgcgcgagtgggtcgggctccggcccggccgcccagacatccgcctg |
| DAO1B_CHK80 | gagcgcgaagatgcgcagctcgacggcaagtccgtacccgtcatccacaactacgggcac |
| DAO1B_CHK22 | gagcgcgaagatgcgcagctcgacggcaagtccgtacccgtcatccacaactacgggcac |
| DAO1B_CHK80 | ggcgggtccgggctcaccctgggctggggctgcgccgcagacgccgtcgcgctcgtgcgc |
| DAO1B_CHK22 | ggcgggtccgggctcaccctgggctggggctgcgccgcagacgccgtcgcgctcgtgcgc |
| DAO1B_CHK80 | agggcgctggccctctgacggtgagagggcctgttgcaggctgtgcggtcccgctcatct |
| DAO1B_CHK22 | agggcgctggccctctgacggtgagagggcctgttgcaggctgtgcggtcccgctcatct |
| DAO1B_CHK80 | caaaggccttggaattggctgggcgaatattctgatgcattgatgtgtatctcttcgctt |
| DAO1B_CHK22 | caaaggccttggaattggctgggcgaatattctgatgcattgatgtgtatctcttcgctt |
| DAO1B_CHK80 | ttcttatgatgcaccactttggagcgagtattctggtgccaattgtgctttgaaatcacg |
| DAO1B_CHK22 | ttcttatgatgcaccactttggagcgagtattctggtgccaattgtgctttgaaatcacg |
| DAO1B_CHK80 | ctgcccacggtggcaaatgcaatgagagccgagtaagtggactggtagttcctaaggcga |
| DAO1B_CHK22 | ctgcccacggtggcaaatgcaatgagagccgagtaagtggactggtagttcctaaggcga |
| DAO1B_CHK80 | cgggcagctcaaggaaagccagcccccgatcccagctcgccacatccttgttgcacgcct |
| DAO1B_CHK22 | cgggcagctcaaggaaagccagcccccgatcccagctcgccacatccttgttgcacgcct |
| DAO1B_CHK80 | gtatctggcggtttgaatgagactcgcgtccacccaggccggccctcgcgctgcggctcc |
| DAO1B_CHK22 | gtatctggcggtttgaatgagactcgcgtccacccaggccggccctcgcgctgcggctcc |
| DAO1B_CHK80 | gcccacaactcgagcagcttgtcctttgcccctggcaacatggtctcgatggtgtcgtac |
| DAO1B_CHK22 | gcccacaactcgagcagcttgtcctttgcccctggcaacatggtctcgatggtgtcgtac |
| DAO1B_CHK80 | acgggggcaggaaccaggtgcaggcctggagaagagggatggaagtgcgcacagattcga |
| DAO1B_CHK22 | acgggggcaggaaccaggtgcaggcctggagaagagggatggaagtgcgcacagattcga |
| DAO1B_CHK80 | gaaggtgatggccctcttgccgaaatgcacaccctctttccccagcctctgcctccaccc |
| DAO1B_CHK22 | gaaggtgatggccctcttgccgaaatgcacaccctctttccccagcctctgcctccaccc |
| DAO1B_CHK80 | accgtctgctcgggaagggatgcggcagtccaggacaacgtccgggttgcgctggtgtcg |
| DAO1B_CHK22 | accgtctgctcgggaagggatgcggcagtccaggacaacgtccgggttgcgctggtgtcg |
| DAO1B_CHK80 | gagctccatatcgcggccccgttctgaatggatggggtgggacagttggtgtgaccgg |
| DAO1B_CHK22 | gagctccatatcgcggccccgttctgaatggatggggtgggacagttggtgtgaccgg |
| FAD2-2_CHK80 | cggctcgctgctttgcgtgccgggtgcagcgatcagatccaagtctgacgacttgtgctg |
| | (SEQ ID NO: 21) |
| FAD2-2_CHK22 | cggctcgctgctttgcgtgccgggtgcagcgatcagatccaagtctgacgacttgtgctg |
| | (SEQ ID NO: 22) |
| FAD2-2_CHK80 | atgtactgtgtcctttgagtccagagcgccggccgcacgcgcttcttccccttcttcttc |
| FAD2-2_CHK22 | atgtactgtgtcctttgagtccagagcgccggccgcacgcgcttcttccccttcttcttc |
| FAD2-2_CHK80 | ctctcgaacatccagcgatgcaagtgcagggcgctgggcggctggcgtcccgaaccggcc |
| FAD2-2_CHK22 | ctctcgaacatccagcgatgcaagtgcagggcgctgggcggctggcgtcccgaaccggcc |
| FAD2-2_CHK80 | tcggcgcacgcggctgaaattgccaatgtcggcaatgtagtgccgctcggcccatccctc |
| FAD2-2_CHK22 | tcggcgcacgcggctgaaattgccaatgtcggcaatgtagtgccgctcggcccatccctc |
| FAD2-2_CHK80 | gatcaagtttttcagcgcgtggttggggatgatctgcgctcatgggaagataaaaggggt |
| FAD2-2_CHK22 | gatcaagtttttcagcgcgtggttggggatgatctgcgctcatgggaagataaaaggggt |
| FAD2-2_CHK80 | tctgaggtggttgattggtactttaccggaagtactcatattcatacatgactgatccca |
| FAD2-2_CHK22 | tctgaggtggttgattggtactttaccggaagtactcatattcatacatgactgatccca |
| FAD2-2_CHK80 | cacaaaaacaaagctcacttcaaagaaccgcgcatgtctactccccagcaatcacttcgc |
| FAD2-2_CHK22 | cacaaaaacaaagctcacttcaaagaaccgcgcatgtctactccccagcaatcacttcgc |
| FAD2-2_CHK80 | tcaccgtcgggttgcttcccacgacaacgccggtgagagggtcggtggcctcgcgacctt |
| FAD2-2_CHK22 | tcaccgtcgggttgcttcccacgacaacgccggtgagagggtcggtggcctcgcgacctt |
| FAD2-2_CHK80 | cgcgggcacatctttccagccatgtctgtataatctcacgctcatacgtctggcccgtcg |
| FAD2-2_CHK22 | cgcgggcacatctttccagccatgtctgtataatctcacgctcatacgtctggcccgtcg |
| FAD2-2_CHK80 | accccaaaatgacgggatcctgcatgatatcgcccgagatggggtccaggcattcctctg |
| FAD2-2_CHK22 | accccaaaatgacgggatcctgcatgatatcgcccgagatggggtccaggcattcctctg |
| FAD2-2_CHK80 | gaggcgtcagccctgcgggagatgccggtcccaccgcattggaaaggcacaaagggggtg |
| FAD2-2_CHK22 | gaggcgtcagccctgcgggagatgccggtcccaccgcattggaaaggcacaaagggggtg |
| FAD2-2_CHK80 | aatcccccatttcatgaaattgttggtcagcgatggtgcgcactcgtgcgcaatgaatat |
| FAD2-2_CHK22 | aatcccccatttcatgaaattgttggtcagcgatggtgcgcactcgtgcgcaatgaatat |
| FAD2-2_CHK80 | ggggtcacgcggtggacgaacgcggagggggcctggccgaatctaggcttgcattcctca |
| FAD2-2_CHK22 | ggggtcacgcggtggacgaacgcggagggggcctggccgaatctaggcttgcattcctca |
| FAD2-2_CHK80 | gatcactttctgccggcggtccggggtttgcgcgtcgcgcaacgctccgtctccctagcc |
| FAD2-2_CHK22 | gatcactttctgccggcggtccggggtttgcgcgtcgcgcaacgctccgtctccctagcc |
| FAD2-2_CHK80 | gctgcgcaccgcgcgtgcgacgcgaaGGTCATTTTCCAGAACAACGACCATGGCTTGTCT |
| FAD2-2_CHK22 | gctgcgcaccgcgcgtgcgacgcgaaGGTCATTTTCCAGAACAACGACCATGGCTTGTCT |
| FAD2-2_CHK80 | TAGCGATCGCTCGAATGACTGCTAGTGAGTCGTACGCTCGACCCAGTCGCTCGCAGGAGA |
| FAD2-2_CHK22 | TAGCGATCGCTCGAATGACTGCTAGTGAGTCGTACGCTCGACCCAGTCGCTCGCAGGAGA |
| FAD2-2_CHK80 | ACGCGGCAACTGCCGAGCTTCGGCTTGCCAGTCGTGACTCGTATGTGATCAGGAATCATT |
| FAD2-2_CHK22 | ACGCGGCAACTGCCGAGCTTCGGCTTGCCAGTCGTGACTCGTATGTGATCAGGAATCATT |
| FAD2-2_CHK80 | GGCATTGGTAGCATTATAATTCGGCTTCCGCGCTGTTTATGGGCATGGCAATGTCTCATG |
| FAD2-2_CHK22 | GGCATTGGTAGCATTATAATTCGGCTTCCGCGCTGTTTATGGGCATGGCAATGTCTCATG |
| FAD2-2_CHK80 | CAGTCGACCTTAGTCAACCAATTCTGGGTGGCCAGCTCCGGGCGACCGGGCTCCGTGTCG |
| FAD2-2_CHK22 | CAGTCGACCTTAGTCAACCAATTCTGGGTGGCCAGCTCCGGGCGACCGGGCTCCGTGTCG |
| FAD2-2_CHK80 | CCGGGCACCACCTCCTGCCATGAGTAACAGGGCCGCCCTCTCCTCCCGACGTTGGCCCAC |
| FAD2-2_CHK22 | CCGGGCACCACCTCCTGCCATGAGTAACAGGGCCGCCCTCTCCTCCCGACGTTGGCCCAC |
| FAD2-2_CHK80 | TGAATACCGTGTCTTGGGGCCCTACATGATGGGCTGCCTAGTCGGGCGGGACGCGCAACT |
| FAD2-2_CHK22 | TGAATACCGTGTCTTGGGGCCCTACATGATGGGCTGCCTAGTCGGGCGGGACGCGCAACT |
| FAD2-2_CHK80 | GCCCGCGCAATCTGGGACGTGGTCTGAATCCTCCAGGCGGGTTTCCCCGAGAAAGAAAGG |
| FAD2-2_CHK22 | GCCCGCGCAATCTGGGACGTGGTCTGAATCCTCCAGGCGGGTTTCCCCGAGAAAGAAAGG |
| FAD2-2_CHK80 | GTGCCGATTTCAAAGCAGAGCCATGTGCCGGGCCCTGTGGCCTGTGTTGGCGCCTATGTA |
| FAD2-2_CHK22 | GTGCCGATTTCAAAGCAGAGCCATGTGCCGGGCCCTGTGGCCTGTGTTGGCGCCTATGTA |
| FAD2-2_CHK80 | GTCACCCCCCCTCACCCAATTGTCGCCAGTTTGCGCAATCCATAAACTCAAAACTGCAGC |
| FAD2-2_CHK22 | GTCACCCCCCCTCACCCAATTGTCGCCAGTTTGCGCAATCCATAAACTCAAAACTGCAGC |
| FAD2-2_CHK80 | TTCTGAGCTGCGCTGTTCAAGAACACCTCTGGGGTTTGCTCACCCGCGAGGTCGACGCCC |
| FAD2-2_CHK22 | TTCTGAGCTGCGCTGTTCAAGAACACCTCTGGGGTTTGCTCACCCGCGAGGTCGACGCCC |
| FAD2-2_CHK80 | AGCATGGCTATCAAGACGAACAGGCAGCCTGTGGAGAAGCCTCCGTTCACGATCGGGACG |
| FAD2-2_CHK22 | AGCATGGCTATCAAGACGAACAGGCAGCCTGTGGAGAAGCCTCCGTTCACGATCGGGACG |
| FAD2-2_CHK80 | CTGCGCAAGGCCATCCCCGCGCACTGTTTCGAGCGCTCGGCGCTTCGTAGCAGCATGTAC |
| FAD2-2_CHK22 | CTGCGCAAGGCCATCCCCGCGCACTGTTTCGAGCGCTCGGCGCTTCGTAGCAGCATGTAC |
| FAD2-2_CHK80 | CTGGCCTTTGACATCGCGGTCATGTCCCTGCTCTACGTCGCGTCGACGTACATCGACCCT |
| FAD2-2_CHK22 | CTGGCCTTTGACATCGCGGTCATGTCCCTGCTCTACGTCGCGTCGACGTACATCGACCCT |
| FAD2-2_CHK80 | GCGCCGGTGCCTACGTGGGTCAAGTATGGCGTCATGTGGCCGCTCTACTGGTTCTTCCAG |
| FAD2-2_CHK22 | GCGCCGGTGCCTACGTGGGTCAAGTATGGCGTCATGTGGCCGCTCTACTGGTTCTTCCAG |
| FAD2-2_CHK80 | GTGTGTGTGAGGGTTGTGGTTGCCCGTATCGAGGTCCTGGTGGCGCGCATGGGGGAGAAG |
| FAD2-2_CHK22 | GTGTGTGTGAGGGTTGTGGTTGCCCGTATCGAGGTCCTGGTGGCGCGCATGGGGGAGAAG |
| FAD2-2_CHK80 | GCGCCTGTCCCGCTGACCCCCCCGGCTACCCTCCCGGCACCTTCCAGGGCGCCTTCGGCA |
| FAD2-2_CHK22 | GCGCCTGTCCCGCTGACCCCCCCGGCTACCCTCCCGGCACCTTCCAGGGCGCCTTCGGCA |
| FAD2-2_CHK80 | CGGGTGTCTGGGTGTGCGCGCACGAGTGCGGCCACCAGGCCTTTTCCTCCAGCCAGGCCA |
| FAD2-2_CHK22 | CGGGTGTCTGGGTGTGCGCGCACGAGTGCGGCCACCAGGCCTTTTCCTCCAGCCAGGCCA |
| FAD2-2_CHK80 | TCAACGACGGCGTGGGCCTGGTGTTCCACAGCCTGCTGCTGGTGCCCTACTACTCCTGGA |
| FAD2-2_CHK22 | TCAACGACGGCGTGGGCCTGGTGTTCCACAGCCTGCTGCTGGTGCCCTACTACTCCTGGA |
| FAD2-2_CHK80 | AGCACTCGCACCGCCGCCACCACTCCAACACGGGGTGCCTGGACAAGGACGAGGTGTTTG |
| FAD2-2_CHK22 | AGCACTCGCACCGCCGCCACCACTCCAACACGGGGTGCCTGGACAAGGACGAGGTGTTTG |
| FAD2-2_CHK80 | TGCCGCCGCACCGCGCAGTGGCGCACGAGGGCCTGGAGTGGGAGGAGTGGCTGCCCATCC |
| FAD2-2_CHK22 | TGCCGCCGCACCGCGCAGTGGCGCACGAGGGCCTGGAGTGGGAGGAGTGGCTGCCCATCC |
| FAD2-2_CHK80 | GCATGGGCAAGGTGCTGGTCACCCTGACCCTGGGCTGGCCGCTGTACCTCATGTTCAACG |
| FAD2-2_CHK22 | GCATGGGCAAGGTGCTGGTCACCCTGACCCTGGGCTGGCCGCTGTACCTCATGTTCAACG |
| FAD2-2_CHK80 | TCGCCTCGCGGCCGTACCCGCGCTTCGCCAACCACTTTGACCCGTGGTCGCCCATCTTCA |
| FAD2-2_CHK22 | TCGCCTCGCGGCCGTACCCGCGCTTCGCCAACCACTTTGACCCGTGGTCGCCCATCTTCA |
| FAD2-2_CHK80 | GCAAGCGCGAGCGCATCGAGGTGGTCATCTCCGACCTGGCGCTGGTGGCGGTGCTCAGCG |
| FAD2-2_CHK22 | GCAAGCGCGAGCGCATCGAGGTGGTCATCTCCGACCTGGCGCTGGTGGCGGTGCTCAGCG |
| FAD2-2_CHK80 | GGCTCAGCGTGCTGGGCCGCACCATGGGCTGGGCCTGGCTGGTCAAGACCTACGTGGTGC |
| FAD2-2_CHK22 | GGCTCAGCGTGCTGGGCCGCACCATGGGCTGGGCCTGGCTGGTCAAGACCTACGTGGTGC |
| FAD2-2_CHK80 | CCTACCTGATCGTGAACATGTGGCTCGTGCTCATCACGCTGCTCCAGCACACGCACCCGG |
| FAD2-2_CHK22 | CCTACCTGATCGTGAACATGTGGCTCGTGCTCATCACGCTGCTCCAGCACACGCACCCGG |
| FAD2-2_CHK80 | CGCTGCCGCACTACTTCGAGAAGGACTGGGACTGGCTGCGCGGCGCCATGGCCACCGTGG |
| FAD2-2_CHK22 | CGCTGCCGCACTACTTCGAGAAGGACTGGGACTGGCTGCGCGGCGCCATGGCCACCGTGG |
| FAD2-2_CHK80 | ACCGCTCCATGGGCCCGCCCTTCATGGACAACATCCTGCACCACATCTCCGACACCCACG |
| FAD2-2_CHK22 | ACCGCTCCATGGGCCCGCCCTTCATGGACAACATCCTGCACCACATCTCCGACACCCACG |
| FAD2-2_CHK80 | TGCTGCACCACCTCTTCAGCACCATCCCGCACTACCACGCCGAGGAGGCCTCCGCCGCCA |
| FAD2-2_CHK22 | TGCTGCACCACCTCTTCAGCACCATCCCGCACTACCACGCCGAGGAGGCCTCCGCCGCCA |
| FAD2-2_CHK80 | TCAGGCCCATCCTGGGCAAGTACTACCAGTCCGACAGCCGCTGGGTCGGCCGCGCCCTGT |
| FAD2-2_CHK22 | TCAGGCCCATCCTGGGCAAGTACTACCAGTCCGACAGCCGCTGGGTCGGCCGCGCCCTGT |
| FAD2-2_CHK80 | GGGAGGACTGGCGCGACTGCCGCTACGTCGTCCCGGACGCGCCCGAGGACGACTCCGCGC |
| FAD2-2_CHK22 | GGGAGGACTGGCGCGACTGCCGCTACGTCGTCCCGGACGCGCCCGAGGACGACTCCGCGC |
| FAD2-2_CHK80 | TCTGGTTCCACAAGTGAGTGAGTGAGTCGCTCACTCAGCGCGCCTGCGCGGGGATGCGGA |
| FAD2-2_CHK22 | TCTGGTTCCACAAGTGAGTGAGTGAGTCGCTCACTCAGCGCGCCTGCGCGGGGATGCGGA |
| FAD2-2_CHK80 | ACGCCGCCGCCGCCTTGTCTTTTGCACGCGCGACTCCGTCGCTTCGCGGGTGGCACCCCC |
| FAD2-2_CHK22 | ACGCCGCCGCCGCCTTGTCTTTTGCACGCGCGACTCCGTCGCTTCGCGGGTGGCACCCCC |
| FAD2-2_CHK80 | ATTGAAAAAAACCTCAATTCTGTTTGTGGAAGACACGGTGTACCCCCAACCACCCACCTG |
| FAD2-2_CHK22 | ATTGAAAAAAACCTCAATTCTGTTTGTGGAAGACACGGTGTACCCCCAACCACCCACCTG |
| FAD2-2_CHK80 | CACCTCTATTATTGGTATTATTGACGCGGGAGCGGGCGTTGTACTCTACAACGTAGCGTC |
| FAD2-2_CHK22 | CACCTCTATTATTGGTATTATTGACGCGGGAGCGGGCGTTGTACTCTACAACGTAGCGTC |
| FAD2-2_CHK80 | TCTGGTTTTCAGCTGGCTCCCACCATTGTAAATTCTTGCTAAAATAGTGCGTGGTTATGT |
| FAD2-2_CHK22 | TCTGGTTTTCAGCTGGCTCCCACCATTGTAAATTCTTGCTAAAATAGTGCGTGGTTATGT |
| FAD2-2_CHK80 | GAGAGGTATGGTGTAACAGGGCGTCAGTCATGTTGGTTTTCGTGCTGATCTCGGGCACAA |
| FAD2-2_CHK22 | GAGAGGTATGGTGTAACAGGGCGTCAGTCATGTTGGTTTTCGTGCTGATCTCGGGCACAA |
| FAD2-2_CHK80 | GGCGTCGTCGACGTGACGTGCCCGTGATGAGAGCAATACCGCGCTCAAAGCCGACGCATG |
| FAD2-2_CHK22 | GGCGTCGTCGACGTGACGTGCCCGTGATGAGAGCAATACCGCGCTCAAAGCCGACGCATG |
| FAD2-2_CHK80 | GCCTTTACTCCGCACTCCAAACGACTGTCGCTCGTATTTTTCGGATATCTATTTTTTAAG |
| FAD2-2_CHK22 | GCCTTTACTCCGCACTCCAAACGACTGTCGCTCGTATTTTTCGGATATCTATTTTTTAAG |
| FAD2-2_CHK80 | AGCGAGCACAGCGCCGGGCATGGGCCTGAAAGGCCTCGCGGCCGTGCTCGTGGTGGGGGC |
| FAD2-2_CHK22 | AGCGAGCACAGCGCCGGGCATGGGCCTGAAAGGCCTCGCGGCCGTGCTCGTGGTGGGGGC |
| FAD2-2_CHK80 | CGCGAGCGCGTGGGGCATCGCGGCAGTGCACCAGGCGCAGACGGAGGAACGCATGGTGAG |
| FAD2-2_CHK22 | CGCGAGCGCGTGGGGCATCGCGGCAGTGCACCAGGCGCAGACGGAGGAACGCATGGTGAG |
| FAD2-2_CHK80 | TGCGCATCACAAGATGCATGTCTTGTTGTCTGTACTATAATGCTAGAGCATCACCAGGGG |
| FAD2-2_CHK22 | TGCGCATCACAAGATGCATGTCTTGTTGTCTGTACTATAATGCTAGAGCATCACCAGGGG |
| FAD2-2_CHK80 | CTTAGTCATCGCACCTGCTTTGGTCATTACAGAAATTGCACAAGGGCGTCCTCCGGGATG |
| FAD2-2_CHK22 | CTTAGTCATCGCACCTGCTTTGGTCATTACAGAAATTGCACAAGGGCGTCCTCCGGGATG |
| FAD2-2_CHK80 | AGGAGATGTACCAGCTCAAGCTGGAGCGGCTTCGAGCCAAGCAGGAGCGCGGCGCATGAC |
| FAD2-2_CHK22 | AGGAGATGTACCAGCTCAAGCTGGAGCGGCTTCGAGCCAAGCAGGAGCGCGGCGCATGAC |
| FAD2-2_CHK80 | GACCTACCCACatgcgacaaaggggtctgggtcgtacgacaaaccagtcaggaggcggcg |
| FAD2-2_CHK22 | GACCTACCCACatgcgacaaaggggtctgggtcgtacgacaaaccagtcaggaggcggcg |
| FAD2-2_CHK80 | gggtccatgagctggcccgctcgcagcttcagcgcttcgagcatcgcggcgttgtccgcg |
| FAD2-2_CHK22 | gggtccatgagctggcccgctcgcagcttcagcgcttcgagcatcgcggcgttgtccgcg |
| FAD2-2_CHK80 | atccaagcctcccgcagcgcctccttggggcccctgtgcggagaggcgggtgccgagagg |
| FAD2-2_CHK22 | atccaagcctcccgcagcgcctccttggggcccctgtgcggagaggcgggtgccgagagg |
| FAD2-2_CHK80 | gctgtgagggcggaggctggaggacaggctgcgaatcgtcgcgctgcctaggcgtctgcc |
| FAD2-2_CHK22 | gctgtgagggcggaggctggaggacaggctgcgaatcgtcgcgctgcctaggcgtctgcc |
| FAD2-2_CHK80 | gaggagaagcacaccacgcgccgccgctttcatttcattgtgcccgttgctgtaatgcat |
| FAD2-2_CHK22 | gaggagaagcacaccacgcgccgccgctttcatttcattgtgcccgttgctgtaatgcat |
| FAD2-2_CHK80 | gcgagcgatcctgcactaaagctgcgatcgccacctcagcgttgttcgtctctagcaccg |
| FAD2-2_CHK22 | gcgagcgatcctgcactaaagctgcgatcgccacctcagcgttgttcgtctctagcaccg |
| FAD2-2_CHK80 | ctcacctgaccacgcgcacggaagagtggcggtcggcgacggcctgctgcacgtggcagt |
| FAD2-2_CHK22 | ctcacctgaccacgcgcacggaagagtggcggtcggcgacggcctgctgcacgtggcagt |
| FAD2-2_CHK80 | gcaggtccacgtggccgaggagcaggtctcgcagctgcagcaccgcgccctcgtgctcgc |
| FAD2-2_CHK22 | gcaggtccacgtggccgaggagcaggtctcgcagctgcagcaccgcgccctcgtgctcgc |
| FAD2-2_CHK80 | cggggtccatccagtccatgccccgcgccgggccctcgagccggacggccagcagccagg |
| FAD2-2_CHK22 | cggggtccatccagtccatgccccgcgccgggccctcgagccggacggccagcagccagg |
| FAD2-2_CHK80 | gccgggggtccgcgccgacgcccggcggccccgccgcgcagctggggtcggccaggaaga |
| FAD2-2_CHK22 | gccgggggtccgcgccgacgcccggcggccccgccgcgcagctggggtcggccaggaaga |
| FAD2-2_CHK80 | gccactgggcggccggcccgggcgagccccgcgccggcgcgggacccacccgcagcacca |
| FAD2-2_CHK22 | gccactgggcggccggcccgggcgagccccgcgccggcgcgggacccacccgcagcacca |
| FAD2-2_CHK80 | ccgcctggtccaggtcgacgtcgctcggcggcaggcccgcgtgcggcgcctgcgccagcg |
| FAD2-2_CHK22 | ccgcctggtccaggtcgacgtcgctcggcggcaggcccgcgtgcggcgcctgcgccagcg |
| FAD2-2_CHK80 | tccagggcaggcgggggcgcgcgtgca |
| FAD2-2_CHK22 | tccagggcaggcgggggcgcgcgtgca |
| FATA1_CHK80 | gcaatggcgctcggtacagggtctgcgtccgtgctgggctccctctcctacgatgcacaa |
| | (SEQ ID NO: 23) |
| FATA1_CHK22 | gcaatggcgctcggtacagggtctgcgtccgtgctgggctccctctcctacgatgcacaa |
| | (SEQ ID NO: 24) |
| FATA1_CHK80 | gggagcgccccggccagctcagcgcgtccacaacctcccctcgtcacacacacacctgcg |
| FATA1_CHK22 | gggagcgccccggccagctcagcgcgtccacaacctcccctcgtcacacacacacctgcg |
| FATA1_CHK80 | gaaccaggccgcccatttgctgcttgagcatgccttgcatcatgtccgggtttcccatca |
| FATA1_CHK22 | gaaccaggccgcccatttgctgcttgagcatgccttgcatcatgtccgggtttcccatca |
| FATA1_CHK80 | tatcgttgaggttcttgggctccagcttctgctccagcacaccatcctgtcgatcgaaga |
| FATA1_CHK22 | tatcgttgaggttcttgggctccagcttctgctccagcacaccatcctgtcgatcgaaga |
| FATA1_CHK80 | gaaggagacatgtgtacattattggtgtgagggcgctgaatcggccatttttta-aatga |
| FATA1_CHK22 | gaaggagacatgtgtacattattggtgtgagggcgctgaatcggccattttttaaaatga |
| FATA1_CHK80 | tcacgctcatgccaatagacgcggcacataacgacgttcaaacccccgccaaagccgcgg |
| FATA1_CHK22 | tcacgctcatgccaatagacgcggcacataacgacgttcaaacccccgccaaagccgcgg |
| FATA1_CHK80 | acaaccccatccctccacaccccccacacaaagaacccgccaccgcttaccttgcccacg |
| FATA1_CHK22 | acaaccccatccctccacaccccccacacaaagaacccgccaccgcttaccttgcccacg |
| FATA1_CHK80 | aggtaggcctttcgttgcgcaaaaccggcctcggtgatgaatgcatgcccgttcctgacg |
| FATA1_CHK22 | aggtaggcctttcgttgcgcaaaaccggcctcggtgatgaatgcatgcccgttcctgacg |
| FATA1_CHK80 | agcgctgcccgggccaacacgctcttttgctgcgtctcctcaggcttgggggcctccttg |
| FATA1_CHK22 | agcgctgcccgggccaacacgctcttttgctgcgtctcctcaggcttgggggcctccttg |
| FATA1_CHK80 | ggcttgggtgccgccatgatctgcgcgcatcagagaaacgttgctggt-aaaaggagcgc |
| FATA1_CHK22 | ggcttgggtgccgccatgatctgcgcgcatcagagaaacgttgctggtaaaaaggagcgc |
| FATA1_CHK80 | ccggctgcgcaatatatatataggcatgccaacacagcccaacctcactcgggagcccgt |
| FATA1_CHK22 | ccggctgcgcaatatatatataggcatgccaacacagcccaacctcactcgggagcccgt |
| FATA1_CHK80 | cccaccacccccaagtcgcgtgccttgacggcatactgctgcagaagcttcatgagaatg |
| FATA1_CHK22 | cccaccacccccaagtcgcgtgccttgacggcatactgctgcagaagcttcatgagaatg |
| FATA1_CHK80 | atgccgaacaagaggggcacgaggacccaatcccggacatccttgtcgataatgatctcg |
| FATA1_CHK22 | atgccgaacaagaggggcacgaggacccaatcccggacatccttgtcgataatgatctcg |
| FATA1_CHK80 | tgagtccccatcgtccgcccgacgctccggggagcccgccgatgctcaagacgagagggc |
| FATA1_CHK22 | tgagtccccatcgtccgcccgacgctccggggagcccgccgatgctcaagacgagagggc |
| FATA1_CHK80 | cctcgaccaggaggggctggcccgggcgggcactggcgtcgaaggtgcgcccgtcgttcg |
| FATA1_CHK22 | cctcgaccaggaggggctggcccgggcgggcactggcgtcgaaggtgcgcccgtcgttcg |
| FATA1_CHK80 | cctgcagtcctatgccacaaaacaagtcttctgacggggtgcgtttgctcccgtgcgggc |
| FATA1_CHK22 | cctgcagtcctatgccacaaaacaagtcttctgacggggtgcgtttgctcccgtgcgggc |
| FATA1_CHK80 | aggcaacagaggtattcaccctggtcatggggagatcggcgatcgagctgggataagaga |
| FATA1_CHK22 | aggcaacagaggtattcaccctggtcatggggagatcggcgatcgagctgggataagaga |
| FATA1_CHK80 | tacggtcccgcgcaaggatcgctcatcctggtctgagccggacagtcattctggcaagca |
| FATA1_CHK22 | tacggtcccgcgcaaggatcgctcatcctggtctgagccggacagtcattctggcaagca |
| FATA1_CHK80 | atgacaacttgtcaggaccggaccgtgccatatatttctcacctagcgccgcaaaaccta |
| FATA1_CHK22 | atgacaacttgtcaggaccggaccgtgccatatatttctcacctagcgccgcaaaaccta |
| FATA1_CHK80 | aCAATTTGGGAGTCACTGTGCCACTGAGTTCGACTGGTAGCTGAATGGAGTCGCTGCTCC |
| FATA1_CHK22 | aCAATTTGGGAGTCACTGTGCCACTGAGTTCGACTGGTAGCTGAATGGAGTCGCTGCTCC |
| FATA1_CHK80 | ACTAAACGAATTGTCAGCACCGCCAGCCGGCCGAGGACCCGAGTCATAGCGAGGGTAGTA |
| FATA1_CHK22 | ACTAAACGAATTGTCAGCACCGCCAGCCGGCCGAGGACCCGAGTCATAGCGAGGGTAGTA |
| FATA1_CHK80 | GCGCGCCATGGCACCGACCAGCCTGCTTGCCAGTACTGGCGTCTCTTCCGCTTCTCTGTG |
| FATA1_CHK22 | GCGCGCCATGGCACCGACCAGCCTGCTTGCCAGTACTGGCGTCTCTTCCGCTTCTCTGTG |
| FATA1_CHK80 | GTCCTCTGCGCGCTCCAGCGCGTGCGCTTTTCCGGTGGATCATGCGGTCCGTGGCGCACC |
| FATA1_CHK22 | GTCCTCTGCGCGCTCCAGCGCGTGCGCTTTTCCGGTGGATCATGCGGTCCGTGGCGCACC |
| FATA1_CHK80 | GCAGCGGCCGCTGCCCATGCAGCGCCGCTGCTTCCGAACAGTGGCGGTCAGGGCCGCACC |
| FATA1_CHK22 | GCAGCGGCCGCTGCCCATGCAGCGCCGCTGCTTCCGAACAGTGGCGGTCAGGGCCGCACC |
| FATA1_CHK80 | CGCGGTAGCCGTCCGTCCGGAACCCGCCCAAGAGTTTTGGGAGCAGCTTGAGCTCTGCAA |
| FATA1_CHK22 | CGCGGTAGCCGTCCGTCCGGAACCCGCCCAAGAGTTTTGGGAGCAGCTTGAGCCCTGCAA |
| FATA1_CHK80 | GATGGCGGAGGACAAGCGCATCTTCCTGGAGGAGCACCGGTGCGTGGAGGTCCGGGGCTG |
| FATA1_CHK22 | GATGGCGGAGGACAAGCGCATCTTCCTGGAGGAGCACCGGTGCGTGGAGGTCCGGGGCTG |
| FATA1_CHK80 | ACCGGCCGTCGCATTCAACGTAATCAATCGCATGATGATCAGAGGACACGAAGTCTTGGT |
| FATA1_CHK22 | ACCGGCCGTCGCATTCAACGTAATCAATCGCATGATGATCAGAGGACACGAAGTCTTGGT |
| FATA1_CHK80 | GGCGGTGGCCAGAAACACTGTCCATTGCAAGGGCATAGGGATGCGTTCCTTCACCTCTCA |
| FATA1_CHK22 | GGCGGTGGCCAGAAACACTGTCCATTGCAAGGGCATAGGGATGCGTTCCTTCACCTCTCA |
| FATA1_CHK80 | TTTCTCATTTCTGAATCCCTCCCTGCTCACTCTTTCTCCTCCTCCTTCCCGTTCACGCAG |
| FATA1_CHK22 | TTTCTCATTTCTGAATCCCTCCCTGCTCACTCTTTCTCCTCCTCCTTCCCGTTCACGCAG |
| FATA1_CHK80 | CATTCGGGGCAACGAGGTGGGCCCCTCGCAGCGGCTGACGATCACGGCGGTGGCCAACAT |
| FATA1_CHK22 | CATTCGGGGCAACGAGGTGGGCCCCTCGCAGCGGCTGACGATCACGGCGGTGGCCAACAT |
| FATA1_CHK80 | CCTGCAGGAGGCGGCGGGCAACCACGCGGTGGCCATGTGGGGCCGGAGCTCGGAGGGTTT |
| FATA1_CHK22 | CCTGCAGGAGGCGGCGGGCAACCACGCGGTGGCCATGTGGGGCCGGAGCTCGGAGGGTTT |
| FATA1_CHK80 | CGCGACGGACCCGGAGCTGCAGGAGGCGGGTCTCATCTTTGTGATGACGCGCATGCAGAT |
| FATA1_CHK22 | CGCGACGGACCCGGAGCTGCAGGAGGCGGGTCTCATCTTTGTGATGACGCGCATGCAGAT |
| FATA1_CHK80 | CCAGATGTACCGCTACCCGCGCTGGGGCGACCTGATGCAGGTGGAGACCTGGTTCCAGAC |
| FATA1_CHK22 | CCAGATGTACCGCTACCCGCGCTGGGGCGACCTGATGCAGGTGGAGACCTGGTTCCAGAC |
| FATA1_CHK80 | GGCGGGCAAGCTGGGCGCGCAGCGCGAGTGGGTGCTGCGCGACAAGCTGACCGGCGAGGC |
| FATA1_CHK22 | GGCGGGCAAGCTGGGCGCGCAGCGCGAGTGGGTGCTGCGCGACAAGCTGACCGGCGAGGC |
| FATA1_CHK80 | GCTGGGCGCGGCCACCTCGAGCTGGGTCATGATCAACATCCGCACGCGCCGGCCGTGCCG |
| FATA1_CHK22 | GCTGGGCGCGGCCACCTCGAGCTGGGTCATGATCAACATCCGCACGCGCCGGCCGTGCCG |
| FATA1_CHK80 | CATGCCGGAGCTCGTCCGCGTCAAGTCGGCCTTCTTCGCGCGCGAGCCGCCGCGCCTGGC |
| FATA1_CHK22 | CATGCCGGAGCTCGTCCGCGTCAAGTCGGCCTTCTTCGCGCGCGAGCCGCCGCGCCTGGC |
| FATA1_CHK80 | GCTGCCGCCCGCGGTCACGCGTGCCAAGCTGCCCAACATCGCGACGCCGGCGCCGCTGCG |
| FATA1_CHK22 | GCTGCCGCCCGCGGTCACGCGTGCCAAGCTGCCCAACATCGCGACGCCGGCGCCGCTGCG |
| FATA1_CHK80 | CGGGCACCGCCAGGTCGCGCGCCGCACCGACATGGACATGAACGGGCACGTGAACAACGT |
| FATA1_CHK22 | CGGGCACCGCCAGGTCGCGCGCCGCACCGACATGGACATGAACGGGCACGTGAACAACGT |
| FATA1_CHK80 | GGCCTACCTGGCCTGGTGCCTGGAGGCCGTGCCCGAGCACGTCTTCAGCGACTACCACCT |
| FATA1_CHK22 | GGCCTACCTGGCCTGGTGCCTGGAGGCCGTGCCCGAGCACGTCTTCAGCGACTACCACCT |
| FATA1_CHK80 | CTACCAGATGGAGATCGACTTCAAGGCCGAGTGCCACGCGGGCGACGTCATCTCCTCCCA |
| FATA1_CHK22 | CTACCAGATGGAGATCGACTTCAAGGCCGAGTGCCACGCGGGCGACGTCATCTCCTCCCA |
| FATA1_CHK80 | GGCCGAGCAGATCCCGCCCCAGGAGGCGCTCACGCACAACGGCGCCGGCCGCAACCCCTC |
| FATA1_CHK22 | GGCCGAGCAGATCCCGCCCCAGGAGGCGCTCACGCACAACGGCGCCGGCCGCAACCCCTC |
| FATA1_CHK80 | CTGCTTCGTCCATAGCATTCTGCGCGCCGAGACCGAGCTCGTCCGCGCGCGAACCACATG |
| FATA1_CHK22 | CTGCTTCGTCCATAGCATTCTGCGCGCCGAGACCGAGCTCGTCCGCGCGCGAACCACATG |
| FATA1_CHK80 | GTCGGCCCCCATCGACGCGCCCGCCGCCAAGCCGCCCAAGGCGAGCCACTGAGGACAGGG |
| FATA1_CHK22 | GTCGGCCCCCATCGACGCGCCCGCCGCCAAGCCGCCCAAGGCGAGCCACTGAGGACAGGG |
| FATA1_CHK80 | TGGTTGGCTGGATGGGGAAACGCTGGTCGCGGGATTCGATCCTGCTGCTTATATCCTCCC |
| FATA1_CHK22 | TGGTTGGCTGGATGGGGAAACGCTGGTCGCGGGATTCGATCCTGCTGCTTATATCCTCCC |
| FATA1_CHK80 | TGGAAGCACACCCACGACTCTGAAGAAGAAAACGTGCACACACACAACCCAACCGGCCGA |
| FATA1_CHK22 | TGGAAGCACACCCACGACTCTGAAGAAGAAAACGTGCACACACACAACCCAACCGGCCGA |
| FATA1_CHK80 | ATATTTGCTTCCTTATCCCGGGTCCAAGAGAGACTGCGATGCCCCCCTCAATCAGCATCC |
| FATA1_CHK22 | ATATTTGCTTCCTTATCCCGGGTCCAAGAGAGACTGCGATGCCCCCCTCAATCAGCATCC |
| FATA1_CHK80 | TCCTCCCTGCCGCTTCAATCTTCCCTGCTTGCCTGCGCCCGCGGTGCGCCGTCTGCCCGC |
| FATA1_CHK22 | TCCTCCCTGCCGCTTCAATCTTCCCTGCTTGCCTGCGCCCGCGGTGCGCCGTCTGCCCGC |
| FATA1_CHK80 | CCAGTCAGTCACTCCTGCACAGGCCCCTTGTGCGCAGTGCTCCTGTACCCTTTACCGCTC |
| FATA1_CHK22 | CCAGTCAGTCACTCCTGCACAGGCCCCTTGTGCGCAGTGCTCCTGTACCCTTTACCGCTC |
| FATA1_CHK80 | CTTCCATTCTGCGAGGCCCCCTATTGAATGTATTCGTTGCCTGTGTGGCCAAGCGGGCTG |
| FATA1_CHK22 | CTTCCATTCTGCGAGGCCCCCTATTGAATGTATTCGTTGCCTGTGTGGCCAAGCGGGCTG |
| FATA1_CHK80 | CTGGGCGCGCCGCCGTCGGGCAGTGCTCGGCGACTTTGGCGGAAGCCGATTGTTCTTCTG |
| FATA1_CHK22 | CTGGGCGCGCCGCCGTCGGGCAGTGCTCGGCGACTTTGGCGGAAGCCGATTGTTCTTCTG |
| FATA1_CHK80 | TAAGCCACGCGCTTGCTGCTTTGGGAAGAGAAGGGGGG-GGTACTGAATGGATGAGGAGG |
| FATA1_CHK22 | TAAGCCACGCGCTTGCTGCTTTGGGAAGAGAAGGGGGG-GGTACTGAATGGATGAGGAGG |
| FATA1_CHK80 | AGAAGGAGGGGTATTGGTATTATCTGAGTTGGGGAGGCAGggagagttggaaaatgtaag |
| FATA1_CHK22 | AGAAGGAGGGGTATTGGTATTATCTGAGTTGGGGAGGCAGggagagttggaaaatgtaag |
| FATA1_CHK80 | tggcacgacgggcaaggagaatggtgagcatgtgcatggtgatgtcgttggtcgaggacg |
| FATA1_CHK22 | tggcacgacgggcaaggagaatggtgagcatgtgcatggtgatgtcgttggtcgaggacg |
| FATA1_CHK80 | atcctgcacgcgtgtatctgatgtagaatacggcaatcaccctagtctacatctatacct |
| FATA1_CHK22 | atcctgcacgcgtgtatctgatgtagaatacggcaatcaccctagtctacatctatacct |
| FATA1_CHK80 | tctccgtataacgccctttccaaatgccctcccgtttctctcctattcttgatccacatg |
| FATA1_CHK22 | tctccgtataacgccctttccaaatgccctcccgtttctctcctattcttgatccacatg |
| FATA1_CHK80 | atgaccctggcactatttcaagggctggacatttcaagaaggtttgcgtatctgaagaag |
| FATA1_CHK22 | atgaccctggcactatttcaagggctggacatttcaagaaggtttgcgtatctgaagaag |
| FATA1_CHK80 | gattggtttggagaggtggccgatgaaagtggggtcaagctgcgtggagccctctgcacg |
| FATA1_CHK22 | gattggtttggagaggtggccgatgaaagtggggtcaagctgcgtggagccctctgcacg |
| FATA1_CHK80 | gatttctatggtaatctgcgtccacgtcatcagtagccgtacgcctctgcggcgtcgccg |
| FATA1_CHK22 | gatttctatggtaatctgcgtccacgtcatcagtagccgtacgcctctgcggcgtcgccg |
| FATA1_CHK80 | tgctcctcagccgccgcttcaccaccagccaggagccccatacgcacgcgctcccagacg |
| FATA1_CHK22 | tgctcctcagccgccgcttcaccaccagccaggagccccatacgcacgcgctcccagacg |
| FATA1_CHK80 | gcgcgcgcgacggggacgatcatgagcagcgcgatctggggcgtgtcgtgcgagctgagc |
| FATA1_CHK22 | gcgcgcgcgacggggacgatcatgagcagcgcgatctggggcgtgtcgtgcgagctgagc |
| FATA1_CHK80 | cgcacaaccacgttgccctggccgcgcgtggtgtactggcagttggtcgagaccatggtg |
| FATA1_CHK22 | cgcacaaccacgttgccctggccgcgcgtggtgtactggcagttggtcgagaccatggtg |
| FATA1_CHK80 | tcggacttgaagtaggagccgtcaaagcgcgggaggcgcgtctcggccgagacgttgccg |
| FATA1_CHK22 | tcggacttgaagtaggagccgtcaaagcgcgggaggcgcgtctcggccgagacgttgccg |
| FATA1_CHK80 | ccgatgaccaggctctggcgcttctggcgcgggtcgcggccctgccactgcgcggagccg |
| FATA1_CHK22 | ccgatgaccaggctctggcgcttctggcgcgggtcgcggccctgccactgcgcggagccg |
| FATA1_CHK80 | cccaggagcaggcgcgactcgtcacggccgcgcagcttgacgtcggccgccagcgccgtg |
| FATA1_CHK22 | cccaggagcaggcgcgactcgtcacggccgcgcagcttgacgtcggccgccagcgccgtg |
| FATA1_CHK80 | cccggctcgtaggccgaccccatcttgacgaagaggcggccgatcgctgcgcggaccttg |
| FATA1_CHK22 | cccggctcgtaggccgaccccatcttgacgaagaggcggccgatcgctgcgcggaccttg |
| FATA1_CHK80 | accgcgtcggacaggcgcaggcgctcgtccagcttgaccccgagcgccgcgttgccgggc |
| FATA1_CHK22 | accgcgtcggacaggcgcaggcgctcgtccagcttgaccccgagcgccgcgttgccgggc |
| FATA1_CHK80 | ttgaacggcagcgagaactcctcgcccagcttggagccgagcaggcccagcgccagcttc |
| FATA1_CHK22 | ttgaacggcagcgagaactcctcgcccagcttggagccgagcaggcccagcgccagcttc |
| FATA1_CHK80 | tgccgcttgccgcgcgcgcgcaggcgcgagtcgacgcgcagcgtgtagag |
| FATA1_CHK22 | tgccgcttgccgcgcgcgcgcaggcgcgagtcgacgcgcagcgtgtagag |
| Thi4_CHK80 | ttgcgattcctcttgacccagccacacgcataaattttaacgcaagctggcccaatatgg |
| | (SEQ ID NO: 25) |
| Thi4_CHK22 | ttgcgattcctcttgacccagccacacgcataaattttaacgcaagctggcccaatatgg |
| | (SEQ ID NO: 26) |
| Thi4_CHK80 | tatccgctgtgctcagaacagcaccctcgtgcaccctctggctctgaatctcaccgtcac |
| Thi4_CHK22 | tatccgctgtgctcagaacagcaccctcgtgcaccctctggctctgaatctcaccgtcac |
| Thi4_CHK80 | ggtcatcctcctcctagcctgccagcgcttgggcgcagccatcgggcctgcatagagaaa |
| Thi4_CHK22 | ggtcatcctcctcctagcctgccagcgcttgggcgcagccatcgggcctgcatagagaaa |
| Thi4_CHK80 | gggtacaaagcaacatatataaatacacgtggtattcataatactcgcgcatgtttgtct |
| Thi4_CHK22 | gggtacaaagcaacatatataaatacacgtggtattcataatactcgcgcatgtttgtct |
| Thi4_CHK80 | tgcgcacgcatgcccacgctcgtcgacgcgctcacctctggccaggcagcccccttgcgg |
| Thi4_CHK22 | tgcgcacgcatgcccacgctcgtcgacgcgctcacctctggccaggcagcccccttgcgg |
| Thi4_CHK80 | ggtcgcccgcagcgggcaccgcgaggcctgaggcgccaccagcagcatgtttgaattatt |
| Thi4_CHK22 | ggtcgcccgcagcgggcaccgcgaggcctgaggcgccaccagcagcatgtttgaattatt |
| Thi4_CHK80 | tatacgcagttgtttgaggtacagagaaaccccttgtcattctcttcttgtgtggggtgt |
| Thi4_CHK22 | tatacgcagttgtttgaggtacagagaaaccccttgtcattctcttcttgtgtggggtgt |
| Thi4_CHK80 | cgagtgccattgcagtggtctggccaccggcatatcggataagattgcctgcggatgtcg |
| Thi4_CHK22 | cgagtgccattgcagtggtctggccaccggcatatcggataagattgcctgcggatgtcg |
| Thi4_CHK80 | ttcgtgccagcgtcatcggggcgagagtttataacgcgtgcccacgccagaagccctcac |
| Thi4_CHK22 | ttcgtgccagcgtcatcggggcgagagtttataacgcgtgcccacgccagaagccctcac |
| Thi4_CHK80 | gctttgcacttggcgaggtacaaaaaataccaaaccgcgtcctgaagcattatggcggcg |
| Thi4_CHK22 | gctttgcacttggcgaggtacaaaaaataccaaaccgcgtcctgaagcattatggcggcg |
| Thi4_CHK80 | aggacgaggctgttcaaggtgcggatcgcgaccttgagtgagagacgctccgatggtcgc |
| Thi4_CHK22 | aggacgaggctgttcaaggtgcggatcgcgaccttgagtgagagacgctccgatggtcgc |
| Thi4_CHK80 | ggccagaaccccagcgcccgtcaaaagccggtcctcacgccccagggctgagtgcgcgcc |
| Thi4_CHK22 | ggccagaaccccagcgcccgtcaaaagccggtcctcacgccccagggctgagtgcgcgcc |
| Thi4_CHK80 | cgcaaaccccttttgcaggaactcaaggaggtggctcgatccaaggacaacaacggcatc |
| Thi4_CHK22 | cgcaaaccccttttgcaggaactcaaggaggtggctcgatccaaggacaacaacggcatc |
| Thi4_CHK80 | gagctcattcccaatgactccaacatcttcctctggcgggcggtgattgaggcaaggggg |
| Thi4_CHK22 | gagctcattcccaatgactccaacatcttcctctggcgggcggtgattgaggcaaggggg |
| Thi4_CHK80 | tctgcttcattgggtggagggagatgagttgtgtgcagggcgctcaaaggaggttttggt |
| Thi4_CHK22 | tctgcttcattgggtggagggagatgagttgtgtgcagggcgctcaaaggaggttttggt |
| Thi4_CHK80 | cgcctattgcccaaggtcatgcatccgtgtgtatcccgtgccccctcccttcaacagggc |
| Thi4_CHK22 | cgcctattgcccaaggtcatgcatccgtgtgtatcccgtgccccctcccttcaacagggc |
| Thi4_CHK80 | cccgcggactcgccctatcaggggggtcactttgaactcagcatccaagtgcctgagcag |
| Thi4_CHK22 | cccgcggactcgccctatcaggggggtcactttgaactcagcatccaagtgcctgagcag |
| Thi4_CHK80 | tacccattggtcccgccgcaagtcaagttcaagaccaagctcttccacccgaacgtgcac |
| Thi4_CHK22 | tacccattggtcccgccgcaagtcaagttcaagaccaagctcttccacccgaacgtgcac |
| Thi4_CHK80 | ttcaaggtgagctgtttggagacaaaggtggcagggagagtgctagcaagcaggggttga |
| Thi4_CHK22 | ttcaaggtgagctgtttggagacaaaggtggcagggagagtgctagcaagcaggggttga |
| Thi4_CHK80 | cgggcgttgctgttgagttttgatgtatagactcggccgggaacttgagtggaagccgac |
| Thi4_CHK22 | cgggcgttgctgttgagttttgatgtatagactcggccgggaacttgagtggaagccgac |
| Thi4_CHK80 | agacctttcatctgcctctctgcccatgcagacgggagagatctgcctcgacattctcaa |
| Thi4_CHK22 | agacctttcatctgcctctctgcccatgcagacgggagagatctgcctcgacattctcaa |
| Thi4_CHK80 | gaacgcctggagcccagcttggacgctgcaggtacgattgtttggtttaggagaggcggc |
| Thi4_CHK22 | gaacgcctggagcccagcttggacgctgcaggtacgattgtttggtttaggagaggcggc |
| Thi4_CHK80 | -cccttggtcgtcccctggcactcacgactcttgggaaaatatgccgatctccgcattcc |
| Thi4_CHK22 | ccccttggtcgtcccctggcactcacgactcttgggaaaatatgccgatctccgcattcc |
| Thi4_CHK80 | tgtctccagacgcctgctctgtgcgcgctgtctcgatgaagcattcttttcctccccaat |
| Thi4_CHK22 | tgtctccagacgcctgctctgtgcgcgctgtctcgatgaagcattcttttcctccccaat |
| Thi4_CHK80 | gcccgcagtcggtgtgccaggccatcgtggcgctcttgacagattctgcgccagactccc |
| Thi4_CHK22 | gcccgcagtcggtgtgccaggccatcgtggcgctcttgacagattctgcgccagactccc |
| Thi4_CHK80 | CCCTCAACTGCGACGCTGGGAACCTTCTCCGGGCAGGCGATGTGCGTGGGTTTGCCTCCT |
| Thi4_CHK22 | CCCTCAACTGCGACGCTGGGAACCTTCTCCGGGCAGGCGATGTGCGTGGGTTTGCCTCCT |
| Thi4_CHK80 | TGGCACGGCTCTACACCGTCGAGTACGCCATGAGGCGGTGATGGCTGTGTCGGTTGCCAC |
| Thi4_CHK22 | TGGCACGGCTCTACACCGTCGAGTACGCCATGAGGCGGTGATGGCTGTGTCGGTTGCCAC |
| Thi4_CHK80 | TTCGTCCAGAGACGGCAAGTCGTCCATCCTCTGCGTGTGTGGCGCGACGCTGCAGCAGTC |
| Thi4_CHK22 | TTCGTCCAGAGACGGCAAGTCGTCCATCCTCTGCGTGTGTGGCGCGACGCTGCAGCAGTC |
| Thi4_CHK80 | CCTCTGCAGCAGATGAGCGTGACTTTGGCCATTTCACGCACTCGAGTGTACACAATCCAT |
| Thi4_CHK22 | CCTCTGCAGCAGATGAGCGTGACTTTGGCCATTTCACGCACTCGAGTGTACACAATCCAT |
| Thi4_CHK80 | TTTTCTTAAAGCAAATGACTGCTGATTGACCAGATACTGTAACGCTGATTTCGCTCCAGA |
| Thi4_CHK22 | TTTTCTTAAAGCAAATGACTGCTGATTGACCAGATACTGTAACGCTGATTTCGCTCCAGA |
| Thi4_CHK80 | TCGCACAGATAGCGACCATGTTGCTGCGTCTGAAAATCTGGATTCCGAATTCGACCCTGG |
| Thi4_CHK22 | TCGCACAGATAGCGACCATGTTGCTGCGTCTGAAAATCTGGATTCCGAATTCGACCCTGG |
| Thi4_CHK80 | CGCTCCATCCATGCAACAGATGGCGACACTTGTTACAATTCCTGTCACCCATCGGCATGG |
| Thi4_CHK22 | CGCTCCATCCATGCAACAGATGGCGACACTTGTTACAATTCCTGTCACCCATCGGCATGG |
| Thi4_CHK80 | AGCAGGTCCACTTAGATTCCCGATCACCCACGCACATCTCGCTAATAGTCATTCGTTCGT |
| Thi4_CHK22 | AGCAGGTCCACTTAGATTCCCGATCACCCACGCACATCTCGCTAATAGTCATTCGTTCGT |
| Thi4_CHK80 | GTCTTCGATCAATCTCAAGTGAGTGTGCATGGATCTTGGTTGACGATGCGGTATGGGTTT |
| Thi4_CHK22 | GTCTTCGATCAATCTCAAGTGAGTGTGCATGGATCTTGGTTGACGATGCGGTATGGGTTT |
| Thi4_CHK80 | GCGCCGCTGGCTGCAGGGTCTGCCCAAGGCAAGCTAACCCAGCTCCTCTCCCCGACAATA |
| Thi4_CHK22 | GCGCCGCTGGCTGCAGGGTCTGCCCAAGGCAAGCTAACCCAGCTCCTCTCCCCGACAATA |
| Thi4_CHK80 | CTCTCGCAGGCAAAGCCGGTCACTTGCCTTCCAGATTGCCAATAAACTCAATTATGGCCT |
| Thi4_CHK22 | CTCTCGCAGGCAAAGCCGGTCACTTGCCTTCCAGATTGCCAATAAACTCAATTATGGCCT |
| Thi4_CHK80 | CTGTCATGCCATCCATGGGTCTGATGAATGGTCACGCTCGTGTCCTGACCGTTCCCCAGC |
| Thi4_CHK22 | CTGTCATGCCATCCATGGGTCTGATGAATGGTCACGCTCGTGTCCTGACCGTTCCCCAGC |
| Thi4_CHK80 | CTCTGGCGTCCCCTGCCCCGCCCACCAGCCCACGCCGCGCGGCAGTCGCTGCCAAGGCTG |
| Thi4_CHK22 | CTCTGGCGTCCCCTGCCCCGCCCACCAGCCCACGCCGCGCGGCAGTCGCTGCCAAGGCTG |
| Thi4_CHK80 | TCTCGGATGCCCAGCGCCATGCCACGCCCTTTGATGGCTTCAAGTACGATTACGGTGTTG |
| Thi4_CHK22 | TCTCGGATGCCCAGCGCCATGCCACGCCCTTTGATGGCTTCAAGTACGATTACGGTGTTG |
| Thi4_CHK80 | GATTGTGTGTTTGTTGCGTAGTGTGCATGGTTTAGAATAATACACTTGATTTCTTGCTCA |
| Thi4_CHK22 | GATTGTGTGTTTGTTGCGTAGTGTGCATGGTTTAGAATAATACACTTGATTTCTTGCTCA |
| Thi4_CHK80 | CGGCAATCTCGGCTTGTCCGCAGGTTCAACCCCATTTCGGAGTCTCAGGTCAGCCGCGCA |
| Thi4_CHK22 | CGGCAATCTCGGCTTGTCCGCAGGTTCAACCCCATTTCGGAGTCTCAGGTCAGCCGCGCA |
| Thi4_CHK80 | ATGACCAGCCGCTACTTCAAGGACTTGCACGACAACGCCGAGGTGAGCTATGTTTAGGAC |
| Thi4_CHK22 | ATGACCAGCCGCTACTTCAAGGACTTGCACGACAACGCCGAGGTGAGCTATGTTTAGGAC |
| Thi4_CHK80 | TTGATTGGAAATTGTCGTCGACGCATATTCGCGCTCCGCGACAGCACCCAAGCAAAATGT |
| Thi4_CHK22 | TTGATTGGAAATTGTCGTCGACGCATATTCGCGCTCCGCGACAGCACCCAAGCAAAATGT |
| Thi4_CHK80 | CAAGTGCGTTCCGATTTGCGTCCGCAGGTCGATGTTGTGATCGTCGGCGCCGGATCCGCC |
| Thi4_CHK22 | CAAGTGCGTTCCGATTTGCGTCCGCAGGTCGATGTTGTGATCGTCGGCGCCGGATCCGCC |
| Thi4_CHK80 | GGTCTGTCCTGCGCTTACGAGCTGACCAAGCACCCTGACGTCCGGGTACGCGAGCTGAGA |
| Thi4_CHK22 | GGTCTGTCCTGCGCTTACGAGCTGACCAAGCACCCTGACGTCCGGGTACGCGAGCTGAGA |
| Thi4_CHK80 | TTCGATTAGACATAAATTGAAGATTAAACCCGTAGAAAAATTTGATGGTCGCGAAACTGT |
| Thi4_CHK22 | TTCGATTAGACATAAATTGAAGATTAAACCCGTAGAAAAATTTGATGGTCGCGAAACTGT |
| Thi4_CHK80 | GCTCGATTGCAAGAAATTGATCGTCCTCCACTCCGCAGGTCGCCATCATCGAGCAGGGCG |
| Thi4_CHK22 | GCTCGATTGCAAGAAATTGATCGTCCTCCACTCCGCAGGTCGCCATCATCGAGCAGGGCG |
| Thi4_CHK80 | TTGCTCC-GGCGGCGGCGCCTGGCTGGGGGGACAGCTGTTCTCGGCCATGTGTGTACGTA |
| Thi4_CHK22 | TTGCTCCCGGCGGCGGCGCCTGGCTGGGGGGACAGCTGTTCTCGGCCATGTGTGTACGTA |
| Thi4_CHK80 | GAAGGATGAATTTCAGCTGGTTTTCGTTGCACAGCTGTTTGTGCATGATTTGTTTCAGAC |
| Thi4_CHK22 | GAAGGATGAATTTCAGCTGGTTTTCGTTGCACAGCTGTTTGTGCATGATTTGTTTCAGAC |
| Thi4_CHK80 | TATTGTTGAATGTTTTTAGATTTCTTAGGATGCATGATTTGTCTGCATGCGACTtttagc |
| Thi4_CHK22 | TATTGTTGAATGTTTTTAGATTTCTTAGGATGCATGATTTGTCTGCATGCGACTtttagc |
| Thi4_CHK80 | tagcacccttacacaccatccaacatcttgctgccttgcctcccctgcgcagatccgcaa |
| Thi4_CHK22 | tagcacccttacacaccatccaacatcttgctgccttgcctcccctgcgcagatccgcaa |
| Thi4_CHK80 | gccggcgcacaagctgatggacgagctcaacatcccttacgacgacgaggtgggtattgg |
| Thi4_CHK22 | gccggcgcacaagctgatggacgagctcaacatcccttacgacgacgaggtgggtattgg |
| Thi4_CHK80 | gcagctagaacgcatgcgtgctgtgcgactgggtcgatccattgtgcgaaacgtgtggcg |
| Thi4_CHK22 | gcagctagaacgcatgcgtgctgtgcgactgggtcgatccattgtgcgaaacgtgtggcg |
| Thi4_CHK80 | gaatacgtgcgcgtccccggccatgcaccgaccccccctcccaccaccccacaaatataa |
| Thi4_CHK22 | gaatacgtgcgcgtccccggccatgcaccgaccccccctcccaccaccccacaaatataa |
| Thi4_CHK80 | cagggtgacatggtggtggtcaagcacgccgccctggtgacgtccacgctgctgtccaag |
| Thi4_CHK22 | cagggtgacatggtggtggtcaagcacgccgccctggtgacgtccacgctgctgtccaag |
| Thi4_CHK80 | gtgctggcggcccccaacatcaagcttttcaacgccaccgcggcggaggacctgatcgtc |
| Thi4_CHK22 | gtgctggcggcccccaacatcaagcttttcaacgccaccgcggcggaggacctgatcgtc |
| Thi4_CHK80 | aagtccaagccggccggcggggcggccgtcccgcacgtggccggggccgtgaccaactgg |
| Thi4_CHK22 | aagtccaagccggccggcggggcggccgtcccgcacgtggccggggccgtgaccaactgg |
| Thi4_CHK80 | accctggtgtccctcaaccacgacacccagatgtgcatggaccccaacaccatcctgagc |
| Thi4_CHK22 | accctggtgtccctcaaccacgacacccagatgtgcatggaccccaacaccatcctgagc |
| Thi4_CHK80 | aaggtcatggtctcctccaccggccacgacggccccatgggcgcctccggggtcaagcgc |
| Thi4_CHK22 | aaggtcatggtctcctccaccggccacgacggccccatgggcgcctccggggtcaagcgc |
| Thi4_CHK80 | ctggccaagctgggcctgatcgagcgcgcgccgggcatgggcgcgctggacatgaacagc |
| Thi4_CHK22 | ctggccaagctgggcctgatcgagcgcgcgccgggcatgggcgcgctggacatgaacagc |
| Thi4_CHK80 | gccgaggacgcggtggtcgaccggacgcgggagatcgtccctggcatggtcatctgcggc |
| Thi4_CHK22 | gccgaggacgcggtggtcgaccggacgcgggagatcgtccctggcatggtcatctgcggc |
| Thi4_CHK80 | atggaggtcgccgagctggacgggtgcccgcgcatgggccccacctttggcgccatgttt |
| Thi4_CHK22 | atggaggtcgccgagctggacgggtgcccgcgcatgggccccacctttggcgccatgttt |
| Thi4_CHK80 | gtgtccggggtcaaggccgcgcacgtggccctggcctcgctgcgccgccagcaggaggag |
| Thi4_CHK22 | gtgtccggggtcaaggccgcgcacgtggccctggcctcgctgcgccgccagcaggaggag |
| Thi4_CHK80 | gaggggctccgcgccaagaccgcagacaccctgcgcagcgcggcgccgtccatggccatg |
| Thi4_CHK22 | gaggggctccgcgccaagaccgcagacaccctgcgcagcgcggcgccgtccatggccatg |
| Thi4_CHK80 | gcgtgacctggtgatccccaagagggcggacatgcgtcgcgtctccctccggtgcccaga |
| Thi4_CHK22 | gcgtgacctggtgatccccaagagggcggacatgcgtcgcgtctccctccggtgcccaga |
| Thi4_CHK80 | gatctgttccacggttgtctgcgatgctttcaaaactgttatttcactaaaatgactcga |
| Thi4_CHK22 | gatctgttccacggttgtctgcgatgctttcaaaactgttatttcactaaaatgactcga |
| Thi4_CHK80 | ttctcctctaaaaaaaacatgatgttgatagtatctccacaaaagcatggcgactcagat |
| Thi4_CHK22 | ttctcctctaaaaaaaacatgatgttgatagtatctccacaaaagcatggcgactcagat |
| Thi4_CHK80 | gcctctactgcactgtactcaagaatacaactctggagacccgggtcccgttcccatttg |
| Thi4_CHK22 | gcctctactgcactgtactcaagaatacaactctggagacccgggtcccgttcccatttg |
| Thi4_CHK80 | gcgccgaatgttgatgcgccacttttgctggccatgcctgaggtattctgtacaaaagaa |
| Thi4_CHK22 | gcgccgaatgttgatgcgccacttttgctggccatgcctgaggtattctgtacaaaagaa |
| Thi4_CHK80 | ggagcggccaatgcgggttttcaagcagacggcgcgctggggcctggaaatcaaaaggcg |
| Thi4_CHK22 | ggagcggccaatgcgggttttcaagcagacggcgcgctggggcctggaaatcaaaaggcg |
| Thi4_CHK80 | gtagtacacgggagagagtcggattgatgatgagggatttaaaaaaggccaggcagtgag |
| Thi4_CHK22 | gtagtacacgggagagagtcggattgatgatgagggatttaaaaaaggccaggcagtgag |
| Thi4_CHK80 | gtgagagttgtacgattgttgccccaggattgcctttttagggggggacggccaggggcg |
| Thi4_CHK22 | gtgagagttgtacgattgttgccccaggattgcctttttagggggggacggccaggggcg |
| Thi4_CHK80 | cagttattggggcaccgcacatatagatccgcgcaagcaaatggcctcgagagcaggcgc |
| Thi4_CHK22 | cagttattggggcaccgcacatatagatccgcgcaagcaaatggcctcgagagcaggcgc |
| Thi4_CHK80 | gatggttaagaaaggaaggacgagtagcggatgacgtgatactgcatacgaagaacgtac |
| Thi4_CHK22 | gatggttaagaaaggaaggacgagtagcggatgacgtgatactgcatacgaagaacgtac |
| Thi4_CHK80 | tagtgaggagagtgcttgaaacaacatatgatgctgccccccctctctgccaac |
| Thi4_CHK22 | tagtgaggagagtgcttgaaacaacatatgatgctgccccccctctctgccaac |

Key:
Background DNA flanking homology arm recognition site: lowercase, non-bold
Homology arm recognition site: CAPITALIZED, NON-BOLD
Genomic region of interest between recognition sites: CAPITAL, BOLD
SNP difference: underlined a c g t

TABLE 13 shows genetic sequences of selectable markers that can be used to generate genetically modified microalgae. The classically improved microalgal strains provided herein do not comprises any of these exogenous sequences. Saccharomyces cerevisiae suc2 (ScSUC2) encodes for sucrose invertase, which allows for selection of strains that grow on sucrose media. Saccharomyces carlbergenesis MEL1 (ScarMEL) encodes for alpha-galactosidase (melibiase), which allows for selection of strains that grow on melibiose media. Arabidopsis thaliana ThiC (AtTHIC) encodes for phosphomethylpyrimidine synthase, an enzyme involved in pyrimidine synthesis in the thiamine biosynthesis pathway.

TABLE 13

| | |
|---|---|
| S. cerevisiae suc2 | ATGCTGCTGCAGGCCTTCCTGTTCCTGCTGGCCGGCTTCGCCGCCAAGATCAGCGC |
| sucrose invertase | CTCCATGACGAACGAGACGTCCGACCGCCCCCTGGTGCACTTCACCCCCAACAAGG |
| (ScSUC2) | GCTGGATGAACGACCCCAACGGCCTGTGGTACGACGAGAAGGACGCCAAGTGGCAC |
| (SEQ ID NO: 27) | CTGTACTTCCAGTACAACCCGAACGACACCGTCTGGGGGACGCCCTTGTTCTGGGG |
| | CCACGCCACGTCCGACGACCTGACCAACTGGGAGGACCAGCCCATCGCCATCGCCC |
| | CGAAGCGCAACGACTCCGGCGCCTTCTCCGGCTCCATGGTGGTGGACTACAACAAC |
| | ACCTCCGGCTTCTTCAACGACACCATCGACCCGCGCCAGCGCTGCGTGGCCATCTG |
| | GACCTACAACACCCCGGAGTCCGAGGAGCAGTACATCTCCTACAGCCTGGACGGCG |
| | GCTACACCTTCACCGAGTACCAGAAGAACCCCGTGCTGGCCGCCAACTCCACCCAG |
| | TTCCGCGACCCGAAGGTCTTCTGGTACGAGCCCTCCCAGAAGTGGATCATGACCGC |
| | GGCCAAGTCCCAGGACTACAAGATCGAGATCTACTCCTCCGACGACCTGAAGTCCT |
| | GGAAGCTGGAGTCCGCGTTCGCCAACGAGGGCTTCCTCGGCTACCAGTACGAGTGC |
| | CCCGGCCTGATCGAGGTCCCCACCGAGCAGGACCCCAGCAAGTCCTACTGGGTGAT |
| | GTTCATCTCCATCAACCCCGGCGCCCCGGCCGGCGGCTCCTTCAACCAGTACTTCG |
| | TCGGCAGCTTCAACGGCACCCACTTCGAGGCCTTCGACAACCAGTCCCGCGTGGTG |
| | GACTTCGGCAAGGACTACTACGCCCTGCAGACCTTCTTCAACACCGACCCGACCTA |
| | CGGGAGCGCCCTGGGCATCGCGTGGGCCTCCAACTGGGAGTACTCCGCCTTCGTGC |
| | CCACCAACCCCTGGCGCTCCTCCATGTCCCTCGTGCGCAAGTTCTCCCTCAACACC |
| | GAGTACCAGGCCAACCCGGAGACGGAGCTGATCAACCTGAAGGCCGAGCCGATCCT |
| | GAACATCAGCAACGCCGGCCCCTGGAGCCGGTTCGCCACCAACACCACGTTGACGA |
| | AGGCCAACAGCTACAACGTCGACCTGTCCAACAGCACCGGCACCCTGGAGTTCGAG |
| | CTGGTGTACGCCGTCAACACCACCCAGACGATCTCCAAGTCCGTGTTCGCGGACCT |
| | CTCCCTCTGGTTCAAGGGCCTGGAGGACCCCGAGGAGTACCTCCGCATGGGCTTCG |
| | AGGTGTCCGCGTCCTCCTTCTTCCTGGACCGCGGGAACAGCAAGGTGAAGTTCGTG |
| | AAGGAGAACCCCTACTTCACCAACCGCATGAGCGTGAACAACCAGCCCTTCAAGAG |
| | CGAGAACGACCTGTCCTACTACAAGGTGTACGGCTTGCTGGACCAGAACATCCTGG |
| | AGCTGTACTTCAACGACGGCGACGTCGTGTCCACCAACACCTACTTCATGACCACC |
| | GGGAACGCCCTGGGCTCCGTGAACATGACGACGGGGGTGGACAACCTGTTCTACAT |
| | CGACAAGTTCCAGGTGCGCGAGGTCAAGTGA |
| S. carlbergenesis | ATGTTCGCGTTCTACTTCCTGACGGCCTGCATCTCCCTGAAGGGCGTGTTCGGCGT |
| MEL1 alpha | CTCCCCCTCCTACAACGGCCTGGGCCTGACGCCCCAGATGGGCTGGGACAACTGGA |
| galactosidase or | ACACGTTCGCCTGCGACGTCTCCGAGCAGCTGCTGCTGGACACGGCCGACCGCATC |
| melibiase (ScarMEL) | TCCGACCTGGGCCTGAAGGACATGGGCTACAAGTACATCATCCTGGACGACTGCTG |
| (SEQ ID NO: 28) | GTCCTCCGGCCGCGACTCCGACGGCTTCCTGGTCGCCGACGAGCAGAAGTTCCCCA |
| | ACGGCATGGGCCACGTCGCCGACCACCTGCACAACAACTCCTTCCTGTTCGGCATG |
| | TACTCCTCCGCGGGCGAGTACACGTGCGCCGGCTACCCCGGCTCCCTGGGCCGCGA |
| | GGAGGAGGACGCCCAGTTCTTCGCGAACAACCGCGTGGACTACCTGAAGTACGACA |
| | ACTGCTACAACAAGGGCCAGTTCGGCACGCCCGAGATCTCCTACCACCGCTACAAG |
| | GCCATGTCCGACGCCCTGAACAAGACGGGCCGCCCCATCTTCTACTCCCTGTGCAA |
| | CTGGGGCCAGGACCTGACCTTCTACTGGGGCTCCGGCATCGCGAACTCCTGGCGCA |
| | TGTCCGGCGACGTCACGGCGGAGTTCACGCGCCCCGACTCCCGCTGCCCCTGCGAC |
| | GGCGACGAGTACGACTGCAAGTACGCCGGCTTCCACTGCTCCATCATGAACATCCT |
| | GAACAAGGCCGCCCCCATGGGCCAGAACGCGGGCGTCGGCGGCTGGAACGACCTGG |
| | ACAACCTGGAGGTCGGCGTCGGCAACCTGACGGACGACGAGGAGAAGGCGCACTTC |
| | TCCATGTGGGCCATGGTGAAGTCCCCCCTGATCATCGGCGCGAACGTGAACAACCT |
| | GAAGGCCTCCTCCTACTCCATCTACTCCCAGGCGTCCGTCATCGCCATCAACCAGG |
| | ACTCCAACGGCATCCCCGCCACGCGCGTCTGGCGCTACTACGTGTCCGACACGGAC |
| | GAGTACGGCCAGGGCGAGATCCAGATGTGGTCCGGCCCCCTGGACAACGGCGACCA |
| | GGTCGTGGCGCTGCTGAACGGCGGCTCCGTGTCCCGCCCCATGAACACGACCCTGG |
| | AGGAGATCTTCTTCGACTCCAACCTGGGCTCCAAGAAGCTGACCTCCACCTGGGAC |
| | ATCTACGACCTGTGGGCGAACCGCGTCGACAACTCCACGGCGTCCGCCATCCTGGG |
| | CCGCAACAAGACCGCCACCGGCATCCTGTACAACGCCACCGAGCAGTCCTACAAGG |
| | ACGGCCTGTCCAAGAACGACACCCGCCTGTTCGGCCAGAAGATCGGCTCCCTGTCC |
| | CCCAACGCGATCCTGAACACGACCGTCCCCGCCCACGGCATCGCGTTCTACCGCCT |
| | GCGCCCCTCCTCCTGA |
| A. thaliana ThiC | ATGGCCGCGTCCGTCCACTGCACCCTGATGTCCGTGGTCTGCAACAACAAGAACCA |
| (AtTHIC) | CTCCGCCCGCCCCAAGCTGCCCAACTCCTCCCTGCTGCCCGGCTTCGACGTGGTGG |
| (SEQ ID NO: 29) | TCCAGGCCGCGGCCACCCGCTTCAAGAAGGAGACGACGACCACCCGCGCCACGCTG |
| | ACGTTCGACCCCCCCACGACCAACTCCGAGCGCGCCAAGCAGCGCAAGCACACCAT |
| | CGACCCCTCCTCCCCCGACTTCCAGCCCATCCCCTCCTTCGAGGAGTGCTTCCCCA |
| | AGTCCACGAAGGAGCACAAGGAGGTGGTGCACGAGGAGTCCGGCCACGTCCTGAAG |
| | GTGCCCTTCCGCCGCGTGCACCTGTCCGGCGGCGAGCCCGCCTTCGACAACTACGA |
| | CACGTCCGGCCCCCAGAACGTCAACGCCCACATCGGCCTGGCGAAGCTGCGCAAGG |
| | AGTGGATCGACCGCCGCGAGAAGCTGGGCACGCCCCGCTACACGCAGATGTACTAC |
| | GCGAAGCAGGGCATCATCACGGAGGAGATGCTGTACTGCGCGACGCGCGAGAAGCT |
| | GGACCCCGAGTTCGTCCGCTCCGAGGTCGCGCGGGGCCGCGCCATCATCCCCTCCA |
| | ACAAGAAGCACCTGGAGCTGGAGCCCATGATCGTGGGCCGCAAGTTCCTGGTGAAG |
| | GTGAACGCGAACATCGGCAACTCCGCCGTGGCCTCCTCCATCGAGGAGGAGGTCTA |
| | CAAGGTGCAGTGGGCCACCATGTGGGGCGCCGACACCATCATGGACCTGTCCACGG |
| | GCCGCCACATCCACGAGACGCGCGAGTGGATCCTGCGCAACTCCGCGGTCCCCGTG |
| | GGCACCGTCCCCATCTACCAGGCGCTGGAGAAGGTGGACGGCATCGCGGAGAACCT |
| | GAACTGGGAGGTGTTCCGCGAGACGCTGATCGAGCAGGCCGAGCAGGGCGTGGACT |
| | ACTTCACGATCCACGCGGGCGTGCTGCTGCGCTACATCCCCCTGACCGCCAAGCGC |
| | CTGACGGGCATCGTGTCCCGCGGCGGCTCCATCCACGCGAAGTGGTGCCTGGCCTA |
| | CCACAAGGAGAACTTCGCCTACGAGCACTGGGACGACATCCTGGACATCTGCAACC |
| | AGTACGACGTCGCCCTGTCCATCGGCGACGGCCTGCGCCCCGGCTCCATCTACGAC |
| | GCCAACGACACGGCCCAGTTCGCCGAGCTGCTGACCCAGGGCGAGCTGACGCGCCG |
| | CGCGTGGGAGAAGGACGTGCAGGTGATGAACGAGGGCCCCGGCCACGTGCCCATGC |
| | ACAAGATCCCCGAGAACATGCAGAAGCAGCTGGAGTGGTGCAACGAGGCGCCCTTC |
| | TACACCCTGGGCCCCCTGACGACCGACATCGCGCCCGGCTACGACCACATCACCTC |
| | CGCCATCGGCGCGGCCAACATCGGCGCCCTGGGCACCGCCCTGCTGTGCTACGTGA |
| | CGCCCAAGGAGCACCTGGGCCTGCCCAACCGCGACGACGTGAAGGCGGGCGTCATC |
| | GCCTACAAGATCGCCGCCCACGCGGCCGACCTGGCCAAGCAGCACCCCCACGCCCA |
| | GGCGTGGGACGACGCGCTGTCCAAGGCGCGCTTCGAGTTCCGCTGGATGGACCAGT |
| | TCGCGCTGTCCCTGGACCCCATGACGGCGATGTCCTTCCACGACGAGACGCTGCCC |
| | GCGGACGGCGCGAAGGTCGCCCACTTCTGCTCCATGTGCGGCCCCAAGTTCTGCTC |
| | CATGAAGATCACGGAGGACATCCGCAAGTACGCCGAGGAGAACGGCTACGGCTCCG |
| | CCGAGGAGGCCATCCGCCAGGGCATGGACGCCATGTCCGAGGAGTTCAACATCGCC |
| | AAGAAGACGATCTCCGGCGAGCAGCACGGCGAGGTCGGCGGCGAGATCTACCTGCC |
| | CGAGTCCTACGTCAAGGCCGCGCAGAAGTGA |

### Example 9. Characterization of High Oleic Acid Triglyceride Oils Produced by Classically Improved Microalgal Strains

The sterol and triterpene alcohol compositions of high oleic refined, bleached, and deodorized (RBD) triglyceride oils of the disclosure were determined using gas chromatography combined with mass spectrometry and flame ionization detection. Samples were prepared using the technique described in the German standardized method F-Ill for sterols in fats and oils from Aitzetmüller, et al. (1998), Analysis of sterol content and composition in fats and oils by capillary-gas liquid chromatography using an internal standard. Comments on the German sterol method. Fett/Lipid, 100: 429-435, which is incorporated herein by reference in its entirety. After addition of an internal standard, the oils were saponified. The unsaponifiable fractions were isolated using solid-phase extraction. Trimethylsilyl (TMS) derivatives of the unsaponifiable fraction were injected on an Agilent 8890 gas chromatograph equipped with a 5977B single quadrupole EI-MS and FID detector. A capillary flow technology (CFT) splitter was used to split the sample between the MS and FID detectors following separation using a DB-5MS Ultra Inert column (60 m length×0.25 mm inner diameter×0.25 μm film thickness). Mass spectra data, in conjunction with retention time comparison with analytical standards, if available, were used to identify each of the sterol species. Quantification was based on the FID response of the sterol TMS ethers compared to that of the internal standard.

The resulting sterol profiles are shown in TABLE 14. Amounts are shown as mg/100 g of the oil and as approximate percentages of total detected sterols. Minor unidentified peaks were not accounted for. Not all sterols were identifiable and are listed as unknown. Ergosterol was the main sterol present in both oils. Various other sterols were present in the high oleic oils in significant amounts, including ergosta-5,8-dien-3-ol, (3β)- and 5.xi.-ergost-7-en-3β-ol, (3β). Campesterol, stigmasterol, and β-sitosterol were not detected in either oils.

TABLE 14
Sterol and triterpene alcohol composition (mg/100 g and %)

| RBD Oil Strain | CHK74 | CHK80 |
|---|---|---|
| Brassicasterol (Ergosta-5,22-dien-3β-ol) | ND | ND |
| | (0%) | (0%) |
| Ergosta-5,7,9(11),22-tetraen-3- | ND | ND |
| ol,(3β,22E) | (0%) | (0%) |
| Ergosta-7,22-dien-3-ol, (3β,22E) | ND | ND |
| | (0%) | (0%) |
| Unknown | ND | ND |
| | (0%) | (0%) |
| Ergosterol | 118.7 | 166.1 |
| | (48%) | (60%) |
| Campesterol | ND | ND |
| | (0%) | (0%) |
| Ergost-8(14)-en-3-ol, (3β) | ND | ND |
| | (0%) | (0%) |
| Stigmasterol | ND | ND |
| | (0%) | (0%) |
| Unknown (MS similar to Ergost-8(14)- | 6.9 | 7.2 |
| en-3-ol, (3β)) | (3%) | (3%) |

| | | |
|---|---|---|
| Ergosta-5,8-dien-3-ol, (3β) | 13.5 | 18.7 |
| | (5%) | (7%) |
| 5.Xi.-Ergost-7-en-3β-ol | 30.4 | 32.0 |
| | (12%) | (11%) |
| β-Sitosterol | ND | ND |
| | (0%) | (0%) |
| 9,19-Cyclolanost-24-en-3-ol, (3β) | 60.6 | 36.1 |
| | (24%) | (13%) |
| 9,19-Cyclolanostan-3-ol, 24-methylene-, | 11.4 | 10.9 |
| (3β) | (5%) | (4%) |
| Unknown | 7.9 | 7.8 |
| | (3%) | (3%) |
| TOTAL | 249.4 | 278.8 |
| | (100%) | (100%) |

ND, not detectable.

TABLE 15 shows fatty acid profiles of a TAG oil produced by strains CHK74 and CHK80. Values are in percentages of total detected fatty acids.

TABLE 15

| Fatty Acid Species | CHK74 | CHK80 |
|---|---|---|
| Titer (g/L) | 143.7 | 148.5 |
| C12:0 | 0 | 0 |
| C14:0 | 0.2 | 0.18 |
| C14:1 | 0 | 0 |
| C15:0 | 0.01 | 0.01 |
| C16:0 | 3.83 | 3.9 |
| C16:1 | 0.17 | 0.14 |
| C17:0 | 0.02 | 0.02 |
| C17:1 | 0.02 | 0.02 |
| C18:0 | 2.11 | 2.51 |
| C18:1 | 85.91 | 86.35 |
| C18:2 | 6.14 | 5.09 |
| C18:3 alpha | 0.14 | 0.16 |
| C20:0 | 0.15 | 0.22 |
| C20:1 | 0.98 | 0.90 |
| C22:0 | 0.03 | 0.05 |
| 12-OH-C18:1 | 0.11 | 0.09 |
| C24:0 | 0.01 | 0.02 |
| TOTAL | 99.83 | 99.84 |

TAG analysis was carried out utilizing Liquid Chromatography/Time of Flight-Mass Spectrometry (LC/TOF-MS), where fractionation of TAG species is carried out on a C18 column followed by interrogation of individual peaks on a TOF LC-MS equipped with an Atmospheric Pressure Chemical Ionization (APCI) source. LC TAG standards from NuChek and samples were run on a Shimadzu Shim-pack XR-ODS III 2.2 micron, 2.0×200 mm column and confirmed by MS on an Agilent TOF LC-MS equipped with an APCI ionization source utilizing a method from Neff, et al. (1995), Soybean Oil Triacylglycerol Analysis by Reversed-Phase High-Performance Liquid Chromatography Coupled with Atmospheric Pressure Chemical Ionization Mass Spectrometry, JAOCS, 72: 1185-1191, which is incorporated herein by reference in its entirety. TABLE 16 shows TAG profiles of a TAG oil produced by strains CHK74 and CHK80. Values are in percentages of total detected TAG species.

TABLE 16

| TAG Species | CHK74 | CHK80 |
|---|---|---|
| LOL | 1.54 | 1.26 |
| OOLn | 0.36 | 0.38 |
| LLP | 0.24 | 0.12 |
| OOL | 12.71 | 11.21 |
| POL | 1.67 | 1.28 |
| OOM | 0.66 | 0.45 |
| LOG | 0.14 | 0.14 |
| OOO | 60.73 | 63.19 |
| SOL | 0.90 | 0.50 |
| OOP | 10.59 | 9.15 |
| POP | 0.58 | 0.44 |
| OOG | 1.03 | 2.22 |
| OOS | 4.45 | 5.54 |
| POS | 0.34 | 0.28 |
| OOA | 0.42 | 0.45 |
| OOB | 0.07 | 0.06 |
| TOTAL | 96.43 | 96.67 |

### Embodiments

Embodiment A1. A non-naturally occurring oil comprising a triacylglyceride (TAG) component and ergosterol, wherein the TAG component has a fatty acid content comprising 80% or more C18:1 fatty acids.

Embodiment A2. The non-naturally occurring oil of embodiment A1, wherein the C18:1 fatty acids comprise oleic acid.

Embodiment A3. The non-naturally occurring oil of embodiment A1 or A2, wherein the C18:1 fatty acids comprise at least 90% of oleic acid.

Embodiment A4. The non-naturally occurring oil of any one of embodiments A1-A3, wherein the fatty acid content of the TAG component comprises more than 85% of C18:1 fatty acids.

Embodiment A5. The non-naturally occurring oil of any one of embodiments A1-A4, wherein the fatty acid content of the TAG component comprises one or more of C14:0, C16:0, C18:2, C18:3, C20:0, or C20:1 fatty acids.

Embodiment A6. The non-naturally occurring oil of any one of embodiments A1-A5, wherein the fatty acid content of the TAG component comprises C16:0 and C18:2 fatty acids.

Embodiment A7. The non-naturally occurring oil of any one of embodiments A1-A6, wherein the fatty acid content of the TAG component comprises 2% or more of C16:0 fatty acids.

Embodiment A8. The non-naturally occurring oil of any one of embodiments A1-A7, wherein the fatty acid content of the TAG component comprises more than 2% of C16:0 fatty acids.

Embodiment A9. The non-naturally occurring oil of any one of embodiments A1-A8, wherein the fatty acid content of the TAG component comprises 1.5% or more of C18:0 fatty acids.

Embodiment A10. The non-naturally occurring oil of any one of embodiments A1-A9, wherein the fatty acid content of the TAG component comprises 2% or more of C18:0 fatty acids.

Embodiment A11. The non-naturally occurring oil of any one of embodiments A1-A10, wherein the fatty acid content of the TAG component comprises less than 3% of C18:0 fatty acids.

Embodiment A12. The non-naturally occurring oil of any one of embodiments A1-All, wherein the fatty acid content of the TAG component comprises 2% or more of C18:2 fatty acids.

Embodiment A13. The non-naturally occurring oil of any one of embodiments A1-A12, wherein the fatty acid content of the TAG component comprises 5% or more of C18:2 fatty acids.

Embodiment A14. The non-naturally occurring oil of any one of embodiments A1-A13, wherein the fatty acid content of the TAG component comprises 6% or more of C18:2 fatty acids.

Embodiment A15. The non-naturally occurring oil of any one of embodiments A1-A14, wherein the fatty acid content of the TAG component comprises less than 1% of C20:1 fatty acids.

Embodiment A16. The non-naturally occurring oil of any one of embodiments A1-A15, wherein the non-naturally occurring oil comprises one or more of brassicasterol, campesterol, stigmasterol, β-sitosterol, delta-5-avenasterol, cycloartenol, or ergosterol.

Embodiment A17. The non-naturally occurring oil of any one of embodiments A1-A16, wherein the non-naturally occurring oil comprises brassicasterol, cycloartenol, and ergosterol.

Embodiment A18. The non-naturally occurring oil of any one of embodiments A1-A17, wherein the non-naturally occurring oil comprises more than 50 mg of ergosterol per 100 g of the non-naturally occurring oil.

Embodiment A19. The non-naturally occurring oil of any one of embodiments A1-A18, wherein the non-naturally occurring oil comprises more than 100 mg of ergosterol per 100 g of the non-naturally occurring oil.

Embodiment A20. The non-naturally occurring oil of any one of embodiments A1-A19, wherein the non-naturally occurring oil comprises more than 150 mg of ergosterol per 100 g of the non-naturally occurring oil.

Embodiment A21. The non-naturally occurring oil of any one of embodiments A1-A20, wherein the non-naturally occurring oil comprises less than 10 mg of cycloartenol per 100 g of the non-naturally occurring oil.

Embodiment A22. The non-naturally occurring oil of any one of embodiments A1-A21, wherein the non-naturally occurring oil comprises more than 5 mg of brassicasterol per 100 g of the non-naturally occurring oil.

Embodiment A23. The non-naturally occurring oil of any one of embodiments A1-A22, wherein the non-naturally occurring oil comprises more than 10 mg of brassicasterol per 100 g of the non-naturally occurring oil.

Embodiment A24. The non-naturally occurring oil of any one of embodiments A1-A23, wherein the non-naturally occurring oil comprises more than 10 mg of stigmasterol per 100 g of the non-naturally occurring oil.

Embodiment A25. The non-naturally occurring oil of any one of embodiments A1-A24, wherein the non-naturally occurring oil comprises less than 30 mg of β-sitosterol per 100 g of the non-naturally occurring oil.

Embodiment A26. A formulation comprising the non-naturally occurring oil of any one of embodiments A1-A25; and one or more excipients.

Embodiment A27. The formulation of embodiment A26, wherein the formulation is a polyol formulation.

Embodiment A28. The formulation of embodiment A26, wherein the formulation is a polyurethane formulation.

Embodiment A29. The formulation of embodiment A26, wherein the formulation is a polyurethane foam.

Embodiment A30. The formulation of embodiment A29, wherein the formulation is a polyurethane hard foam.

Embodiment A31. The formulation of embodiment A29, wherein the formulation is a polyurethane soft foam.

Embodiment A32. The formulation of embodiment A26, wherein the formulation is a cast polyurethane formulation.

Embodiment A33. The formulation of embodiment A26, wherein the formulation is a polyurethane dispersion.

Embodiment A34. The formulation of embodiment A26, wherein the formulation is a sporting goods equipment.

Embodiment A35. The formulation of embodiment A26, wherein the formulation is a personal care product.

Embodiment A36. The formulation of embodiment A35, wherein the personal care product is a cosmetic.

Embodiment A37. The formulation of embodiment A35, wherein the personal care product is a soap.

Embodiment A38. The formulation of embodiment A35, wherein the personal care product is an emollient.

Embodiment A39. The formulation of embodiment A35, wherein the personal care product is a hair care product.

Embodiment A40. The formulation of embodiment A35, wherein the personal care product is a skin care product.

Embodiment A41. The formulation of embodiment A26, wherein the formulation is a food product.

Embodiment A42. The formulation of embodiment A41, wherein the food product is a condiment.

Embodiment A43. The formulation of embodiment A41, wherein the food product is a nutritional supplement.

Embodiment A44. The formulation of embodiment A43, wherein the nutritional supplement is an infant formula.

Embodiment A45. The formulation of embodiment A41, wherein the food product is a food additive.

Embodiment A46. The formulation of embodiment A41, wherein the food additive is a flavoring agent.

Embodiment A47. The formulation of embodiment A41, wherein the food product is a beverage.

Embodiment A48. An oleaginous, non-naturally occurring microorganism that produces the non-naturally occurring oil of any one of embodiments A1-A25.

Embodiment A49. The oleaginous, non-naturally occurring microorganism of embodiment A48, wherein the microorganism is a microalgae.

Embodiment A50. The oleaginous, non-naturally occurring microorganism of embodiment A48 or A49, wherein the microalgae is of the genus Prototheca.

Embodiment A51. The oleaginous, non-naturally occurring microorganism of any one of embodiments A48-A50, wherein the microorganism does not comprise an exogenous gene.

Embodiment A52. The oleaginous, non-naturally occurring microorganism of any one of embodiments A48-A50, wherein the microorganism does not comprise an exogenous gene encoding an active ketoacyl-ACP synthase.

Embodiment A53. A bioreactor comprising the oleaginous, non-naturally occurring microorganism of any one of embodiments A48-A52.

Embodiment A54. A method for producing a non-naturally occurring oil, the method comprising: culturing in a bioreactor the oleaginous, non-naturally occurring microorganism of any one of embodiments A48-A52, thereby producing the non-naturally occurring oil.

Embodiment A55. The method of embodiment A54, wherein the non-naturally occurring oil is the non-naturally occurring oil of any one of embodiments A1-A25.

1-122. (canceled)

123. A method of producing a cell from a microbial base strain, the method comprising: obtaining the microbial base strain; and subjecting the microbial base strain to a classical strain improvement method to induce random or semi-random mutagenesis to produce the cell, wherein the cell produces a triacylglyceride (TAG) oil comprising 80% or more C18:1 fatty acids, wherein the cell does not comprise an exogenous gene.

124. The method of claim 123, further comprising exposing the microbial base strain to UV radiation.

125. The method of claim 123, further comprising exposing the microbial base strain to one or more chemical mutagens.

126. The method of claim 125, wherein the one or more chemical mutagens is selected from the group consisting of ICR-191, ethyl methanesulfonate (EMS), and 4-nitroquinoline-1-oxide (4-NQO).

127. The method of claim 123, further comprising exposing the microbial base strain to one or more enrichment agents.

128. The method of claim 127, wherein the one or more enrichment agents is selected from the group consisting of L-canavanine, cerulenin, triparanol, clomiphene, clomiphene citrate, clotrimazole, terfenadine, fluphenazine, AZD8055, BASF 13-338, cafenstrole, PF-042110, and phenethyl alcohol.

129. The method of claim 123, further comprising determining a total lipid titer of the cell.

130. The method of claim 123, further comprising determining a fatty acid profile of the TAG oil.

131. The method of claim 123, wherein the microbial base strain is of the genus Prototheca.

132. The method of claim 123, wherein the microbial base strain comprises a 23S ribosomal DNA sequence that has at least 80% identity to SEQ ID NO:30.

133. The method of claim 123, wherein the microbial base strain comprises a 23S ribosomal DNA sequence that has at least 80% identity to SEQ ID NO:31.

134. The method of claim 123, wherein the cell comprises a 23S ribosomal DNA sequence that has at least 80% identity to SEQ ID NO:30.

135. The method of claim 123, wherein the cell comprises a 23S ribosomal DNA sequence that has at least 80% identity to SEQ ID NO:31.

136. The method of claim 123, wherein the microbial base strain produces a TAG oil comprising less than 65% C18:1 fatty acids.

137. The method of claim 123, wherein the cell further comprises a DNA sequence that has at least 80% identity to any one of SEQ ID NOs: 1, 3, 5, 7, 9, 11, 13, 15, 18, 19, 21, 23, and 25.

138. A method of producing a non-genetically modified microalgal cell, wherein the cell produces a non-naturally occurring high oleic acid content oil, the method comprising:
a) subjecting microalgal cells of a microalgal base strain in a log phase of growth to mutagenesis to form mutagenized cells;
b) sub-culturing the mutagenized cells or derivatives thereof in a first lipid production medium to obtain sub-cultured mutagenized cells;
c) subjecting the sub-cultured mutagenized cells or derivatives thereof to at least one selection strategy to obtain cells meeting or surpassing a selected threshold for glucose consumption rate to obtain lead clones;
d) selecting the lead clones;
e) culturing the selected lead clones or derivatives thereof in a solid medium to obtain clonal isolates;
f) culturing the clonal isolates or derivatives thereof in a second lipid production medium;
g) identifying clonal isolates consuming glucose at a rate of at least about 6.0 grams/liter*day in the second lipid production medium to obtain high glucose consuming isolates;
h) culturing the high glucose consuming isolates or derivatives thereof in a third lipid production medium to obtain validated isolates;
i) sub-culturing the validated isolates or derivatives thereof to obtain stabilized validated isolates;
j) culturing the stabilized validated clonal isolates or derivatives thereof in a fourth lipid production medium to obtain phenotypically stable clones and phenotypically variable clones,
wherein the phenotypically stable clones are capable of producing the non-naturally occurring high oleic acid content oil, wherein the non-naturally occurring high oleic acid content oil comprises a triacylglyceride (TAG) component having a fatty acid content comprising at least 80% C18:1 fatty acids; and
k) performing fermentation validation and analysis on the phenotypically stable clones to produce the non-genetically modified microalgal cell.

139. The method of claim 138, wherein the mutagenesis is selected from the group consisting of exposure to UV radiation, exposure to a chemical mutagen, exposure to a selective or enrichment agent, and a combination thereof.

140. The method of claim 138, further comprising:
i) sub-culturing non-mutagenized cells of the microalgal base strain or derivatives thereof in a lipid production medium similar to the first lipid production medium to obtain sub-cultured non-mutagenized cells;
ii) subjecting the sub-cultured non-mutagenized cells or derivatives thereof to the at least one selection strategy to obtain cells meeting or surpassing an additional threshold for glucose consumption rate to obtain lead non-mutagenized clones;
iii) selecting the lead non-mutagenized clones;
iv) culturing the selected lead non-mutagenized clones or derivatives thereof in an additional solid medium similar to the solid medium to obtain non-mutagenized clonal isolates; and
v) culturing the non-mutagenized clonal isolates or derivatives thereof in a lipid production medium similar to the second lipid production medium to obtain non-mutagenized clonal isolates consuming glucose at a rate that exceeds that of the non-mutagenized cells to obtain high glucose consuming non-mutagenized isolates.

141. The method of claim 140, further comprising:
vi) subjecting the high glucose consuming non-mutagenized isolates or derivatives thereof to mutagenesis to form mutagenized control cells;
vii) sub-culturing the mutagenized control cells in a lipid production medium similar to the first lipid production medium to obtain sub-cultured mutagenized control cells;
viii) subjecting the sub-cultured mutagenized control cells or derivatives thereof to the at least one selection strategy to obtain cells meeting or surpassing a second additional threshold for glucose consumption rate to obtain lead mutagenized control clones;
ix) selecting the lead mutagenized control clones; and
x) culturing the selected lead mutagenized control clones or derivatives thereof in a second additional solid medium similar to the solid medium to obtain mutagenized control clonal isolates consuming glucose at a rate that exceeds that of the non-mutagenized clonal isolates.

142. The method of claim 138, wherein the microalgal base strain comprises a 23S ribosomal DNA sequence that has at least 80% sequence identity to SEQ ID NO:30.

143. The method of claim 138, wherein the microalgal base strain comprises a 23S ribosomal DNA sequence that has at least 80% sequence identity to SEQ ID NO:31.

144. The method of claim 138, wherein the microalgal base strain is of the genus Prototheca.

145. The method of claim 138, wherein the non-genetically modified microalgal cell comprises a DNA sequence that has at least 80% sequence identity to any one of SEQ ID NO: 1, 3, 5, 7, 9, 11, 13, 15, 18, 19, 21, 23, and 25.

146. The method of claim 138, wherein the TAG component further comprises at least 60% triolein (OOO).

147. The method of claim 138, wherein the at least one selection strategy in c) further comprises obtaining cells that produce an oil meeting or surpassing selected thresholds for a specified fatty acid profile to obtain the lead clones.

148. The method of claim 138, further comprising:
subjecting the phenotypically variable clones in j) through a)-j) to obtain additional phenotypically stable clones; and
performing fermentation validation and analysis on the additional phenotypically stable clones.

149. The method of claim 138, wherein the non-genetically modified microalgal cell does not comprise an exogenous gene.


SEQUENCE LISTING (31 sequences)

SEQ ID NO: 1 - DNA, 4218 bp, synthetic construct
  source 1..4218: mol_type = other DNA
cggcgcgctgcttcgcgtgccgggtgcagcaatcagatccaagtctgacgacttgcgcgcacgcgccggatccttcaattccaaagtgtcgtccgcgtgcgcttcttcgccttcgtcctcttgaacatccagcgacgcaagcgcagggcgctgggcggctggcgtcccgaaccggcctcggcgcacgcggctgaaattgccgatgtcggcaatgtagtgccgctccgcccacctctcaattaagtttttcagcgcgtggttgggaatgatctgcgctcatggggcgaaagaaggggttcagaggtgctttattgttactcgactgggcgtaccagcattcgtgcatgactgattatacatacaaaagtacagctcgcttcaatgccctgcgattcctactcccgagcgagcactcctctcaccgtcgggttgcttcccacgaccacgccggtaagagggtctgtggcctcgcgcccctcgcgagcgcatctttccagccacgtctgtatgattttgcgctcatacgtctggcccgtcgaccccaaaatgacgggatcctgcataatatcgcccgaaatgggatccaggcattcgtcaggaggcgtcagccccgcgggagatgccggtcccgccgcattggaaaggtgtagagggggtgaatcccccatttcatgaaatgcttggtcaacgatggtgcgcattcgtgcaaagtgaatatggggtcacgcggtggacgaacgcggagggggcatgaccgaatctaggctcgcattcctcagatcacttcatgccggcggtccggggtttgcgcgtcgcgcaaggctacgtctccctagccgctgcgcaccacgcgtgcgacgcggaggccatcttccggagcaacgaccatggattgtcttagcgatcgcacgaatgagtgctagtgagtcgtacgctcgacccagtcgctcgcaggagaaggcggcagctgccgagcttcggcttaccagtcgtgactcgtatgtgatcaggaatcattggcattggtagcattataattcggcttccgcgctgcgtatgggcatggcaatgtctcatgcagtcgatcttagtcaaccaattttgggtggccaggtccgggcgaccgggctccgtgtcgccgggcaccacctcctgccaggagtagcagggccgccctctcgtcccgacgttggcccactgaataccgtggcttcgagccctacatgatgggctgcctagtcgggcgggacgcgcaactgcccgcgcgatctgggggctggtctgaatccttcaggcgggtgttacccgagaaagaaagggtgccgatttcaaagcagacccatgtgccgggccctgtggcctgtgttggcgcctatgtagtcaccccccctcacccaattgtcgccagtttgcgcactccataaactcaaaacagcagcttctgagctgcgctgttcaagaacacctctggggtttgctcacccgcgaggtcgacgcccagcatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccgccgaccctccccccaccttccagggcgccttcggcacgggtgtctgggtgtgcgcgcacgagtgcggtcaccaggccttttcctccagccaggccatcaacgacggcgtgggcctggtgttccacagcctgctgctggtgccctactactcctggaagcactcgcaccgccgccaccactccaacacggggtgcctggacaaggacgaggtgtttgtgccgccgcaccgtgcggtggcgcacgagggcctggagtgggaggagtggctgcccatccgcatgggcaaggtgctggtcaccttgaccctgggctggccgctgtacctcatgttcaacgtcgcctcccgcccttacccgcgcttcgccaaccactttgacccgtggtcgcccatcttcagcaagcgcgagcgcatcgaggtggtcatctccgacctcgcgttggtggcggtgctcagcgggctcagcgtgctgggccgcaccatgggctgggcctggctggtcaagacctacgtggtgccctacatgatcgtgaacatgtggctggtgctcatcacgctgctccagcacacgcacccggccctgccgcactacttcgagaaggactgggactggctacgcggcgccatggccaccgtcgaccgctccatgggcccgcccttcatggacagcatcctgcaccacatctccgacacccacgtgctgcaccacctcttcagcaccatcccgcactaccacgccgaggaggcctccgccgccatccggcccatcctgggcaagtactaccaatccgacagccgctgggtcggccgcgccctgtgggaggactggcgcgactgccgctacgtcgtccccgacgcgcccgaggacgactccgcgctctggttccacaagtgagcgcgcctgcgcgaggacgcagaacaacgctgccgccgtgtcttttgcacgcgcgactccggcgcttcgctggtggcacccccataaagaaaccctcaattctgtttgtggaagacacggtgtacccccacccacccacctgcacctctattattggtattattgacgcgggagtgggcgttgtaccctacaacgtagcttctctagttttcagctggctcccaccattgtaaattcatgctagaatagtgcgtggttatgtgagaggtatagtgtgtctgagcagacggggcgggatgcatgtcgtggtggtgatctttggctcaaggcgtcgtcgacgtgacgtgcccgatcatgagagcaataccgcgctcaaagccgacgcatagcctttactccgcaatccaaacgactgtcgctcgtattttttggatatctattttaaagagcgagcacagcgccgggcatgggcctgaaaggcctcgcggccgtgctcgtggtgggggccgcgagcgcgtggggcatcgcggcagtgcaccaggcgcagacggaggaacgcatggtgcgtgcgcaatataagatacatgtattgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctatccacatgtgacaaaggggtctgggtcgtacgaatcgacaaaccagtcaggagacagcggggtccatgagttggcccgctcgcagcttcagcgctttgagcatcgcggcattgtccacgatccaaggctcccgcagcgcctccttgggacccctgtgcggagaagcgtgtgccgagagggctgtgagggcggaggctggaggagaggctgcgaatcattgcgctgcctaggcgtctgccgagtagtagcacaccacgcgccggtgctttcatttcattgtgtccgttgctgtaatgcatgcgagcaatcctgcactaaaagctgcgatcaccacctcggcgttgttggtctctagtaccgctcacctgaccacgcgcacggaagtatggcggtcggcgacggcctgctgcacgtggcagtgcagatccacgtggcccaggagcaggtctcgcagctgcaacaccgcgccctcgtgctcgccggggtccatccagtccataccccgcgccgggccctcgagccggacggccagcagccagggccgggggtccgcgccgacgcccggcggccccgccgcgcagctggggtcggccaggaagagccactggacggccggcccgggcgagccccgcgccggcgtgggacccacccgcagcaccaccgcctgctccaggtccacgtcgctcggcggcaggcccgcgtgcggcgcctgcgccagcgtccagggcaggcggggccgggcgtgcatg

SEQ ID NO: 2 - DNA, 4217 bp, unidentified
  source 1..4217: mol_type = unassigned DNA; note = Prototheca wickerhamii
cggcgcgctgcttcgcgtgccgggtgcagcaatcagatccaagtctgacgacttgcgcgcacgcgccggatccttcaattccaaagtgtcgtccgcgtgcgcttcttcgccttcgtcctcttgaacatccagcgacgcaagcgcagggcgctgggcggctggcgtcccgaaccggcctcggcgcacgcggctgaaattgccgatgtcggcaatgtagtgccgctccgcccacctctcaattaagtttttcagcgcgtggttgggaatgatctgcgctcatggggcgaaagaaggggttcagaggtgctttattgttactcgactgggcgtaccagcattcgtgcatgactgattatacatacaaaagtacagctcgcttcaatgccctgcgattcctactcccgagcgagcactcctctcaccgtcgggttgcttcccacgaccacgccggtaagagggtctgtggcctcgcgcccctcgcgagcgcatctttccagccacgtctgtatgattttgcgctcatacgtctggcccgtcgaccccaaaatgacgggatcctgcataatatcgcccgaaatgggatccaggcattcgtcaggaggcgtcagccccgcgggagatgccggtcccgccgcattggaaaggtgtagagggggtgaatcccccatttcatgaaatgcttggtcaacgatggtgcgcattcgtgcaaagtgaatatggggtcacgcggtggacgaacgcggagggggcatgaccgaatctaggctcgcattcctcagatcacttcatgccggcggtccggggtttgcgcgtcgcgcaaggctacgtctccctagccgctgcgcaccacgcgtgcgacgcggaggccatcttccggagcaacgaccatggattgtcttagcgatcgcacgaatgagtgctagtgagtcgtacgctcgacccagtcgctcgcaggagaaggcggcagctgccgagcttcggcttaccagtcgtgactcgtatgtgatcaggaatcattggcattggtagcattataattcggcttccgcgctgcgtatgggcatggcaatgtctcatgcagtcgatcttagtcaaccaattttgggtggccaggtccgggcgaccgggctccgtgtcgccgggcaccacctcctgccaggagtagcagggccgccctctcgtcccgacgttggcccactgaataccgtggcttcgagccctacatgatgggctgcctagtcgggcgggacgcgcaactgcccgcgcgatctgggggctggtctgaatccttcaggcgggtgttacccgagaaagaaagggtgccgatttcaaagcagacccatgtgccgggccctgtggcctgtgttggcgcctatgtagtcaccccccctcacccaattgtcgccagtttgcgcactccataaactcaaaacagcagcttctgagctgcgctgttcaagaacacctctggggtttgctcacccgcgaggtcgacgcccagcatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccgccgaccctcccccaccttccagggcgccttcggcacgggtgtctgggtgtgcgcgcacgagtgcggtcaccaggccttttcctccagccaggccatcaacgacggcgtgggcctggtgttccacagcctgctgctggtgccctactactcctggaagcactcgcaccgccgccaccactccaacacggggtgcctggacaaggacgaggtgtttgtgccgccgcaccgtgcggtggcgcacgagggcctggagtgggaggagtggctgcccatccgcatgggcaaggtgctggtcaccttgaccctgggctggccgctgtacctcatgttcaacgtcgcctcccgcccttacccgcgcttcgccaaccactttgacccgtggtcgcccatcttcagcaagcgcgagcgcatcgaggtggtcatctccgacctcgcgttggtggcggtgctcagcgggctcagcgtgctgggccgcaccatgggctgggcctggctggtcaagacctacgtggtgccctacatgatcgtgaacatgtggctggtgctcatcacgctgctccagcacacgcacccggccctgccgcactacttcgagaaggactgggactggctacgcggcgccatggccaccgtcgaccgctccatgggcccgcccttcatggacagcatcctgcaccacatctccgacacccacgtgctgcaccacctcttcagcaccatcccgcactaccacgccgaggaggcctccgccgccatccggcccatcctgggcaagtactaccaatccgacagccgctgggtcggccgcgccctgtgggaggactggcgcgactgccgctacgtcgtccccgacgcgcccgaggacgactccgcgctctggttccacaagtgagcgcgcctgcgcgaggacgcagaacaacgctgccgccgtgtcttttgcacgcgcgactccggcgcttcgctggtggcacccccataaagaaaccctcaattctgtttgtggaagacacggtgtacccccacccacccacctgcacctctattattggtattattgacgcgggagtgggcgttgtaccctacaacgtagcttctctagttttcagctggctcccaccattgtaaattcatgctagaatagtgcgtggttatgtgagaggtatagtgtgtctgagcagacggggcgggatgcatgtcgtggtggtgatctttggctcaaggcgtcgtcgacgtgacgtgcccgatcatgagagcaataccgcgctcaaagccgacgcatagcctttactccgcaatccaaacgactgtcgctcgtattttttggatatctattttaaagagcgagcacagcgccgggcatgggcctgaaaggcctcgcggccgtgctcgtggtgggggccgcgagcgcgtggggcatcgcggcagtgcaccaggcgcagacggaggaacgcatggtgcgtgcgcaatataagatacatgtattgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctatccacatgtgacaaaggggtctgggtcgtacgaatcgacaaaccagtcaggagacagcggggtccatgagttggcccgctcgcagcttcagcgctttgagcatcgcggcattgtccacgatccaaggctcccgcagcgcctccttgggacccctgtgcggagaagcgtgtgccgagagggctgtgagggcggaggctggaggagaggctgcgaatcattgcgctgcctaggcgtctgccgagtagtagcacaccacgcgccggtgctttcatttcattgtgtccgttgctgtaatgcatgcgagcaatcctgcactaaaagctgcgatcaccacctcggcgttgttggtctctagtaccgctcacctgaccacgcgcacggaagtatggcggtcggcgacggcctgctgcacgtggcagtgcagatccacgtggcccaggagcaggtctcgcagctgcaacaccgcgccctcgtgctcgccggggtccatccagtccataccccgcgccgggccctcgagccggacggccagcagccagggccgggggtccgcgccgacgcccggcggccccgccgcgcagctggggtcggccaggaagagccactggacggccggcccgggcgagccccgcgccggcgtgggacccacccgcagcaccaccgcctgctccaggtccacgtcgctcggcggcaggcccgcgtgcggcgcctgcgccagcgtccagggcaggcggggccgggcgtgcatg

SEQ ID NO: 3 - DNA, 4227 bp, synthetic construct
  source 1..4227: mol_type = other DNA
cggctcgctgctttgcgtgccgggtgcagcgatcagatccaagtctgacgacttgtgctgatgtactgtgtcctttgagtccagagcgccggccgcacgcgcttcttccccttcttcttcctctcgaacatccagcgatgcaagtgcagggcgctgggcggctggcgtcccgaaccggcctcggcgcacgcggctgaaattgccaatgtcggcaatgtagtgccgctcggcccatccctcgatcaagtttttcagcgcgtggttggggatgatctgcgctcatgggaagataaaaggggttctgaggtggttgattggtactttaccggaagtactcatattcatacatgactgatcccacacaaaaacaaagctcacttcaaagaaccgcgcatgtctactccccagcaatcacttcgctcaccgtcgggttgcttcccacgacaacgccggtgagagggtcggtggcctcgcgaccttcgcgggcacatctttccagccatgtctgtataatctcacgctcatacgtctggcccgtcgaccccaaaatgacgggatcctgcatgatatcgcccgagatggggtccaggcattcctctggaggcgtcagccctgcgggagatgccggtcccaccgcattggaaaggcacaaagggggtgaatcccccatttcatgaaattgttggtcagcgatggtgcgcactcgtgcgcaatgaatatggggtcacgcggtggacgaacgcggagggggcctggccgaatctaggcttgcattcctcagatcactttctgccggcggtccggggtttgcgcgtcgcgcaacgctccgtctccctagccgctgcgcaccgcgcgtgcgacgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggcccactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacgcccagcatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcgccggtgcctacgtgggtcaagtatggcgtcatgtggccgctctactggttcttccaggtgtgtgtgagggttgtggttgcccgtatcgaggtcctggtggcgcgcatgggggagaaggcgcctgtcccgctgacccccccggctaccctcccggcaccttccagggcgccttcggcacgggtgtctgggtgtgcgcgcacgagtgcggccaccaggccttttcctccagccaggccatcaacgacggcgtgggcctggtgttccacagcctgctgctggtgccctactactcctggaagcactcgcaccgccgccaccactccaacacggggtgcctggacaaggacgaggtgtttgtgccgccgcaccgcgcagtggcgcacgagggcctggagtgggaggagtggctgcccatccgcatgggcaaggtgctggtcaccctgaccctgggctggccgctgtacctcatgttcaacgtcgcctcgcggccgtacccgcgcttcgccaaccactttgacccgtggtcgcccatcttcagcaagcgcgagcgcatcgaggtggtcatctccgacctggcgctggtggcggtgctcagcgggctcagcgtgctgggccgcaccatgggctgggcctggctggtcaagacctacgtggtgccctacctgatcgtgaacatgtggctcgtgctcatcacgctgctccagcacacgcacccggcgctgccgcactacttcgagaaggactgggactggctgcgcggcgccatggccaccgtggaccgctccatgggcccgcccttcatggacaacatcctgcaccacatctccgacacccacgtgctgcaccacctcttcagcaccatcccgcactaccacgccgaggaggcctccgccgccatcaggcccatcctgggcaagtactaccagtccgacagccgctgggtcggccgcgccctgtgggaggactggcgcgactgccgctacgtcgtcccggacgcgcccgaggacgactccgcgctctggttccacaagtgagtgagtgagtcgctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgacaaaggggtctgggtcgtacgacaaaccagtcaggaggcggcggggtccatgagctggcccgctcgcagcttcagcgcttcgagcatcgcggcgttgtccgcgatccaagcctcccgcagcgcctccttggggcccctgtgcggagaggcgggtgccgagagggctgtgagggcggaggctggaggacaggctgcgaatcgtcgcgctgcctaggcgtctgccgaggagaagcacaccacgcgccgccgctttcatttcattgtgcccgttgctgtaatgcatgcgagcgatcctgcactaaagctgcgatcgccacctcagcgttgttcgtctctagcaccgctcacctgaccacgcgcacggaagagtggcggtcggcgacggcctgctgcacgtggcagtgcaggtccacgtggccgaggagcaggtctcgcagctgcagcaccgcgccctcgtgctcgccggggtccatccagtccatgccccgcgccgggccctcgagccggacggccagcagccagggccgggggtccgcgccgacgcccggcggccccgccgcgcagctggggtcggccaggaagagccactgggcggccggcccgggcgagccccgcgccggcgcgggacccacccgcagcaccaccgcctggtccaggtcgacgtcgctcggcggcaggcccgcgtgcggcgcctgcgccagcgtccagggcaggcgggggcgcgcgtgca

SEQ ID NO: 4 - DNA, 4227 bp, unidentified; identical to SEQ ID NO: 3
  source 1..4227: mol_type = unassigned DNA; note = Prototheca wickerhamii

SEQ ID NO: 5 - DNA, 4067 bp, synthetic construct
  source 1..4067: mol_type = other DNA
gcaatggcgctcggtacagggtctgcgtccgtgctgggctccctctcctacgatgcacaagggagcgccccggccagctcagcgcgtccacaacctcccctcgtcacacacacacctgcggaaccaggccgcccatttgctgcttgagcatgccttgcatcatgtccgggtttcccatcatatcgttgaggttcttgggctccagcttctgctccagcacaccatcctgtcgatcgaagagaaggagacatgtgtacattattggtgtgagggcgctgaatcggccattttttaaatgatcacgctcatgccaatagacgcggcacataacgacgttcaaacccccgccaaagccgcggacaaccccatccctccacaccccccacacaaagaacccgccaccgcttaccttgcccacgaggtaggcctttcgttgcgcaaaaccggcctcggtgatgaatgcatgcccgttcctgacgagcgctgcccgggccaacacgctcttttgctgcgtctcctcaggcttgggggcctccttgggcttgggtgccgccatgatctgcgcgcatcagagaaacgttgctggtaaaaggagcgcccggctgcgcaatatatatataggcatgccaacacagcccaacctcactcgggagcccgtcccaccacccccaagtcgcgtgccttgacggcatactgctgcagaagcttcatgagaatgatgccgaacaagaggggcacgaggacccaatcccggacatccttgtcgataatgatctcgtgagtccccatcgtccgcccgacgctccggggagcccgccgatgctcaagacgagagggccctcgaccaggaggggctggcccgggcgggcactggcgtcgaaggtgcgcccgtcgttcgcctgcagtcctatgccacaaaacaagtcttctgacggggtgcgtttgctcccgtgcgggcaggcaacagaggtattcaccctggtcatggggagatcggcgatcgagctgggataagagatacggtcccgcgcaaggatcgctcatcctggtctgagccggacagtcattctggcaagcaatgacaacttgtcaggaccggaccgtgccatatatttctcacctagcgccgcaaaacctaacaatttgggagtcactgtgccactgagttcgactggtagctgaatggagtcgctgctccactaaacgaattgtcagcaccgccagccggccgaggacccgagtcatagcgagggtagtagcgcgccatggcaccgaccagcctgcttgccagtactggcgtctcttccgcttctctgtggtcctctgcgcgctccagcgcgtgcgcttttccggtggatcatgcggtccgtggcgcaccgcagcggccgctgcccatgcagcgccgctgcttccgaacagtggcggtcagggccgcacccgcggtagccgtccgtccggaacccgcccaagagttttgggagcagcttgagctctgcaagatggcggaggacaagcgcatcttcctggaggagcaccggtgcgtggaggtccggggctgaccggccgtcgcattcaacgtaatcaatcgcatgatgatcagaggacacgaagtcttggtggcggtggccagaaacactgtccattgcaagggcatagggatgcgttccttcacctctcatttctcatttctgaatccctccctgctcactctttctcctcctccttcccgttcacgcagcattcggggcaacgaggtgggcccctcgcagcggctgacgatcacggcggtggccaacatcctgcaggaggcggcgggcaaccacgcggtggccatgtggggccggagctcggagggtttcgcgacggacccggagctgcaggaggcgggtctcatctttgtgatgacgcgcatgcagatccagatgtaccgctacccgcgctggggcgacctgatgcaggtggagacctggttccagacggcgggcaagctgggcgcgcagcgcgagtgggtgctgcgcgacaagctgaccggcgaggcgctgggcgcggccacctcgagctgggtcatgatcaacatccgcacgcgccggccgtgccgcatgccggagctcgtccgcgtcaagtcggccttcttcgcgcgcgagccgccgcgcctggcgctgccgcccgcggtcacgcgtgccaagctgcccaacatcgcgacgccggcgccgctgcgcgggcaccgccaggtcgcgcgccgcaccgacatggacatgaacgggcacgtgaacaacgtggcctacctggcctggtgcctggaggccgtgcccgagcacgtcttcagcgactaccacctctaccagatggagatcgacttcaaggccgagtgccacgcgggcgacgtcatctcctcccaggccgagcagatcccgccccaggaggcgctcacgcacaacggcgccggccgcaacccctcctgcttcgtccatagcattctgcgcgccgagaccgagctcgtccgcgcgcgaaccacatggtcggcccccatcgacgcgcccgccgccaagccgcccaaggcgagccactgaggacagggtggttggctggatggggaaacgctggtcgcgggattcgatcctgctgcttatatcctccctggaagcacacccacgactctgaagaagaaaacgtgcacacacacaacccaaccggccgaatatttgcttccttatcccgggtccaagagagactgcgatgcccccctcaatcagcatcctcctccctgccgcttcaatcttccctgcttgcctgcgcccgcggtgcgccgtctgcccgcccagtcagtcactcctgcacaggccccttgtgcgcagtgctcctgtaccctttaccgctccttccattctgcgaggccccctattgaatgtattcgttgcctgtgtggccaagcgggctgctgggcgcgccgccgtcgggcagtgctcggcgactttggcggaagccgattgttcttctgtaagccacgcgcttgctgctttgggaagagaaggggggggtactgaatggatgaggaggagaaggaggggtattggtattatctgagttggggaggcagggagagttggaaaatgtaagtggcacgacgggcaaggagaatggtgagcatgtgcatggtgatgtcgttggtcgaggacgatcctgcacgcgtgtatctgatgtagaatacggcaatcaccctagtctacatctataccttctccgtataacgccctttccaaatgccctcccgtttctctcctattcttgatccacatgatgaccctggcactatttcaagggctggacatttcaagaaggtttgcgtatctgaagaaggattggtttggagaggtggccgatgaaagtggggtcaagctgcgtggagccctctgcacggatttctatggtaatctgcgtccacgtcatcagtagccgtacgcctctgcggcgtcgccgtgctcctcagccgccgcttcaccaccagccaggagccccatacgcacgcgctcccagacggcgcgcgcgacggggacgatcatgagcagcgcgatctggggcgtgtcgtgcgagctgagccgcacaaccacgttgccctggccgcgcgtggtgtactggcagttggtcgagaccatggtgtcggacttgaagtaggagccgtcaaagcgcgggaggcgcgtctcggccgagacgttgccgccgatgaccaggctctggcgcttctggcgcgggtcgcggccctgccactgcgcggagccgcccaggagcaggcgcgactcgtcacggccgcgcagcttgacgtcggccgccagcgccgtgcccggctcgtaggccgaccccatcttgacgaagaggcggccgatcgctgcgcggaccttgaccgcgtcggacaggcgcaggcgctcgtccagcttgaccccgagcgccgcgttgccgggcttgaacggcagcgagaactcctcgcccagcttggagccgagcaggcccagcgccagcttctgccgcttgccgcgcgcgcgcaggcgcgagtcgacgcgcagcgtgtagag

SEQ ID NO: 6 - DNA, 4069 bp, unidentified
  source 1..4069: mol_type = unassigned DNA; note = Prototheca wickerhamii
gcaatggcgctcggtacagggtctgcgtccgtgctgggctccctctcctacgatgcacaagggagcgccccggccagctcagcgcgtccacaacctcccctcgtcacacacacacctgcggaaccaggccgcccatttgctgcttgagcatgccttgcatcatgtccgggtttcccatcatatcgttgaggttcttgggctccagcttctgctccagcacaccatcctgtcgatcgaagagaaggagacatgtgtacattattggtgtgagggcgctgaatcggccattttttaaaatgatcacgctcatgccaatagacgcggcacataacgacgttcaaacccccgccaaagccgcggacaaccccatccctccacaccccccacacaaagaacccgccaccgcttaccttgcccacgaggtaggcctttcgttgcgcaaaaccggcctcggtgatgaatgcatgcccgttcctgacgagcgctgcccgggccaacacgctcttttgctgcgtctcctcaggcttgggggcctccttgggcttgggtgccgccatgatctgcgcgcatcagagaaacgttgctggtaaaaaggagcgcccggctgcgcaatatatatataggcatgccaacacagcccaacctcactcgggagcccgtcccaccacccccaagtcgcgtgccttgacggcatactgctgcagaagcttcatgagaatgatgccgaacaagaggggcacgaggacccaatcccggacatccttgtcgataatgatctcgtgagtccccatcgtccgcccgacgctccggggagcccgccgatgctcaagacgagagggccctcgaccaggaggggctggcccgggcgggcactggcgtcgaaggtgcgcccgtcgttcgcctgcagtcctatgccacaaaacaagtcttctgacggggtgcgtttgctcccgtgcgggcaggcaacagaggtattcaccctggtcatggggagatcggcgatcgagctgggataagagatacggtcccgcgcaaggatcgctcatcctggtctgagccggacagtcattctggcaagcaatgacaacttgtcaggaccggaccgtgccatatatttctcacctagcgccgcaaaacctaacaatttgggagtcactgtgccactgagttcgactggtagctgaatggagtcgctgctccactaaacgaattgtcagcaccgccagccggccgaggacccgagtcatagcgagggtagtagcgcgccatggcaccgaccagcctgcttgccagtactggcgtctcttccgcttctctgtggtcctctgcgcgctccagcgcgtgcgcttttccggtggatcatgcggtccgtggcgcaccgcagcggccgctgcccatgcagcgccgctgcttccgaacagtggcggtcagggccgcacccgcggtagccgtccgtccggaacccgcccaagagttttgggagcagcttgagccctgcaagatggcggaggacaagcgcatcttcctggaggagcaccggtgcgtggaggtccggggctgaccggccgtcgcattcaacgtaatcaatcgcatgatgatcagaggacacgaagtcttggtggcggtggccagaaacactgtccattgcaagggcatagggatgcgttccttcacctctcatttctcatttctgaatccctccctgctcactctttctcctcctccttcccgttcacgcagcattcggggcaacgaggtgggcccctcgcagcggctgacgatcacggcggtggccaacatcctgcaggaggcggcgggcaaccacgcggtggccatgtggggccggagctcggagggtttcgcgacggacccggagctgcaggaggcgggtctcatctttgtgatgacgcgcatgcagatccagatgtaccgctacccgcgctggggcgacctgatgcaggtggagacctggttccagacggcgggcaagctgggcgcgcagcgcgagtgggtgctgcgcgacaagctgaccggcgaggcgctgggcgcggccacctcgagctgggtcatgatcaacatccgcacgcgccggccgtgccgcatgccggagctcgtccgcgtcaagtcggccttcttcgcgcgcgagccgccgcgcctggcgctgccgcccgcggtcacgcgtgccaagctgcccaacatcgcgacgccggcgccgctgcgcgggcaccgccaggtcgcgcgccgcaccgacatggacatgaacgggcacgtgaacaacgtggcctacctggcctggtgcctggaggccgtgcccgagcacgtcttcagcgactaccacctctaccagatggagatcgacttcaaggccgagtgccacgcgggcgacgtcatctcctcccaggccgagcagatcccgccccaggaggcgctcacgcacaacggcgccggccgcaacccctcctgcttcgtccatagcattctgcgcgccgagaccgagctcgtccgcgcgcgaaccacatggtcggcccccatcgacgcgcccgccgccaagccgcccaaggcgagccactgaggacagggtggttggctggatggggaaacgctggtcgcgggattcgatcctgctgcttatatcctccctggaagcacacccacgactctgaagaagaaaacgtgcacacacacaacccaaccggccgaatatttgcttccttatcccgggtccaagagagactgcgatgcccccctcaatcagcatcctcctccctgccgcttcaatcttccctgcttgcctgcgcccgcggtgcgccgtctgcccgcccagtcagtcactcctgcacaggccccttgtgcgcagtgctcctgtaccctttaccgctccttccattctgcgaggccccctattgaatgtattcgttgcctgtgtggccaagcgggctgctgggcgcgccgccgtcgggcagtgctcggcgactttggcggaagccgattgttcttctgtaagccacgcgcttgctgctttgggaagagaaggggggggtactgaatggatgaggaggagaaggaggggtattggtattatctgagttggggaggcagggagagttggaaaatgtaagtggcacgacgggcaaggagaatggtgagcatgtgcatggtgatgtcgttggtcgaggacgatcctgcacgcgtgtatctgatgtagaatacggcaatcaccctagtctacatctataccttctccgtataacgccctttccaaatgccctcccgtttctctcctattcttgatccacatgatgaccctggcactatttcaagggctggacatttcaagaaggtttgcgtatctgaagaaggattggtttggagaggtggccgatgaaagtggggtcaagctgcgtggagccctctgcacggatttctatggtaatctgcgtccacgtcatcagtagccgtacgcctctgcggcgtcgccgtgctcctcagccgccgcttcaccaccagccaggagccccatacgcacgcgctcccagacggcgcgcgcgacggggacgatcatgagcagcgcgatctggggcgtgtcgtgcgagctgagccgcacaaccacgttgccctggccgcgcgtggtgtactggcagttggtcgagaccatggtgtcggacttgaagtaggagccgtcaaagcgcgggaggcgcgtctcggccgagacgttgccgccgatgaccaggctctggcgcttctggcgcgggtcgcggccctgccactgcgcggagccgcccaggagcaggcgcgactcgtcacggccgcgcagcttgacgtcggccgccagcgccgtgcccggctcgtaggccgaccccatcttgacgaagaggcggccgatcgctgcgcggaccttgaccgcgtcggacaggcgcaggcgctcgtccagcttgaccccgagcgccgcgttgccgggcttgaacggcagcgagaactcctcgcccagcttggagccgagcaggcccagcgccagcttctgccgcttgccgcgcgcgcgcaggcgcgagtcgacgcgcagcgtgtagag

SEQ ID NO: 7 - DNA, 4083 bp, synthetic construct
  source 1..4083: mol_type = other DNA
gcaatggcgctctgtgcagggtctgtgtccggtctccctctcctacgatgcacaaggcaacgcctttgctgactcagcgcgtccacaacctcccctcgtcacacacacctgcggaaccaggccgcccatttgctgcttgagcatgccttgcatcatgtccgggttgcccatcatatcgttgaggttcttgggctccagtttctgctccagcacgccatcctgtcggtcgaagaggaggagacatgtaagcgttgttgggatgagggttgctaaattggccattatttttaatgatcacgctcgtgccaatagacgcggcacattgcgatgttcaaactcccgtcaaagccgcggacaaccacatccctccacaccccacacacaaagaacccgccaccgctcaccttgcccacgaggtaggcctttcgttgcgcaaacccggcctcggtgatgaatgcgtgcccgttcctgacgagcgctgcccgggccaacacgctcttttgctgcgtctcctcaggcttgggggcctccttgggcttgggggccgccatgatctgcgcgcaccagagagaagtcattggtgaaaaggagcgcccggctgcgctatatatatatatatatgctcaggccaataaagtccaacctcactggggagccccgtcccaccacccccaagtcgcgtaccttgacggcatactgctgcagaagcttcatgaggatgatgccgaacaagaggggcacaaggacccaatcccggacatccttgtcgataatgatctcgtgagtccccatcgtccgcccgacgctgcggggagcccgccgatgctcaagacgagagggccctcgaccaggaggggctggcccgggcgggcactggcgtcgaaggtgcgcccgtcgttcgcctgcagtccgatgacacaaaacaagtcttctgacggggtgcgtttgctcccgtgcgggcaggcaacagaggtattcaacctggtcatggcgagatcggcgatcgagctgggataagagatacggtcccgcgcgaggatcgctcatcctggtctgagccggacagtcattctggcaagcaatgacaacttgccaggaccggaccgtgccatatatttctcacctagcgcggcaaaacctaacaatttggaagtcactgtgccactgagctcgactggtagctgaatggagtcgctgctccactaatcgaattgtcagcaccgccagccggccgaggacccgagtcatagcgagggtagtagcgcgccatggcaccgaccagcctgcttgcccgtactggcgtctcttccgcttctctgtgctcctctacgcgctccggcgcgtgcgcttttccggtggatcatgcggtccgtggcgcaccgcagcggccgctgcccatgcagcgccgctgcttccgaacagtggctgtcagggccgcacccgcagtagccgtccgtccggaacccgcccaagagttttgggagcagcttgagccctgcaagatggcggaggacaagcgcatcttcctgtaggagcaccggtgcgcggaggtccggggctgaccggccgtcgcattcaacgtaatcaatcgcatgatgatcacaggacgcgacgtcttggtggcggtggccagggacactgcccattgcacaggcataggaatgcgttccttctcatttctcagttttctgagcccctccctcttcactctttctcctcctcctcccctctcacgcagcattcgtggcaacgaggtgggcccctcgcagcggctgacgatcacggcggtggccaacatcctgcaggaggcggcgggcaaccacgcggtggccatgtggggtcggagctcggagggtttcgcgacggacccggagctgcaggaggcgggcctcatctttgtgatgacgcgcatgcagatccaaatgtaccgctacccgcgctggggcgacctgatgcaggtggagacctggttccagacggcgggcaagctaggcgcgcagcgcgagtgggtgctgcgcgacaagctgaccggcgaggcgctgggcgcggccacctccagctgggtcatgatcaacatccgcacgcgccggccgtgccgcatgcccgagctcgtccgcgtcaagtcggccttcttcgcgcgcgagccgccgcgcctggcgctgccgcccacggtcacgcgcgccaagctgcccaacatcgcgacgccggcgccgctgcgcgggcaccgccaggtcgcgcgccgcaccgacatggacatgaacggccacgtgaacaacgttgcctacctggcctggtgcctggaggccgtgcccgagcacgtcttcagcgactaccacctctaccagatggagatcgacttcaaggccgagtgccacgcgggcgacgtcatctcctcccaggccgagcagatcccgccccaggaggcgctcacgcacaacggcgccggccgcaacccctcctgcttcgtccacagcattctgcgcgccgagaccgagctcgtccgcgcgcgcaccacctggtcggcccccgtcgacgcgcccgccgccaagccgcccaaggcgagccactgagcagaggagagggtggctggtagttgcgggatggctggtcgcccgtcgatcctgctgctgctattgtctcctcctgcacaagcccacccacgactccgaagaagaagaagaaaacgcgcacacacacaacccaaccggccgaatatttgcttccttatcccgggtccaagagagacggcgatgcccccctcaatcagcctcctcctccctgccgctccaatcttccctgcttgcatgcgcccgcgagaggctgtctgcgcgccccgtcagtcactccccgtgcagacgcctcgtgctcggtgctcctgtatcctttaccgctcctttcattctgcgaggccccctgttgaatgtattcgttgcctgtgtggccaagcgcgctgctgggcgcgccgccgtcgggcggtgctcggcgactctggcggaagccggttgttcttctgtaagccacgcgcttgctgcttttggaaaagaggggggtttactgaatggaggaggagcaggataattggtagtatctgagttgttggggaggcaggaagagttgggaaatggaagtggcacgatgggcaaggagaatggtgagcatgcgcagggtgatgtcgttggccgagaacgatcctgcactcccgtaagtagaatacggcaatcacgttgatctacatctctaacttctccatttaaagccctttccacatgccctcgcgtttgcatcctacaccctttgatccacatgattgatgaccctggcactatcttaagggctggacatttcaagaaggtttgcgtatctgaagaagggctggtttgaaggggtcgccgatgagagtgggaacaagctgcgaggatgcctctgcgcgaattcctaaaggagtaatctgcgtccacgtcatcagtagccgtaggcctctgcgacgtcgccgtgctcctcggccgccgcttcgccgccgcccagcagccccagacgcacgcgctcccagacggcgcgcgcgacggggacgatcatgagcagcgcgatctggggcgtgtcgtgcgagctgagccggacgaccacgttgccctggccgcgcgtggtgtactggcagtttgccgagaccatggtgtctgacttgaagtaggagccgtcaaagcgcgggaggcgcgtctcggccgagacgttgccgccgacgaccaggctctggcgcttctgacgcgggtcgcggccctgccactgcgcggagcctcccaggagcaggcgcgcctcgtcgcggctgcgcagcttgaggtcggccgccagcgccgtgcccggctcgtaggccgaccccatcttgacgaagaggcggccgaccgccgcgcggagcttgaccgcgtcggacaggcgcaggcgctcgtccagcttgaccccgagcgccgcgttgccgggcttgaacggcagcgagaactcctcgcccagcttggagccgagcaggcccagcgccagcttctgccgcttgccgcgcgcgcgcaggcgcgagtcgacgcgcagcgtgtagag

SEQ ID NO: 8 - DNA, 4083 bp, unidentified
  source 1..4083: mol_type = unassigned DNA; note = Prototheca wickerhamii
gcaatggcgctctgtgcagggtctgtgtccggtctccctctcctacgatgcacaaggcaacgcctttgctgactcagcgcgtccacaacctcccctcgtcacacacacctgcggaaccaggccgcccatttgctgcttgagcatgccttgcatcatgtccgggttgcccatcatatcgttgaggttcttgggctccagtttctgctccagcacgccatcctgtcggtcgaagaggaggagacatgtaagcgttgttgggatgagggttgctaaattggccattatttttaatgatcacgctcgtgccaatagacgcggcacattgcgatgttcaaactcccgtcaaagccgcggacaaccacatccctccacaccccacacacaaagaacccgccaccgctcaccttgcccacgaggtaggcctttcgttgcgcaaacccggcctcggtgatgaatgcgtgcccgttcctgacgagcgctgcccgggccaacacgctcttttgctgcgtctcctcaggcttgggggcctccttgggcttgggggccgccatgatctgcgcgcaccagagagaagtcattggtgaaaaggagcgcccggctgcgctatatatatatatatatgctcaggccaataaagtccaacctcactggggagccccgtcccaccacccccaagtcgcgtaccttgacggcatactgctgcagaagcttcatgaggatgatgccgaacaagaggggcacaaggacccaatcccggacatccttgtcgataatgatctcgtgagtccccatcgtccgcccgacgctgcggggagcccgccgatgctcaagacgagagggccctcgaccaggaggggctggcccgggcgggcactggcgtcgaaggtgcgcccgtcgttcgcctgcagtccgatgacacaaaacaagtcttctgacggggtgcgtttgctcccgtgcgggcaggcaacagaggtattcaacctggtcatggcgagatcggcgatcgagctgggataagagatacggtcccgcgcgaggatcgctcatcctggtctgagccggacagtcattctggcaagcaatgacaacttgccaggaccggaccgtgccatatatttctcacctagcgcggcaaaacctaacaatttggaagtcactgtgccactgagctcgactggtagctgaatggagtcgctgctccactaatcgaattgtcagcaccgccagccggccgaggacccgagtcatagcgagggtagtagcgcgccatggcaccgaccagcctgcttgcccgtactggcgtctcttccgcttctctgtgctcctctacgcgctccggcgcgtgcgcttttccggtggatcatgcggtccgtggcgcaccgcagcggccgctgcccatgcagcgccgctgcttccgaacagtggctgtcagggccgcacccgcagtagccgtccgtccggaacccgcccaagagttttgggagcagcttgagccctgcaagatggcggaggacaagcgcatcttcctggaggagcaccggtgcgcggaggtccggggctgaccggccgtcgcattcaacgtaatcaatcgcatgatgatcacaggacgcgacgtcttggtggcggtggccagggacactgcccattgcacaggcataggaatgcgttccttctcatttctcagttttctgagcccctccctcttcactctttctcctcctcctcccctctcacgcagcattcgtggcaacgaggtgggcccctcgcagcggctgacgatcacggcggtggccaacatcctgcaggaggcggcgggcaaccacgcggtggccatgtggggtcggagctcggagggtttcgcgacggacccggagctgcaggaggcgggcctcatctttgtgatgacgcgcatgcagatccaaatgtaccgctacccgcgctggggcgacctgatgcaggtggagacctggttccagacggcgggcaagctaggcgcgcagcgcgagtgggtgctgcgcgacaagctgaccggcgaggcgctgggcgcggccacctccagctgggtcatgatcaacatccgcacgcgccggccgtgccgcatgcccgagctcgtccgcgtcaagtcggccttcttcgcgcgcgagccgccgcgcctggcgctgccgcccacggtcacgcgcgccaagctgcccaacatcgcgacgccggcgccgctgcgcgggcaccgccaggtcgcgcgccgcaccgacatggacatgaacggccacgtgaacaacgttgcctacctggcctggtgcctggaggccgtgcccgagcacgtcttcagcgactaccacctctaccagatggagatcgacttcaaggccgagtgccacgcgggcgacgtcatctcctcccaggccgagcagatcccgccccaggaggcgctcacgcacaacggcgccggccgcaacccctcctgcttcgtccacagcattctgcgcgccgagaccgagctcgtccgcgcgcgcaccacctggtcggcccccgtcgacgcgcccgccgccaagccgcccaaggcgagccactgagcagaggagagggtggctggtagttgcgggatggctggtcgcccgtcgatcctgctgctgctattgtctcctcctgcacaagcccacccacgactccgaagaagaagaagaaaacgcgcacacacacaacccaaccggccgaatatttgcttccttatcccgggtccaagagagacggcgatgcccccctcaatcagcctcctcctccctgccgctccaatcttccctgcttgcatgcgcccgcgagaggctgtctgcgcgccccgtcagtcactccccgtgcagacgcctcgtgctcggtgctcctgtatcctttaccgctcctttcattctgcgaggccccctgttgaatgtattcgttgcctgtgtggccaagcgcgctgctgggcgcgccgccgtcgggcggtgctcggcgactctggcggaagccggttgttcttctgtaagccacgcgcttgctgcttttggaaaagaggggggtttactgaatggaggaggagcaggataattggtagtatctgagttgttggggaggcaggaagagttgggaaatggaagtggcacgatgggcaaggagaatggtgagcatgcgcagggtgatgtcgttggccgagaacgatcctgcactcccgtaagtagaatacggcaatcacgttgatctacatctctaacttctccatttaaagccctttccacatgccctcgcgtttgcatcctacaccctttgatccacatgattgatgaccctggcactatcttaagggctggacatttcaagaaggtttgcgtatctgaagaagggctggtttgaaggggtcgccgatgagagtgggaacaagctgcgaggatgcctctgcgcgaattcctaaaggagtaatctgcgtccacgtcatcagtagccgtaggcctctgcgacgtcgccgtgctcctcggccgccgcttcgccgccgcccagcagccccagacgcacgcgctcccagacggcgcgcgcgacggggacgatcatgagcagcgcgatctggggcgtgtcgtgcgagctgagccggacgaccacgttgccctggccgcgcgtggtgtactggcagtttgccgagaccatggtgtctgacttgaagtaggagccgtcaaagcgcgggaggcgcgtctcggccgagacgttgccgccgacgaccaggctctggcgcttctgacgcgggtcgcggccctgccactgcgcggagcctcccaggagcaggcgcgcctcgtcgcggctgcgcagcttgaggtcggccgccagcgccgtgcccggctcgtaggccgaccccatcttgacgaagaggcggccgaccgccgcgcggagcttgaccgcgtcggacaggcgcaggcgctcgtccagcttgaccccgagcgccgcgttgccgggcttgaacggcagcgagaactcctcgcccagcttggagccgagcaggcccagcgccagcttctgccgcttgccgcgcgcgcgcaggcgcgagtcgacgcgcagcgtgtagag

SEQ ID NO: 9 - DNA, 4768 bp, synthetic construct
  source 1..4768: mol_type = other DNA
gcctgcctcgccaactgcgagttctgcgcgcgcggcgggcgcgagggccgcgacggcgcgccggggttcaccgcctcggtcgaggagctctgggagcggcggcgaacagagtttgtgcacctcccggagcaggagtttgccggcatcatgctcgtcatcatgccctgccgctctgacaagtcgaaaaaaggcgtcgccaagtacgcaagcgggggtggggtgggttgggggtggtggcgatgcgatgaaaatggtggtgttggtggttccgattggcgtgtcggaattttgtgaactgagcggcactctgcggctctgccggcgcatgcctagtccagttttcgatgtgcgctatcatgtaccacgccgcatgcgaatctcaccctttcttctccgccccaggtatggtcacctgagcgacggcctgttgcacctggtgctcatcaggcggtgcagccgcctccagtacctcaagtttctgctcaggatgtcgcacatcggccttgaggcgggcgggcagcacgggtcctacatccaggtcttgcctgcccacgcggtgcacatcgaagcggtacgagcgtggatcgggaggcacttgacagtcgcgtcgccgaacaagggacgggtaccctgaggagcttgttggaccgctgattcactccctgacctcccttttctctttccaaaacaggttggccaggagagccactggaacgtcgatggggagctcattcaaagccgcacgataaacgcgcagctccaccgcggagtgatcgatgtgtttgcccgaggcgtggagggctgacgtgcgcgaaactagctgggggcccccattcccgccctttaaacgcctgcctcctggtccccgaccggtgctggcggccctgatcaattcgtccattccgttttattctttgacaatgagcgcctcatcccagtgccaccgcccatccccaaattgttcctctcaaacctctcagatacctcccttcaaactgctcccaagagtgcacgagtacttgtaatgttatgcgaccgctgttcacaatgtagtcggcatgcttgtgtgagcgcgttcgaaactcgctcagcccgtcggcctgcccatggacgtcccctgatcgctttatgcccaccgcatggaccgcgacacgccagttttcggattcaatagcaacgaatacgcatcgataattctgtgacattgcatgcctcaccgcgtgaattgctgtcccaaacgtaagcattatcatggctcggtcacgcgatcctggatccggggatcctggaccgctggtggagagcgctgccgtcggattggtggcaagcaagattgcgcaggttggcgaagggagagaccaaaaccggaggctggaagcgggcacaacatcgtattattgcgtatagtagagcagtggcagtcgcatttcgaggtccgcaacggatctcgcaagctcgctacgctcacagtaggagataggggaccactgcccctgccagaatggtcgcgaccctgtccctcgccggccccgcctgcaacacgcagtgcgtatccagcaagcgggttgtcgccttcaaccgcccccatgttggcgtccgggctcgatcaggtgcgctgaggggggtttggtgggcccgcgcctctgggcccgtgtcggccgtgcggacgtggggcccggggtagtggatcagcaggggttgcatgcaaatgcctataccggcgattgaatagcgatgaacgggatacggttgcgctcactccatgcccatgcgaccccgtttctgtccgccagccgtggtcgcccgagctgcgaagcgggaccccacccagcgcattgtgatcaccggaatgggcgtggcctccgtgtttggcaacgatgtcgagaccttttacgacaagcttctggaaggaacgagcggcgtggacctgatttccaggtgcgtaggtccttggatgaatgcgtctaggttgcgaggtgactggccaggaagcagcaggcttggggtttggtgttctgatttctggtaatttgaggtttcattataagattctgtacggtcttgtttcgaaaacatgcaacaactccacacacacacactcctctcaactgagtctgcaggtttgacatctccgagttcccgaccaagtttgcggcgcagatcaccggcttctccgtggaggactgcgtggacaagaagaacgcgcggcggtacgacgacgcgctgtcgtacgcgatggtggcctccaagaaggccctgcgccaggcaggcctggagaaggacaagtgccccgagggctacggggcgctggacaagacgcgcacgggcgtgctggtcggctcgggcatgggcgggctgacggtcttccaggacggcgtcaaggcgctggtggagaagggctacaagaagatgagccccttcttcatcccctacgccatcaccaacatgggctccgcgctggtgggcatcgaccagggcttcatgggccccaactactccgtctccacagcctgcgcgacgtccaactacgcatttgtgaacgcggccaaccacatccgcaagggcgacgcggacgtcatggtcgtcggcggcaccgaggcctccatcgtgcccgtgggcctgggcggctttgtggcctgccgcgcgctgtccacgcgcaacgacgagcccaagcgcgcgagccggccgtgggacgagggccgcgacggctttgtgatgggcgagggcgcggccgtgctggtcatggagtcgctggagcacgcgcagaagcgtggcgcgaccatcctgggcgagtacctgggcggcgccatgacctgcgacgcgcaccacatgacggacccgcaccccgagggcctgggcgtgagcacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggtatcaagatgaacgccaccaagagtatgatcgggcactgcctgggcgccgccggcggcatggaggccgtcgcgacgctcatggccatccgcaccggctgggtgcaccccaccatcaaccacgacaaccccatcgccgaggtcgatggcctggacgtcgtcgccaacgccaaggcccagcacgacatcaacgtcgccatctccaactccttcggctttggcgggcacaactccgtcgtcgcctttgcgcccttccgcgagtaggtgaagcgagcgtgctttgctgaggagggaggcggggtgcgagcgctctggccgtgcgcgcgatactctccccgcatgagcagactcctcgtgccacgcccgaatctacttgtcaacgagcaactgtgtgttttgtccgtggccaatcttattatttctccgactgtggccgtactctgtttggctgtgcaagcaccccaggaactcttttgttgagcggggggttatcgtagagagggtttgcgaaatgggcaccgatcggaaggccttgcaaacgtggcggtcattggccgacatgatttccgtggattcactgatgcggcattgcttaccattcatattgaaaagacagccttgcaacctacaatttgatggagcaaatacatatatatacggggccgacgtgtcagatggccgttgcgctcttgagctccacggcccgacacagctgctcgatagcttcctccagcggcaggcagccgcgctggccgtcagcgtacgtgcgcaccgccagggtcgagctcccggcctcttccgggccgatgaccgccatgacggggactttggccctctcggcgttgcgaacgagcttgcccatggaggcgccgccgggcgcgagctccacgcgcaggccgcgcgcgcgcatggcggctgccaccgtctccatgtacggccgcaccgcgtccgtcgtgggcagcagtcgcacctgctccggggccagccagagcgggaaggcgcccgcgtagtgctcgatcaggatgccgaagaagcgctccagcgagccgagcagggcgcggtgcaccatgatgggccgctggcggctgccgtcggccgccgcgtactcgagcccgaaccgctcgggcaggttgaagtccagctgcaccgtggagcactgccacttgcgcccgagcgcgtcctggatcttgacgtcgatcttcgggccgtaaaaggcgccgccgccgacgtccaccgtgaaggcccagccctgtcgatgaatgattgttgggagggggggtttcgaggttggatgacaccacaataatatagtcagcgacacgcgtgctgcacgataattaccagtacagactcggcgatgataacaccacccttttcatcaaccgaaatgagccgttccctgttgacaaacacacacgcacacacaccttgtcgttgagcgcttcagcgagcgccgcctcggccctggcccagtcctcgtcggacccgaccgactcgtccggccgcgtggagaggttgacctcgaaccggcacgcgtcgaagccaaaggcgcggaagacgcgctccgtgaggtccagcacggcccggatctcggccgcgatctgctccggcaggcagaagatgtgggcgtcgtcctgcgtgaagccgcgcacgcggaagaggccgtgcatggtgccggagcgctcgtagcggtagacagtgcccagctccgcccagcgcagcggcaggtcgcggtagctgacgggcctggccgcgtagacgctga

SEQ ID NO: 10 - DNA, 4768 bp, unidentified; identical to SEQ ID NO: 9
  source 1..4768: mol_type = unassigned DNA; note = Prototheca wickerhamii

SEQ ID NO: 11 - DNA, 4792 bp, synthetic construct
  source 1..4792: mol_type = other DNA
gcctgcctcgccaactgcgagttctgcgcgcgcggcgggcgcgagggccgcgacggcgcggcggggttcaccgcctcggccgaggagctctgggagcggcggcgagcagagtttgtgcacctcccggagcaggagtttgcaggcatcatgctcgtcatcatgccctgccgctctgacaagtcgaaaaagggcgtcgccaagtacgcaagcacagggggtggggtggggtgggggtggtggcggtgcgatgaaaatggtggtgtttggtggttcacattggcgtgtcggagttttgtgaagtgagcttcactctgcggctctcccggcgcatgcctagtccagttttcgatgtgcgctatcgtgtaccacgccgcgtgcgaatcccaccctttcttctccgccccaggtatggtcacctgagcgacggcctgctgcacctggtgctcatcaggcggtgcagccgcctccagtacctcaagtttctgctcaggatgtcgcacatcggcctcgaggcgggcgggcagcacgggtcctacatccaggtcctgcccgcccacgcggtgcacatcgaagcggtacgagcgtggatcgggaggcacttgacagtcgcgtcgcccgacaaggcacgggcgccctgaggagcttgttgggccgctgattcacttccctggcctcccctttctctttccaaaccaggttggccaggagagccactggaacgtcgatggggagctcattcaaagccgcacgatcaacgcgcagctccaccgcggagtgatcgacgtgtttgccagaggcgtggagggctgacgagcgcgaaactagctgggggccccattcccgcccttgaaacgcctgcctcctgggccctggccggtgctggcggccctgaccgattcgtccattccgttttattctttgacaatgagcgcctcatcccagtgcctcatcccagtgccaccgcccatccccaaattgttcctctcaaacctctcagatacccccttcaaactgctcccaagagtgcacgagtatttgtaatgttatacgaccgctgttcacaatgtagtcggcatgcttgtatgagcctgttcgaaactcgctcagcccgtcggcctgcccatggacctcccctgatcgcttcacgcccaccgcatggactgcgacacgccagttttgggattcaatatcaaaaaatacgcttcggtaattctgtaacgttgcatgcctcaccgcgtgaattgctgtcccaaacgtaagcatcatcgtggctcggtcacgcgatcctggatccggggatcctagaccgctggtggagagcgctgccgtcggattggtggcaagtaagattgcgcaggttggcgaagggagagaccaaaaccggaggctggaagcgggcacaacatcgtattattgcgtatagtagagcagtggcagtcgcatttcgaggtccgcaacggatctcgcaagctcgctacgctcacagtaggagaaaggggaccactgcccctgccagaatggtcgcgaccctctccctcgccggccccgcctgcaacacgcagtgcgtatccggcaagcgggctgtcgccttcaaccgcccccatgttggcgtccgggctcgatcaggtgcgctgaggggggtttggtgtgcccgcgcctctgggcccgtgtcggccgtgcggacgtggggccctgggcagtggatcagcagggtttgcgtgcaaatgcctataccggcgattgaatagcgatgaacgggatacggttgcgctcactccatgcccatgcgaccccgtttctgtccgccagccgtggtcgcccgggctgcgaagcgggaccccacccagcgcattgtgatcaccggaatgggcgtggcctccgtgtttggcaacgatgtcgagaccttttacaacaagcttctggaaggaacgagcggcgtggacctgatttccaggtgcgtaggtccttggatgcatgcgtctaggttgggaggcggctggcgaggaagcagcaggcttggggtttggtgttccgatttctggcaatttgaggtttcattgtgagattctatgcggtcttgtttcgaaaacatgcaacaactccacacacacacactcctctccaccaactctgcaggtttgacatctccgagttcccgaccaagtttgcggcgcagatcaccggcttctccgtggaggactgcgtggacaagaagaacgcgcggcggtacgacgacgcgctgtcgtacgcgatggtggcctccaagaaggccctgcgccaggcgggactggagaaggacaagtgccccgagggctacggagcgctggataagacgcgcgcgggcgtgctggtcggctcgggcatgggcgggctgacggtcttccaggacggcgtcaaggcgctggtggagaagggctacaagaagatgagccccttcttcatcccctacgccatcaccaacatgggctccgcgctggtgggcatcgaccagggcttcatggggcccaactactccgtctccacggcctgcgcgacctccaactacgcctttgtgaacgcggccaaccacatccgcaagggcgacgcggacgtcatggtcgtgggcggcaccgaggcctccatcgtgcccgtgggcctgggcggctttgtggcctgccgcgcgctgtccacgcgcaacgacgagcccaagcgcgcgagccggccgtgggacgagggccgcgacggcttcgtgatgggcgagggcgcggccgtgctggtcatggagtcgctggagcacgcgcagaagcgcggcgcgaccatcctgggcgagtacctggggggcgccatgacctgcgacgcgcaccacatgacggacccgcaccccgagggcctgggcgtgagcacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggcatcaagatgaacgccaccaagtccatgatcgggcactgcctgggcgccgccggcggcatggaggccgtcgccacgctcatggccatccgcaccggctgggtgcaccccaccatcaaccacgacaaccccatcgccgaggtcgacggcctggacgtcgtcgccaacgccaaggcccagcacaaaatcaacgtcgccatctccaactccttcggcttcggcgggcacaactccgtcgtcgcctttgcgcccttccgcgagtaggcggagcgagcgcgcttggctgaggagggaggcggggtgcgagccctttggctgcgcgcgatactctccccgcacgagcagactccacgcgcctgaatctacttgtcaacgagcaaccgtgtgttttgtccgtggccattcttattatttctccgactgtggccgtactctgtttggctgtgcaagcaccccaggaactcttttgtcgagcggggggtgtcgtagagagggtccgcgaaacaggcaccgatcgcgagcctcgtggtggtcattgttcgacatgattcccggagattcactgatgcggcattgcttaccattcatttgaaaagacagcttgcaacctacaattcgaaggagcgaatacatatatatacggggccggcgtgtcagatcgccgtttcgctcttgagctccacggcccgacacagctgctcgatgacttcctccagcggcaggcagccgcgctggccgtcggcgtacgtgcgcaccgccagggtcgagctcccggcctcttccgggccgatgaccgccatgacggggaccttggccctctcggcgttgcggatgagcttgcccatggaggccccgccgggcgcgagctccacgcgcaggccgcgcgcgcgcatggcgtctgccaccgtctccatgtagggccgcaccgcgtccgtcgtgggcagcaggcgcacctgctccggggccagccagagcgggaaggcgcccgcgtagtgctcgatcaggatgccgaagaagcgctccagcgagccgagcagggcgcggtgaatcatgatgggccgctggcggctgccgtcggcggccgcgtactcgagcccaaaccgctcgggcaaattgaagtccagctgcaccgtggagcactgccacttgcgcccgagcgcgtcctggatcttgacgtcgatcttcgggccgtaaaaggcgccgccgccggcgtccaccgtgaaggcccagccctgtcgatgaatgattcgtggttgagagggcggatttttcgaggtctggatgatgatacggtatgcgccacaataatactacactcgcgccagtcagcgacacgcgtgctgcacaatgcctactactatagactcggtgatgatgataccacccttttcatcgaacgagatgggccattccctactgacaaacacacacgcaccttgtcgttcagcgcttcggcgagcgccgcctcggccctggcccagtcctcgtcggacccgaccgactcgtccggccgcgtggagaggttgacctcgaaccggcgcgcgtcgaagccaaaggcgcggaagacgcgctccgtgaggtccagcacggcccggatctcggccgcgatctgctccggcaggcagaagacgtgggcgtcgtcctgcgtgaagccgcgcacgcggaagaggccgtgcatggtgccggagcgctcgtagcggtagaccgtgcccagctccgcccagcgcagcggcaggtcgcggtagctgacgggccgggccgcgtagacgctga

SEQ ID NO: 12 - DNA, 4793 bp, unidentified
  source 1..4793: mol_type = unassigned DNA; note = Prototheca wickerhamii
gcctgcctcgccaactgcgagttctgcgcgcgcggcgggcgcgagggccgcgacggcgcggcggggttcaccgcctcggccgaggagctctgggagcggcggcgagcagagtttgtgcacctcccggagcaggagtttgcaggcatcatgctcgtcatcatgccctgccgctctgacaagtcgaaaaagggcgtcgccaagtacgcaagcacagggggtggggtggggtgggggtggtggcggtgcgatgaaaatggtggtgtttggtggttcacattggcgtgtcggagttttgtgaagtgagcttcactctgcggctctcccggcgcatgcctagtccagttttcgatgtgcgctatcgtgtaccacgccgcgtgcgaatcccaccctttcttctccgccccaggtatggtcacctgagcgacggcctgctgcacctggtgctcatcaggcggtgcagccgcctccagtacctcaagtttctgctcaggatgtcgcacatcggcctcgaggcgggcgggcagcacgggtcctacatccaggtcctgcccgcccacgcggtgcacatcgaagcggtacgagcgtggatcgggaggcacttgacagtcgcgtcgcccgacaaggcacgggcgccctgaggagcttgttgggccgctgattcacttccctggcctcccctttctctttccaaaccaggttggccaggagagccactggaacgtcgatggggagctcattcaaagccgcacgatcaacgcgcagctccaccgcggagtgatcgacgtgtttgccagaggcgtggagggctgacgagcgcgaaactagctgggggccccattcccgcccttgaaacgcctgcctcctgggccctggccggtgctggcggccctgaccgattcgtccattccgttttattctttgacaatgagcgcctcatcccagtgcctcatcccagtgccaccgcccatccccaaattgttcctctcaaacctctcagataccccccttcaaactgctcccaagagtgcacgagtatttgtaatgttatacgaccgctgttcacaatgtagtcggcatgcttgtatgagcctgttcgaaactcgctcagcccgtcggcctgcccatggacctcccctgatcgcttcacgcccaccgcatggactgcgacacgccagttttgggattcaatatcaaaaaatacgcttcggtaattctgtaacgttgcatgcctcaccgcgtgaattgctgtcccaaacgtaagcatcatcgtggctcggtcacgcgatcctggatccggggatcctagaccgctggtggagagcgctgccgtcggattggtggcaagtaagattgcgcaggttggcgaagggagagaccaaaaccggaggctggaagcgggcacaacatcgtattattgcgtatagtagagcagtggcagtcgcatttcgaggtccgcaacggatctcgcaagctcgctacgctcacagtaggagaaaggggaccactgcccctgccagaatggtcgcgaccctctccctcgccggccccgcctgcaacacgcagtgcgtatccggcaagcgggctgtcgccttcaaccgcccccatgttggcgtccgggctcgatcaggtgcgctgaggggggtttggtgtgcccgcgcctctgggcccgtgtcggccgtgcggacgtggggccctgggcagtggatcagcagggtttgcgtgcaaatgcctataccggcgattgaatagcgatgaacgggatacggttgcgctcactccatgcccatgcgaccccgtttctgtccgccagccgtggtcgcccgggctgcgaagcgggaccccacccagcgcattgtgatcaccggaatgggcgtggcctccgtgtttggcaacgatgtcgagaccttttacaacaagcttctggaaggaacgagcggcgtggacctgatttccaggtgcgtaggtccttggatgcatgcgtctaggttgggaggcggctggcgaggaagcagcaggcttggggtttggtgttccgatttctggcaatttgaggtttcattgtgagattctatgcggtcttgtttcgaaaacatgcaacaactccacacacacacactcctctccaccaactctgcaggtttgacatctccgagttcccgaccaagtttgcggcgcagatcaccggcttctccgtggaggactgcgtggacaagaagaacgcgcggcggtacgacgacgcgctgtcgtacgcgatggtggcctccaagaaggccctgcgccaggcgggactggagaaggacaagtgccccgagggctacggagcgctggataagacgcgcgcgggcgtgctggtcggctcgggcatgggcgggctgacggtcttccaggacggcgtcaaggcgctggtggagaagggctacaagaagatgagccccttcttcatcccctacgccatcaccaacatgggctccgcgctggtgggcatcgaccagggcttcatggggcccaactactccgtctccacggcctgcgcgacctccaactacgcctttgtgaacgcggccaaccacatccgcaagggcgacgcggacgtcatggtcgtgggcggcaccgaggcctccatcgtgcccgtgggcctgggcggctttgtggcctgccgcgcgctgtccacgcgcaacgacgagcccaagcgcgcgagccggccgtgggacgagggccgcgacggcttcgtgatgggcgagggcgcggccgtgctggtcatggagtcgctggagcacgcgcagaagcgcggcgcgaccatcctgggcgagtacctggggggcgccatgacctgcgacgcgcaccacatgacggacccgcaccccgagggcctgggcgtgagcacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggcatcaagatgaacgccaccaagtccatgatcgggcactgcctgggcgccgccggcggcatggaggccgtcgccacgctcatggccatccgcaccggctgggtgcaccccaccatcaaccacgacaaccccatcgccgaggtcgacggcctggacgtcgtcgccaacgccaaggcccagcacaaaatcaacgtcgccatctccaactccttcggcttcggcgggcacaactccgtcgtcgcctttgcgcccttccgcgagtaggcggagcgagcgcgcttggctgaggagggaggcggggtgcgagccctttggctgcgcgcgatactctccccgcacgagcagactccacgcgcctgaatctacttgtcaacgagcaaccgtgtgttttgtccgtggccattcttattatttctccgactgtggccgtactctgtttggctgtgcaagcaccccaggaactcttttgtcgagcggggggtgtcgtagagagggtccgcgaaacaggcaccgatcgcgagcctcgtggtggtcattgttcgacatgattcccggagattcactgatgcggcattgcttaccattcatttgaaaagacagcttgcaacctacaattcgaaggagcgaatacatatatatacggggccggcgtgtcagatcgccgtttcgctcttgagctccacggcccgacacagctgctcgatgacttcctccagcggcaggcagccgcgctggccgtcggcgtacgtgcgcaccgccagggtcgagctcccggcctcttccgggccgatgaccgccatgacggggaccttggccctctcggcgttgcggatgagcttgcccatggaggccccgccgggcgcgagctccacgcgcaggccgcgcgcgcgcatggcgtctgccaccgtctccatgtagggccgcaccgcgtccgtcgtgggcagcaggcgcacctgctccggggccagccagagcgggaaggcgcccgcgtagtgctcgatcaggatgccgaagaagcgctccagcgagccgagcagggcgcggtgaatcatgatgggccgctggcggctgccgtcggcggccgcgtactcgagcccaaaccgctcgggcaaattgaagtccagctgcaccgtggagcactgccacttgcgcccgagcgcgtcctggatcttgacgtcgatcttcgggccgtaaaaggcgccgccgccggcgtccaccgtgaaggcccagccctgtcgatgaatgattcgtggttgagagggcggatttttcgaggtctggatgatgatacggtatgcgccacaataatactacactcgcgccagtcagcgacacgcgtgctgcacaatgcctactactatagactcggtgatgatgataccacccttttcatcgaacgagatgggccattccctactgacaaacacacacgcaccttgtcgttcagcgcttcggcgagcgccgcctcggccctggcccagtcctcgtcggacccgaccgactcgtccggccgcgtggagaggttgacctcgaaccggcgcgcgtcgaagccaaaggcgcggaagacgcgctccgtgaggtccagcacggcccggatctcggccgcgatctgctccggcaggcagaagacgtgggcgtcgtcctgcgtgaagccgcgcacgcggaagaggccgtgcatggtgccggagcgctcgtagcggtagaccgtgcccagctccgcccagcgcagcggcaggtcgcggtagctgacgggccgggccgcgtagacgctga

SEQ ID NO: 13 - DNA, 4205 bp, synthetic construct
  source 1..4205: mol_type = other DNA
aaacccgatgatgcccagcgcggagctcagcatgtcgccgagcatcgctggataggacgaattggcagggtaccacctgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaatggcgtctgccgtcacctttgcgtgcgcccctccccgcggcgcggtcgccgcgccgggtcgccgcgctgcctcgcgtcccctggtggtgcgcgcggtcgccagcgaggccccgctgggcgttccgccctcggtgcagcgcccctcccccgtggtctactccaagctggacaagcagcaccgcctgacgcccgagcgcctggagctggtgcagagcatggggcagtttgcggaggagagggtgctgcccgtgctgcaccccgtggacaagctgtggcagccgcaggactttttgcccgaccccgagtcgcccgacttcgaggatcaggtggcggagctgcgcgcgcgcgccaaggacctgcccgacgagtactttgtggtgctggtgggggacatgatcacggaggaggcgctgccgacctacatggccatgctcaacacgctggacggcgtgcgcgacgacacgggcgcggccgaccacccgtgggcgcgctggacgcggcagtgggtggccgaggagaaccggcacggcgacctgctgaacaagtactgctggctgacggggcgcgtcaacatgcgggccgtggaggtgaccatcaacaacctgatcaagagcggcatgaacccgcagacggacaacaacccttatttggggttcgtctacacctccttccaggagcgcgccaccaagtacagccacggcaacaccgcgcgccttgcggccgagcacggcgacaagaacctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgcggtcgccgagaagatcgacgtctacgacgccgaggactactgccgcatcctggagcacctcaacgcgcgctggaaggtggacgagcgccaggtcagcggccaggccgccgcggaccaggagtacgtcctgggcctgccccagcgcttccggaaactcgccgagaagaccgccgccaagcgcaagcgcgtcgcgcgcaggcccgtcgccttctcctggatctccgggcgcgagatcatggtctagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtcctgggcgaagaacgagggaatttgtgggtaaaacaagcatcgtctctcaggccccggcgcagtggccgttaaagtccaagaccgtgaccaggcagcgcagcgcgtccgtgtgcgggccctgcctggcggctcggcgtgccaggctcgagagcagctccctcaggtcgccttggacggcctctgcgaggccggtgagggcctgcaggagcgcctcgagcgtggcagtggcggtcgtatccgggtcgccggtcaccgcctgcgactcgccatccgaagagccatcgtcgtcgtctcccagactggacgcgctcgccatcggccccgccgatgcagaacacgcctctgcgctcgccgtgcgcagcgcggcgcgcagcgctgcgcgcgccagggccgcgtccagcaggcccatcgccagctcccggccgcgagggccggccagcgcctcaaagccgccggcgcgggcggccgccaccaccgaggccagcgcggcgcgcgcgccctccagggtcggcgcccgggtcacggcgtcgcgcgcccaggcggccagcgccgccccccgtagaacgcagtgcgcctccatcgaccgcacaaagtgggagacccggtggctcgtggggtcgtagggccgcacgcggcggcgcgcgggcctcgtggttgcgcgcgtgctgctccgcagccccgcaatcgtcacgctcccttgccgatccacgctcgcggcggccagcctggcccaggccagctggaggcccaggcgcatgctggcgcggacctgctccagaaaggggccgtcgcccagcatgcacagcggccaggggagcgtcaggtcgaaccgcaggcgctccagcgtggcgacgcgcggaggctcgcccgggagcggccagggacgagcgggcgattgggtggccggcggcgaataaccagggtgcactccgtcagcagcctcctccgacctcacacccccctgcctcttgagctggcggcaccgggccgcgatggccgcaccggcgctgtccagcacgcccgcttccacagccacgtgcagctcgatgccggcgacgtccgtctc

SEQ ID NO: 14 - DNA, 4205 bp, unidentified; identical to SEQ ID NO: 13
  source 1..4205: mol_type = unassigned DNA; note = Prototheca wickerhamii

SEQ ID NO: 15 - DNA, 4184 bp, synthetic construct
  source 1..4184: mol_type = other DNA
ggagctcagcatgtcgccagcatcgctggataagacgaattggcagggtaccacctgaagaatgggaggcaggtgttgctgattacgagtgtgtaaaagaaaggggtagagagccgtcctcagatccgaccactatgcaggtagccgctcgcccatgcccgcctggctgaatattgacacatgcccatcaaggcaggcaggcatttctgtgcatgcaccaagcccacaatcttccacaacacacagcatgtaccaacgcacgcgtaaaagttggggtgctgccagtgcgtcatgccaggcatgatgtgctcctgcacatcggccatgatctcctccatcgtctcgggtgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggaggagagggaggagagaggggggggggatgatcacacgccagtctcacaacgcatgtaagacccgtttgattatgagtacaatcatgcactactagttggatgagcgccaggcataaggcacaccgacgtcgatggcacgagcaactcccgcatcatatttcctattgtcctcacgccaagccggtcaccatccgcatgctcgtattacagcgcacgcaccgcttcgtgatccaccgggtgaacgtagtcctcgacggaaacatctggttcgggcctcgtgctggcactccctcccatgccgacaacctttctgctgtcaccacgacccacgatgcaacgcgacacgaccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtggcctgggtgtttcgtcgaaaggccagcaaccccaaatcgcaggcgatccggagattgggatctgatccgagttggaccagatccccccgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgtgtgtctgttttcggctgcacaattcaatagtcggatgggcgacggtagaattgggtgttgcgctcgcgtgcatgcctcgccccgtcgggtgtcatgaccgggactggaatcccccctcgcgaccctcttgctaacgctcccgactctcccgcccgcgcgcaggatagactctagttcaaccaatcgacaatggcgtctgccgtcacctttgcgtgcgcccctccccgcggcgcggtcgccgcgccgggtcgccgcgctgcctcgcgtcccctggtggtgcgcgccgtcgccagcgaggccccgctgggcgtgccgccctcggtgcagcgcccctcccccgtggtctactccaagctggacaagcaacaccgcctgacgcccgagcgcctggagctggtgcagagcatgggtcagtttgcggaggagagggtgctccccgtgctgcaccccgtggacaagctgtggcagccgcaggactttttgcccgaccccgagtcgcccgacttcgaggaccaggtggcggagctgcgcgcgcgcgccaaggacctgcccgacgagtactttgtggtgctggtgggcgacatgatcacggaggaggcgctgccgacctacatggccatgctcaacaccttggacggtgtgcgcgacgacacgggcgcggctgaccacccgtgggcgcgctggacgcggcagtgggtggccgaggagaaccggcacggcgacctgctgaacaagtactgttggctgacggggcgcgtcaacatgcgggccgtggaggtgaccatcaacaacctgatcaagagcggcatgaacccgcagacggacaacaacccttacttgggcttcgtctacacctccttccaggagcgcgcgaccaagtacagccacggcaacaccgcgcgcctggcggccgagcacggcgacaagggcctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgccgtcgccgagaagatcgacgtctacgacgccgaggactactgccgcatcctggagcacctcaacgcgcgctggaaggtggacgagcgccaggtcagcggccaggccgccgcggaccaggagtacgttctgggcctgccccagcgcttccggaaactcgccgagaagaccgccgccaagcgcaagcgcgtcgcgcgcaggcccgtcgccttctcctggatctccggacgcgagattatggtctagggaggtacgagcgcgcgcgagggattggtgggagtgggacgcgctcgtcgctcctttctattctgaagggaagattggccaccccgctccacgcgccacgcatcgagcaacgaagaaaaccccccgatgataggttgcagtggctgccgagatatagatccggctgcacgtcaaagggcccctcggccagagaagaagctcttttccagcgaccgcagactccttctgccaaaaacactcttctctgtccacagcaacaccaatggatggacagatcaacttgtgtcttcgcgtagcttcctcggctagcgtgcttgcaacaggtccctgcactattatcctcctgctttcctctgaattatgcggcaggcgagcgctcgctcttgcgagcgctccttcgcgccgccctcgctgatcgagtgtacagtcaatgaatggtcctgggcgaagaacgagggaatttgtgggcgagagagcatcgtctctcaggccccagcgcagtggccgttaaagtccaagaccgtgaccaggcagcgcatcgcgtccgtgtgcgggccctgcctggcggctcggcgcgccaggctcgagagcagctccctcaggtcgccttggacagcctctgcgaggccggtgagggtctgcaggagcgcctcgagcgtggcagtggcggttgtatccgggtcgccggtcatcgactgcgactcgccatccgaagagccatcgtcgtcgtctcccagactggaagcgctcgccatcggccccgccgatacagaaaacgcctctgcgctcgccgtgcgcagcgcggcgcgcaccagggccgcgtccagcagacccatcgccagctcccggccgcgagggccggccagcgcctcaaagccgccggcgcgggcggccgccgccgccgaggccagcgcggcgcgcgcgccctccagggtcggcgcccgggtcacggcgtcgcgcgcccaggcggccagcgccgccccccgcaggacgcagtgcgcctccatcgaccgcacaaagtgggagacccggtggctcgtggggtcgtagggccgcacgcggcggcgcgcgggcctcgtgaacgtgcgtttgccattccgcagccctgcggtcgtcacactcccttgccgatccacgctcgcggcggccagcctggcccaggccagctggaggcccaggagcatgccggcgcggacctgctccagaaaggggccgttgcccagcatgcacagtggccaggggagcgtcaggtggaaccgcaggcgctccagcgtggcgacgcgcggaggctcgcccgggagcggccagggacgcgtggacgattgggtggcggcatcgaagtgctggagcgcactccgtcagcggcctccgccgacgccgcacccccctgcctcttgagctggcggcaccgggccgcgatggccgcaccggcgctgtccagcacgcccgcttccacagccacgtgcagctcgatgccggcgacgtctgtctccatca

SEQ ID NO: 16 - DNA, 4202 bp, unidentified
  source 1..4202: mol_type = unassigned DNA; note = Prototheca wickerhamii
ggagctcagcatgtcgcccagcatcgccggataagacgaattggcagggtaccacctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagagggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaatggcgtctgccgtcacctttgcgtgcgcccctccccgcggcgcggtcgccgcgccgggtcgccgcgctgcctcgcgtcccctggtggtgcacgccgtcgccagcgaggccccgctgggcgtgccgccctcggtgcagcgcccctcccccgtggtctactccaagctggacaagcaacaccgcctgacgcccgagcgcctggagctggtgcagagcatgggtcagtttgcggaggagagggtgctccccgtgctgcaccccgtggacaagctgtggcagccgcaggacttcctgcccgaccccgagtcgcccgacttcgaggaccaggtggcggagctgcgcgcgcgcgccaaggacctgcccgacgagtactttgtggtgctggtgggcgacatgatcacggaggaggcgctgccgacctacatggccatgctcaacaccttggacggtgtgcgcgacgacacgggcgcggctgaccacccgtgggcgcgctggacgcggcagtgggtggccgaggagaaccggcacggcgacctgctgaacaagtactgttggctgacggggcgcgtcaacatgcgggccgtggaggtgaccatcaacaacctgatcaagagcggcatgaacccgcagacggacaacaacccttacttgggcttcgtctacacctccttccaggagcgcgcgaccaagtacagccacggcaacaccgcgcgcctggcggccgagcacggcgacaagggcctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgccgtcgccgagaagatcgacgtctacgacgccgaggactactgccgcatcctggagcacctcaacgcgcgctggaaggtggacgagcgccaggtcagcggccaggccgccgcggaccaggagtacgttctgggcctgccccagcgcttccggaaactcgccgagaagaccgccgccaagcgcaagcgcgtcgcgcgcaggcccgtcgccttctcctggatctccggacgcgagattatggtctagggaggtacgagcgcgcgcgagggattggtgggagtgggacgcgctcgtcgctcctttctattctgaagggaagattggccaccccgctccacgcgccacgcatcgagcaacgaagaaaaccccccgatgataggttgcagtggctgccgagatatagatccggctgcacgtcaaagggcccctcggccagagaagaagctcttttcccagcgaccgcagactccttctgccaaaaacactcttctctgtccacagcaacaccaatggatggacagatcaacatgtgtcttcgcgtagcttcctcggctagcgtgcttgcaacaggtccctgcactattatcctcctgctttcctctgaattatgcggcaggcgagcgctcgctcttgcgagcgctccttcgcgccgccctcgctgatcgagtgtacagtcaatgaatggtcctgggcgaagaacgagggaatttgtgggcgagagagcatcgtctctcaggccccagcgcagtggccgttaaagtccaagaccgtgaccaggcagcgcatcgcgtccgtgtgcgggccctgcctggcggctcggcgcgccaggctcgagagcagctccctcaggtcgccttggacagcctctgcgaggccggtgagggtctgcaggagcgcctcgagcgtggcagtggcggttgtatccgggtcgccggtcatcgactgcgactcgccatccgaagagccatcgtcgtcgtctcccagactggaagcgctcgccatcggccccgccgatacagaaaacgcctctgcgctcgccgtgcgcagcgcggcgcgcaccagggccgcgtccagcagacccatcgccagctcccggccgcgagggccggccagcgcctcaaagccgccggcgcgggcggccgccgccgccgaggccagcgcggcgcgcgcgccctccagggtcggcgcccgggtcacggcgtcgcgcgcccaggcggccagcgccgccccccgcaggacgcagtgcgcctccatcgaccgcacaaagtgggagacccggtggctcgtggggtcgtagggccgcacgcggcggcgcgcgggcctcgtgaacgtgcgtttgccattccgcagccctgcggtcgtcacactcccttgccgatccacgctcgcggcggccagcctggcccaggccagctggaggcccaggagcatgccggcgcggacctgctccagaaaggggccgttgcccagcatgcacagtggccaggggagcgtcaggtggaaccgcaggcgctccagcgtggcgacgcgcggaggctcgcccgggagcggccagggacgcgtggacgattgggtggcgggcatcgaagtgctggagcgcactccgtcagcggcctccgccgacgccgcacccccctgcctcttgagctggcggcaccgggccgcgatggccgcaccggcgctgtccagcacgcccgcttccacagccacgtgcagctcgatgccggcgacgtctgtctccatca

SEQ ID NO: 17 - DNA, 4448 bp, unidentified
  source 1..4448: mol_type = unassigned DNA; note = Prototheca wickerhamii
acgaattgttgctttgaagggtgctgaatggcaccagatactgggtcggtggcatttgctcaggcgtcatttgtaggttctgccaataataattccatgaggacagcaagccattgacaagcccactgactcgaattttagagcaacctacgagaccacaccatccgtgcacgcacctcgaggctgtttttggatacgtgcagccgagcgcatggcccgtcgccgtcctcggtagagggagggcagcacagggccctctctttctttttgactggccaagctggcacgctttcggctgccgggatcaggtgagctttcgtcggcgggggcgagcagggccaccaggtcgaggacctccctctccaggcagccaatggtgtggagcaggcctctatgctggtgatgagccgcttgctgctcctggctgtcggcaccggtcctcctggatggcggaggtggcgcaagaacacccgtgtgtcgcacgttcatcaacacgtcttgcgcagtgtcgcacaccgcaaggacgcaggtactttaaaaagacacaagcgtgacatgtgcgtgatcattgcaacggctgccgcagaggacttaccccagctcagcaagcagtgttgagacggccttggccaccccatcgagctcagcgagcagcttttcgtgctggaaggccaggacctcgttttcttgctgggtctccacggctcgccgtcgggattcctggagggaggagggagcccaggggagagtggtgttgtgatgacgacgcgtgtgcgcagaggcagtaggatgctcatgagtgcagcctctttgcagccaggtttgcctgtcgcacagacttgagaggcaccacccaaagtctactatccaaataatgctccagagcatcagcaatgttggccaccttcagtaccaacgctcccaccctcgtctgcgcacctgcgctgcccgcccccgcgcggcctcggcagcgacggacgcgcccgccgcgccctcggcggcctccgcctgcgcctcgagcgcctcacgttccctttccagcgccgccagcgaggcctggacgtccaccaggtgcttgaaccgagcaaagttgcgctcctgcagcccggccaggttggccaccagctcctccatgctggagtagcctgtggccgtggagatcttctggaaagcgtcggtgatggcctgctcgcgctgcgcgtgcgacgcaaagtggacgcctggtgaggccgcgccctggccgtcgagggaggaccccccgcccggcccggcctcggcggcgcccgccgcgccgcccagcgccctcgcccccgccggcgcgccgccgtgcgcgtccagccgcacgggcgctgggtagtcctgcgagaagagctgggccatctcctgctcgcgcgcggcacgctccgcgcggcgatcggcgtcccgggtcgcgcgctcctgctggatgagcgccgccagccgccgccactcctgctcgagcgcgcccgactcgcgctccgcctgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcagcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagctcgcagcatcgcttgcacgggggctatcttcaagtagtcgggaagcatgggtcgcgacgtcgagacgggcgagcgggcctatgggtgagtcggccgtgcatggggagggctagcgccagggctcgggtcgccccgatggccggggtctttgctcggggtgcacgcggccgtgggtgcgagacaggttgcgtgacgaatttcttggcacccccccacggatgcctcacgcgggcgcccccctccctcgcaggtacgtccgcaaggtgtccggcccggtggtggtggccagcagcatgagcgggtctgccatgtacgagctggtccgcgtgggcgcggacaagctgattggcgagatcattcgcctcgagggagacacggcgaccatccaggtgaggtggcgggggaggctgcttgagtggaagtggggcgcacaaccctccattggttggcggccaaggatgcagggaagcgaatgtcgagcattgcgcgcttttgcttgctgacatatcgttgcttgcaccccatgtattctccaggtgtacgaggatacctcgggcctgacggtgggcgacacggtcgtccgctccggcaaggtgtggagggtgtgagagttgatgtatatgctttggtcgcccccttgctctggcacgtcagaggcgcaccttcagtgctcacaagtggtgccgttagtgaaactgaacaagtgcttgactgtgtaggatttccgctcgcccctttcttaccctctcccctttctctctttctccacccctcaaaatctccagcccctgtctgtggagctgggccccggcatcatgggcaccatctttgacggcatccagcgcccgctcaagtcgatcgcggtggacagcgattcgtgcttcatcccgcgcggcgtggacgtgccggccctggaccgcaaggcctcgtgggagtttgacccggtgtcaagcttcaaggtgggcgaccgcatcacgggcggcgacatctacggcgtggtgcacgagaacacgctgatggagcacaaggtgctgcttccgccgggcgcgcgcggcaccatctcctacatcgcgccggcggggagctacagcatcacggacaagatcatcgaggtcgagtttggcggcgcgcgcaaggagtactccatgctgcagctctggcccgtgcgcgcgccgcgccccgtggcgcagaagctgctggccaacacgccgctgctgacggggcagcgcgtgctggacgcgctcttccccggcgtgctgggcgggacgtgcgccatcccgggcgcgtttggctgcggcaagacggtcatctcgcaggcgctgtcaaagtacagtaactcggagggcatcatctacgtcggctgcggcgagcgcggcaacgagatggccgaggtgctcatggacttccccgcgctgaccatgaccatgccggacgggcgcgaggagagcatcatgcagcgcaccacgctcgtggccaacacctccaacatgcccgtcgccgcgcgcgaggccagcatctaca

SEQ ID NO: 18 - DNA, 4448 bp, synthetic construct; identical to SEQ ID NO: 17
  source 1..4448: mol_type = other DNA

SEQ ID NO: 19 - DNA, 4318 bp, synthetic construct
  source 1..4318: mol_type = other DNA
gcaatctcccgcccaacctccaccagccggcgcaggacctggagcgcgaaagaggatttcgggggtgtgggggagagtgagcgcgatggaagacactgatttgcataaacaacctttggccgctttcaaagcaccacctcaccaccataaaaccctccctcccatgcataccttgatgcctgccgagatggcctgggcttggtggaccatggccatgtagtccggcgtggtcgcgatggacgccgcacgttgtgcgatgagcgccttgcccccgtcctcgtgcccgagcccgcgtccgggcggctcggccagccccgccagcagccccgggtccagctcggcgttgagggagcgctttgccagcgcacggatggactcgtacggcagcgtgatgaggtggagcaggctgtcgagcaggcccggctgcgaccagagcatggtgaggatgcgcgccgccgcgggcgtgccgggctgcaccgccagcagcaacgcggcgaccgtctcggccgcaaacaccgccaggaagcagccctccgaggtcgggccgggctccagcaggggaccgcaggcgtgcactatgtccaggcgctccgtgatgatgtgcatggccagcggcagcagcgagcgcagcacgctgctggccaggtgcgtgagcgtgctcgtgccggccgagtccgaggtctcgtccgtggccaggtccatgggcggcagcgtgcgctcgctcgcctggtgcgccgtccagctgcgccggtggcacagcagccgcagcaccgcgtggaagagcggcaggcagcccgcctccaccgccgcggcggcggccggggcgccgccctcctgcaggctggcgcgcaggatggccgccgactgccgcgccgccgacagctgctgcctgtgtctcaaaagctgcgacagcgtcggagagatgtcctgcggctccggctccgggatgtgctggatgccacgcacaccggtcgcaatcacctgttggataatgggtggcaggatggttggttaggttctgatgcggtcggggaggcggataaggtcacagccgcgtgatcttgctgaccaaacactgctggccacccaacaaaagtcgattgccaccgatgcacacggcatggcgcccagacagaagctcagcataccctcaggagggccagggccaggccaggccacatgtgcagctggaagccgggctgggctgaagcatgggccagcacgctcagggcatggttttgaaaggcaggaacgtggcttgtcacgtcaacaaccagggaagaggcctgcgacatcaaggggcaaagttggaatgttcagcatcatcagcctgggaccaatactctgctcacggttcagggcagaaaggtggcatagtcagggcctcgttcagaatttcaagtcccagtttaaacttcgtacctccgcgaggctgatcccctcgccctgagcggactggggccggggagccggggtgggaccggtggtcacctgcgccatggctagcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccagccgtagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccacttttcgccgcctgggcaaggcagagctcctggcctacgaccccagcggcaagtcgatcgacggctacggcttcaccaccatcatcacgtacgatgctcaatgaaaggggagttcattgccttcactagagaaacatacatgggtctatgtaaacttgatcgatctccgaatttcctcttgttgtgtctctggttgtgcagggaaggtcgcctctacctgccctggctgatgcagcagattcaaggcttgggggggacctttgaacggcgccgcatctccagcctgtcggagctgaaggagtatgatgccatcgtcaattgcacaggtgggttggagttagtccaggagatgtcgatagaccaatgcaaatcgtgtgcaacgcattagacccaatcactgcctccctcgccgctctcaggcctggaggcgccaaagctggtgcaggacgagtccatgtacccggtgcgcgggcacgttctgcgcgtacgggcgccctgggtccgccactacatcaaccgcgacgggggcacctacatcatccccaacacggacacggtggtgctgggcggcatcacgcaaaagggcaactggtccctcgagccgaccgaggaggatcggcgcgggatcctggagcgctgctacgagatcctgcccagcctgcgcaaggcgccgatcctgcgcgagtgggtcgggctccggcccggccgcccagacatccgcctggagcgcgaagatgcgcagctcgacggcaagtccgtacccgtcatccacaactacgggcacggcgggtccgggctcaccctgggctggggctgcgccgcagacgccgtcgcgctcgtgcgcagggcgctggccctctgacggtgagagggcctgttgcaggctgtgcggtcccgctcatctcaaaggccttggaattggctgggcgaatattctgatgcattgatgtgtatctcttcgcttttcttatgatgcaccactttggagcgagtattctggtgccaattgtgctttgaaatcacgctgcccacggtggcaaatgcaatgagagccgagtaagtggactggtagttcctaaggcgacgggcagctcaaggaaagccagcccccgatcccagctcgccacatccttgttgcacgcctgtatctggcggtttgaatgagactcgcgtccacccaggccggccctcgcgctgcggctccgcccacaactcgagcagcttgtcctttgcccctggcaacatggtctcgatggtgtcgtacacgggggcaggaaccaggtgcaggcctggagaagagggatggaagtgcgcacagattcgagaaggtgatggccctcttgccgaaatgcacaccctctttccccagcctctgcctccacccaccgtctgctcgggaagggatgcggcagtccaggacaacgtccgggttgcgctggtgtcggagctccatatcgcggccccgttctgaatggatggggtgggacagttggtgtgaccgg

SEQ ID NO: 20 - DNA, 4317 bp, unidentified
  source 1..4317: mol_type = unassigned DNA; note = Prototheca wickerhamii
gcaatctcccgcccaacctccaccagccggcgcaggacctggagcgcgaaagaggatttcggggtgtgggggagagtgagcgcgatggaagacactgatttgcataaacaacctttggccgctttcaaagcaccacctcaccaccataaaaccctccctcccatgcataccttgatgcctgccgagatggcctgggcttggtggaccatggccatgtagtccggcgtggtcgcgatggacgccgcacgttgtgcgatgagcgccttgcccccgtcctcgtgcccgagcccgcgtccgggcggctcggccagccccgccagcagccccgggtccagctcggcgttgagggagcgctttgccagcgcacggatggactcgtacggcagcgtgatgaggtggagcaggctgtcgagcaggcccggctgcgaccagagcatggtgaggatgcgcgccgccgcgggcgtgccgggctgcaccgccagcagcaacgcggcgaccgtctcggccgcaaacaccgccaggaagcagccctccgaggtcgggccgggctccagcaggggaccgcaggcgtgcactatgtccaggcgctccgtgatgatgtgcatggccagcggcagcagcgagcgcagcacgctgctggccaggtgcgtgagcgtgctcgtgccggccgagtccgaggtctcgtccgtggccaggtccatgggcggcagcgtgcgctcgctcgcctggtgcgccgtccagctgcgccggtggcacagcagccgcagcaccgcgtggaagagcggcaggcagcccgcctccaccgccgcggcggcggccggggcgccgccctcctgcaggctggcgcgcaggatggccgccgactgccgcgccgccgacagctgctgcctgtgtctcaaaagctgcgacagcgtcggagagatgtcctgcggctccggctccgggatgtgctggatgccacgcacaccggtcgcaatcacctgttggataatgggtggcaggatggttggttaggttctgatgcggtcggggaggcggataaggtcacagccgcgtgatcttgctgaccaaacactgctggccacccaacaaaagtcgattgccaccgatgcacacggcatggcgcccagacagaagctcagcataccctcaggagggccagggccaggccaggccacatgtgcagctggaagccgggctgggctgaagcatgggccagcacgctcagggcatggttttgaaaggcaggaacgtggcttgtcacgtcaacaaccagggaagaggcctgcgacatcaaggggcaaagttggaatgttcagcatcatcagcctgggaccaatactctgctcacggttcagggcagaaaggtggcatagtcagggcctcgttcagaatttcaagtcccagtttaaacttcgtacctccgcgaggctgatcccctcgccctgagcggactggggccggggagccggggtgggaccggtggtcacctgcgccatggctagcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccagccgtagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccacttttcgccgcctgggcaaggcagagctcctggcctacgaccccagcggcaagtcgatcgacggctacggcttcaccaccatcatcacgtacgatgctcaatgaaaggggagttcattgccttcactagagaaacatacatgggtctatgtaaacttgatcgatctccgaatttcctcttgttgtgtctctggttgtgcagggaaggtcgcctctacctgccctggctgatgcagcagattcaaggcttgggggggacctttgaacggcgccgcatctccagcctgtcggagctgaaggagtatgatgccatcgtcaattgcacaggtgggttggagttagtccaggagatgtcgatagaccaatgcaaatcgtgtgcaacgcattagacccaatcactgcctccctcgccgctctcaggcctggaggcgccaaagctggtgcaggacgagtccatgtacccggtgcgcgggcacgttctgcgcgtacgggcgccctgggtccgccactacatcaaccgcgacgggggcacctacatcatccccaacacggacacggtggtgctgggcggcatcacgcaaaagggcaactggtccctcgagccgaccgaggaggatcggcgcgggatcctggagcgctgctacgagatcctgcccagcctgcgcaaggcgccgatcctgcgcgagtgggtcgggctccggcccggccgcccagacatccgcctggagcgcgaagatgcgcagctcgacggcaagtccgtacccgtcatccacaactacgggcacggcgggtccgggctcaccctgggctggggctgcgccgcagacgccgtcgcgctcgtgcgcagggcgctggccctctgacggtgagagggcctgttgcaggctgtgcggtcccgctcatctcaaaggccttggaattggctgggcgaatattctgatgcattgatgtgtatctcttcgcttttcttatgatgcaccactttggagcgagtattctggtgccaattgtgctttgaaatcacgctgcccacggtggcaaatgcaatgagagccgagtaagtggactggtagttcctaaggcgacgggcagctcaaggaaagccagcccccgatcccagctcgccacatccttgttgcacgcctgtatctggcggtttgaatgagactcgcgtccacccaggccggccctcgcgctgcggctccgcccacaactcgagcagcttgtcctttgcccctggcaacatggtctcgatggtgtcgtacacgggggcaggaaccaggtgcaggcctggagaagagggatggaagtgcgcacagattcgagaaggtgatggccctcttgccgaaatgcacaccctctttccccagcctctgcctccacccaccgtctgctcgggaagggatgcggcagtccaggacaacgtccgggttgcgctggtgtcggagctccatatcgcggccccgttctgaatggatggggtgggacagttggtgtgaccgg

SEQ ID NO: 21 - DNA, 4227 bp, synthetic construct; identical to SEQ ID NO: 3
  source 1..4227: mol_type = other DNA

SEQ ID NO: 22 - DNA, 4227 bp, unidentified; identical to SEQ ID NO: 3
  source 1..4227: mol_type = unassigned DNA; note = Prototheca wickerhamii

SEQ ID NO: 23 - DNA, 4067 bp, synthetic construct; identical to SEQ ID NO: 5
  source 1..4067: mol_type = other DNA

SEQ ID NO: 24 - DNA, 4069 bp, unidentified; identical to SEQ ID NO: 6
  source 1..4069: mol_type = unassigned DNA; note = Prototheca wickerhamii

SEQ ID NO: 25 - DNA, 4492 bp, synthetic construct
  source 1..4492: mol_type = other DNA
ttgcgattcctcttgacccagccacacgcataaattttaacgcaagctggcccaatatggtatccgctgtgctcagaacagcaccctcgtgcaccctctggctctgaatctcaccgtcacggtcatcctcctcctagcctgccagcgcttgggcgcagccatcgggcctgcatagagaaagggtacaaagcaacatatataaatacacgtggtattcataatactcgcgcatgtttgtcttgcgcacgcatgcccacgctcgtcgacgcgctcacctctggccaggcagcccccttgcggggtcgcccgcagcgggcaccgcgaggcctgaggcgccaccagcagcatgtttgaattatttatacgcagttgtttgaggtacagagaaaccccttgtcattctcttcttgtgtggggtgtcgagtgccattgcagtggtctggccaccggcatatcggataagattgcctgcggatgtcgttcgtgccagcgtcatcggggcgagagtttataacgcgtgcccacgccagaagccctcacgctttgcacttggcgaggtacaaaaaataccaaaccgcgtcctgaagcattatggcggcgaggacgaggctgttcaaggtgcggatcgcgaccttgagtgagagacgctccgatggtcgcggccagaaccccagcgcccgtcaaaagccggtcctcacgccccagggctgagtgcgcgcccgcaaaccccttttgcaggaactcaaggaggtggctcgatccaaggacaacaacggcatcgagctcattcccaatgactccaacatcttcctctggcgggcggtgattgaggcaagggggtctgcttcattgggtggagggagatgagttgtgtgcagggcgctcaaaggaggttttggtcgcctattgcccaaggtcatgcatccgtgtgtatcccgtgccccctcccttcaacagggccccgcggactcgccctatcaggggggtcactttgaactcagcatccaagtgcctgagcagtacccattggtcccgccgcaagtcaagttcaagaccaagctcttccacccgaacgtgcacttcaaggtgagctgtttggagacaaaggtggcagggagagtgctagcaagcaggggttgacgggcgttgctgttgagttttgatgtatagactcggccgggaacttgagtggaagccgacagacctttcatctgcctctctgcccatgcagacgggagagatctgcctcgacattctcaagaacgcctggagcccagcttggacgctgcaggtacgattgtttggtttaggagaggcggccccttggtcgtcccctggcactcacgactcttgggaaaatatgccgatctccgcattcctgtctccagacgcctgctctgtgcgcgctgtctcgatgaagcattcttttcctccccaatgcccgcagtcggtgtgccaggccatcgtggcgctcttgacagattctgcgccagactcccccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggatgcccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgacttttagctagcacccttacacaccatccaacatcttgctgccttgcctcccctgcgcagatccgcaagccggcgcacaagctgatggacgagctcaacatcccttacgacgacgaggtgggtattgggcagctagaacgcatgcgtgctgtgcgactgggtcgatccattgtgcgaaacgtgtggcggaatacgtgcgcgtccccggccatgcaccgaccccccctcccaccaccccacaaatataacagggtgacatggtggtggtcaagcacgccgccctggtgacgtccacgctgctgtccaaggtgctggcggcccccaacatcaagcttttcaacgccaccgcggcggaggacctgatcgtcaagtccaagccggccggcggggcggccgtcccgcacgtggccggggccgtgaccaactggaccctggtgtccctcaaccacgacacccagatgtgcatggaccccaacaccatcctgagcaaggtcatggtctcctccaccggccacgacggccccatgggcgcctccggggtcaagcgcctggccaagctgggcctgatcgagcgcgcgccgggcatgggcgcgctggacatgaacagcgccgaggacgcggtggtcgaccggacgcgggagatcgtccctggcatggtcatctgcggcatggaggtcgccgagctggacgggtgcccgcgcatgggccccacctttggcgccatgtttgtgtccggggtcaaggccgcgcacgtggccctggcctcgctgcgccgccagcaggaggaggaggggctccgcgccaagaccgcagacaccctgcgcagcgcggcgccgtccatggccatggcgtgacctggtgatccccaagagggcggacatgcgtcgcgtctccctccggtgcccagagatctgttccacggttgtctgcgatgctttcaaaactgttatttcactaaaatgactcgattctcctctaaaaaaaacatgatgttgatagtatctccacaaaagcatggcgactcagatgcctctactgcactgtactcaagaatacaactctggagacccgggtcccgttcccatttggcgccgaatgttgatgcgccacttttgctggccatgcctgaggtattctgtacaaaagaaggagcggccaatgcgggttttcaagcagacggcgcgctggggcctggaaatcaaaaggcggtagtacacgggagagagtcggattgatgatgagggatttaaaaaaggccaggcagtgaggtgagagttgtacgattgttgccccaggattgcctttttagggggggacggccaggggcgcagttattggggcaccgcacatatagatccgcgcaagcaaatggcctcgagagcaggcgcgatggttaagaaaggaaggacgagtagcggatgacgtgatactgcatacgaagaacgtactagtgaggagagtgcttgaaacaacatatgatgctgccccccctctctgccaac

SEQ ID NO: 26 - DNA, 4494 bp, unidentified
  source 1..4494: mol_type = unassigned DNA; note = Prototheca wickerhamii
ttgcgattcctcttgacccagccacacgcataaattttaacgcaagctggcccaatatggtatccgctgtgctcagaacagcaccctcgtgcaccctctggctctgaatctcaccgtcacggtcatcctcctcctagcctgccagcgcttgggcgcagccatcgggcctgcatagagaaagggtacaaagcaacatatataaatacacgtggtattcataatactcgcgcatgtttgtcttgcgcacgcatgcccacgctcgtcgacgcgctcacctctggccaggcagcccccttgcggggtcgcccgcagcgggcaccgcgaggcctgaggcgccaccagcagcatgtttgaattatttatacgcagttgtttgaggtacagagaaaccccttgtcattctcttcttgtgtggggtgtcgagtgccattgcagtggtctggccaccggcatatcggataagattgcctgcggatgtcgttcgtgccagcgtcatcggggcgagagtttataacgcgtgcccacgccagaagccctcacgctttgcacttggcgaggtacaaaaaataccaaaccgcgtcctgaagcattatggcggcgaggacgaggctgttcaaggtgcggatcgcgaccttgagtgagagacgctccgatggtcgcggccagaaccccagcgcccgtcaaaagccggtcctcacgccccagggctgagtgcgcgcccgcaaaccccttttgcaggaactcaaggaggtggctcgatccaaggacaacaacggcatcgagctcattcccaatgactccaacatcttcctctggcgggcggtgattgaggcaagggggtctgcttcattgggtggagggagatgagttgtgtgcagggcgctcaaaggaggttttggtcgcctattgcccaaggtcatgcatccgtgtgtatcccgtgccccctcccttcaacagggccccgcggactcgccctatcaggggggtcactttgaactcagcatccaagtgcctgagcagtacccattggtcccgccgcaagtcaagttcaagaccaagctcttccacccgaacgtgcacttcaaggtgagctgtttggagacaaaggtggcagggagagtgctagcaagcaggggttgacgggcgttgctgttgagttttgatgtatagactcggccgggaacttgagtggaagccgacagacctttcatctgcctctctgcccatgcagacgggagagatctgcctcgacattctcaagaacgcctggagcccagcttggacgctgcaggtacgattgtttggtttaggagaggcggcccccttggtcgtcccctggcactcacgactcttgggaaaatatgccgatctccgcattcctgtctccagacgcctgctctgtgcgcgctgtctcgatgaagcattcttttcctccccaatgcccgcagtcggtgtgccaggccatcgtggcgctcttgacagattctgcgccagactcccccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggatgcccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgacttttagctagcacccttacacaccatccaacatcttgctgccttgcctcccctgcgcagatccgcaagccggcgcacaagctgatggacgagctcaacatcccttacgacgacgaggtgggtattgggcagctagaacgcatgcgtgctgtgcgactgggtcgatccattgtgcgaaacgtgtggcggaatacgtgcgcgtccccggccatgcaccgaccccccctcccaccaccccacaaatataacagggtgacatggtggtggtcaagcacgccgccctggtgacgtccacgctgctgtccaaggtgctggcggcccccaacatcaagcttttcaacgccaccgcggcggaggacctgatcgtcaagtccaagccggccggcggggcggccgtcccgcacgtggccggggccgtgaccaactggaccctggtgtccctcaaccacgacacccagatgtgcatggaccccaacaccatcctgagcaaggtcatggtctcctccaccggccacgacggccccatgggcgcctccggggtcaagcgcctggccaagctgggcctgatcgagcgcgcgccgggcatgggcgcgctggacatgaacagcgccgaggacgcggtggtcgaccggacgcgggagatcgtccctggcatggtcatctgcggcatggaggtcgccgagctggacgggtgcccgcgcatgggccccacctttggcgccatgtttgtgtccggggtcaaggccgcgcacgtggccctggcctcgctgcgccgccagcaggaggaggaggggctccgcgccaagaccgcagacaccctgcgcagcgcggcgccgtccatggccatggcgtgacctggtgatccccaagagggcggacatgcgtcgcgtctccctccggtgcccagagatctgttccacggttgtctgcgatgctttcaaaactgttatttcactaaaatgactcgattctcctctaaaaaaaacatgatgttgatagtatctccacaaaagcatggcgactcagatgcctctactgcactgtactcaagaatacaactctggagacccgggtcccgttcccatttggcgccgaatgttgatgcgccacttttgctggccatgcctgaggtattctgtacaaaagaaggagcggccaatgcgggttttcaagcagacggcgcgctggggcctggaaatcaaaaggcggtagtacacgggagagagtcggattgatgatgagggatttaaaaaaggccaggcagtgaggtgagagttgtacgattgttgccccaggattgcctttttagggggggacggccaggggcgcagttattggggcaccgcacatatagatccgcgcaagcaaatggcctcgagagcaggcgcgatggttaagaaaggaaggacgagtagcggatgacgtgatactgcatacgaagaacgtactagtgaggagagtgcttgaaacaacatatgatgctgccccccctctctgccaac

SEQ ID NO: 27 - DNA, 1599 bp, Saccharomyces cerevisiae
  source 1..1599: mol_type = genomic DNA
atgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtga

SEQ ID NO: 28 - DNA, 1416 bp, Saccharomyces carlsbergensis
  source 1..1416: mol_type = genomic DNA
atgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctga

SEQ ID NO: 29 - DNA, 1935 bp, Arabidopsis thaliana
  source 1..1935: mol_type = genomic DNA
atggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtga

SEQ ID NO: 30 - DNA, 573 bp, unidentified
  source 1..573: mol_type = unassigned DNA; note = Prototheca wickerhamii
tgttgaagaatgagccggcgacttaaaataaatggcaggctaagagaattaataactcgaaacctaagcgaaagcaagtcttaatagggcgctaatttaacaaaacattaaataaaatctaaagtcatttattttagacccgaacctgagtgatctaaccatggtcaggatgaaacttgggtgacaccaagtggaagtccgaaccgaccgatgttgaaaaatcggcggatgaactgtggttagtggtgaaataccagtcgaactcagagctagctggttctccccgaaatgcgttgaggcgcagcaatatatctcgtctatctaggggtaaagcactgtttcggtgcgggctatgaaaatggtaccaaatcgtggcaaactctgaatactagaaatgacgatatattagtgagactatgggggataagctccatagtcgagagggaaacagcccagaccaccagttaaggccccaaaatgataatgaagtggtaaaggaggtgaaaatgcaaatacaaccaggaggttggcttagaagcagccatcctttaaagagtgcgtaatagctcactg

SEQ ID NO: 31 - DNA, 531 bp, unidentified
  source 1..531: mol_type = unassigned DNA; note = Prototheca moriformis
ttaaaataaatggcaggctaagagaattaataactcgaaacctaagcgaaagcaagtcttaatagggcgctaatttaacaaaacattaaataaaatctaaagtcatttattttagacccgaacctgagtgatctaaccatggtcaggatgaaacttgggtgacaccaagtggaagtccgaaccgaccgatgttgaaaaatcggcggatgaactgtggttagtggtgaaataccagtcgaactcagagctagctggttctccccgaaatgcgttgaggcgcagcaatatatctcgtctatctaggggtaaagcactgtttcggtgcgggctatgaaaatggtaccaaatcgtggcaaactctgaatactagaaatgacgatatattagtgagactatgggggataagctccatagtcgagagggaaacagcccagaccaccagttaaggccccaaaatgataatgaagtggtaaaggaggtgaaaatgcaaatacaaccaggaggttggcttagaagcagccatcctttaaa